SEQUENCE LISTING (2 sequences)

SEQ ID NO: 1 - DNA, 28 bp, Artificial sequence
  Primer
ttttccatggagatggaaaaggagttcg

SEQ ID NO: 2 - DNA, 34 bp, Artificial sequence
  Primer
ttttccatggctaattgtgtggctccaggattcg

(19) United States
(12) Patent Application Publication
(10) Pub. No.: US 2007/0129281 A1
(43) Pub. Date: Jun. 7, 2007

(54) Pharmaceutical compounds

(76) Inventors: Robin Arthur Ellis Carr, Cambridge (GB); Miles Stuart Congreve, Cambridge (GB); Dominic John Giuseppe Tisi, Bishops Stortford (GB); Robert Leon Marie Van Montfort, Ely (GB); Nicola Gail Wallis, Cambridge (GB); Glyn Williams, Great Grandsden (GB); Harren Jhoti, Gt Chester Ford (GB)

Correspondence Address: HESLIN ROTHENBERG FARLEY & MESITI PC, 5 COLUMBIA CIRCLE, ALBANY, NY, 12203, US

(21) Appl. No.: 10/551,499

(22) PCT Filed: Apr. 1, 2004
(86) PCT No.: PCT/GB04/01446

§ 371 Date: Feb. 6, 2007

Related U.S. Application Data
(60) Provisional application No. 60/459,749, filed on Apr. 2, 2003.
(60) Provisional application No. 60/468,543, filed on May 7, 2003.

(51) Int. Cl. A61K 38/17; C07K 14/47

(52) U.S. Cl. 514/2; 530/408

(57) ABSTRACT

The invention provides novel inhibitors of protein tyrosine phosphatase sulfenyl amide and their use in medicine, for example in the treatment or prevention of disease states such as cancer, diabetes, rheumatoid arthritis and hypertension. Also provided are novel crystal structures and the use of the crystal structures and their X-ray coordinates in the development of new drugs.

This invention relates to novel inhibitors of protein tyrosine phosphatase (PTP) activity, to a novel PTP form and crystal structures thereof, and to the uses of the crystal structures and novel form intermediate in the design of new drug molecules. The invention also provides the use of the inhibitors in medicine and in particular the treatment of disease states mediated by PTP activity, and to pharmaceutical compositions containing the compounds.

### BACKGROUND OF THE INVENTION

Protein tyrosine phosphatases (PTPs) are crucial in regulating signal transduction pathways involving tyrosine phosphorylation¹ and have been implicated in cancer, diabetes, rheumatoid arthritis, and hypertension².

Protein tyrosine phosphorylation plays a major role in regulation of many cell functions including response to hormones, growth factors and cytokines as well as in cell proliferation and apoptosis. Protein tyrosine phosphatases (PTPases) therefore represent an important control point in these regulatory mechanisms. Atypical tyrosine phosphorylation of specific proteins or components of signal transduction pathways has been implicated in a variety of human diseases including diabetes (diabetes type I and II) obesity, autoimmune diseases, acute and chronic inflammation, osteoporosis, proliferative disorders including various forms of cancer, growth disorders, response to infection and defective platelet aggregation. Such atypical tyrosine phosphorylation can result from dysregulation of both the kinases and or phosphatases controlling the process. In many cases PTPase activity is the major mechanism limiting the extent of phosphorylation and therefore such phosphatases represent key targets for therapeutic agents intended to exert pharmacological control over such processes.

Type 2 diabetes is characterized by abnormalities of insulin secretion and by insulin resistance of the major target tissues producing a diminished uptake and metabolism of glucose.

Protein-tyrosine phosphatases (PTPases) play a key role in the regulation of reversible tyrosine phosphorylation in the insulin action pathway. The receptor for insulin is an integral membrane protein with tyrosine kinase activity and insulin signal transduction is initiated by the phosphorylation of specific tyrosyl residues receptor. This initiates a complex signalling cascade leading to the phosphorylation of several key substrates including the IRS proteins on specific tyrosine residues. These activation steps are balanced, in turn, by specific cellular PTPases that dephosphorylate and inactivate the receptor kinase and reverse the adapter function of the receptor substrate proteins. PTP1B is a key component of this network and levels of PTP1B have been reported to be increased in diabetes associated with insulin resistance. Inhibition of PTP1B would therefore be expected to increase the strength of the signal initiated by the insulin resceptor and reverse the insulin resistance in such patients.

Other cellular responses dependent on the action of tyrosine kinases are similarly dependent on the phosphatases which limit the strength of the response. For example the growth factors EGF, VEGf and PDGF all initiate a network of signalling cascades dependent on tyrosine phosphorylation by their specific receptor tyrosine kinases. The response of lymphocytes to specific antigen activation and of other immune cells to cytokines such as IL-6 also use non-receptor tyrosine kinases as key components in their signal transduction pathways. Therefore inhibition of other members of the PTP family would be expected to control cell growth, cellular transformation, tumor formation, lymphocyte activation, cell migration, and inflammatory responses.

PTP1B belongs to a large family of PTPs characterised by an 11 residue signature sequence (I/V)HCXAGXXR(S/T)G which includes the catalytic cysteine (Cys215)^{1,10}. Its catalytic mechanism involves a nucleophilic attack by Cys215 on a phosphotyrosine substrate resulting in a covalent phosphocysteine intermediate, which is subsequently hydrolysed by an activated water molecule¹¹. The crystal structure of PTP1B shows that the PTP signature motif adopts a cradle-like conformation forming the base of the active site (FIG. 1a)¹². Its backbone amide atoms point to the centre of the cradle, which together with the invariant Arg221 provides an excellent environment to stabilise the negatively charged Cys215 side chain and bind the phosphate moiety of an incoming substrate¹³ (FIG. 1a). A hydrogen bond between the hydroxyl group of Ser222 and the sulphur atom of Cys215 further stabilises the cysteine conformation and helps to maintain its reduced pKa (˜5.4)¹⁴. One side of the active site is flanked by the so-called WPD-loop, which adopts an open conformation in the unliganded enzyme and closes over a bound phosphotyrosine residue¹³. The opposite side is formed by the phosphotyrosine (pTyr) recognition loop containing Tyr46, which mainly determines specificity for phosphotyrosine substrates (FIG. 1a)¹⁵.

Increasing literature evidence suggests that the cellular redox state is involved in regulating PTP activity by reversibly oxidizing their catalytic cysteines. Current literature describes the role of the sulfenic acids (Cys-SOH)³⁻⁶. Further oxidation to the sulfinic (Cys-SO₂H) and sulfonic (Cys-SO₃H) forms causes irreversible inhibition.

Further, there are examples of sulphur-nitrogen bonds in the literature. The reaction of a sulfenic acid derivative of glyceraldehyde-3-phosphate dehydrogenase with the small molecule, benzylamine, to form a sulfenamide has been reported by Allison et al²⁶.

Reich et al.²⁷ has described studies involving the formation of cyclic selenenamide structure in a small molecule model system and suggested that in its oxidized form mammalian glutathione peroxidase, a selenoenzyme, may have a cyclic selenenamide structure.

### SUMMARY OF THE INVENTION

The Applicants have found that oxidation of the catalytic cysteine at the active site of PTP by oxidants leads to the formation of a sulfenyl amide moiety at the active site. The ‘sulfenyl amide’ is an isothiazolidin-3-one ring system, which has not been previously observed in proteins. The sulfenyl amide moiety is believed to be a protective intermediate in the oxidative inhibition of PTPs that prevents further irreversible oxidation to sulfinic and sulfonic acids. Formation of the sulfenyl amide moiety at the active site leads to a loss of the enzyme's catalytic activity. Reduction of the sulfenyl amide moiety with a physiological reducing agent such as glutathione leads to regeneration of the active form of the enzyme.

This invention is based in part on recognition that compounds that stabilize the sulfenyl amide form or effect reversible or irreversible covalent modification of the sulfenyl amide form will be useful as therapeutic agents. Thus, by preventing or inhibiting the reversion of the inactive or less active sulfenyl amide form to the active form of PTP, the overall level of activity of PTP within a cellular environment can be substantially reduced.

Accordingly, the invention provides compounds that inhibit reversion of the PTP sulfenyl amide to the active form of PTP and their use in therapy.

Also covered by the invention are cysteine-containing proteins which have a suitably nucleophilic cysteine in the active site to facilitate formation of the sulfenyl amide. This includes phosphatases and phosphatase-like proteins which are structurally homologous to the PTPs such as rhodanese and bacterial phosphotransferases e.g. IIBcel (also known as IIBchb).

The term “cysteine-containing proteins” includes all proteins characterised by the HC(X5)R signature motif and other proteins belonging and related to this family, for example, those that have a remnant of this motif capable of adopting a conformation similar to the PTP phosphate binding cradle and which have a catalytic cysteine. A preferred set of proteins is the set in which there is an active site cysteine and an unusually polarised peptide bond between the active site cysteine and the following residue, in particular those with the HC(X5)R signature motif. One hypothesis is that in the HC(X5)R signature motif this bond is polarised by the conserved His in the signature motif. The HC(X5)R phosphatase family includes classical PTPs as well as the more distantly related low molecular weight (LMW) phosphatases, dual specificity phosphatases and rhodanese/CDC25 superfamily. A preferred subset is the set of those cysteine-containing proteins that do not have more than one cysteine in the active site.

PTPs of the invention characterised by the above structural motif include all PTPs. In a preferred aspect of the invention this refers to PTPs without a second active site cysteine in close proximity of the catalytic cysteine, and more preferably those PTPs with one cysteine residue in the binding site. Preferred PTPs are PTPs characterised by the 11 residue signature sequence (I/V)HCXAGXXR(S/T)G. Preferred PTPs include LAR, T-cell PTP, PTP-α and PTP1b, more preferably PTP1b.

The sulfenyl amide form of PTB1B has been prepared by the Applicants under controlled conditions and its structure determined by X-ray diffraction analysis. The structural data can be used in methods of rational drug design to provide compounds that inhibit reversion of the PTP sulfenyl amide to the active form of PTP.

Although the invention is specifically illustrated herein by reference to PTP1B, it is considered to be applicable also to other PTPs where the cellular redox state is involved in regulating PTP activity by reversibly oxidizing their catalytic cysteines to form sulfenyl amides. The terms “protein tyrosine phosphatase sulfenyl amide”, “PTP sulfenyl amide” and “sulfenyl amide” used herein refer generally to PTPs in which a cysteine moiety at the catalytic site has been oxidized to form a sulfenyl amide, unless the context indicates otherwise. For the avoidance of doubt, it is noted that the terms “protein tyrosine phosphatase sulfenyl amide”, “sulfenyl amide protein tyrosine phosphatase”, “PTP sulfenyl amide” and “sulfenyl amide PTP” as used herein are interchangeable and refer to the same entity unless the context requires otherwise.

The various aspects and embodiments of the invention are described in more detail below and defined in the claims appended hereto.

### BRIEF DESCRIPTION OF THE DRAWINGS

FIG. 1 provides a comparison of the structures of native and sulfenyl-amide PTP1B.

FIG. 1a is a ribbon diagram of PTP1B showing the phosphate-binding cradle, the WPD-loop and the pTyr recognition loop.

FIG. 1b shows the superimposition of the structure of native PTP1B ( light grey) and the sulfenyl-amide containing structure (dark grey) showing the different conformations of the pTyr recognition loop and the phosphate-binding cradle.

FIG. 1c shows the electron density of the novel sulfenyl-amide derivative and its neighbouring residues. The electron density map in FIG. 1c is contoured at 1σ. All figures are generated using Aesop (Martin Noble, unpublished).

FIG. 2 shows a putative mechanism of sulfenyl-amide formation and subsequent reactivation. As illustrated, the catalytic cysteine of PTP1B (E-SH) is oxidised to a sulfenic acid (E-S—OH). The sulfenyl-amide may be formed by a direct mechanism involving a nucleophilic attack of the backbone nitrogen of Ser216 on the Sγ atom of Cys215 and subsequent release of water. Alternatively the sulfenic acid may be oxidised to a highly reactive intermediate by an oxidising agent e.g. by peroxide e.g. H₂O₂^{24, 25} or an oxidised thiol, which then reacts to give the sulfenyl-amide. Reactivation of the enzyme occurs via mixed disulfide formation with a thiol. R, denotes glutathione or DTT, X the leaving groups OOH (sulfenoperoxoic acid) or OS(O)R (sulfinothioic acid).

FIG. 3 illustrates the different oxidation states of the catalytic cysteine. The structures shown are the sulfonic (A), sulfinic (B) and sulfenic acid derivatives (C) of Cys215. The phosphate-binding cradle comprising residues 215 to 222 is shown in ball-and-stick representation. Hydrogen bonds are shown as dashed lines. The maps are contoured at 3σ and in all maps the peaks are higher than 5σ.

### DETAILED DESCRIPTION OF THE INVENTION

The PTP Sulfenyl Amides of the Invention

In one aspect, the present invention contemplates an isolated PTP sulfenyl amide. The sulfenyl amides of the invention have a variety of uses, as described herein.

The terms “PTP sulfenyl amide”, “sulfenyl amide PTP” and (in the context of PTP) “sulfenyl amide” alone are used herein as generic terms to define any PTP (as defined below) in which a cysteine moiety at the catalytic site is oxidized to form a sulfenyl amide.

The term “PTP” is used generally herein (and in particular in the context of the PTP sulfenyl amides of the invention) as a generic term to include all members of the PTP protein family, whether natural, synthetic or recombinant. Preferred are PTPs without a second active site cysteine in close proximity to the catalytic cysteine, and more particularly preferred are those PTPs with only one cysteine residue in the binding site. Such preferred PTPs include LAR, T-cell PTP, PTP-α and PTP1B (the latter specifically exemplified herein).

The term “PTP” is also intended to encompass PTP homologues, analogues, allelic forms, species variants, derivatives, muteins or equivalents, whether natural, synthetic or recombinant (as hereinbelow defined).

The term “homologue” is used herein in two distinct senses. It is used sensu stricto to define proteins that share a common ancestor to the PTP. In this sense it covers orthologues (species variants which have diverged in different organisms following a speciation event) and paralogues (variants which have diverged within the same organism after a gene duplication event). Thus, there is a direct evolutionary relationship between the PTP and such homologues and this may be reflected in structural and/or functional similarities. For example, orthologues may perform the same role in each organism in which they are found, while paralogues may perform functionally related (but distinct) roles within the same organism.

The term is also used herein sensu lato to define a PTP which is to some extent structurally similar (i.e. not necessarily evolutionary related and/or structurally and functionally equivalent) to a given (reference) PTP (for example, to any one of LAR, T-cell PTP, PTP-α and PTP1B). In this sense, homology is recognised on the basis of purely structural criteria by the presence of amino acid sequence identities and/or conservative amino acid changes and/or similar secondary, tertiary or quaternary structures.

In this context, a conservative amino acid substitution is one in which the amino acid residue is replaced with an amino acid residue having a similar side chain. Families of amino acid residues having similar side chains have been defined in the art (as set out for example by Dayhoff et alia, Atlas of protein structure vol. 5, National BioMed Fd'n, Washington D.C., 1979). These families include amino acids with basic side chains (e.g., lysine, arginine, histidine), acidic side chains (e.g. aspartic acid, glutamic acid), non-charged polar side chains (e.g. glycine, asparagine, glutamine, serine, threonine, tyrosine, cysteine), non-polar side chains (e.g. alanine, valine, leucine, isoleucine, proline, phenylalanine, methionine, tryptophan), beta-branched side chains (e.g. threonine, valine, isoleucine), and aromatic side chains (e.g. tyrosine, phenylalanine, tryptophan, histidine).

The homologues of the invention therefore include proteins having at least 50%, 55%, 60%, 65%, 70%, 75%, 80%, 85%, 90% or 95% sequence identity with the reference PTP, and include truncated forms of naturally-occurring PTP proteins. Such truncates are preferably at least 25%, 35%, 50% or 75% of the length of the corresponding wild-type PTP and may have at least 50%, 55%, 60% or 65% sequence identity (more preferably, at least 70%, 75%, 80%, 85%, 90% or 95% sequence identity) with that wild-type PTP. Particularly preferred homologues are truncates that contain a segment preferably comprising at least 8, 15, 20 or 30 contiguous amino acids that share at least 75%, 80%, 85%, 90% or 95% sequence identity with that reference PTP.

Preferred truncates for PTP1b are residues 1-321 or residues 1-298 of the reference wild-type sequence. A particularly preferred truncate for PTP1b is the one defined by residues 1-321 of reference PTP1b sequence accession number P18031 [SwissProt: PTN1_HUMAN].

For the avoidance of doubt, full length sulfenyl amide wild-type PTPs are within the scope of the invention as well as truncated versions of sulfenyl amide wild-type PTPs.

For the purposes of the invention, homologues may also be recognised as those proteins the corresponding DNAs of which are capable of specifically or selectively cross-hybridising, or which can cross-hybridise under selective, appropriate and/or appropriately stringent hybridisation conditions.

The term “selectively or specifically (cross)hybridisable” in this context indicates that the sequences of the corresponding ssDNAs are such that binding to a unique (or small class) of homologous sequences can be obtained under more or less stringent hybridisation conditions. Exemplary stringent conditions can be found in, for example, Current Protocols in Molecular Biology, John Wiley & Sons, N. Y. (1989), 6.3.1-6.3.6. A preferred, non-limiting example of stringent hybridization conditions is hybridization in 6×sodium chloride/sodium citrate (SSC) at about 45° C., followed by one or more washes in 0.2×SSC, 0.1 SDS at a temperature of from about 50° C. to 65° C.

The term “allelic form” is used herein to define a naturally-occurring alternative form (allelic variant) of a wild-type PTP sequence which reflects naturally-occurring differences in the PTP gene pool. Allelic forms may be isolated and identified by making suitable probes or primers from the sequences provided herein and screening a suitable nucleic acid source from individuals of the appropriate species.

The term “analogue” is used herein to define proteins with similar functions and/or structures and which are not necessarily evolutionarily related. PTP analogues which share function but which have no or little structural similarities are likely to have arisen by convergent evolution. Conversely, PTP analogues which share structural similarities but which exhibit few or no functional similarities are likely to have arisen by divergent evolution. PTP analogues may be identified, for example, by screening a library of proteins to detect those with similar function(s) but different physical properties, or by screening for proteins which share structural features but not necessarily any functions (e.g. by immunological screening).

The term “species variant” is used herein to define the corresponding PTP from a different organism. Thus, species variants share a direct evolutionary relationship.

Species variants may be isolated and identified by making suitable probes or primers from the sequences provided herein and screening a suitable nucleic acid source from the desired species. Preferably, species variants are those isolated from mammalian species. Most preferably, species variants are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulus griseus, Felis catus, Mustela vison, Canis familiaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuanez, 1988, Ann. Rev. Genet. 22: 323-351; O'Brien et al., 1993, Nature Genetics 3: 103-112; Johansson et al., 1995, Genomics 25: 682-690; Lyons et al., 1997, Nature Genetics 15: 47-56; O'Brien et al., 1997, Trends in Genetics 13 (10): 393-399; Carver and Stubbs, 1997, GenomeResearch7: 1123-1137; all of which are incorporated by reference herein).

The term “derivative” as applied herein to the PTPs of the invention is used to define PTPs which are modified versions of any wild-type or truncated PTP. Such derivatives may include fusion proteins, in which the proteins of the invention have been fused to one or more different proteins, peptides or amino acid tags (for example an antibody or a protein domain conferring a biochemical activity, to act as a label, or to facilitate purification). Particularly preferred are derivatives in which the PTP proteins or peptides are modified by a polyHis (6× His) tag to facilitate purification of the peptide derivative on Ni²⁺ agarose beads. It is further preferred that the proteins are derivatives of truncated PTP proteins.

The derivatives may also be products of synthetic processes that use a wild-type PTP as a starting material or reactant.

The term “mutein” is used herein to define PTPs that are mutant forms of a wild-type PTP, i.e. PTP proteins in which one or more amino acids have been added, altered, deleted, replaced, inserted or substituted. The muteins of the invention therefore include fragments, truncates and fusion peptides (e.g. comprising fused immunoglobulin, receptor, tag, label or enzyme moieties).

The muteins of the invention therefore include truncated forms of a wild-type PTP. Such truncates are preferably least 25%, 35%, 50% or 75% of the length of the corresponding wild-type PTP and may have at least 65% sequence identity (more preferably, at least 70%, 75%, 80%, 85%, 90% or 95% sequence identity) with that PTP.

The muteins of the invention also include PTPs in which mutations have been introduced which effectively promote or impair one or more activities of the PTP, for example mutations which promote or impair the function of the active site.

Muteins may be produced by any convenient method. Conveniently, site-directed mutagenesis with mutagenic oligonucleotides may be employed using a double stranded template (pBluescript KS II construct containing a PTP gene), (e.g. Chameleon'M or QuikChange'M—Stratagene'M). After verifying each mutant derivative by sequencing, the mutated gene is excised and inserted into a suitable vector so that the modified protein can be over-expressed and purified.

Preferred mutant forms are truncates consisting (or consisting essentially) of the PTP 11-residue signature sequence described herein. Particularly preferred are truncates that contain a segment preferably comprising at least 8, 15, 20 or 30 contiguous amino acids that share at least 75%, 80%, 85%, 90% or 95% sequence identity with the PTP from which they are derived by truncation.

The term “equivalent” is used herein to define those PTP analogues which exhibit substantially the same function(s) and which share at least some structural features (e.g. functional domains), but which have not evolved from a common ancestor. Such equivalents are typically synthetic proteins (see below) and may be generated, for example, by identifying sequences of functional importance (e.g. by identifying conserved or canonical sequences, functional domains or by mutagenesis followed by functional assay), selecting an amino acid sequence on that basis and then synthesising a peptide based on the selected amino acid sequence. Such synthesis can be achieved by any of many different methods known in the art, including solid phase peptide synthesis (to generate synthetic peptides) and the assembly (and subsequent cloning) of oligonucleotides. Some synthetic protein analogues may be chimaeras, and such equivalents can be designed and assembled for example by concatenation of two or more different structural and/or functional peptide domains from different proteins using recombinant DNA techniques.

The homologues, analogues, fragments, muteins, equivalents or derivatives of the PTPs of the invention may also be defined inter alia as those proteins which cross-react with antibodies to one or more wild-type PTPs, and in particular those which cross-react with antibodies directed against a PTP lacking a second active site cysteine in close proximity of the catalytic cysteine (for example a PTP with only one cysteine residue in the binding site). Thus, the homologues, fragments, muteins, equivalents or derivatives of the PTPs of the invention include proteins which cross-react with antibodies to one or more of LAR, T-cell PTP, PTP-α and PTP1B.

For the purposes of the present invention, sequence identity is determined by comparing the amino acid sequences of the protein when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877.

Another example of a mathematical algorithm used for comparison of sequences is the algorithm of Myers and Miller (1988) CABIOS 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85: 2444-2448.

Preferred for use according to the present invention is the WU-BLAST (Washington University BLAST) version 2.0 software. WU-BLAST version 2.0 executable programs for several UNIX platforms can be downloaded from ftp://blast.wust1.edu/blast/executables. This program is based on WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

The term “isolated” is used herein to indicate that the PTP sulfenyl amide exists in a physical milieu distinct from that in which it occurs in nature. For example, the isolated sulfenyl amide may be substantially isolated with respect to the complex cellular milieu in which it naturally occurs. The absolute level of purity is not critical, and those skilled in the art can readily determine appropriate levels of purity according to the use to which the PTP sulfenyl amide is to be put. The term “isolating” when used a step in a process is to be interpreted accordingly.

In many circumstances, the isolated PTP sulfenyl amide will form part of a composition, for example a more or less crude extract containing many other molecules and substances, buffer systems, matrices or excipients, which may for example contain other components (including assay reagents and proteins, such as albumin).

In other circumstances, the isolated PTP sulfenyl amide may be purified to essential homogeneity, for example as determined by PAGE or column chromatography (for example HPLC or mass spectrometry). In preferred embodiments, the isolated PTP sulfenyl amide is essentially the sole protein in a given composition.

The isolated PTP sulfenyl amide of the invention may be crystallized. Crystals of the isolated PTP sulfenyl amide find particular utility in some applications of the invention (for example, for the in silico analyses described below).

The PTP sulfenyl amides of the invention need not be isolated in the sense defined above, however. For example, more or less crude preparations derived from spent media used to culture host cells expressing PNP or PNP sulfenyl amide may be used. Such supernatants may be treated in various ways, for example by oxidation, concentration, filtration, centrifugation, spray drying, dialysis and/or lyophilisation.

The term “natural” is used herein to define a PTP that has been derived from a naturally-occurring wild-type protein source by chemical or enzymic treatment. Naturally occurring PTPs may be obtained by purification (e.g. by column chromatography) from cellular material in which the native PTP is expressed.

The term “synthetic PTP” is used herein to define a PTP that has been chemically synthesised in vitro (for example by any of the commercially available solid-phase peptide-synthesis systems).

The term “recombinant” is used herein to define a PTP that has been produced by that body of techniques collectively known as “recombinant DNA technology”.

Thus, although this invention is based (at least in part) on the identification and characterization of a novel sulfenyl amide intermediate arising from oxidation of a cysteine moiety at the active site of PTP1B (see below), any of a large number of different PTP sulfenyl amides are contemplated by the invention and each finds general application in the various methods and processes described herein.

Use of the PTP Sulfenyl Amides of the Invention per se in Drug Discovery

The PTP sulfenyl amides of the invention may be used inter alia in various drug screening processes.

For example, the invention provides a process for screening for a PTP inhibitor comprising the steps of: (a) providing the PTP sulfenyl amide (or a homologue, allelic form, species variant, derivative or mutein thereof); (b) contacting the sulfenyl amide of step (a) with a test compound; and (c) determining whether the test compound binds to the sulfenyl amide.

The screening processes of the invention as described above are preferably high throughput processes. The screens identify and/or select compounds with PTP sulfenyl amide binding activity. Such compounds are candidate PTP modulators, and can be subjected to further analysis and/or screening in order to determine their activity as therapeutic agents (see for example the section headed “Assays for Screening for Active Compounds”, below). Alternatively, or in addition, they may be crystallized with PTP1B sulfenyl amide (e.g. by co-crystallization or by soaking) for X-ray analysis. The resulting X-ray structure may be compared with that of Table 1 or Table 2 for a variety of purposes.

For example, the PTP sulfenyl amides of the invention may be used in a process for producing a PTP inhibitor comprising the steps of: (a) providing a PTP sulfenyl amide (or a homologue, allelic form, species variant, derivative or mutein thereof); (b) contacting the sulfenyl amide of step (a) with a test compound; (c) determining whether the test compound binds to the sulfenyl amide; and (d) identifying the test compound as a PTP inhibitor on the basis of its ability to prevent or inhibit the reductive activation of the PTP sulfenyl amide to active PTP.

In such processes, at least two chemically distinct test compounds may be identified in step (d) and the process may then further comprise the step of linking two or more of the chemically distinct compounds to produce a multimeric PTP inhibitor. Such processes embody the linked fragment approaches described in more detail in the section headed “Linked fragment and fragment growing approaches”, below.

The PTP sulfenyl amides of the invention may therefore be used in a process for producing a pharmaceutical composition comprising the steps of: (a) providing a PTP sulfenyl amide (or a homologue, allelic form, species variant, derivative or mutein thereof); (b) contacting the sulfenyl amide of step (a) with a test compound; (c) determining whether the test compound binds to the sulfenyl amide; (d) identifying the test compound as a PTP inhibitor on the basis of its ability to prevent or inhibit the reductive activation of the PTP sulfenyl amide to active PTP; and (e) incorporating the inhibitor identified in step (d) into a pharmaceutical excipient.

The invention contemplates PTP inhibitors, drugs and pharmaceutical compositions obtainable by, or obtained by, the process of the invention described above.

Identification and Characterization of the Three Dimensional Structure of Sulfenyl Amide PTP

This invention is based on the identification and characterization of a novel sulfenyl amide intermediate arising from oxidation of a cysteine moiety at the active site of a protein tyrosine phosphatase.

The catalytic domain (residues 1-321) of PTP1B was expressed in E. coli cells according to known procedures and was purified and crystallized. The oxidation state of the catalytic cysteine of PTP1B was probed by means of soaking experiments using various oxidizing agents and crystal structures were subsequently obtained for a novel sulfenyl-amide intermediate of PTP1B, as well as sulfenic, sulfinic and sulfonic PTP1B derivatives.

Soaking crystals of the catalytic domain of PTB1B with 2-phenyl-isoxazolidine-3,5-dione gave rise to a modified crystal structure (the PTP1B sulfenyl amide), the structure for which has been determined by X-ray diffraction analysis. Atomic coordinates of the catalytic domain are set out in Table 1 and Table 2.

Accordingly, in one aspect, the invention provides a crystal of sulfenyl amide protein tyrosine phosphatase 1B.

Crystals

In another aspect, the invention provides a crystal of sulfenyl amide protein tyrosine phosphatase 1B having a Unit cell dimensions: a=87.686 Å, b=87.686 Å, c=103.721 Å, α=90.00°, β=90.00°, γ=120.00° and a space group: P3₁2 1. Unit cell variability of 5% may be observed in all dimensions.

The invention also provides a crystal of sulfenyl amide protein tyrosine phosphatase 1B having a resolution better than, i.e. numerically lower than, 3.0 Å, preferably lower than 2.6 Å.

The invention also provides crystals of sulfenyl amide protein tyrosine phosphatase 1B capable of being soaked with compound(s) to form co-complex structures.

Table 1 Coordinates

The invention also provides a crystal of sulfenyl amide protein tyrosine phosphatase 1B having the structure defined by the coordinates of Table 1.

Table 1 gives atomic coordinate data for sulfenyl amide protein tyrosine phosphatase 1B. In Table 1 the third column denotes the atom type, the fourth the residue type, the fifth the chain identification, the sixth the residue number. The seventh, eighth and ninth columns are the X, Y, Z coordinates respectively of the atom in question, the tenth column defines the occupancy of the atom, the eleventh column gives the temperature factor of the atom.

Table 2 Coordinates

The invention further provides a crystal of sulfenyl amide protein tyrosine phosphatase 1B having the structure defined by the coordinates of Table 2.

The coordinates of Table 2 are defined as the coordinates of Table 1 as amended in the manner outlined in Table 2. Table 2 varies in six ways from Table 1. These changes do not affect the positioning of the atoms of sulfenyl amide protein tyrosine phosphatase 1B. The co-ordinates of Table 2 represent the same spatial distribution of atoms of sulfenyl amide protein tyrosine phosphatase 1B as contained in Table 1 but in a format consistent with that of the EBI Macromolecular Structure Database (Hinxton, UK). Thus, the invention covers all co-ordinate files that essentially represent the same spatial distribution of sulfenyl amide protein tyrosine phosphatase 1B atoms independent of file format.

The coordinates of Table 1 or Table 2 provide a measure of atomic location in Angstroms, to a third decimal place. The coordinates are a relative set of positions that define a shape in three dimensions, so it is possible that an entirely different set of coordinates having a different origin and/or axes could define a similar or identical shape. Furthermore, varying the relative atomic positions of the atoms of the structure so that the root mean square deviation of the residue backbone atoms (i.e. the nitrogen-carbon-carbon backbone atoms of the protein amino acid residues) or the C-alpha atoms is less than 1.5 Å (preferably less than 1.0 Å, more preferably less than 0.5 Å and even more preferably less than 0.47 Å) when superimposed on the coordinates provided in Table 1 or Table 2 for the residue backbone atoms, will generally result in a structure which is substantially the same as the structure of Table 1 or Table 2 in terms of both its structural characteristics and potency for structure-based design of PTP1B inhibitors.

Likewise changing the number and/or positions of the water molecules and/or substrate molecules of Table 1 or Table 2 will not generally affect the potency of the structure for structure-based design of PTP1B inhibitors. Thus for the purposes described herein as being aspects of the present invention, it is within the scope of the invention if: the Table 1 or Table 2 coordinates are transposed to a different origin and/or axes; the relative atomic positions of the atoms of the structure are varied so that the root mean square deviation of residue backbone atoms or the C-alpha atoms is less than 1.5 Å (preferably less than 1.0 Å, more preferably less than 0.5 Å and even more preferably less than 0.47 Å) when superimposed on the coordinates provided in Table 1 or Table 2 for the residue backbone atoms; and/or the number and/or positions of water molecules and/or substrate molecules is varied. References herein to the coordinate data of Table 1 or Table 2 thus include the coordinate data in which one or more individual values of the Tables are varied in this way. By “root mean square deviation” we mean the square root of the arithmetic mean of the squares of the deviations from the mean.

It is also within the scope of the invention if the coordinate file represents the same spatial distribution of sulfenyl amide protein tyrosine phosphatase 1B atoms but in a different file format. Alternative file formats (e.g. such as a format consistent with that of the EBI Macromolecular Structure Database (Hinxton, UK)) which may include a different ordering of these atoms, or a different designation of the residues or residue molecule atoms, may be used or preferred by others of skill in the art. However it will be apparent that the use of a different file format to present or manipulate the coordinates of the Tables is within the scope of the present invention. Thus for the purposes described herein as being aspects of the present invention, it is within the scope of the invention if the coordinates are essentially the same as Table 1 or Table 2, essentially comprise the coordinates of Table 1 or Table 2, or are a set of coordinates that materially correspond to those of Table 1 or Table 2.

Other crystals of the invention include crystals which have selected coordinates of the binding pocket, wherein the amino acid residues associated with those selected coordinates are located in a protein framework which holds these amino acids in a relative spatial configuration corresponding to the spatial configuration of those amino acids in Table 1 or Table 2. By “corresponding to”, it is meant within an r.m.s.d. of less than 2.0 Å, preferably less than 1.5 Å, more preferably less than 1.0 Å, even more preferably less than 0.5 Å and most preferably less than 0.47 Å. In a further embodiment it is it within an r.m.s.d. of less than 0.3 Å, less than 0.25 Å, or less than 0.2 Å, and most preferably less than 0.1 Å. The amino acids which provide the selected coordinates are preferably selected from amino acids which form part of at least one sulfenyl amide protein tyrosine phosphatase 1B binding cavity, where these are residues 1 to 56 as described herein or combinations thereof as defined further herein below.

Those of skill in the art will appreciate that in many applications of the invention, it is not necessary to utilise all the coordinates of Table 1 or Table 2, but merely a portion of them. For example, as described below, in methods of modelling candidate compounds with PTP sulfenyl amide, selected coordinates of PTP sulfenyl amide may be used, for example at least 5, preferably at least 10, more preferably at least 20 and even more preferably at least 100 atoms of the sulfenyl amide structure. Likewise, the other applications of the invention described herein, including homology modelling and structure solution, and data storage and computer assisted manipulation of the coordinates, may also utilise all or a portion of the coordinates of Table 1 or Table 2. A preferred aspect of the invention is where the portion of the coordinates relates to the selected coordinates of the binding pocket. The amino acids which provide the selected coordinates are preferably selected from amino acids which form part of at least one sulfenyl amide protein tyrosine phosphatase 1B binding cavity, where these are residues 1 to 56 as described herein or combinations thereof as defined further herein below.

It will also be appreciated that the invention also includes within its scope crystals of PTP sulfenyl amide comprising amino acids having the atomic coordinates of Tables 1 or 2, but wherein the crystal comprises further amino acids in addition to those for which the coordinates are given. Therefore, unless explicitly set out to the contrary, or otherwise made clear from the context, references throughout the present specification to the use of all or selected coordinates of or from Tables 1 or 2 does not exclude the use of additional coordinates due to the presence of further amino acids.

Comparison of Protein Structures

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only. For the purposes of this invention, the r.m.s.d. can be calculated over any of these, using any of the methods outlined below.

Methods of comparing protein structures are discussed in Methods of Enzymology, vol. 115, pg 397-420. The necessary least-squares algebra to calculate r.m.s.d. has been given by Rossman and Argos (J. Biol. Chem. , vol. 250, pp 7525 (1975)) although faster methods have been described by Kabsch (Acta Crystallogr., Section A, A92, 922 (1976); Acta Cryst. A34, 827-828 (1978)), Hendrickson (Acta Crystallogr., Section A, A35, 158 (1979)); McLachan (J. Mol. Biol., vol 128, pp 49 (1979) and Kearsley (Acta Crystallogr., Section A, A45, 208 (1989)). Some algorithms use an iterative procedure in which the one molecule is moved relative to the other, such as that described by Ferro and Hermans (Ferro and Hermans, Acta Crystallographic, A33, 345-347 (1977)). Other methods, e.g. Kabsch's algorithm, locate the best fit directly.

Programs for determining r.m.s.d include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfo1.mbfys.lu.se/TOP/maps.html)).

It is usual to consider C-alpha atoms and the r.m.s.d. can then be calculated using programs such as LSQKAB (Collaborative Computational Project 4. The CCP4 Suite: Programs for Protein Crystallography, Acta Crystallographica, D50, (1994), 760-763), QUANTA (commercially available from Accelrys, San Diego, Calif.), Insight (commercially available from Accelrys, San Diego, Calif.), Sybyl® (commercially available from Tripos, Inc., St Louis), O (Jones et al., Acta Crystallographica, A47, (1991), 110-119), and other coordinate fitting programs.

In, for example, the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail above. The atomic coordinates can then be superimposed according to this alignment and an r.m.s.d. value calculated. The program Sequoia (C. M. Bruns, I. Hubatsch, M. Ridderström, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Once aligned, the r.m.s.d. can be calculated using programs detailed above. For sequences identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the r.m.s.d. value over C-alpha atoms only. It is particularly useful when analysing side chain movement to calculate the r.m.s.d. over all atoms and this can be done using LSQKAB and other programs.

Mutants

Also, modifications in the sulfenyl amide protein tyrosine phosphatase 1B crystal structure due to e.g. mutations, additions, substitutions, and/or deletions of amino acid residues could account for variations in the atomic coordinates. However, atomic coordinate data of sulfenyl amide protein tyrosine phosphatase 1B modified so that a ligand that bound to one or more binding sites of sulfenyl amide protein tyrosine phosphatase 1B would be expected to bind to the corresponding binding sites of the modified sulfenyl amide protein tyrosine phosphatase 1B are, for the purposes described herein as being aspects of the present invention, also within the scope of the invention. References herein to the coordinates of Table 1 or Table 2 thus include the coordinates modified in this way. Preferably, the modified coordinate data define at least one sulfenyl amide protein tyrosine phosphatase 1B binding cavity.

Crystals of the invention also include crystals of sulfenyl amide protein tyrosine phosphatase 1B mutants. In addition, sulfenyl amide protein tyrosine phosphatase 1B mutants may be crystallized in co-complex with known sulfenyl amide protein tyrosine phosphatase 1B substrates or inhibitors or novel compounds.

As explained herein, a mutant sulfenyl amide protein tyrosine phosphatase 1B is a sulfenyl amide protein tyrosine phosphatase 1B protein characterized by the replacement or deletion of at least one amino acid from the wild type PTP1B. Such a mutant may be prepared for example by site-specific mutagenesis, or incorporation of natural or unnatural amino acids.

As explained herein, the present invention therefore contemplates sulfenyl amide protein tyrosine phosphatase 1B mutants as hereinbefore defined.

For example, the sulfenyl amide protein tyrosine phosphatase 1B mutants may define a polypeptide which is obtained by replacing at least one amino acid residue in a native or synthetic sulfenyl amide protein tyrosine phosphatase 1B with a different amino acid residue and/or by adding and/or deleting amino acid residues within the native polypeptide or at the N- and/or C-terminus of a polypeptide corresponding to sulfenyl amide protein tyrosine phosphatase 1B, and which has substantially the same three-dimensional structure as sulfenyl amide protein tyrosine phosphatase 1B from which it is derived. By having substantially the same three-dimensional structure is meant having a set of atomic structure co-ordinates that have a root mean square deviation (r.m.s.d.) of less than or equal to about 1.5 Å, preferably less than 0.47 Å, when superimposed with the atomic structure co-ordinates of the sulfenyl amide protein tyrosine phosphatase 1B from which the mutant is derived when at least about 50% to 100% of the C_{α} atoms of the sulfenyl amide protein tyrosine phosphatase 1B are included in the superposition. A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, MBP tag, and epitope tags.

Amino acid substitutions, deletions and additions that do not significantly interfere with the three-dimensional structure of the sulfenyl amide protein tyrosine phosphatase 1B will depend, in part, on the region of the sulfenyl amide protein tyrosine phosphatase 1B where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

As explained earlier, conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues to a sulfenyl amide protein tyrosine phosphatase 1B binding pocket or catalytic residue in order to provide convenient cloning sites in the cDNA encoding the polypeptide, to aid in purification of the polypeptide, to modify compound binding etc. Such substitutions, deletions and/or additions which do not substantially alter the three dimensional structure of sulfenyl amide protein tyrosine phosphatase 1B will be apparent to those having skills in the art.

It should be noted that the mutants contemplated herein need not exhibit enzymatic activity. Indeed, amino acid substitutions, additions or deletions that interfere with the catalytic activity of the protein tyrosine phosphatase 1B but which do not significantly alter the three-dimensional structure of the catalytic region are specifically contemplated by the invention. Such crystalline polypeptides, or the atomic structure co-ordinates obtained there from, can be used to identify compounds that bind to the protein.

The crystallization of such mutants and the determination of the three-dimensional structures by X-ray crystallography rely on the ability of the mutant proteins to yield crystals that diffract at high resolution. The mutant protein could then be used to obtain information on compound binding through the determination of mutant protein/ligand complex structures, which may be characterized using the sulfenyl amide protein tyrosine phosphatase 1B crystal structure of Table 1 or Table 2.

The mutations can be introduced by site-directed mutagenesis e.g. using a Stratagene QuikChange™ Site-Directed Mutagenesis Kit or cassette mutagenesis methods (see e.g. Ausubel et al., eds., Current Protocols in Molecular Biology, John Wiley & Sons, Inc., New York, and Sambrook et al., Molecular Cloning: a Laboratory Manual, 2nd ed., Cold Spring Harbor Laboratory Press, Cold Spring Harbor, N.Y., (1989)).

To the extent that the present invention relates to sulfenyl amide protein tyrosine phosphatase 1B-ligand complexes and mutant and homologue proteins of sulfenyl amide protein tyrosine phosphatase 1B, crystals of such proteins may be formed. The skilled person would recognize that the conditions provided herein for crystallizing sulfenyl amide protein tyrosine phosphatase 1B may be used to form such crystals. Alternatively, the skilled person would use the conditions as a basis for identifying modified conditions for forming the crystals.

Thus the aspects of the invention relating to crystals of sulfenyl amide protein tyrosine phosphatase 1B, may be extended to crystals of a mutant, analogue or homologue, or mutein

Homology Modelling

The invention also provides a means for homology modelling of other proteins (referred to below as target sulfenyl amide protein tyrosine phosphatase proteins).

By “homology modelling”, it is meant the prediction of related sulfenyl amide protein tyrosine phosphatase structures based either on X-ray crystallographic data or computer-assisted de novo prediction of structure, based upon manipulation of the coordinate data of Table 1 or Table 2.

“Homology modeling” extends to target sulfenyl amide protein tyrosine phosphatase proteins, which are analogues or homologues of the sulfenyl amide protein tyrosine phosphatase 1B protein whose structure has been determined in the accompanying examples.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

In general, the method involves comparing the amino acid sequences of the sulfenyl amide protein tyrosine phosphatase 1B protein of Table 1 or Table 2 with a target sulfenyl amide protein tyrosine phosphatase protein by aligning the amino acid sequences (Dunbrack et al., Folding and Design, 2, (1997), 27-42). Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms. The programs BLAST, GAPPED BLAST, BLASTN, PSI-BLAST AND BLAST 2 sequences (provided by the National Center for Biotechnology Information) are widely used in the art for this purpose, and can align homologous regions of two amino acid sequences. These may be used with default parameters to determine the degree of homology between the amino acid sequence of the Table 1 or Table 2 protein and other target sulfenyl amide protein tyrosine phosphatase proteins, which are to be modeled.

Analogues are defined as proteins with similar three-dimensional structures and/or functions with little evidence of a common ancestor at a sequence level.

Homologues are defined as previously as proteins with evidence of a common ancestor, i.e. likely to be the result of evolutionary divergence and are divided into remote, medium and close sub-divisions based on the degree (usually expressed as a percentage) of sequence identity.

A homologue is defined here as a protein with at least 15% sequence identity or which has at least one functional domain, which is characteristic of sulfenyl amide protein tyrosine phosphatase 1B.

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

The homologues could also be mutants as described above.

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modeling as such is a technique that is well known to those skilled in the art (see e.g. Greer, Science, vol. 228, (1985), 1055, and Blundell et al., Eur. J Biochem, vol. 172, (1988), 513). The techniques described in these references, as well as other homology modeling techniques, generally available in the art, may be used in performing the present invention.

Thus the invention provides a method of homology modeling comprising the steps of: (a) aligning a representation of an amino acid sequence of a target sulfenyl amide protein tyrosine phosphatase protein of unknown three-dimensional structure with the amino acid sequence of the sulfenyl amide protein tyrosine phosphatase 1B of Table 1 or Table 2 to match homologous regions of the amino acid sequences; (b) modeling the structure of the matched homologous regions of said target sulfenyl amide protein tyrosine phosphatase of unknown structure on the corresponding regions of the sulfenyl amide protein tyrosine phosphatase 1B structure as defined by Table 1 or Table 2; and (c) determining a conformation (e.g. so that favorable interactions are formed within the target sulfenyl amide protein tyrosine phosphatase of unknown structure and/or so that a low energy conformation is formed) for said target sulfenyl amide protein tyrosine phosphatase of unknown structure which substantially preserves the structure of said matched homologous regions.

Preferably one or all of steps (a) to (c) are performed by computer modeling.

The aspects of the invention described herein which utilize the sulfenyl amide protein tyrosine phosphatase 1B structure in silico may be equally applied to homologue models of sulfenyl amide protein tyrosine phosphatase obtained by the above aspect of the invention, and this application forms a further aspect of the present invention. Thus having determined a conformation of a sulfenyl amide protein tyrosine phosphatase by the method described above, such a conformation may be used in a computer-based method of rational drug design as described herein.

In a preferred aspect of this invention the co-ordinates are used to model the structure of target sulfenyl amide protein tyrosine phosphatases, particularly homologues of sulfenyl amide protein tyrosine phosphatase 1B, for example PTP-α, T-cell PTP, or LAR.

Structure Solution

The structure of the human sulfenyl amide protein tyrosine phosphatase 1B can also be used to solve the crystal structure of other target sulfenyl amide protein tyrosine phosphatase proteins including other crystal forms of sulfenyl amide protein tyrosine phosphatase 1B, mutants, and co-complexes of sulfenyl amide protein tyrosine phosphatase 1B, where X-ray diffraction data or NMR spectroscopic data of these target sulfenyl amide protein tyrosine phosphatase proteins have been generated and require interpretation in order to provide a structure.

In the case of sulfenyl amide protein tyrosine phosphatase 1B, this protein may crystallize in more than one crystal form. The structure coordinates of sulfenyl amide protein tyrosine phosphatase 1B, or portions thereof, as provided by this invention are particularly useful to solve the structure of those other crystal forms of sulfenyl amide protein tyrosine phosphatase 1B. They may also be used to solve the structure of sulfenyl amide protein tyrosine phosphatase 1B mutants, sulfenyl amide protein tyrosine phosphatase 1B co-complexes, or the structure of the crystalline form of any other protein with significant amino acid sequence homology to sulfenyl amide protein tyrosine phosphatase 1B.

In the case of other target sulfenyl amide protein tyrosine phosphatase proteins, particularly the mutant sulfenyl amide protein tyrosine phosphatase proteins referred to above, the present invention allows the structures of such targets to be obtained more readily where raw X-ray diffraction data are generated.

Thus, where X-ray crystallographic or NMR spectroscopic data are provided for a target sulfenyl amide protein tyrosine phosphatase 1B-ligand complex, or a sulfenyl amide protein tyrosine phosphatase 1B homologue or analogue of unknown three-dimensional structure, the structure of sulfenyl amide protein tyrosine phosphatase 1B, as defined by Table 1 or Table 2, may be used to interpret the data to provide a likely structure for the other sulfenyl amide protein tyrosine phosphatases by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of sulfenyl amide protein tyrosine phosphatase 1B, a sulfenyl amide protein tyrosine phosphatase 1B mutant, or a sulfenyl amide protein tyrosine phosphatase 1B co-complex, or the crystal of a target sulfenyl amide protein tyrosine phosphatase protein with amino acid sequence homology to protein tyrosine phosphatase 1B, may be determined using the sulfenyl amide protein tyrosine phosphatase 1B structure coordinates of this invention as provided herein. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelrys San Diego, Calif.) or Amore (Navaza, J. (1994). Amore: An Automated Package for Molecular Replacement. Acta Cryst. A50, 157-163).

Thus, in a further aspect of the invention provides a method for determining the structure of a protein, which method comprises providing the co-ordinates of Table 1 or Table 2, and either (a) positioning the co-ordinates in the crystal unit cell of said protein so as to provide a structure for said protein or (b) assigning NMR spectra peaks of said protein by manipulating the coordinates of Table 1 or Table 2.

In a preferred aspect of this invention the co-ordinates are used to solve the structure of target sulfenyl amide protein tyrosine phosphatase, particularly homologues of sulfenyl amide protein tyrosine phosphatase 1B, for example PTP-α, T-cell PTP, or LAR.

Computer Systems

In another aspect, the present invention provides systems, particularly a computer system, the systems containing either (a) atomic coordinate data according to Table 1 or Table 2, said data defining the three-dimensional structure of sulfenyl amide protein tyrosine phosphatase 1B or at least selected coordinates thereof; (b) structure factor data (where a structure factor comprises the amplitude and phase of the diffracted wave) for sulfenyl amide protein tyrosine phosphatase 1B, said structure factor data being derivable from the atomic coordinate data of Table 1 or Table 2; (c) atomic coordinate data of a target sulfenyl amide protein tyrosine phosphatase protein generated by homology of the target based on the data of Table 1 or Table 2; (d) atomic coordinate data of a target sulfenyl amide protein tyrosine phosphatase protein generated by interpreting X-ray crystallographic data or NMR data by reference to the data of Table 1 or Table 2; or (e) structure factor data derivable from the atomic coordinate data of (c) or (d).

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to said central-processing unit for displaying said structures.

The invention also provides such systems containing atomic coordinate data of target sulfenyl amide protein tyrosine phosphatase proteins wherein such data have been generated according to the methods of the invention described herein based on the starting data provided by Table 1 or Table 2.

Such data are useful for a number of purposes, including the generation of structures to analyze the mechanisms of action of sulfenyl amide protein tyrosine phosphatase 1B proteins and/or to perform rational drug design of compounds which interact with sulfenyl amide protein tyrosine phosphatase 1B, such as compounds which are inhibitors of sulfenyl amide protein tyrosine phosphatase 1B.

In another aspect, the invention provides a computer-readable storage medium, comprising a data storage material encoded with computer readable data, wherein the data are defined by all or a portion (e.g. selected coordinates as defined herein) of the structure coordinates of sulfenyl amide protein tyrosine phosphatase 1B of Table 1 or Table 2, or a homologue of sulfenyl amide protein tyrosine phosphatase 1B, wherein said homologue comprises backbone atoms that have a root mean square deviation from the backbone atoms (nitrogen-carbon-carbon) of Table 1 or Table 2 of not more than 1.5 Å.

The invention also provides a computer-readable data storage medium comprising a data storage material encoded with a first set of computer-readable data comprising a Fourier Transform of at least a portion (e.g. selected coordinates as defined herein) of the structural coordinates for sulfenyl amide protein tyrosine phosphatase 1B according to Table 1 or Table 2; which, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

In a further aspect, the present invention provides computer readable media with at least one of: (a) atomic coordinate data according to Table 1 or Table 2 recorded thereon, said data defining the three-dimensional structure of sulfenyl amide protein tyrosine phosphatase 1B, or at least selected coordinates thereof; (b) structure factor data for sulfenyl amide protein tyrosine phosphatase 1B recorded thereon, the structure factor data being derivable from the atomic coordinate data of Table 1 or Table 2; (c) atomic coordinate data of a target sulfenyl amide protein tyrosine phosphatase protein generated by homology modeling of the target based on the data of Table 1 or Table 2; (d) atomic coordinate data of a sulfenyl amide protein tyrosine phosphatase 1B-ligand complex or a sulfenyl amide protein tyrosine phosphatase 1B homologue or analogue generated by interpreting X-ray crystallographic data or NMR data by reference to the data of Table 1 or Table 2; and (e) structure factor data derivable from the atomic coordinate data of (c) or (d).

By providing such computer readable media, the atomic coordinate data can be routinely accessed to model sulfenyl amide protein tyrosine phosphatase 1B or selected coordinates thereof. For example, Rasmol (Sayle et al., TIBS, vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976)), are particularly useful for calculating e.g. difference Fourier electron density maps.

A further aspect of the invention provides a method of providing data for generating structures and/or performing rational drug design for sulfenyl amide protein tyrosine phosphatase 1B, sulfenyl amide protein tyrosine phosphatase 1B homologues or analogues, complexes of sulfenyl amide protein tyrosine phosphatase 1B with a candidate modulator, or complexes of sulfenyl amide protein tyrosine phosphatase 1B homologues or analogues with candidate modulators, the method comprising:

(i) establishing communication with a remote device containing computer-readable data comprising at least one of: (a) atomic coordinate data according to Table 1 or Table 2, said data defining the three-dimensional structure of sulfenyl amide protein tyrosine phosphatase 1B, at least one sub-domain of the three-dimensional structure of sulfenyl amide protein tyrosine phosphatase 1B, or the coordinates of a portion of atoms of sulfenyl amide protein tyrosine phosphatase 1B; (b) structure factor data for sulfenyl amide protein tyrosine phosphatase 1B, said structure factor data being derivable from the atomic coordinate data of Table 1 or Table 2; (c) atomic coordinate data of a target sulfenyl amide protein tyrosine phosphatase 1B homologue or analogue generated by homology modeling of the target based on the data of Table 1 or Table 2; (d) atomic coordinate data of a protein generated by interpreting X-ray crystallographic data or NMR data by reference to the data of Table 1 or Table 2; and (e) structure factor data derivable from the atomic coordinate data of (c) or (d); and (ii) receiving said computer-readable data from said remote device.

Thus the remote device may comprise e.g. a computer system or computer readable media of one of the previous aspects of the invention. The device may be in a different country or jurisdiction from where the computer-readable data is received. The communication may be via the internet, intranet, e-mail etc. Typically the communication will be electronic in nature, but some or all of the communication pathway may be optical, for example, over optical fibre transmission lines.

Drug Discovery

Determination of the 3D structure of PTP1B provides important information about the nature of the changes to the active site of PTPs upon oxidation, in particular the changes in PTP1B upon oxidation to the PTP1B sulfenyl amide. In particular, the X-ray data provide information about new binding sites created by distortion of the active site as a consequence of the formation of the sulfenyl amide. Information about the new binding sites can then be used for rational design of compounds that bind to PTPsulfenyl amide, especially PTP1B sulfenyl amide. This can be achieved by e.g. computational techniques which identify possible binding ligands for the active sites, by enabling linked-fragment approaches to drug design, and by enabling the identification and location of bound ligands using X-ray crystallographic analysis. These techniques are discussed in more detail below.

In silico Analysis

The provision of the crystal structure of PTP1B sulfenyl amide allows a novel approach for drug discovery for modulators of this inactive form of PTP and in particular of PTP1B. Accordingly, the invention provides a computer-based method of rational drug design which comprises:
providing the structure of the PTP1b sulfenyl amide as defined by the coordinates of Table 1 or Table 2;
providing the structure of a candidate modulator molecule; and
fitting the structure of candidate to the structure of the sulfenyl amide of Table 1 or Table 2.

More particularly, the crystal structure of the sulfenyl amide can be used to design drug molecules that bind to the sulfenyl amide of PTP1B to inhibit or prevent its conversion to the active form of PTP1B and hence another aspect of the invention comprises a computer-based method of rational drug design which comprises;
providing the structure of the PTP1B sulfenyl amide as defined by the coordinates of Table 1 or Table 2;
providing the structure of a candidate compound; and
fitting the structure of the candidate compound to the structure of the sulfenyl amide as defined by the coordinates of Table 1 or Table 2.

The invention further provides a method of identifying by rational drug design a compound capable of reducing the level of activity of a protein tyrosine phosphatase (PTP) in a cellular environment, the PTP being one which is convertible in a cellular environment between an active form and an inactive or less active form, the inactive form or less active form being characterised by the presence of a sulfenyl amide moiety formed at the active site of the PTP between the sulphur atom of a cysteine group and a backbone nitrogen atom of a neighbouring amino acid;
which method comprises:

(a) designing a ligand that will (i) bind to the active site in the region of the sulfenyl amide moiety to inhibit conversion of the inactive form or less active form back to the active form, or (ii) modify the sulfenyl amide moiety to inhibit conversion of the inactive form or less active form of the PTP to the active form;

(b) synthesizing the ligand; and

(c) determining whether the ligand reduces the level of activity of a protein tyrosine phosphate (PTP) in a cellular environment.

In an alternative aspect, the method of the invention may utilise the coordinates of atoms of interest of the PTP1B which are in the vicinity of a putative binding region in order to model the pocket in which the a ligand will bind. These coordinates may be used to define a space which is then screened “in silico” against a candidate modulator molecule.

Thus the invention provides a computer-based method of rational drug design which comprises:
providing the coordinates of at least two atoms of Table 1 or Table 2 of the PTP1B sulfenyl amide (“selected coordinates”);
providing the structure of a candidate modulator molecule; and
fitting the structure of candidate to the selected coordinates of the PTP1B sulfenyl amide.

In practice, it will be desirable to model a sufficient number of atoms of the PTP1B sulfenyl amide as defined by the coordinates of Table 1 or Table 2 which represent a binding pocket. Binding pockets and other features of the interaction of PTP1B sulfenyl amide with a putative compound of the invention are described below. Thus, in this embodiment of the invention, there will preferably be provided the coordinates of at least 5, preferably at least 10, more preferably at least 50 and even more preferably at least 100 atoms of the PTP sulfenyl amide structure.

By “fitting”, it is meant determining by automatic, or semi-automatic means, interactions between at least one atom of the candidate and at least one atom of the PTP1B sulfenyl amide, and calculating the extent to which such an interaction is stable. Interactions include attraction and repulsion, brought about by charge, steric considerations and the like. Various computer-based methods for fitting are described further herein.

By “binding site” we mean a site (such as an atom, a functional group of an amino acid residue or a plurality of such atoms and/or groups) in a PTP1B sulfenyl amide binding cavity which may bind to a candidate ligand. Depending on the particular molecule in the cavity, sites may exhibit attractive or repulsive binding interactions, brought about by charge, steric considerations and the like.

As a result of the determination of the PTP1B sulfenyl amide 3D structure, more purely computational techniques for rational drug design may also be used to design PTP sulfenyl amide ligands (for an overview of these techniques see e.g Walters et al (Drug Discovery Today, Vol.3, No.4, (1998), 160-178; Abagyan, R.; Totrov, M. Curr. Opin. Chem. Biol. 2001, 5, 375-382)). For example, automated ligand-receptor docking programs (discussed e.g. by Jones et al. in Current Opinion in Biotechnology, Vol.6, (1995), 652-656 and Halperin, I.; Ma, B.; Wolfson, H.; Nussinov, R. Proteins 2002, 47, 409-443) which require accurate information on the atomic coordinates of target receptors may be used to design potential PTP1B sulfenyl amide ligands.

The step of providing the structure of a candidate ligand molecule may involve selecting the compound by computationally screening a database of compounds for interaction with the active site. For example, a 3-D descriptor for the candidate modulator may be derived, the descriptor including geometric and functional constraints derived from the architecture and chemical nature of the binding site. The descriptor may then be used to interrogate the compound database, a candidate ligand being a compound that has a good match to the features of the descriptor. In effect, the descriptor is a type of virtual pharmacophore.

In any event, the determination of the three-dimensional structure of PTP1B sulfenyl amide provides a basis for the design of new and specific ligands for PTP1B sulfenyl amide. For example, knowing the three-dimensional structure of PTP1B sulfenyl amides, computer modelling programs may be used to design different molecules expected to interact with possible or confirmed active sites, such as binding sites or other structural or functional features of PTP1B sulfenyl amide.

More specifically, a candidate ligand for PTP1B sulfenyl amide can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J. Mol. Biol. 1982, 161, 269-288, Makino et al, J. Comput. Chem. 1997, 18, 1812-1825), AUTODOCK (Goodsell et al, Proteins 1990, 8, 195-202, Morris et al, J. Comput. Chem. 1998, 19, 1639-1662.), FlexX, (Rarey et al, J. Mol. Biol. 1996, 261, 470-489) or ICM (Abagyan et al, J. Comput. Chem. 1994, 15, 488-506). This procedure can include computer fitting of candidate ligands to PTP1B sulfenyl amide to ascertain how well the shape and the chemical structure of the candidate ligand will bind to the enzyme.

Also computer-assisted, manual examination of the active site structure of PTP1B may be performed. The use of programs such as GRID (Goodford, J. Med. Chem., 28, (1985), 849-857)—a program that determines probable interaction sites between molecules with various functional groups and the enzyme surface—may also be used to analyse the active site to predict partial structures of ligands.

Computer programs can be employed to estimate the attraction, repulsion, and steric hindrance of the two binding partners (e.g. the PTP1B sulfenyl amide and a candidate ligand). Generally the tighter the fit, the fewer the steric hindrances, and the greater the attractive forces, the more potent the candidate ligand since these properties are consistent with a tighter binding constant. Furthermore, the more specificity in the design of a candidate ligand, the more likely it is that it will not interact with other proteins as well. This will tend to minimise potential side-effects due to unwanted interactions with other proteins.

Linked Fragment and Fragment Growing Approaches.

If more than one PTP1B sulfenyl amide binding site is characterised and a plurality of respective compounds are designed or selected, the candidate ligand may be formed by linking the respective compounds into a larger compound which maintains the relative positions and orientations of the respective compounds at the active sites. The larger compound may be formed as a real molecule or by computer modelling.

Linked-fragment approaches to drug design also require accurate information on the atomic coordinates of target receptors. Small compounds which have the potential to bind to regions of PTP1B sulfenyl amide which in themselves may not be modulator compounds may be assembled by chemical linkage to provide candidate modulators. Thus the basic idea behind these approaches is to determine the binding locations of plural ligands to a target molecule, and then construct a molecular scaffold to connect the ligands together in such a way that their relative binding positions are preserved. The ligands may be provided computationally and modelled in a computer system, or provided in an experimental setting, wherein crystals according to the invention are provided and a plurality of ligands soaked separately or in mixed pools into the crystal prior to X-ray analysis and determination of their location.

For example, the binding of one or more molecular fragments can be determined in the protein binding cavity by X-ray crystallography. Molecular fragments are typically compounds with a molecular weight between 100 and 200 Da. This can then provide a starting point for medicinal chemistry to optimize the interactions using a structure-based approach. The fragments can be combined onto a template or used as the starting point for “growing out” a modulator into other cavities of the protein. The fragments can be positioned in the binding cavity or cavities of PTP1B sulfenyl amide and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical synthesis.

At one or more stages in the fragment growing approach, the compound may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure which may have the desired properties.

Thus the binding site of two of more ligands are determined and may be connected to thus form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design PTP1B sulfenyl amide modulators is made possible by the determination of the PTP1B sulfenyl amide structure.

Generation and Analysis of Ligand-PTP1B Sulfenyl Amide Complexes

In a further aspect, the invention provides a method for determining the structure of a compound bound to sulfenyl amide PTP1B. The methods above may comprise the further steps of:
obtaining or synthesising a candidate modulator;
forming a complex of PTP1B sulfenyl amide and said candidate modulator; and
analysing said complex by X-ray crystallography to determine the ability of said candidate modulator to interact with PTP1B sulfenyl amide.

The invention also provides a method for determining the structure of a compound bound to sulfenyl amide PTP1b, said method comprising: (a) providing a crystal of sulfenyl amide PTP1b according to the invention; (b) soaking the crystal with said compound; and (c) determining the structure of said sulfenyl amide PTP1b compound complex by employing the data of Table 1 or Table 2.

Alternatively, the sulfenyl amide PTP1B and compound may be co-crystallized. Thus the invention provides a method for determining the structure of a compound bound to sulfenyl amide PTP1b, said method comprising; mixing the protein with the compound(s), crystallizing the protein-compound(s) complex; and determining the structure of said sulfenyl amide PTP1b -compound(s) complex by reference to the data of Table 1 or Table 2.

A mixture of compounds may be soaked or co-crystallized with the crystal, wherein only one or some of the compounds may be expected to bind to the sulfenyl amide PTP1b. As well as the structure of the complex, the identity of the complexing compound(s) is/are then determined.

In either case, substrate or a substrate analogue thereof may optionally be present.

The method may comprise the further steps of: (a) obtaining or synthesising said compound; (b) forming a complex of sulfenyl amide PTP1B and said compound; and (c) analysing said complex by X-ray crystallography or NMR spectroscopy to determine the ability of said compound to interact with sulfenyl amide PTP1b.

This information may thus be used to design and synthesize novel classes of sulfenyl amide PTP1B inhibitors.

Detailed structural information can then be obtained about the binding of the candidate modulator to PTP1B sulfenyl amide, and in the light of this information adjustments can be made to the structure or functionality of the candidate modulator, e.g. to improve binding to the active site. The above steps may be repeated and re-repeated as necessary.

In another aspect, the invention provides a method of analysing a complex of PTP 1B sulfenyl amide and a candidate modulator comprising the step of employing (i) X-ray crystallographic diffraction data from the complex and (ii) a three-dimensional structure of PTP1B sulfenyl amide, or at least one sub-domain thereof, to generate a difference Fourier electron density map of the complex, the three-dimensional structure being defined by atomic coordinate data according to Table 1 or Table 2.

Therefore, such complexes can be crystallised and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallised PTP1B sulfenyl amide and the solved structure of uncomplexed PTP1B sulfenyl amide. These maps can then be used to determine whether and where a particular candidate modulator binds to PTP1B sulfenyl amide and/or changes the conformation of PTP1B sulfenyl amide.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualisation and model building programs such as “O” (Jones et al., Acta Crystallograhy, A47, (1991), 110-119) or QUANTA” (1994, San Diego, Calif.: Molecular Simulations, Jones et al., Acta Crystallography A47 (1991), 110-119) can be used.

Greer et al. mentioned above describes an iterative approach to ligand design based on repeated sequences of computer modelling, protein-ligand complex formation and X-ray analysis. Thus novel thymidylate synthase inhibitor series were designed de novo by Greer et al., and PTP1B sulfenyl amide inhibitors may also be designed in the this way. More specifically, using e.g. GRID on the solved 3D structure of PTP1B, a candidate modulator for PTP1B sulfenyl amide may be designed that complements the functionalities of the PTP1B sulfenyl amide binding site(s). The candidate modulator compound can then be synthesised, formed into a complex with PTP1B sulfenyl amide, and the complex then analysed by X-ray crystallography to identify the actual position of the bound compound.

Determination of the position of the candidate modulator in the complex allows determination of the interactions of it with PTP1B sulfenyl amide. This will allow those of skill in the art to analyse the affinity and specificity of the compound for PTP1B sulfenyl amide, and to propose modifications to the compound to increase or decrease either or both of these properties. Thus the structure and/or functional groups of the compound can then be adjusted, if necessary, in view of the results of the X-ray analysis, and the synthesis and analysis sequence repeated until an optimised compound is obtained. Related approaches to structure-based drug design are also discussed in Bohacek et al., Medicinal Research Reviews, Vol.16, (1996), 3-50.

Many of the techniques and approaches to structure-based drug design described above require X-ray analysis to identify the binding position of a candidate modulator in a complex with a protein. A common way of doing this is to perform X-ray crystallography on the complex, produce a difference Fourier electron density map, and associate a particular pattern of electron density with the candidate modulator. However, in order to produce the map (as explained e.g. by Blundell et al. mentioned above) it is necessary to know beforehand the protein 3D structure (or at least the protein structure factors).

Therefore, determination of the PTP1B sulfenyl amide structure also allows difference Fourier electron density maps of complexes of PTP1B sulfenyl amide with a candidate modulator to be produced, which can greatly assist the process of rational drug design.

The approaches to structure-based drug design described above all require initial identification of possible compounds for interaction with target bio-molecule (in this case PTP1B sulfenyl amide). Sometimes these compounds are known e.g. from the research literature. However, when they are not, or when novel compounds are wanted, a first stage of the drug design program may involve computer-based in silico screening of compound databases (such as the Cambridge Structural Database) with the aim of identifying compounds which interact with the binding site or sites of the target bio-molecule (see Martin, J. Med. Chem., vol 35, 2145-2154 (1992)). Screening selection criteria may be based on pharmacokinetic properties such as metabolic stability and toxicity. However, determination of the PTP1B sulfenyl amide structure allows the architecture and chemical nature of each PTP1B active site to be identified, which in turn allows the geometric and functional constraints of a descriptor for the potential inhibitor to be derived. The descriptor is, therefore, a type of virtual 3-D pharmacophore, which can also be used as selection criteria or filter for database screening.

The Crystal Structure of the Catalytic Domain of PTP1B Sulfenyl Amide

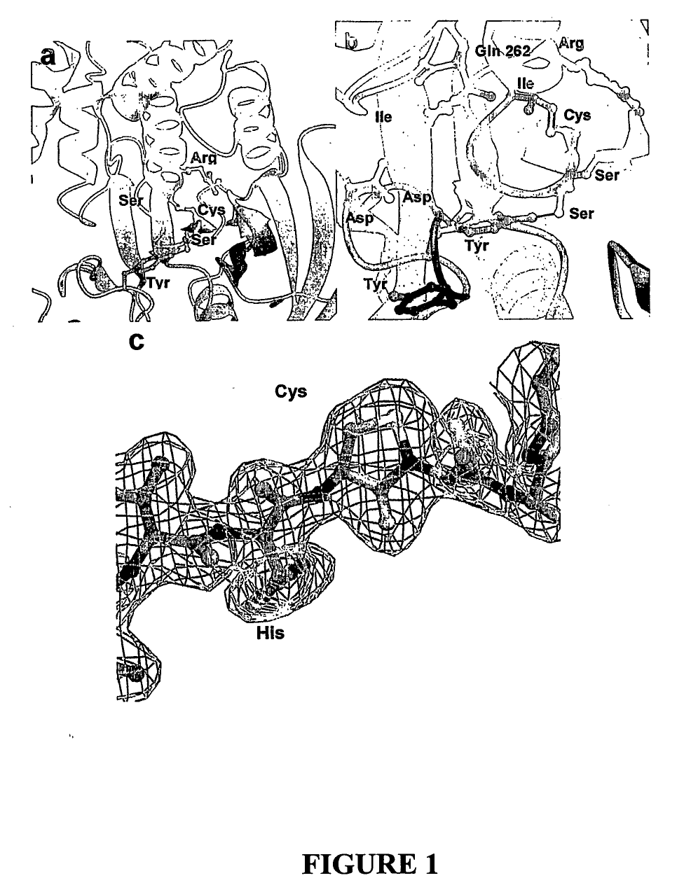

The structure of the portion of the PTP sulfenyl amide corresponding to the catalytic domain of PTP1B is defined by the atomic coordinates set out in Table 1 or Table 2. The three dimensional structure of the binding sites of the PTP1B sulfenyl amide are shown schematically in FIGS. 1b and 1c.

The crystal structure shows electron density close to the side chain of the catalytic cysteine characteristic of the presence of a covalent bond between the sulphur Sγ atom of Cys215 and the backbone nitrogen atom of Ser216 (see FIG. 1c). The sulfenyl-amide bond has a bond length of 1.7 Å and results in a five-membered puckered ring that has not been previously observed in proteins. In conjunction with the formation of the sulfenyl-amide derivative the phosphate-binding cradle adopts a novel conformation, distinct from the structure of the known inactive C215S PTP1B mutant¹⁶. The cradle has shifted into the phosphotyrosine binding site and stabilises the sulfenyl-amide by a hydrophobic interaction with the side chain of Ile219 (FIG. 1b). In addition, the side chain of Gln262 moves out of the active site and also the pTyr loop adopts a unique conformation (FIG. 1b). The more exposed conformation of the pTyr loop results from the loss of the hydrogen bond between the hydroxyl groups of Tyr46 and Ser216, which anchors the pTyr loop in native PTP1B and is stabilised by a network of water molecules mediating interactions between Asp48 and the rest of the protein.

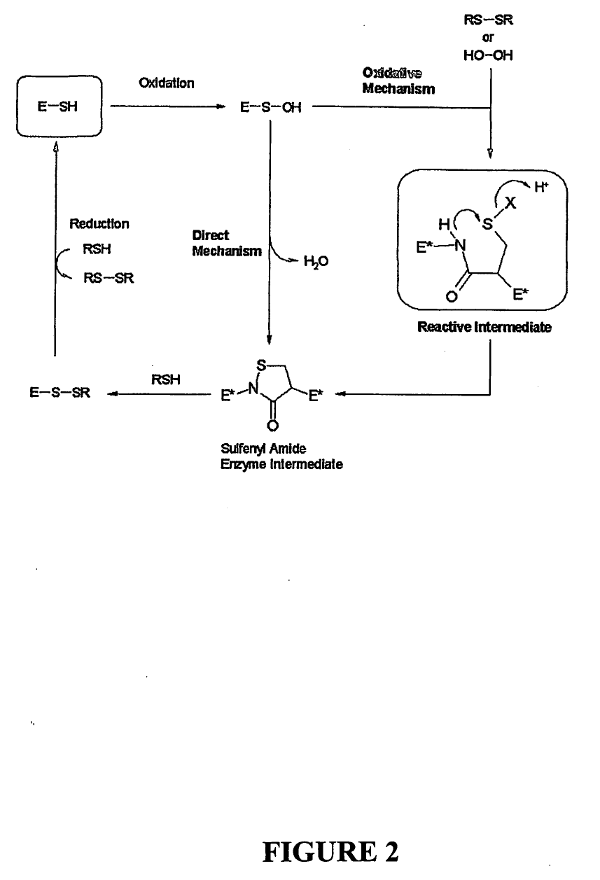

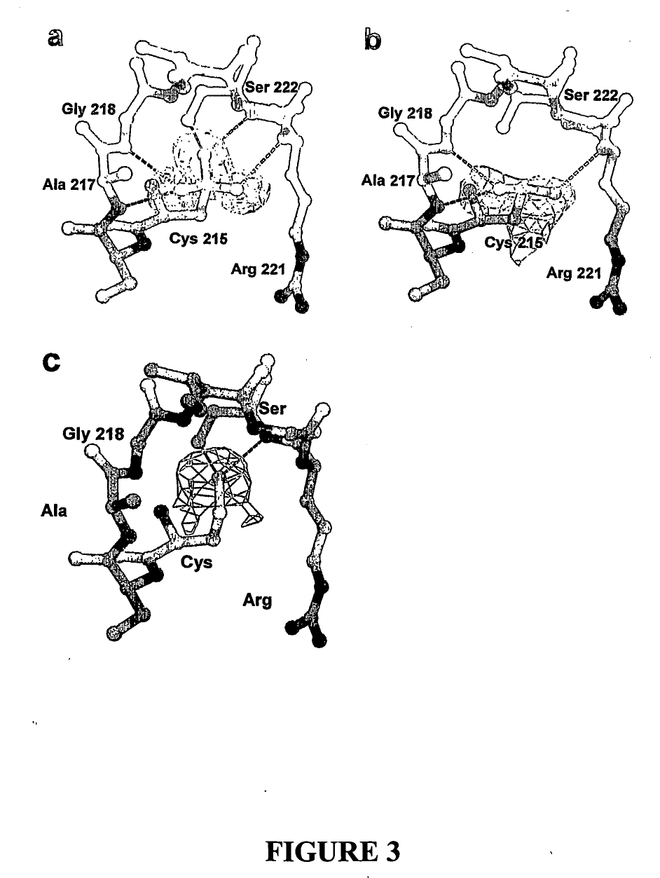

Formation of the sulfenyl-amide arises from oxidation of the active site Cys215, most likely via oxidation of Cys215 to sulfenic acid, followed by a nucleophilic attack of the backbone nitrogen atom of Ser216 on the Sγ atom of Cys215. Indeed, it has been postulated that the hydrogen bond interaction between the carbonyl oxygen atom of Cys215 and the N1 atom of the invariant His214 side chain in native PTP1B increases the partial charge on the backbone nitrogen atom of Ser216¹², enhancing its reactivity and supporting a nucleophilic substitution mechanism. In vivo the sulfenic acid can be formed by oxidation with hydrogen peroxide (H₂O₂)¹⁷, but under our experimental conditions it is most likely triggered by redox-cycling of 2-Phenyl-isoxazolidine-3,5-dione (FIG. 2). The leaving group in the cyclisation reaction could be a water molecule in an SN₂ substitution reaction (direct mechanism, FIG. 2). Alternatively, the sulfenic acid derivative might be oxidised under the experimental soaking conditions, or by physiological oxidants such as hydrogen peroxide or oxidised glutathione in vivo, to form a highly reactive intermediate. This would result in faster formation of the sulfenyl-amide species (oxidative mechanism, FIG. 2).

The role for the sulfenyl-amide intermediate in oxidative regulation of PTP1B in cells is likely to be the prevention of irreversible oxidation of Cys215 and thus facilitation of its thiol-mediated reactivation (FIG. 2). To demonstrate the reversibility of the S—N bond, reducing conditions were employed in an attempt to reduce the sulfenyl-amide derivative in the crystals. First, two PTP1B crystals were soaked in a solution containing 2-Phenyl-isoxazolidine-3,5-dione. X-ray data collected from one of the crystals confirmed the formation of the sulfenyl-amide bond and the concomitant conformational changes in the active site. The second crystal was back-soaked in 20 mM reduced glutathione in an attempt to reduce the Cys215 sulfenyl-amide derivative back to its native form. Indeed, X-ray data collected from the back-soaked crystal showed the entire active site back in its native conformation, thus structurally confirming reactivation of the sulfenyl-amide PTP1B derivative by a biologically relevant reducing agent and strengthening the hypothesis of a protective role in PTP1B redox-regulation.

The studies carried out provide a detailed structural understanding of the intermediates involved in redox-regulation of PTP1B and reveal a novel oxidation state of its catalytic cysteine. The formation of the sulfenyl-amide intermediate is an elegant mechanism to protect Cys215 from further oxidation, and the concomitant conformational changes of the phosphate-binding cradle and pTyr loop may serve to signal the inactive state of the enzyme. The structures of the sulfenic acid and sulfenyl-amide derivative indicate that reactivation of PTP1B appears to be facilitated by the sulfenyl-amide form.

The sulfenyl-amide form of PTPs is an important regulatory intermediate of these proteins. It is stable to oxidation to the sulfinic or sulfonic protein forms that are irreversibly inhibited and therefore prevents permanent inactivation of the protein. It can then subsequently be converted to the reduced, active form of the protein by physiological reducing agents such as thiols. The sulfenyl amide is an isothiazolidin-3-one ring system, which has not been previously observed in proteins. It is an electrophilic species in the active site of an enzyme, and this is also very unusual as most enzymes display nucleophiles in their catalytic machinery.

Therapeutic and Medical Uses of the Inhibitors of PTP Sulfenyl Amide

It is envisaged that compounds that stabilize the sulfenyl amide form or effect reversible or irreversible covalent modification of the sulfenyl amide form will be useful as therapeutic agents in the treatment of disease states or conditions mediated by protein tyrosine phosphatases.

Accordingly, in one aspect, the invention provides the use of a compound for the manufacture of a medicament for the treatment of a disease or condition mediated by protein tyrosine phosphatase, wherein the compound is one that binds to protein tyrosine phosphatase sulfenyl amide to prevent or inhibit conversion of the protein tyrosine phosphatase sulfenyl amide to an active reduced form of the protein tyrosine phosphatase.

In a further aspect, the invention provides a method of reducing the activity of a protein tyrosine phosphotase (PTP), the PTP being one which is convertible between an active form and an inactive or less active form, the inactive or less active form being characterised by the presence of a sulfenyl amide moiety formed at the active site of the PTP between the sulphur atom of a cysteine group and a backbone nitrogen atom of a neighbouring amino acid, whereby the sulfenyl amide moiety distorts and inactivates the active site of the PTP and wherein the sulfenyl amide moiety is disruptible to restore the inactive or less active form of the PTP to the active form thereof;
which method comprises inhibiting disruption of the sulfenyl amide moiety, or modifying the sulfenyl amide moiety to prevent restoration of the inactive or less active form of the PTP to the active form.

In another aspect, the invention provides a method of inhibiting or preventing the reduction of sulfenyl amide PTP1B to PTP1B in a cellular environment by exposing the PTP1B to a ligand capable of binding to the sulfenyl amide PTP1B in the region of the sulfenyl amide moiety so as to prevent reduction of the sulfenyl amide moiety by an intracellular reducing agent.

The invention also provides a method of inhibiting or preventing the reduction of sulfenyl amide PTP1B to PTP1B in a cellular environment by exposing the PTP1B to a ligand capable of binding to the sulfenyl amide PTP1B in the region of the sulfenyl amide moiety, the ligand having a nucleophilic moiety capable of modifying the sulfenyl amide moiety so as to prevent its reduction by an intracellular reducing agent.

The Compounds of the Invention

The invention provides novel compounds per se that inhibit protein tyrosine phosphatases by interacting with the sulfenyl amide PTP to prevent or inhibit conversion of the PTP sulfenyl amide to an active reduced form of the protein tyrosine phosphatase.

In addition to compounds per se, the invention provides compounds of the aforesaid type for use in therapy or for use in medicine, for example for use in the treatment of diseases or conditions mediated by protein tyrosine phosphatase.

Conversion of the PTP sulfenyl amide to the corresponding active reduced form can be inhibited in several ways by small molecule ligands.

Mode 1. Non-covalent binding inhibitors that stabilise the sulfenyl-amide protein form. These inhibitors are designed to prevent physiological cell cycling of the protein form into its active reduced form by preventing binding to the protein.

Mode 2. Reversible covalent binding inhibitors that modify the sulfenyl-amide form of the protein. These inhibitors are designed to react with the active site sulfenyl amide, and in so doing, prevent its reactivation by physiological cell cycling.

Mode 3. Irreversible covalent binding inhibitors that modify the sulfenyl-amide form of the protein. These inhibitors are designed to react with the active site sulfenyl amide, and in so doing, prevent its reactivation by physiological cell cycling.

The concerted distortion of the phosphate binding cradle and phosphotyrosine recognition loop upon sulfenyl-amide formation destroys the normal phosphotyrosine binding site and creates a new groove (referred to herein as “the first binding site”) in which small molecules could bind. This groove is lined by residues 41-47 of the phosphotyrosine recognition loop, residues 88-90, 115 to 120, residues 179 to 184 of the WPD-loop, residues 215 to 219 of the phosphate-binding cradle, and residues 262-266.

Compounds having Mode 1 activity include compounds that can make polar interactions at the first binding site with one or more of:

(1) Lys41

(2) Asn42

(3) Arg45

(4) Tyr46

(5) Arg47

(6) Asn90

(7) Gln115

(8) Lys116

(9) Ser118

(10) Lys120

(11) Trp179

(12) Ser216

(13) Arg221

(14) Gln262

(15) Thr263

(16) Asp265, and

(17) Gln266

The amino acid numbering convention used above refers to the numbering of PTP1B.

Preferably, the compounds make polar interactions with two or more of the listed moieties (1) to (17), more preferably three or more, for example four or more, and more particularly five or more.

The compounds can make hydrophobic interactions with one or more of:

(18) Leu88

(19) Pro89

(20) Leu119

(21) Phe182

(22) Gly183

(23) Val184

(24) Ala217

(25) Ile219

(26) the apolar part of Arg221, and

(27) the apolar part of Gln262.

Preferably, the compounds make hydrophobic interactions with two or more of the listed moieties, more preferably three or more, for example five or more.

Additional hydrogen bonds and hydrophobic interactions may be formed between a bound molecule and the protein backbone.

A second shallow depression in which a small molecule could bind (referred to hereinafter as “the second binding site”) is located on the other side of the distorted protein tyrosine recognition loop and includes residues from the second phosphate binding site in PTP1B. This potential binding area is roughly defined by residues of the WPD-loop, the pTyr recognition loop and the loop containing residues 28-32.

Compounds having Mode 1 activity include compounds that can make polar interactions at the second binding site with one or more of:

(44) Arg24

(14) Gln262

(45) Arg254

(46) Asn 44

(5) Arg47

(4) Tyr46

(1) Lys 41

(47) Lys36

(48) Asp29

(49) Cys32

(50) Ser50

The compounds can make hydrophobic interactions with one or more of:

(51) Leu250

(14) Gln262

(41) Met258

(35) Val49

(4) Tyr46

(39) Gly218

(52) Gly259

(53) Phe52

(42) Leu260

(54) Leu261

(55) Ala35 and

(56) the backbone of Asp48.

In the middle of this binding area a third potential binding site (hereinafter referred to as “the third binding site”) has been created as a result of the distortion of the phosphate-binding cradle. This water filled cavity is located directly under the distorted phosphate-binding cradle and has a narrow entrance between residues Val49, Gly218 and Gln 262. The cavity walls are formed by Asp48, Val49, Leu83, Gly218, Gly220, Ser222, Arg257, Gly259, Gln262 and the sulfenyl-amide.

Accordingly, compounds having Mode 1 activity include compounds that can make polar interactions at the third binding site with one or more of:

(3) Arg45

(29) Asp48

(30) Ser222

(31) Arg257

(14) Gln262

(33) the protein backbone of one or more of (i) Thr84, (ii) Gly218, (iii) Gly220, (iv) Gly223, (v) Met258, (vi) and Gly259;

(34) and the sulfenyl-amide residue.

Preferably the compounds can make polar interactions at two or more (more preferably three or more, four or more, or five or more) of the residues (3), (29) to (31), (14), (33) and (34).

Hydrophobic interactions with the compound can be made at the third binding site by:

(35) Val49

(36) Leu83

(37) Gln85

(38) Gly86

(39) Gly218

(40) Gly220

(41) Met258

(42) Leu260 and

(43) the main chain of His214.

Preferred interactions between the compounds of the invention and the three binding sites described above are as follows:

First Binding Site:

Polar: Trp79, Arg221, Lys46, Glu266, Arg45, Ser118

Hydrophobic: Ile219, Leu88, Ile120

Second Binding Site:

Polar: Arg254, Lys36, Asp29, Gln262

Hydrophobic: Met258, Val49

Third Binding Site (cavity):

Polar: Arg257, Asp48, Ser222

Hydrophobic: sulfenyl amide, Val 49, Leu83

As described above, compounds useful in the invention include those that bind to the sulfenyl amide PTP in the region of the sulfenyl amide moiety thereby to prevent reduction or other reaction of the sulfenyl amide with an endogenous intracellular molecule such as glutathione and conversion back to the active form of the PTP. However, in an alternative embodiment of the invention, the compounds can be ligands that possess a nucleophilic functional group that can react either reversibly (Mode 2) or irreversibly (Mode 3) with the electrophilic sulfenyl amide active. Scheme 1 below illustrates how ligands inhibit the action of the protein in the cell by preventing it from being converted back to an active form.

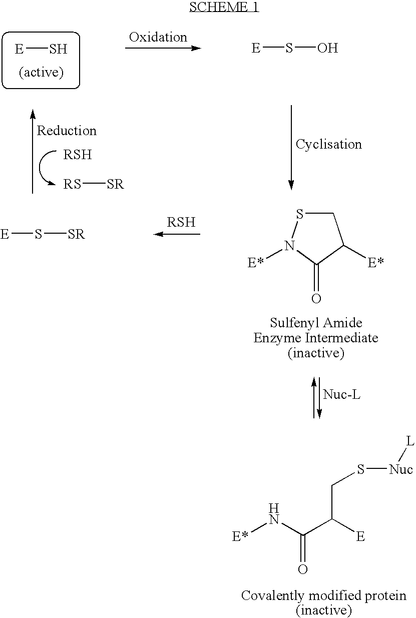

The compounds of the invention can thus take the form of nucleophilic ligands, having a nucleophilic group that will react with the sulfenyl amide moiety, and a binding region for binding to the sulfenyl amide PTP in the region of the sulfenyl amide moiety. The binding region can be one that exhibits one or more of the polar and non-polar interactions 1 to 56 set out above in relation to Mode 1 compounds.

The nucleophilic group will typically contain a heteroatom (e.g. selected from nitrogen, sulphur, oxygen and phosphorus) that is either neutral or negatively charged, and which may be located adjacent a carbon atom or another heteroatom, which is capable of reacting with the sulfenyl amide species. Nitrogen, oxygen and sulfur nucleophiles are preferred.

Thus, the sulfenyl amide moiety can be modified by reaction with a nucleophilic ligand to prevent it from reverting to the active form of the enzyme.

The nucleophilic group is selected from the group consisting of a thiol, disulfane, primary thioamide, secondary thioamide, primary thiourea, secondary thiourea, primary amine, secondary amine, primary hydrazine, secondary hydrazine, primary hydrazide, secondary hydrazide, primary hydrazone, secondary hydrazone, primary amide, secondary amide, primary urea, secondary urea, primary sulfonamide, secondary sulfonamide, 5-membered ring heterocycle containing NH, alcohol, hydroxylamine, oxime, hydroxamic acid, carboxylic acid (preferably other than an oxalamic acid), sulfoxide, sulfate and a nitrone.

Particular examples of nucleophiles are set out in Table 3 below.

Table of Nucleophiles

TABLE 3

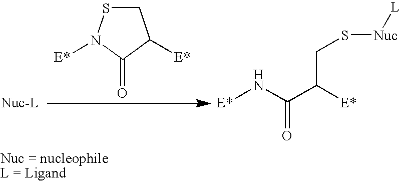

| Type of nucleophile | Structure | Name |
|---|---|---|
| Sulphur | L—SH | Thiol |
| | L—S—SH | Disulfane |
| | 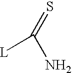 | Primary Thioamide |
| | 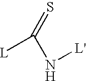 | Secondary Thioamide |
| | 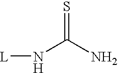 | Primary thiourea |
| | 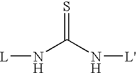 | Secondary thiourea |
| Nitrogen | L—NH₂ | Primary amine |
| | 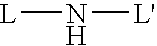 | Secondary amine |
| | 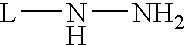 | Primary Hydrazine |
| | 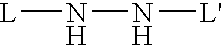 | Secondary Hydrazine |
| | 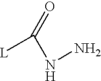 | Primary Hydrazide |
| | 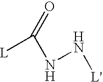 | Secondary Hydrazide |
| | L═N—NH₂ | Primary Hydrazone |
| | 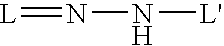 | Secondary Hydrazone |
| | 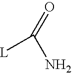 | Primary amide |
| | 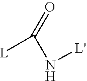 | Seoncary amide |
| | 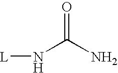 | Primary urea |
| | 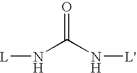 | Secondary urea |
| | 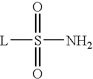 | Primary Sulfonamide |
| | 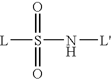 | Secondary Sulfonamide |
| | 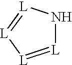 | 5-membered ring heterocycle containing NH |
| Oxygen | L—OH | Alcohol |
| | 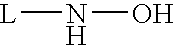 | Hydroxylamine |
| | L═N—OH | Oxime |
| | 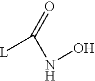 | Hydroxamic acid |
| | 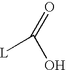 | Carboxylic acid (preferably not oxalamic acids) |
| | L—S⁺—O⁻ | Sulfoxide |
| | 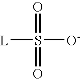 | Sulfate |
| | L═N⁺—O⁻ | Nitrone |

In the table, the symbols L and L′ represents the residue of the ligand, other than the nucleophilic group. The residue may of course contain one or more further nucleophilic groups of the type shown.

Compounds of the invention that are nucleophilic ligands will form new covalently bound protein-ligand species. In some cases (Mode 2), the protein-ligand species is capable of undergoing the reverse reaction to reform the sulfenyl amide. In other cases (Mode 3), the nucleophiles will form a covalently bound protein-ligand complex in which the reverse reaction does not occur in the environment of the active site, or occurs very slowly, so that the complex is formed irreversibly. Additionally, certain covalent protein-ligand complexes, formed by reaction of nucleophilic ligands with the sulfenyl amide protein, may undergo additional reactions that prevent the reverse reaction from occurring, resulting in irreversible inhibition.

For example, where the nucleophilic heteroatom is an oxygen atom, the resulting covalent protein-ligand complex will contain a sulfur oxygen bond which is therefore oxidised. Subsequent further oxidation under cellular conditions could lead to oxidation of the protein to sulfinyl or sulfonic acid oxidation states, irreversibly modifying the protein.

The compounds of the invention are typically synthetic compounds that are not normally encountered in a cellular environment, although naturally occurring compounds derived from plant sources, marine sources or other non-mammalian sources may be used where appropriate.

The compounds of the invention are typically organic compounds and can be non-peptides, peptides or modified peptides. In one embodiment, the compounds are not peptides.

The compounds of the invention may comprise a scaffold formed from one or more optionally substituted carbocyclic or heterocyclic ring systems, the ring systems and/or the substituents having one or more polar or non-polar moieties for interacting with one or more, preferably a plurality of the binding sites 1 to 43 listed above.

The carbocyclic and heterocyclic ring systems can be aromatic or non-aromatic ring systems. When the carbocyclic or heterocyclic groups are aryl or heteroaryl groups, they can have, for example, from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. The aryl or heteroaryl groups can be monocyclic or bicyclic groups and can be unsubstituted or substituted with one or more substituents. Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl groups include but are not limited to pyridyl, pyrrolyl, furanyl, thiophenyl, imidazolyl, oxazolyl, oxadiazolyl, oxatriazolyl, isoxazolyl, thiazolyl, isothiazolyl, pyrazolyl, pyrazinyl, pyrimidinyl, triazinyl, triazolyl, tetrazolyl, quinolinyl, isoquinolinyl, benzfuranyl, benzthiophenyl, chromanyl, thiochromanyl, benzimidazolyl, benzoxazolyl, benzisoxazole, benzthiazolyl and benzisothiazole, isobenzofuranyl, isoindolyl, indolizinyl, indolinyl, isoindolinyl, purinyl (e.g., adenine, guanine), indazolyl, benzodioxolyl, chromenyl, isochromenyl, chroman, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl.

Examples of carbocyclic aryl groups include phenyl, naphthyl, indenyl, and tetrahydronaphthyl.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur. The heterocylic groups can contain, for example, cyclic ether moieties (e.g as in tetrahydrofuran and dioxane), cyclic thioether moieties (e.g. as in tetrahydrothiophene), cyclic amine moieties (e.g. as in pyrrolidine), cyclic sulphones (e.g. as in sulfolane and sulfolene)), cyclic sulphoxides, cyclic sulphonamides and combinations thereof.

Particular examples include morpholine, piperidine, pyrrolidine, pyrrolidone, tetrahydrofuran, tetrahydrothiophene, dioxan, tetrahydropyran, imidazoline, imidazolidinone, oxazoline, thiazoline, piperazine, and N-alkyl piperazines such as N-methyl piperazine. In general, preferred non-aromatic heterocyclic groups include tetrahydrofuran, morpholine, piperazine, piperidine, pyrrolidine and pyrrolidone.

The carbocyclic and heterocyclic groups can each be unsubstituted or substituted by one or more substituent groups selected from halogen, hydroxy, trifluoromethyl, cyano, nitro, carboxy, amino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}R^{d}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} and R^{d} are the same or different and each is hydrogen or C₁₋₄ hydrocarbyl;
X¹ is O, S or NR^{c} and X² is ═O, ═S or ═NR^{c}.

Where the substituent group comprises or includes a carbocyclic or heterocyclic group, the said carbocyclic or heterocyclic group may be unsubstituted or may itself be substituted with one or more further substituent groups. In one sub-group of compounds of the formula (1), such further substituent groups may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of the substituents.

Examples of halogen substituents include fluorine, chlorine, bromine and iodine. Fluorine and chlorine are particularly preferred.

In the definition of the compounds above and as used hereinafter, the term “hydrocarbyl” is a generic term encompassing aliphatic, alicyclic and aromatic groups having an all-carbon backbone, except where otherwise stated. Examples of such groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or substituted by one or more substituents as defined herein. The examples and preferences expressed below apply to each of the hydrocarbyl substituent groups or hydrocarbyl-containing substituent groups referred to in the various definitions of substituents for compounds of the invention unless the context indicates otherwise.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers.

Examples of cycloalkyl groups are those derived from cyclopropane, cyclobutane, cyclopentane, cyclohexane and cycloheptane.

Examples of alkenyl groups include, but are not limited to, ethenyl(vinyl), 1-propenyl, 2-propenyl(allyl), isopropenyl, butenyl, buta-1,4-dienyl, pentenyl, and hexenyl.

Examples of cycloalkenyl groups include, but are not limited to, cyclopropenyl, cyclobutenyl, cyclopentenyl, cyclopentadienyl and cyclohexenyl.

Examples of alkynyl groups include, but are not limited to, ethynyl and 2-propynyl(propargyl) groups.

Examples of carbocyclic aryl groups include substituted and unsubstituted phenyl.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

The definition “R^{a}-R^{b}” as used herein, includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂NR^{c}R^{d}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined.

Examples of hydrocarbyl, carbocyclic and heterocyclic groups are as set out above.

When present, the hydrocarbyl group can be substituted by one or more substituents selected from hydroxy, oxo, halogen, cyano, nitro, amino, mono- or di-C₁₋₄ hydrocarbylamino, and monocyclic carbocyclic and heterocyclic groups having from 3 to 12 (typically 3 to 10 and more usually 5 to 10) ring members. Preferred substituents include halogen such as fluorine. Thus, for example, the substituent can be a partially fluorinated or perfluorinated group such as trifluoromethyl.

One or more carbon atoms of the C₁₋₈ hydrocarbyl group may optionally be replaced by O, S, SO, SO₂, NR^{c}, X¹C(X²), C(X²)X¹ or X¹C(X²)X¹ wherein X¹ and X² are as hereinbefore defined. For example, 1, 2, 3 or 4 carbon atoms of the hydrocarbyl group may be replaced by one of the atoms or groups listed, and the replacing atoms or groups may be the same or different. Examples of groups in which a carbon atom of the hydrocarbyl group has been replaced by a replacement atom or group as defined above include ethers and thioethers (C replaced by O or S), amides, esters, thioamides and thioesters (C replaced by X¹C(X²) or C(X2)X¹), sulphones and sulphoxides (C replaced by SO or SO₂) and amines (C replaced by NR^{c}).

The combination of ring system and substituents is chosen so as to give a desired level of interaction with residues (1) to (56) in the three binding sites defined above. The extent of the interaction between the compound and the binding sites of the PTP sulfenyl amide can be gauged using the computer based modelling methods discussed above based on the atomic coordinates set out in Table 1 or Table 2.

In general, the number of interactions between the compound and the PTP sulfenyl amide may be chosen so as to optimise the binding of the compound to the PTP sulfenyl amide. For Mode 1 inhibitor compounds, in one embodiment, it is preferred to maximise the number of interactions between the compound and the first and/or second and/or third binding sites so as to provide enhanced binding to the PTP sulfenyl amide.

For example, where the compound is designed to bind to the first binding site, it is preferred that the compound makes polar interactions with at least seven, more usually at least ten, and preferably at least twelve of the residues (1) to (17) and hydrophobic interactions with at least two and more preferably at least four of the residues (18) to (27).

Where the compound is designed to bind to the second binding site, it is preferred that the compound forms polar interactions with at least two, more usually three, and preferably four of the residues (44), (14), (45), (46), (5), (4), (1), (47), (48), (49) and (50), and hydrophobic interactions with at least one or two of the residues (51), (14), (41), (35), (4), (39), (52), (53), (42), (54), (55) and (56).

Where the compound is designed to bind to the third binding site, it is preferred that the compound forms polar interactions with at least two, more usually three, and preferably at least four of the residues (3), (29), (30), (31), (14), (33) and (34), and preferably at least two and more usually at least three hydrophobic interactions with the residues (35), (36), 37), (38), (39), (40), (41), (42) and (43).

In one embodiment of the invention, the compound forms interactions at two or more of the first, second and third binding sites, for example, (i) the first and second binding sites, or (ii) the first and third, or (iii) the second and third binding sites.

In another embodiment, the compound forms interactions with only the first binding site.

In a further embodiment, the compound forms interactions with only the second binding site.

In a further embodiment, the compound forms interactions with only the third binding site.

In a still further embodiment, the compound forms interactions with all three binding sites.

A compound is considered to have formed an interaction with a given residue at a binding site if the proximity between a compound or portions thereof to the molecule or portions thereof wherein the juxtaposition is energetically favored by electrostatic or van der Waals interactions. The distance will depend on the type of interaction made-hydrogen bond, salt bridge or stacking interaction

The term hydrogen bond refers to a favorable interaction that occurs whenever a suitable donor atom, Q^{X}, bearing a proton, H, and a suitable acceptor atom, Q^{Y}, have a separation of <3.5 Å and where the angle Q^{X}-H-Q^{Y} is greater than 90 degrees. Sometimes, a single proton on a donor atom Q^{X} may form a plurality of suitable acceptor atoms, Q^{Y}. For example, the proton on a —NH-group may form a separate hydrogen bond with each of the two oxygen atoms in a carboxylate anion. Suitable donor and acceptor atoms are well understood in medicinal chemistry (G. C. Pimentel and A. L. McClellan, The Hydrogen Bond, Freeman, San Francisco, 1960; R. Taylor and 0. Kennard, Hydrogen Bond Geometry in organic Crystals, Accounts of Chemical Research, 17, pp. 320-326 (1984)).

The term “hydrogen bonding moiety” refers to a chemical structure containing one or more suitable hydrogen bond donor moieties or hydrogen bond acceptor moieties.

The term “hydrogen bonding donor moiety” refers to a chemical structure containing a suitable hydrogen bond donor atom bearing one or more protons. Examples of donor atoms having one proton are —OH, —SH and —NH—.

Examples of donor atoms having more than one proton are —NH₂, [—NH₃]— and [—NH₂¹⁺

The term hydrogen bonding acceptor moiety refers to a chemical structure containing a suitable hydrogen bond acceptor atom. Examples of acceptor atoms include fluorine, oxygen, sulfur and nitrogen.

The term stacking interaction refers to the favorable attractive interactions between two aromatic ring systems, wherein the two rings are juxtaposed such that they are oriented either face-to-face, perpendicular or at an intermediate angle to each other.

Face-to-face stacking interactions are usually between 3.5-4.5 Ångstrom. Face-edge stackings are usually to be between 3.5 and 4 Å. Most aromatic protein interactions involve separation distances of 3.6 to 3.8 Ångstrom. Fully stacked interactions are not usually observed. Most common are staggered stacked structures with tilted rings. Perpendicular stacking may be face-edge or cogwheel. Both are common. (Protein-protein recognition via side-chain interactions; Thornton et al.; Biochemical society transactions.; 927-930 (1988)).

The term salt bridge refers to the non-covalent attractive interaction between a positively charged moiety (P) and a negatively charged moiety (N) when the distance between the centers of mass of P and N is between 2 and 6 Angstroms. In calculating the center of mass, atoms which may contain a formal charge and atoms immediately adjacent to these are included. For example, a salt bridge may be formed between the positively charged guanidinium side chain of an arginine residue and the negatively charged carboxylate side chain of a glutamate residue. Salt bridges are well known in medicinal chemistry (L. Stryer, Biochemistr, Freeman, San Francisco, (1975); K. A. Dill, Dominant Forces in Protein Folding, Biochemistry, 29, No. 31, pp. 7133-7155, (1990)).

The term center of mass refers to a point in three-dimensional space that represents a weighted average position of the masses that make up an object.

In order to form a desired number of binding interactions with the first binding site, the compound preferably has a binding domain no longer than about 35 Ångstrom long (the length of the first binding groove). Typically compounds will be, for example, 5-30, 5-25, 5-15, 10-15 Å in length.

In order to form a desired number of binding interactions with the second binding site, the compound preferably has a binding domain that can fit into an area of about 30 by 30 Ångstrom (the area of the second binding site). In order optimise interactions at this binding site, compounds will typically be, for example, 5-30, 5-25, 5-15, 10-15 Å in length.

The third binding site, the cavity underneath the cradle, is about 9 by 15 Ångstrom so only small binding domains or small molecules will fit in this cavity, for example those that are 5-15, 10-15 Å in length.

A compound of the invention can possess only a single binding domain or can have binding domains enabling it to bind to two or three binding sites.

Compounds exhibiting Type 2 or Type 3 activity typically have a binding domain enabling them to bind to the first and/or second and/or third binding sites so as to bring the nucleophilic group into reactive proximity of the sulfenyl amide group. The nucleophilic groups can form part of the scaffold described above or can take the form of substituents attached to the scaffold.

Screening of Compounds

In another aspect, in place of in silico methods, high or low throughput screening of compounds to select compounds with binding activity may be undertaken, and those compounds which show binding activity may be selected as possible candidate modulators, and further crystallized with PTP1B sulfenyl amide (e.g. by co-crystallization or by soaking) for X-ray analysis. The resulting X-ray structure may be compared with that of Table 1 or Table 2 for a variety of purposes. The screen may utilise any of the assays detailed below.

Assays for Screening for Active Compounds

Compounds may be identified in high-throughput or low-throughput screening as outlined above, utilizing the assays detailed below. Compounds screened may include those available from commercially available sources, compounds generated by standard synthetic chemistry methods, or those that are part of a corporate compound collection.

Alternatively, once a candidate inhibitor compound has been identified, for example by computer based rational drug design techniques as described above, the compounds are synthesized and tested. Whether or not the compounds are inhibitors of the PTP sulfenyl amide can be determined by one of a number of assays. Consequently, all the methods of compound design and identification above (e.g. in silico analysis, ligand-sulfenyl amide PTP structure determination etc) preferably further comprise the further steps of:
obtaining or synthesising the candidate modulator; and
contacting the candidate modulator with PTP1B sulfenyl amide to determine the ability of the candidate modulator to interact with PTP1B sulfenyl amide.

For example, in one assay, the oxidized form (sulfenyl amide form) of a recombinant or extracted protein tyrosine phosphatase is incubated with a candidate binding compound and a determination is made as to whether the compound is able to interact with the oxidized (sulfenyl amide) form of the protein tyrosine phosphatase.

Such assays require the formation of the oxidized form of a protein tyrosine phosphatase. The oxidized form can be produced by incubating the protein tyrosine phosphatase in the presence of oxidizing agents such as a reactive oxygen species in a cellular environment ^{24, 25}, organic peracids e.g. MCPBA, peroxides e.g. hydrogen peroxide ^{24,25} or compound(s) as described in the examples below.

The assay can be a binding assay. Such a binding assay can be competitive or non-competitive and can accommodate the screening of a large number of compounds to determine if the compounds are capable of binding to the oxidized protein tyrosine phosphatase. Subsequently other assays can be carried out with compounds found to bind to determine the mode of binding of these compounds.

Alternatively, the assay can be a functional assay that identifies compounds that trap the oxidized form of the protein tyrosine phosphatase and so change its ability to regain its functional activity on reduction. Such an assay can involve incubating potential trapping compounds with the oxidized form of the protein tyrosine phosphatase and determining if protein tyrosine phosphatase activity can be regained upon reduction.

In a further alternative, the assay can be cell-based assay for identifying compounds which modulate the cell-based activity of a protein tyrosine phosphatase, through binding to the oxidized form of protein tyrosine phosphatases in cells.

Particularly preferred types of assays include binding assays, functional assays and cell-based assays, which may be performed as follows:

Binding Assays

Purified, oxidized protein tyrosine phosphatases can be used for binding studies. Oxidized protein tyrosine phosphatase can be used in conventional filter-binding assays or in a high throughput scintillation proximity-type binding assay to detect binding of a radio-labelled ligand and its displacement by compounds which compete for the binding site. Radioactivity can be measured with a Packard Topcount or similar instrumentation capable of making rapid measurements for 96-, 384- or 1536-well microtitre plate formats.

Binding to oxidized protein tyrosine phosphatases could also be measured using a fluorescently labelled ligand, which could be displaced by compounds, competing for the binding site. Binding could be detected by fluorescent polarisation methods using an instrument such as the Packard Fusion reader to monitor fluorescence in 96-, 384 or 1536-well microtitre plate formats.

Another method for studying the binding of compounds to the oxidized protein tyrosine phosphatase makes use of a surface plasmon resonance effect, measured by a Biacore instrument. Oxidized protein tyrosine phosphatase could be attached to the biosensor chip of a Biacore and binding of test compounds could be monitored. Examples of the use of the surface plasmon resonance effect may be found in Parsons et al (1995) Nucleic Acids Res. 23, 211-216 and Parsons et al (1997) Anal. Biochem. 254(1), 82-87.

Binding of test compounds to an oxidized protein tyrosine phosphatase could also be monitored using NMR techniques. The difference between NMR spectra of a test compound with and without the oxidized protein tyrosine phosphatase could be analysed to determine if the compound bound the protein tyrosine phosphatase. Competition between test compounds and a known ligand for a binding site on the protein tyrosine phosphatase could also be monitored in this way.

Preferred compounds are those that have a Kd value of less than 1 mM, more preferably less than 1 uM and most preferably less than 100 nM. The term “Kd” is used herein in its normal sense to mean the dissociation constant, which describes the ratio of the concentrations at equilibrium between the free individual components and the complex formed. For a complex between two components A and B, Kd=[A][B]/[AB].

Functional Assays

The oxidized form of a protein tyrosine phosphatase is inactive, but can be reactivated by reduction. An example of a functional assay to monitor compounds that trap the oxidized form of the protein tyrosine phosphatase could involve measuring the time taken for an oxidized form of a protein tyrosine phosphatase to regain activity in the presence of the candidate inhibitor under reducing conditions.

Candidate trapping compounds could then be screened against the oxidized form of the enzyme, by incubating candidate inhibitors and enzyme for a period of time and then adding a reducing agent such as DTT or glutathione. The activity of the enzyme could then be monitored at time intervals after this addition of reducing agent. Enzyme which has been trapped in the oxidized form by candidate inhibitors should take longer to regain activity than enzyme that has not been trapped. Time taken to regain activity could be measured against controls containing no test compound and so correlated to potency of the inhibitor.

Assays for monitoring the activity of protein tyrosine phosphatases have been described in the literature and can use known substrates such as p-nitrophenyl phosphate or phosphorylated peptides (Hoppe et al., 1994, Eur. J. Biochem., 223, 1069-1077; Bleasdale et al., 2001, Biochemistry, 40, 5642-5654; Wang et al., 1999, Biochim Biophys Acta, 1431, 14-23). Assays can be performed in 96- or 384-well microtitre format using a Molecular Devices Spectramax plate reader, allowing screening of a large number of compounds. The dephosphorylation of p-nitrophenyl phosphate by a PTP can be monitored be an increase in absorbance at 405 nm. The dephosphorylation of a phosphorylated peptide can be monitored by measuring phosphate release by the malachite green method.

Cell-Based Assays

Compounds can also be screened in cell-based assays, specific to the protein tyrosine phosphatase of interest.

Compounds that affect PTP1B can be screened using assays that monitor the effects of insulin on cells. Examples of such assays are as follows:

Cells such as 3T3-L1 can be differentiated into adipocytes and induced to be insulin resistant. Effect of compounds on glucose transport into these cells can be monitored by measuring the rate of uptake of 2-[³H]deoxyglucose when the cells are stimulated by insulin.

Another cell-based assay that can be used to monitor effects of compounds on ptp1b is an assessment of insulin receptor tyrosine kinase activity. In this case the tyrosine kinase activity of the insulin receptor captured from cells, e.g. 3T3-L1, treated with test compounds is measured. Tyrosine kinase activity can be measured using a peptide substrate and [γ³³P]-ATP.

A further assay involves monitoring the tyrosine phosphorylation of insulin signalling molecules in cells that have been treated with insulin and test compounds. Phosphorylation of molecules can be detected for example by Western blotting cell extracts using monoclonal antibodies (Bleasdale et al., 2001, Biochemistry, 40, 5642-5654).

Preferred compounds of the invention are those that have IC₅₀ values in a cellular assay of less than 1 uM, more preferably less than 100 nM and most preferably less than 10 nM.

Pharmaceutical Formulations

The compounds of the invention can be presented in the form of pharmaceutical compositions.

In another aspect, therefore, the invention provides a pharmaceutical composition comprising a compound that binds to protein tyrosine phosphatase sulfenyl amide to prevent or inhibit conversion of the protein tyrosine phosphatase sulfenyl amide to an active reduced form of the protein tyrosine phosphatase.

The pharmaceutical compositions can be in any form suitable for oral, parenteral, topical, intranasal, intra-articular, ophthalmic, otic, rectal, intra-vaginal, or transdermal administration. Where the compositions are intended for parenteral administration, they can be formulated for intravenous, intramuscular or subcutaneous administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the invention can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a celluloses or derivative thereof such as methyl cellulose, ethyl cellulose, hydroxypropyl methyl cellulose, and starches such as corn starch. Tablets may also contain such standard ingredients as binding and granulating agents agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the invention will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams. The active compound will be administered to a patient in need thereof (for example a human or animal patient) in an amount sufficient to achieve the desired therapeutic effect.

In another aspect, the invention provides a method of preparing a composition comprising (a) identifying a the PTP sulfenyl amide modulator molecule (which may thus be termed a lead compound) by a method of any one of the other aspects of the invention disclosed herein and admixing the molecule with a carrier.

Also provided is a method of preparing a composition comprising (a) identifying a the PTP sulfenyl amide modulator molecule (which may thus be termed a lead compound) by a method of any one of the other aspects of the invention disclosed herein and admixing an optimised structure of the modulator molecule with a carrier.

The invention further provides a process for producing a medicament, pharmaceutical composition or drug, the process comprising: (a) identifying a the PTP sulfenyl amide modulator molecule (which may thus be termed a lead compound) by a method of any one of the other aspects of the invention disclosed herein; and (b) preparing a medicament, pharmaceutical composition or drug containing the modulator molecule.

A further aspect of the present invention provides a method for preparing a medicament, pharmaceutical composition or drug, the method comprising: (a) identifying a the PTP sulfenyl amide modulator molecule (which may thus be termed a lead compound) by a method of any one of the other aspects of the invention disclosed herein; (b) optimising the structure of the modulator molecule; and (c) preparing a medicament, pharmaceutical composition or drug containing the optimised modulator molecule.

The above-described processes of the invention may be iterative in that the modified compound may itself be the basis for further compound design. Detailed structural information can be obtained about the binding of the candidate modulator to PTP sulfenyl amide, and in the light of this information adjustments can be made to the structure or functionality of the candidate modulator, e.g. to improve binding to the binding cavity or cavities. The above steps may be repeated and re-repeated as necessary.

By “optimising the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds.

Modifications typically will be those conventional in the art known to the skilled medicinal chemist, and will include, for example, substitutions or removal of groups containing residues which interact with the amino acid side chain groups of a the PTP sulfenyl amide structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Methods of Treatment

It is envisaged that the compounds of the invention will be useful in the prophylaxis or treatment of a range of disease states or conditions mediated by protein tyrosine phosphatases. Examples of such disease states and conditions are set out above and include the treatment of cancers, diabetes (diabetes type I and II) obesity, autoimmune diseases, acute and chronic inflammation, rheumatoid arthritis, osteoporosis, proliferative disorders including various forms of cancer, growth disorders and hypertension

Compounds of the invention are generally administered to a subject in need of such administration, for example a human or animal patient, preferably a human. The compounds will typically be administered in amounts that are therapeutically or prophylactically useful and which generally are non-toxic. However, in certain situations (for example in the case of life threatening diseases), the benefits of administering a compound of the invention may outweigh the disadvantages of any toxic effects or side effects, in which case it may be considered desirable to administer compounds in amounts that are associated with a degree of toxicity.

A typical daily dose of the compound can be in the range from 100 picograms to 100 milligrams per kilogram of body weight, more typically 10 nanograms to 10 milligrams per kilogram of bodyweight although higher or lower doses may be administered where required. Ultimately, the quantity of compound administered will be commensurate with the nature of the disease or physiological condition being treated and will be at the discretion of the physician.

The compounds of the invention can be administered as the sole therapeutic agent or they can be administered in combination therapy with one of more other compounds for treatment of a particular disease state, for example a neoplastic disease such as a cancer as hereinbefore defined.

### EXAMPLES

The invention will now be illustrated in greater detail by reference to the specific embodiments described in the following non-limiting examples.

### Example 1

PTP1B Expression, Purification, Crystallisation and Structure Determination

Expression, purification and crystallisation of the catalytic domain of PTP1B (residues 1-321) were based on literature conditions—see Barford et al.²⁰, the entire disclosure in which is incorporated herein by reference.

PTP1B Expression, Purification and Crystallization Protocol

Using the DNA sequence of human PTP1B (Genbank nm_{—}002827), a fragment encoding the N-terminal 321 residues was generated and cloned into the expression vector Pet19b (Novagen) at the Nco1 site enabling the initiation of translation at Met1. Primers used to generate the plasmid were:

(SEQ.ID. NO: 1)

5′-TTTTCCATGGAGATGGAAAAGGAGTTCG-3′

(SEQ ID. NO: 2)

5′-TTTTCCATGGCTAATTGTGTGGCTCCAGGATTCG-3′.

E. coli b121 (de3) cells transformed with Pet19b-PTP1B were grown overnight at 37° C. in LB medium plus 100 μg ampicillin/ml. Typically, 10 mls of this overnight culture was used to inoculate 1 litre of LB plus 100 μg ampicillin/ml. Cultures were grown at 37° C. for 3 hours prior to induction by addition of isopropyl-thio-β-d-galactopyranoside (IPTG) to a final concentration of 1 mM. The cultures were grown for a further 3 hours before being harvested by centrifugation.

All purification steps were performed at 4° C. and all buffers unless stated otherwise contained a 1/1000 dilution of protease inhibitor cocktail III (Calbiochem).

Bacterial pellets were resuspended on ice in 20 mM imidazole, pH7.5, 1 mM EDTA, 3 mM DTT, 10% (v/v) glycerol and lysed by sonication (2 mins, 20 second pulses). The lysed cells were incubated with DNAase 1 (Sigma) for 10 minutes at 4° C. Following this, the lysate was clarified by centrifugation at 25,000 rpm for 30 minutes. Protein was applied to a Q-sepharose fast flow column incorporated into an Akta fplc system (Amersham Biosciences) at a flow rate of 4 ml/min. a linear salt gradient (0-0.5 m NaCl in 20 mM imidazole, pH7.5, 1 mM EDTA, 3 mM DTT, 10% (v/v) glycerol) was applied to the column and PTP1B eluted at ˜300 mM NaCl.

Fractions containing PTP1B were pooled and buffer exchanged using a 26/10 desalting column (Amersham Biosciences) pre-equilibrated in 25 mM NaH₂PO₄ pH 6.5, 1 mM EDTA, 3 mM DTT, 10% (v/v) glycerol. PTP1B fractions were applied to a 10/10 mono s column previously equilibrated in 25 mM NaH₂PO₄ pH 6.5, 1 mM EDTA, 3 mM DTT, 10% (v/v) glycerol. Protein fractions were eluted by applying a linear salt gradient (0-0.5 m NaCl) to the column with PTP1B eluting at ˜75 mM NaCl. PTP1B was buffer exchanged into 10 mM Tris pH7.5, 25 mM NaCl, 0.2 mM EDTA and 3 mM DTT. Protein purity was assessed by SDS PAGE and was observed to be >95% pure. This protein was subsequently concentrated to 10 mg/ml and used for crystallisation.

Using the hanging drop method (Crystallization of nucleic acids and proteins. A practical approach. A. Ducruix and R. Giege. Oxford University press 1999. and Principles of Protein X-ray Crystallography. Jan Drenth. Springer Verlag 1994.), PTP1B crystals were grown at 4° C. from 4 μl drops (1:1 ratio protein:reservoir solution) and equilibrated against 1 ml reservoir solution consisting of 12-18% (v/v) PEG 4000, 0.1 m HEPES (pH 7.5) and 0.2 M magnesium acetate.

Method for Assaying PTP1B Activity:

PTP1B enzyme activity was assayed by measuring the dephosphorylation of p-nitrophenyl phosphate to p-nitrophenol. The reaction was monitored by following the increase in absorbance at 405 nm as p-nitrophenol was produced. Standard assays typically contained 0.25 mM p-nitrophenyl phosphate substrate and 25 nM PTP1B enzyme in 50 mM HEPES, pH 6.5, 1 mM DTT, 1 mM EDTA, 0.01% CHAPS buffer. Assays were carried out in Costar 3696 half-area plates in an assay volume of 100 ul on a Spectramax plate reader (Molecular Devices). Reactions were typically monitored at 20 second intervals for 30 minutes. For kinetic measurements of km the concentration of the p-nitrophenyl phosphate substrate was varied between 31 uM and 5 mM.

Activity of PTP1B was also monitored by measuring the phosphate produced in the reaction. Standard assays were set up as described above, except that 50 ul of malachite green reagent was added to quench the reaction and the absorbance at 620 nm was then measured on a Spectramax plate reader (Molecular Devices) to determine the amount of phosphate produced. Malachite green reagent is made from 0.2% w/v malachite green and 4.2% w/v ammonium molybdate mixed in a ratio of 3:1.

For overnight soaking experiments crystals were transferred into a standard mother liquor containing 16% PEG 4000, 0.1 M HEPES (pH 7.5), 0.2 M magnesium acetate, 10 mM DTT and the compound of interest (See Table 3 below).

All data were collected at 100 K. Data sets of sulfenic- sulfinic- and sulfonic acid PTP1B derivatives were automatically collected on a Jupiter140 CCD detector mounted on a RU3HR rotating anode generator equipped with an ACTOR sample-changing robot (RigakuMSC, The Woodlands Tex., USA). They were processed and scaled using D*TREK²¹ and converted to structure factors using programs from the CCP4 suite²².

Oxidation of Cys215 to the sulfenyl-amide derivative was carried out by soaking crystals for approximately 24 hours in mother liquor without DTT, but containing 100 mM 2-phenyl-isoxazolidine-3,5-dione. Data were collected at station 14.1 (SRS, Daresbury) and processed and scaled using MOSFLM/SCALA²².

Reversibility of the sulfenyl-amide derivative was checked by first soaking two crystals in 100 mM 2-phenyl-isoxazolidine-3,5-dione for 24 hours. Sulfenyl-amide formation was confirmed by an in-house data set of one of the crystals. After back soaking for 24 hours in mother liquor containing 20 mM reduced glutathione, data collected from the other crystal confirmed the active site in its native conformation.

All results were evaluated using the graphics programs QUANTA (Accelrys, San Diego Calif., USA) and Astexviewer™²³. Data collection statistics are summarised in Table 2. Initial refinement was always carried out using the CCP4-based Astex automatic refinement scripts, followed by rounds of positional and B-factor refinement with REFMAC5²² alternated with manual rebuilding steps using QUANTA.

Monomer libraries for the different oxidation states were generated using REFMAC5 in combination with the monomer sketcher of the CCP4GUI²².

TABLE 2
Crystallographic data collection and refinement statistics

| | Sulfenic | Sulfinic | Sulfonic | Sulfenyl- | Back soaking experiment | |
|---|---|---|---|---|---|---|
| | acid | acid | acid | amide | Compound | Glutathione |
| Data collection | | | | | | |
| Beamline | In-house | In-house | In-house | SRS 14.1 | In-house | In-house |
| λ (Å) | 1.54 | 1.54 | 1.54 | 1.488 | 1.54 | 1.54 |
| Resolution (Å) | 2.3 | 2.6 | 2.2 | 2.2 | 2.4 | 2.2 |
| No. Observations | 43942 | 29624 | 53452 | 83434 | 103350 | 108793 |
| No. Unique | 20755 | 16237 | 23367 | 23591 | 18514 | 23322 |
| reflections | | | | | | |
| Completeness (%)* | 96.0 (94.9) | 95.9 (95.3) | 96.0 (86.9) | 98.9 (97.4) | 99.8 (100) | 100 (100) |
| Rmerge¹,* | 0.092 (0.25) | 0.172 (0.37) | 0.060 (0.252) | 0.050 (0.26) | 0.078 (0.273) | 0.054 (0.32) |
| I/σ<I>* | 7.3 (2.8) | 4.6 (1.9) | 11.2 (4.3) | 7.8 (2.8) | 8.4 (2.8) | 12.9 (2.2) |
| Refinement | | | | | | |
| Rcryst/Rfree | 0.187/0.248 | 0.204/0.263 | 0.202/0.242 | 0.243/0.283 | 0.215/0.275 | 0.228/0.277 |
| Rmsd Bond | 0.013 | 0.018 | 0.012 | 0.009 | 0.006 | 0.006 |
| lengths (Å) | | | | | | |
| Rmsd Bond | 1.4 | 1.7 | 1.3 | 1.4 | 1.3 | 1.3 |
| angles (°) | | | | | | |

*Numbers in parentheses indicate the highest shell values
¹Rmerge = ΣₕΣᵢ|I(h, i) − <I>(h)|ΣhSi<I>(h); I(h, i) is the scaled intensity of the ith observation of reflection h and <I>(h) is the mean value. Summation is over all measurements.
Rcryst = Σ_{hkl,work}||F_{obs}|−k|F_{calc}||/Σhkl|F_{obs}I, where Fobs and Fcalc are the observed and calculated structure factors, k is a weighting factor and work denotes the working set of 95% of the reflections used in the refinement.
Rfree = Σ_{hkl,test}||F_{obs}|−k|F_{calc}||/Σₕₖₗ|F_{obs}|, where Fobs and Fcalc are the observed and calculated structure factors, k is a weighting factor and test denotes the test set of 5% of the reflections used in cross validation of the refinement.
λ refers to wavelength,
Rmsd to root mean square deviations.

TABLE 3
Compounds Used in Soaking Experiments
Structure of 2-Phenyl-issoxazolidine-3,5-dione:

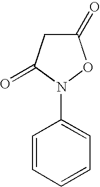

Composition of cocktails used in soaking experiments:
Cocktail composition used in the soaking experiment resulting in the
sulfenic acid (Cys-OH) PTP1B derivative:

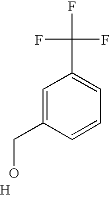

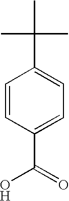

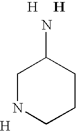

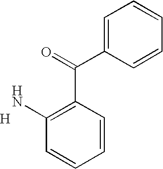

Cocktail composition used in the soaking experiment resulting in the
sulfunic acid (Cys-SO2H) PTP1B derivative:

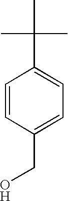

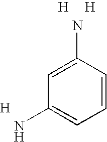

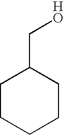

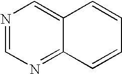

Cocktail composition used in the soaking experiment resulting in the
sulfonic acid (Cys-SO3H) PTP1B derivative:

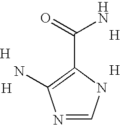

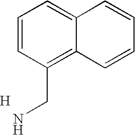

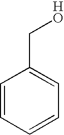

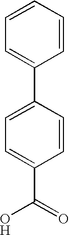

Pharmaceutical Formulations

### Example 2

(i) Tablet Formulation

A tablet composition containing a compound of the invention is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the invention with 100 mg lactose and filling the resulting mixture into standard opaque hard gelatin capsules.

Equivalents

It will readily be apparent that numerous modifications and alterations may be made to the specific embodiments of the invention described above without departing from the principles underlying the invention. All such modifications and alterations are intended to be embraced by this application.

### REFERENCES

1. Neel, B. G. & Tonks, N. K. Protein tyrosine phosphatases in signal transduction. Curr. Opin. Cell Biol. 9, 193-204 (1997).

2. Zhang, Z. Y. Protein tyrosine phosphatases: structure and function, substrate specificity, and inhibitor development. Annu. Rev. Pharmacol. Toxicol. 42, 209-234 (2002).

3. Denu, J. M. & Tanner, K. G. Specific and reversible inactivation of protein tyrosine phosphatases by hydrogen peroxide: evidence for a sulfenic acid intermediate and implications for redox regulation. Biochemistry 37, 5633-5642 (1998).

4. Denu, J. M. & Dixon, J. E. Protein tyrosine phosphatases: mechanisms of catalysis and regulation. Curr. Opin. Chem. Biol. 2, 633-641 (1998).

5. Meng, T. C., Fukada, T. & Tonks, N. K. Reversible oxidation and inactivation of protein tyrosine phosphatases in vivo. Mol. Cell 9, 387-399 (2002).

6. Lee, S. R., Kwon, K. S., Kim, S. R. & Rhee, S. G. Reversible inactivation of protein-tyrosine phosphatase 1B in A431 cells stimulated with epidermal growth factor. J. Biol. Chem. 273, 15366-15372 (1998).

7. Elchebly, M. et al. Increased insulin sensitivity and obesity resistance in mice lacking the protein tyrosine phosphatase-1B gene. Science 283, 1544-1548 (1999).

8. Klaman, L. D. et al. Increased energy expenditure, decreased adiposity, and tissue-specific insulin sensitivity in protein-tyrosine phosphatase 1B-deficient mice. Mol. Cell. Biol. 20, 5479-5489 (2000).

9. Goldstein, B. J. Protein-tyrosine phosphatase 1B (PTP1B): a novel therapeutic target for type 2 diabetes mellitus, obesity and related states of insulin resistance. Curr. Drug. Targets. Immune. Endocr. Metabol. Disord. 1, 265-275 (2001).

10. Barford, D. Protein phosphatases. Curr. Opin. Struct. Biol. 5, 728-734 (1995).

11. Pannifer, A. D., Flint, A. J., Tonks, N. K. & Barford, D. Visualization of the cysteinyl-phosphate intermediate of a protein-tyrosine phosphatase by x-ray crystallography. J. Biol. Chem. 273, 10454-10462 (1998).

12. Barford, D., Flint, A. J. & Tonks, N. K. Crystal structure of human protein tyrosine phosphatase 1B. Science 263, 1397-1404 (1994).

13. Jia, Z., Barford, D., Flint, A. J. & Tonks, N. K. Structural basis for phosphotyrosine peptide recognition by protein tyrosine phosphatase 1B. Science 268, 1754-1758 (1995).

14. Lohse, D. L., Denu, J. M., Santoro, N. & Dixon, J. E. Roles of aspartic acid-181 and serine-222 in intermediate formation and hydrolysis of the mammalian protein-tyrosine-phosphatase PTP1. Biochemistry 36, 4568-4575 (1997).

15. Sarmiento, M., Zhao, Y., Gordon, S. J. & Zhang, Z. Y. Molecular basis for substrate specificity of protein-tyrosine phosphatase 1B. J. Biol. Chem. 273, 26368-26374 (1998).

16. Scapin, G., Patel, S., Patel, V., Kennedy, B. & Asante-Appiah, E. The structure of apo protein-tyrosine phosphatase 1B C215S mutant: more than just an S→O change. Protein Sci. 10, 1596-1605 (2001).

17. Mahadev, K., Zilbering, A., Zhu, L. & Goldstein, B. J. Insulin-stimulated hydrogen peroxide reversibly inhibits protein-tyrosine phosphatase 1b in vivo and enhances the early insulin action cascade. J. Biol. Chem. 276, 21938-21942 (2001).

20. Barford, D., Kelly, J., Flint, A. J. & Tonks, N. K. Purification and crystallization of the catalytic domain of human protein tyrosine phosphatase 1B expressed in Escherichia coli. J Mol. Biol. 239, 726-730 (1994).

21. Pflugrath, J. W. The finer things in X-ray diffraction data collection. Acta Crystallogr D55, 1718-1725 (1999).

22. Collaborative Computational Project, N.4. The CCP4 suite: programs for protein crystallography. Acta Crystallogr. D50, 760-763 (1994).

23. Hartshorn, M. J. A Visualisation Aid for Structure-Based Drug Design. J. Computer-Aided Mol. Design in press (2003).

24. Meng, T.-C., Fukada, T., Tonks, N. K.; Reversible oxidation and inactivation of protein tyrosine phosphatases in vivo.; Mol Cell. February 2002;9(2):387-99.

25. Lee S R, Kwon K S, Kim S R, Rhee S G.; Reversible inactivation of protein-tyrosine phosphatase 1B in A431 cells stimulated with epidermal growth factor; J Biol Chem. Jun. 19, 1998;273(25):15366-72.

26. Allison W S, Benitez L V, Johnson C L. The formation of a protein sulfenamide during the inactivation of the acyl phosphatase activity of oxidized glyceraldehyde-3-phosphate dehydrogenase by benzylamine. Biochem Biophys Res Commun. 1973; 52(4); 1403-9.

27. Organoselenium chemistry. Redox chemistry of selenocysteine model systems, Hans J. Reich, Craig P. Jasperse; J. Am. Chem. Soc.; 1987; 109(18); 5549-5551.

The disclosures of all papers, articles and documents referred to in this application, including the papers and articles numbered 1 to 27 above, are incorporated herein by reference in their entirety.

TABLE 1
Unit cell dimensions:
a = 87.686 Å, b = 87.686 Å, c = 103.721 Å, α = 90.00°,
β = 90.00°, γ = 120.00°
Space group:
P3₁ 2 1
Table 1

| | | | | | | | | | | |
|---|---|---|---|---|---|---|---|---|---|---|
| ATOM | 1 | N | GLU | A | 2 | 17.300 | 16.060 | 47.467 | 1.00 | 63.20 |
| ATOM | 2 | CA | GLU | A | 2 | 18.497 | 16.936 | 47.513 | 1.00 | 61.62 |
| ATOM | 3 | C | GLU | A | 2 | 19.027 | 17.081 | 48.938 | 1.00 | 61.51 |
| ATOM | 4 | O | GLU | A | 2 | 18.389 | 16.661 | 49.905 | 1.00 | 60.93 |
| ATOM | 5 | CB | GLU | A | 2 | 18.218 | 18.300 | 46.856 | 1.00 | 61.28 |
| ATOM | 6 | CG | GLU | A | 2 | 18.055 | 19.504 | 47.780 | 1.00 | 61.69 |
| ATOM | 7 | CD | GLU | A | 2 | 18.744 | 20.732 | 47.230 | 1.00 | 66.10 |
| ATOM | 8 | OE1 | GLU | A | 2 | 18.034 | 21.666 | 46.811 | 1.00 | 66.32 |
| ATOM | 9 | OE2 | GLU | A | 2 | 20.002 | 20.766 | 47.202 | 1.00 | 74.14 |
| ATOM | 10 | N | MET | A | 3 | 20.191 | 17.713 | 49.043 | 1.00 | 60.00 |
| ATOM | 11 | CA | MET | A | 3 | 21.011 | 17.652 | 50.240 | 1.00 | 57.25 |
| ATOM | 12 | C | MET | A | 3 | 20.614 | 18.678 | 51.265 | 1.00 | 55.29 |
| ATOM | 13 | O | MET | A | 3 | 20.747 | 18.412 | 52.452 | 1.00 | 50.01 |
| ATOM | 14 | CB | MET | A | 3 | 22.479 | 17.882 | 49.882 | 1.00 | 55.54 |
| ATOM | 15 | CG | MET | A | 3 | 23.423 | 17.093 | 50.752 | 1.00 | 55.32 |
| ATOM | 16 | SD | MET | A | 3 | 25.071 | 17.182 | 50.098 | 1.00 | 54.46 |
| ATOM | 17 | CE | MET | A | 3 | 25.773 | 18.277 | 51.201 | 1.00 | 54.00 |
| ATOM | 18 | N | GLU | A | 4 | 20.198 | 19.858 | 50.800 | 1.00 | 54.87 |
| ATOM | 19 | CA | GLU | A | 4 | 19.740 | 20.939 | 51.680 | 1.00 | 58.22 |
| ATOM | 20 | C | GLU | A | 4 | 18.449 | 20.513 | 52.380 | 1.00 | 60.58 |
| ATOM | 21 | O | GLU | A | 4 | 18.315 | 20.650 | 53.597 | 1.00 | 59.53 |
| ATOM | 22 | CB | GLU | A | 4 | 19.514 | 22.229 | 50.883 | 1.00 | 58.04 |
| ATOM | 23 | CG | GLU | A | 4 | 19.295 | 23.466 | 51.743 | 1.00 | 58.46 |
| ATOM | 24 | CD | GLU | A | 4 | 19.539 | 24.760 | 50.984 | 1.00 | 61.10 |
| ATOM | 25 | OE1 | GLU | A | 4 | 19.208 | 24.828 | 49.782 | 1.00 | 65.91 |
| ATOM | 26 | OE2 | GLU | A | 4 | 20.064 | 25.717 | 51.587 | 1.00 | 62.36 |
| ATOM | 27 | N | LYS | A | 5 | 17.514 | 19.985 | 51.594 | 1.00 | 63.68 |
| ATOM | 28 | CA | LYS | A | 5 | 16.304 | 19.358 | 52.121 | 1.00 | 65.50 |
| ATOM | 29 | C | LYS | A | 5 | 16.669 | 18.372 | 53.236 | 1.00 | 64.36 |
| ATOM | 30 | O | LYS | A | 5 | 16.160 | 18.486 | 54.354 | 1.00 | 62.53 |
| ATOM | 31 | CB | LYS | A | 5 | 15.546 | 18.637 | 50.994 | 1.00 | 67.62 |
| ATOM | 32 | CG | LYS | A | 5 | 14.134 | 18.140 | 51.362 | 1.00 | 72.06 |
| ATOM | 33 | CD | LYS | A | 5 | 13.886 | 16.663 | 50.965 | 1.00 | 74.03 |
| ATOM | 34 | CE | LYS | A | 5 | 12.516 | 16.450 | 50.305 | 1.00 | 75.34 |
| ATOM | 35 | NZ | LYS | A | 5 | 12.393 | 17.128 | 48.976 | 1.00 | 74.88 |
| ATOM | 36 | N | GLU | A | 6 | 17.567 | 17.434 | 52.927 | 1.00 | 61.33 |
| ATOM | 37 | CA | GLU | A | 6 | 17.992 | 16.400 | 53.876 | 1.00 | 60.50 |
| ATOM | 38 | C | GLU | A | 6 | 18.602 | 17.026 | 55.129 | 1.00 | 59.07 |
| ATOM | 39 | O | GLU | A | 6 | 18.240 | 16.671 | 56.244 | 1.00 | 59.12 |
| ATOM | 40 | CB | GLU | A | 6 | 19.003 | 15.439 | 53.232 | 1.00 | 60.37 |
| ATOM | 41 | CG | GLU | A | 6 | 19.396 | 14.260 | 54.116 | 1.00 | 60.01 |
| ATOM | 42 | CD | GLU | A | 6 | 20.566 | 13.453 | 53.572 | 1.00 | 61.12 |
| ATOM | 43 | OE1 | GLU | A | 6 | 20.848 | 13.512 | 52.359 | 1.00 | 61.84 |
| ATOM | 44 | OE2 | GLU | A | 6 | 21.205 | 12.737 | 54.368 | 1.00 | 62.14 |
| ATOM | 45 | N | PHE | A | 7 | 19.529 | 17.957 | 54.927 | 1.00 | 57.25 |
| ATOM | 46 | CA | PHE | A | 7 | 20.140 | 18.727 | 56.009 | 1.00 | 56.47 |
| ATOM | 47 | C | PHE | A | 7 | 19.079 | 19.264 | 56.981 | 1.00 | 58.79 |
| ATOM | 48 | O | PHE | A | 7 | 19.212 | 19.105 | 58.189 | 1.00 | 55.70 |
| ATOM | 49 | CB | PHE | A | 7 | 20.941 | 19.886 | 55.407 | 1.00 | 54.93 |
| ATOM | 50 | CG | PHE | A | 7 | 21.692 | 20.708 | 56.409 | 1.00 | 52.66 |
| ATOM | 51 | CD1 | PHE | A | 7 | 21.264 | 21.981 | 56.736 | 1.00 | 53.50 |
| ATOM | 52 | CD2 | PHE | A | 7 | 22.852 | 20.221 | 56.994 | 1.00 | 53.27 |
| ATOM | 53 | CE1 | PHE | A | 7 | 21.967 | 22.747 | 57.649 | 1.00 | 54.98 |
| ATOM | 54 | CE2 | PHE | A | 7 | 23.560 | 20.973 | 57.904 | 1.00 | 50.40 |
| ATOM | 55 | CZ | PHE | A | 7 | 23.126 | 22.237 | 58.234 | 1.00 | 54.44 |
| ATOM | 56 | N | GLU | A | 8 | 18.028 | 19.876 | 56.433 | 1.00 | 59.80 |
| ATOM | 57 | CA | GLU | A | 8 | 16.966 | 20.489 | 57.228 | 1.00 | 62.23 |
| ATOM | 58 | C | GLU | A | 8 | 16.125 | 19.451 | 57.962 | 1.00 | 61.71 |
| ATOM | 59 | O | GLU | A | 8 | 15.778 | 19.648 | 59.117 | 1.00 | 64.03 |
| ATOM | 60 | CB | GLU | A | 8 | 16.082 | 21.388 | 56.351 | 1.00 | 63.16 |
| ATOM | 61 | CG | GLU | A | 8 | 16.657 | 22.792 | 56.189 | 1.00 | 64.89 |
| ATOM | 62 | CD | GLU | A | 8 | 16.174 | 23.521 | 54.943 | 1.00 | 68.05 |
| ATOM | 63 | OE1 | GLU | A | 8 | 15.280 | 23.002 | 54.235 | 1.00 | 69.31 |
| ATOM | 64 | OE2 | GLU | A | 8 | 16.696 | 24.631 | 54.671 | 1.00 | 70.62 |
| ATOM | 65 | N | GLN | A | 9 | 15.823 | 18.340 | 57.303 | 1.00 | 62.72 |
| ATOM | 66 | CA | GLN | A | 9 | 15.137 | 17.223 | 57.948 | 1.00 | 64.42 |
| ATOM | 67 | C | GLN | A | 9 | 15.898 | 16.765 | 59.201 | 1.00 | 64.36 |
| ATOM | 68 | O | GLN | A | 9 | 15.284 | 16.393 | 60.191 | 1.00 | 63.25 |
| ATOM | 69 | CB | GLN | A | 9 | 14.961 | 16.052 | 56.961 | 1.00 | 67.37 |
| ATOM | 70 | CG | GLN | A | 9 | 14.474 | 14.725 | 57.579 | 1.00 | 71.31 |
| ATOM | 71 | CD | GLN | A | 9 | 15.601 | 13.710 | 57.822 | 1.00 | 75.81 |
| ATOM | 72 | OE1 | GLN | A | 9 | 15.778 | 13.226 | 58.948 | 1.00 | 77.61 |
| ATOM | 73 | NE2 | GLN | A | 9 | 16.353 | 13.384 | 56.768 | 1.00 | 77.69 |
| ATOM | 74 | N | ILE | A | 10 | 17.230 | 16.802 | 59.152 | 1.00 | 63.29 |
| ATOM | 75 | CA | ILE | A | 10 | 18.070 | 16.360 | 60.264 | 1.00 | 60.87 |
| ATOM | 76 | C | ILE | A | 10 | 18.214 | 17.468 | 61.297 | 1.00 | 58.42 |
| ATOM | 77 | O | ILE | A | 10 | 18.294 | 17.193 | 62.482 | 1.00 | 56.02 |
| ATOM | 78 | CB | ILE | A | 10 | 19.477 | 15.920 | 59.760 | 1.00 | 62.50 |
| ATOM | 79 | CG1 | ILE | A | 10 | 19.366 | 14.830 | 58.693 | 1.00 | 61.57 |
| ATOM | 80 | CG2 | ILE | A | 10 | 20.341 | 15.400 | 60.909 | 1.00 | 61.51 |
| ATOM | 81 | CD1 | ILE | A | 10 | 20.531 | 14.838 | 57.723 | 1.00 | 62.38 |
| ATOM | 82 | N | ASP | A | 11 | 18.242 | 18.717 | 60.846 | 1.00 | 57.74 |
| ATOM | 83 | CA | ASP | A | 11 | 18.500 | 19.839 | 61.740 | 1.00 | 60.17 |
| ATOM | 84 | C | ASP | A | 11 | 17.331 | 20.064 | 62.715 | 1.00 | 66.19 |
| ATOM | 85 | O | ASP | A | 11 | 17.542 | 20.152 | 63.934 | 1.00 | 67.55 |
| ATOM | 86 | CB | ASP | A | 11 | 18.810 | 21.119 | 60.946 | 1.00 | 57.92 |
| ATOM | 87 | CG | ASP | A | 11 | 20.141 | 21.745 | 61.337 | 1.00 | 57.04 |
| ATOM | 88 | OD1 | ASP | A | 11 | 20.949 | 21.072 | 61.995 | 1.00 | 51.82 |
| ATOM | 89 | OD2 | ASP | A | 11 | 20.474 | 22.910 | 61.039 | 1.00 | 58.59 |
| ATOM | 90 | N | LYS | A | 12 | 16.111 | 20.143 | 62.177 | 1.00 | 69.33 |
| ATOM | 91 | CA | LYS | A | 12 | 14.906 | 20.286 | 62.998 | 1.00 | 71.11 |
| ATOM | 92 | C | LYS | A | 12 | 14.553 | 18.991 | 63.749 | 1.00 | 70.45 |
| ATOM | 93 | O | LYS | A | 12 | 14.050 | 19.045 | 64.870 | 1.00 | 70.33 |
| ATOM | 94 | CB | LYS | A | 12 | 13.709 | 20.810 | 62.172 | 1.00 | 72.97 |
| ATOM | 95 | CG | LYS | A | 12 | 13.231 | 19.924 | 61.005 | 1.00 | 74.96 |
| ATOM | 96 | CD | LYS | A | 12 | 11.701 | 19.746 | 60.978 | 1.00 | 76.87 |
| ATOM | 97 | CE | LYS | A | 12 | 10.968 | 20.982 | 60.455 | 1.00 | 77.63 |
| ATOM | 98 | NZ | LYS | A | 12 | 9.535 | 20.997 | 60.876 | 1.00 | 77.36 |
| ATOM | 99 | N | SER | A | 13 | 14.846 | 17.838 | 63.147 | 1.00 | 70.77 |
| ATOM | 100 | CA | SER | A | 13 | 14.634 | 16.538 | 63.802 | 1.00 | 69.85 |
| ATOM | 101 | C | SER | A | 13 | 15.676 | 16.234 | 64.886 | 1.00 | 70.13 |
| ATOM | 102 | O | SER | A | 13 | 15.508 | 15.281 | 65.651 | 1.00 | 70.93 |
| ATOM | 103 | CB | SER | A | 13 | 14.653 | 15.408 | 62.773 | 1.00 | 69.58 |
| ATOM | 104 | OG | SER | A | 13 | 14.288 | 14.167 | 63.350 | 1.00 | 70.26 |
| ATOM | 105 | N | GLY | A | 14 | 16.747 | 17.028 | 64.942 | 1.00 | 69.41 |
| ATOM | 106 | CA | GLY | A | 14 | 17.810 | 16.840 | 65.914 | 1.00 | 68.23 |
| ATOM | 107 | C | GLY | A | 14 | 18.455 | 15.461 | 65.892 | 1.00 | 67.73 |
| ATOM | 108 | O | GLY | A | 14 | 18.794 | 14.929 | 66.952 | 1.00 | 68.22 |
| ATOM | 109 | N | SER | A | 15 | 18.651 | 14.896 | 64.697 | 1.00 | 65.75 |
| ATOM | 110 | CA | SER | A | 15 | 19.148 | 13.520 | 64.550 | 1.00 | 64.50 |
| ATOM | 111 | C | SER | A | 15 | 20.604 | 13.416 | 64.048 | 1.00 | 61.83 |
| ATOM | 112 | O | SER | A | 15 | 21.027 | 12.350 | 63.591 | 1.00 | 59.16 |
| ATOM | 113 | CB | SER | A | 15 | 18.219 | 12.709 | 63.634 | 1.00 | 64.41 |
| ATOM | 114 | OG | SER | A | 15 | 17.326 | 13.550 | 62.921 | 1.00 | 68.90 |
| ATOM | 115 | N | TRP | A | 16 | 21.377 | 14.495 | 64.158 | 1.00 | 59.81 |
| ATOM | 116 | CA | TRP | A | 16 | 22.779 | 14.466 | 63.720 | 1.00 | 58.20 |
| ATOM | 117 | C | TRP | A | 16 | 23.563 | 13.374 | 64.419 | 1.00 | 58.30 |
| ATOM | 118 | O | TRP | A | 16 | 24.215 | 12.568 | 63.768 | 1.00 | 56.78 |
| ATOM | 119 | CB | TRP | A | 16 | 23.458 | 15.813 | 63.939 | 1.00 | 57.31 |
| ATOM | 120 | CG | TRP | A | 16 | 23.063 | 16.794 | 62.905 | 1.00 | 57.69 |
| ATOM | 121 | CD1 | TRP | A | 16 | 22.356 | 17.937 | 63.091 | 1.00 | 58.78 |
| ATOM | 122 | CD2 | TRP | A | 16 | 23.327 | 16.707 | 61.501 | 1.00 | 59.24 |
| ATOM | 123 | NE1 | TRP | A | 16 | 22.164 | 18.576 | 61.892 | 1.00 | 59.34 |
| ATOM | 124 | CE2 | TRP | A | 16 | 22.754 | 17.841 | 60.897 | 1.00 | 61.47 |
| ATOM | 125 | CE3 | TRP | A | 16 | 23.985 | 15.774 | 60.685 | 1.00 | 59.52 |
| ATOM | 126 | CZ2 | TRP | A | 16 | 22.818 | 18.073 | 59.513 | 1.00 | 60.24 |
| ATOM | 127 | CZ3 | TRP | A | 16 | 24.057 | 16.009 | 59.318 | 1.00 | 56.01 |
| ATOM | 128 | CH2 | TRP | A | 16 | 23.479 | 17.149 | 58.749 | 1.00 | 57.24 |
| ATOM | 129 | N | ALA | A | 17 | 23.480 | 13.340 | 65.746 | 1.00 | 60.14 |
| ATOM | 130 | CA | ALA | A | 17 | 24.142 | 12.305 | 66.538 | 1.00 | 58.74 |
| ATOM | 131 | C | ALA | A | 17 | 23.755 | 10.901 | 66.084 | 1.00 | 57.85 |
| ATOM | 132 | O | ALA | A | 17 | 24.603 | 10.012 | 66.004 | 1.00 | 57.84 |
| ATOM | 133 | CB | ALA | A | 17 | 23.812 | 12.490 | 68.011 | 1.00 | 60.75 |
| ATOM | 134 | N | ALA | A | 18 | 22.473 | 10.709 | 65.787 | 1.00 | 58.13 |
| ATOM | 135 | CA | ALA | A | 18 | 21.955 | 9.394 | 65.403 | 1.00 | 58.42 |
| ATOM | 136 | C | ALA | A | 18 | 22.355 | 8.979 | 63.980 | 1.00 | 58.15 |
| ATOM | 137 | O | ALA | A | 18 | 22.669 | 7.813 | 63.731 | 1.00 | 55.17 |
| ATOM | 138 | CB | ALA | A | 18 | 20.442 | 9.366 | 65.555 | 1.00 | 57.99 |
| ATOM | 139 | N | ILE | A | 19 | 22.324 | 9.931 | 63.049 | 1.00 | 59.40 |
| ATOM | 140 | CA | ILE | A | 19 | 22.723 | 9.674 | 61.659 | 1.00 | 59.74 |
| ATOM | 141 | C | ILE | A | 19 | 24.223 | 9.420 | 61.620 | 1.00 | 56.93 |
| ATOM | 142 | O | ILE | A | 19 | 24.698 | 8.520 | 60.924 | 1.00 | 54.95 |
| ATOM | 143 | CB | ILE | A | 19 | 22.354 | 10.877 | 60.730 | 1.00 | 61.02 |
| ATOM | 144 | CG1 | ILE | A | 19 | 20.831 | 10.977 | 60.537 | 1.00 | 61.51 |
| ATOM | 145 | CG2 | ILE | A | 19 | 23.059 | 10.760 | 59.375 | 1.00 | 61.84 |
| ATOM | 146 | CD1 | ILE | A | 19 | 20.244 | 9.940 | 59.571 | 1.00 | 62.30 |
| ATOM | 147 | N | TYR | A | 20 | 24.961 | 10.220 | 62.382 | 1.00 | 56.23 |
| ATOM | 148 | CA | TYR | A | 20 | 26.397 | 10.045 | 62.497 | 1.00 | 55.96 |
| ATOM | 149 | C | TYR | A | 20 | 26.708 | 8.642 | 62.985 | 1.00 | 54.08 |
| ATOM | 150 | O | TYR | A | 20 | 27.584 | 7.975 | 62.444 | 1.00 | 50.49 |
| ATOM | 151 | CB | TYR | A | 20 | 27.020 | 11.083 | 63.440 | 1.00 | 55.69 |
| ATOM | 152 | CG | TYR | A | 20 | 28.491 | 10.821 | 63.664 | 1.00 | 57.45 |
| ATOM | 153 | CD1 | TYR | A | 20 | 29.392 | 10.873 | 62.604 | 1.00 | 58.07 |
| ATOM | 154 | CD2 | TYR | A | 20 | 28.973 | 10.463 | 64.916 | 1.00 | 56.79 |
| ATOM | 155 | CE1 | TYR | A | 20 | 30.731 | 10.608 | 62.791 | 1.00 | 57.90 |
| ATOM | 156 | CE2 | TYR | A | 20 | 30.307 | 10.201 | 65.110 | 1.00 | 57.86 |
| ATOM | 157 | CZ | TYR | A | 20 | 31.181 | 10.279 | 64.045 | 1.00 | 56.10 |
| ATOM | 158 | OH | TYR | A | 20 | 32.501 | 10.016 | 64.241 | 1.00 | 53.46 |
| ATOM | 159 | N | GLN | A | 21 | 25.965 | 8.189 | 63.991 | 1.00 | 55.74 |
| ATOM | 160 | CA | GLN | A | 21 | 26.229 | 6.890 | 64.588 | 1.00 | 57.04 |
| ATOM | 161 | C | GLN | A | 21 | 25.867 | 5.725 | 63.673 | 1.00 | 54.95 |
| ATOM | 162 | O | GLN | A | 21 | 26.488 | 4.680 | 63.776 | 1.00 | 56.01 |
| ATOM | 163 | CB | GLN | A | 21 | 25.547 | 6.749 | 65.960 | 1.00 | 59.46 |
| ATOM | 164 | CG | GLN | A | 21 | 26.326 | 5.856 | 66.946 | 1.00 | 62.63 |
| ATOM | 165 | CD | GLN | A | 21 | 27.747 | 6.370 | 67.254 | 1.00 | 64.64 |
| ATOM | 166 | OE1 | GLN | A | 21 | 27.933 | 7.540 | 67.624 | 1.00 | 65.76 |
| ATOM | 167 | NE2 | GLN | A | 21 | 28.741 | 5.492 | 67.106 | 1.00 | 64.52 |
| ATOM | 168 | N | ASP | A | 22 | 24.893 | 5.891 | 62.777 | 1.00 | 55.58 |
| ATOM | 169 | CA | ASP | A | 22 | 24.578 | 4.833 | 61.807 | 1.00 | 58.11 |
| ATOM | 170 | C | ASP | A | 22 | 25.732 | 4.660 | 60.830 | 1.00 | 55.90 |
| ATOM | 171 | O | ASP | A | 22 | 26.113 | 3.537 | 60.499 | 1.00 | 54.23 |
| ATOM | 172 | CB | ASP | A | 22 | 23.314 | 5.143 | 61.001 | 1.00 | 61.30 |
| ATOM | 173 | CG | ASP | A | 22 | 22.053 | 5.090 | 61.833 | 1.00 | 67.69 |
| ATOM | 174 | OD1 | ASP | A | 22 | 21.876 | 4.115 | 62.604 | 1.00 | 71.22 |
| ATOM | 175 | OD2 | ASP | A | 22 | 21.174 | 5.985 | 61.769 | 1.00 | 71.52 |
| ATOM | 176 | N | ILE | A | 23 | 26.271 | 5.783 | 60.361 | 1.00 | 53.69 |
| ATOM | 177 | CA | ILE | A | 23 | 27.378 | 5.770 | 59.408 | 1.00 | 53.37 |
| ATOM | 178 | C | ILE | A | 23 | 28.566 | 5.069 | 60.033 | 1.00 | 50.71 |
| ATOM | 179 | O | ILE | A | 23 | 29.214 | 4.250 | 59.391 | 1.00 | 44.98 |
| ATOM | 180 | CB | ILE | A | 23 | 27.768 | 7.215 | 58.997 | 1.00 | 53.19 |
| ATOM | 181 | CG1 | ILE | A | 23 | 26.722 | 7.799 | 58.054 | 1.00 | 53.10 |
| ATOM | 182 | CG2 | ILE | A | 23 | 29.136 | 7.243 | 58.315 | 1.00 | 54.93 |
| ATOM | 183 | CD1 | ILE | A | 23 | 26.799 | 9.301 | 57.933 | 1.00 | 55.17 |
| ATOM | 184 | N | ARG | A | 24 | 28.839 | 5.401 | 61.291 | 1.00 | 53.62 |
| ATOM | 185 | CA | ARG | A | 24 | 29.949 | 4.790 | 62.027 | 1.00 | 58.86 |
| ATOM | 186 | C | ARG | A | 24 | 29.814 | 3.265 | 62.157 | 1.00 | 58.90 |
| ATOM | 187 | O | ARG | A | 24 | 30.803 | 2.548 | 62.062 | 1.00 | 54.97 |
| ATOM | 188 | CB | ARG | A | 24 | 30.104 | 5.425 | 63.412 | 1.00 | 61.20 |
| ATOM | 189 | CG | ARG | A | 24 | 31.536 | 5.398 | 63.933 | 1.00 | 67.63 |
| ATOM | 190 | CD | ARG | A | 24 | 31.711 | 4.776 | 65.313 | 1.00 | 73.89 |
| ATOM | 191 | NE | ARG | A | 24 | 31.711 | 5.781 | 66.377 | 1.00 | 77.54 |
| ATOM | 192 | CZ | ARG | A | 24 | 31.977 | 5.523 | 67.654 | 1.00 | 80.87 |
| ATOM | 193 | NH1 | ARG | A | 24 | 32.264 | 4.285 | 68.052 | 1.00 | 82.17 |
| ATOM | 194 | NH2 | ARG | A | 24 | 31.950 | 6.509 | 68.543 | 1.00 | 82.16 |
| ATOM | 195 | N | HIS | A | 25 | 28.593 | 2.774 | 62.357 | 1.00 | 62.10 |
| ATOM | 196 | CA | HIS | A | 25 | 28.359 | 1.330 | 62.436 | 1.00 | 64.65 |
| ATOM | 197 | C | HIS | A | 25 | 28.473 | 0.658 | 61.059 | 1.00 | 61.06 |
| ATOM | 198 | O | HIS | A | 25 | 28.941 | −0.480 | 60.956 | 1.00 | 58.40 |
| ATOM | 199 | CB | HIS | A | 25 | 26.993 | 1.028 | 63.070 | 1.00 | 67.93 |
| ATOM | 200 | CG | HIS | A | 25 | 26.859 | −0.384 | 63.559 | 1.00 | 74.44 |
| ATOM | 201 | ND1 | HIS | A | 25 | 25.893 | −1.248 | 63.087 | 1.00 | 77.50 |
| ATOM | 202 | CD2 | HIS | A | 25 | 27.588 | −1.090 | 64.457 | 1.00 | 77.23 |
| ATOM | 203 | CE1 | HIS | A | 25 | 26.026 | −2.422 | 63.680 | 1.00 | 78.94 |
| ATOM | 204 | NE2 | HIS | A | 25 | 27.046 | −2.352 | 64.517 | 1.00 | 79.41 |
| ATOM | 205 | N | GLU | A | 26 | 28.059 | 1.374 | 60.012 | 1.00 | 58.65 |
| ATOM | 206 | CA | GLU | A | 26 | 28.156 | 0.890 | 58.632 | 1.00 | 56.89 |
| ATOM | 207 | C | GLU | A | 26 | 29.616 | 0.814 | 58.155 | 1.00 | 55.60 |
| ATOM | 208 | O | GLU | A | 26 | 29.943 | 0.026 | 57.270 | 1.00 | 52.37 |
| ATOM | 209 | CB | GLU | A | 26 | 27.372 | 1.814 | 57.687 | 1.00 | 58.61 |
| ATOM | 210 | CG | GLU | A | 26 | 25.848 | 1.721 | 57.748 | 1.00 | 61.50 |
| ATOM | 211 | CD | GLU | A | 26 | 25.167 | 2.522 | 56.631 | 1.00 | 64.68 |
| ATOM | 212 | OE1 | GLU | A | 26 | 25.149 | 2.040 | 55.469 | 1.00 | 66.62 |
| ATOM | 213 | OE2 | GLU | A | 26 | 24.658 | 3.639 | 56.901 | 1.00 | 62.82 |
| ATOM | 214 | N | ALA | A | 27 | 30.489 | 1.625 | 58.756 | 1.00 | 53.91 |
| ATOM | 215 | CA | ALA | A | 27 | 31.836 | 1.875 | 58.225 | 1.00 | 53.38 |
| ATOM | 216 | C | ALA | A | 27 | 32.749 | 0.659 | 58.215 | 1.00 | 51.05 |
| ATOM | 217 | O | ALA | A | 27 | 32.577 | −0.266 | 59.007 | 1.00 | 54.77 |
| ATOM | 218 | CB | ALA | A | 27 | 32.502 | 3.012 | 58.998 | 1.00 | 52.70 |
| ATOM | 219 | N | SER | A | 28 | 33.744 | 0.695 | 57.333 | 1.00 | 47.02 |
| ATOM | 220 | CA | SER | A | 28 | 34.641 | −0.446 | 57.115 | 1.00 | 47.65 |
| ATOM | 221 | C | SER | A | 28 | 35.608 | −0.626 | 58.270 | 1.00 | 48.65 |
| ATOM | 222 | O | SER | A | 28 | 35.990 | 0.348 | 58.930 | 1.00 | 47.16 |
| ATOM | 223 | CB | SER | A | 28 | 35.488 | −0.260 | 55.843 | 1.00 | 46.74 |
| ATOM | 224 | OG | SER | A | 28 | 34.741 | 0.238 | 54.748 | 1.00 | 44.10 |
| ATOM | 225 | N | ASP | A | 29 | 36.035 | −1.866 | 58.473 | 1.00 | 49.97 |
| ATOM | 226 | CA | ASP | A | 29 | 36.999 | −2.195 | 59.508 | 1.00 | 52.38 |
| ATOM | 227 | C | ASP | A | 29 | 37.986 | −3.217 | 58.977 | 1.00 | 49.09 |
| ATOM | 228 | O | ASP | A | 29 | 37.690 | −4.404 | 58.902 | 1.00 | 56.60 |
| ATOM | 229 | CB | ASP | A | 29 | 36.288 | −2.718 | 60.760 | 1.00 | 56.80 |
| ATOM | 230 | CG | ASP | A | 29 | 37.183 | −2.697 | 61.994 | 1.00 | 59.43 |
| ATOM | 231 | OD1 | ASP | A | 29 | 38.114 | −1.863 | 62.046 | 1.00 | 62.50 |
| ATOM | 232 | OD2 | ASP | A | 29 | 37.020 | −3.471 | 62.960 | 1.00 | 62.02 |
| ATOM | 233 | N | PHE | A | 30 | 39.161 | −2.733 | 58.606 | 1.00 | 44.75 |
| ATOM | 234 | CA | PHE | A | 30 | 40.168 | −3.529 | 57.925 | 1.00 | 41.06 |
| ATOM | 235 | C | PHE | A | 30 | 41.380 | −3.665 | 58.830 | 1.00 | 40.54 |
| ATOM | 236 | O | PHE | A | 30 | 41.525 | −2.882 | 59.763 | 1.00 | 43.29 |
| ATOM | 237 | CB | PHE | A | 30 | 40.562 | −2.828 | 56.620 | 1.00 | 39.31 |
| ATOM | 238 | CG | PHE | A | 30 | 39.502 | −2.878 | 55.551 | 1.00 | 36.76 |
| ATOM | 239 | CD1 | PHE | A | 30 | 39.243 | −4.061 | 54.868 | 1.00 | 35.27 |
| ATOM | 240 | CD2 | PHE | A | 30 | 38.781 | −1.733 | 55.203 | 1.00 | 32.94 |
| ATOM | 241 | CE1 | PHE | A | 30 | 38.281 | −4.114 | 53.868 | 1.00 | 33.02 |
| ATOM | 242 | CE2 | PHE | A | 30 | 37.816 | −1.773 | 54.190 | 1.00 | 32.39 |
| ATOM | 243 | CZ | PHE | A | 30 | 37.562 | −2.961 | 53.519 | 1.00 | 34.23 |
| ATOM | 244 | N | PRO | A | 31 | 42.255 | −4.637 | 58.573 | 1.00 | 39.38 |
| ATOM | 245 | CA | PRO | A | 31 | 43.447 | −4.816 | 59.409 | 1.00 | 41.61 |
| ATOM | 246 | C | PRO | A | 31 | 44.463 | −3.686 | 59.285 | 1.00 | 43.42 |
| ATOM | 247 | O | PRO | A | 31 | 44.527 | −3.030 | 58.242 | 1.00 | 43.43 |
| ATOM | 248 | CB | PRO | A | 31 | 44.075 | −6.107 | 58.875 | 1.00 | 40.50 |
| ATOM | 249 | CG | PRO | A | 31 | 43.576 | −6.239 | 57.495 | 1.00 | 40.37 |
| ATOM | 250 | CD | PRO | A | 31 | 42.181 | −5.658 | 57.516 | 1.00 | 40.35 |
| ATOM | 251 | N | CYS | A | 32 | 45.244 | −3.506 | 60.354 | 1.00 | 43.17 |
| ATOM | 252 | CA | CYS | A | 32 | 46.340 | −2.545 | 60.435 | 1.00 | 43.74 |
| ATOM | 253 | C | CYS | A | 32 | 47.577 | −3.203 | 61.052 | 1.00 | 44.00 |
| ATOM | 254 | O | CYS | A | 32 | 48.220 | −2.627 | 61.924 | 1.00 | 41.19 |
| ATOM | 255 | CB | CYS | A | 32 | 45.937 | −1.372 | 61.325 | 1.00 | 42.18 |
| ATOM | 256 | SG | CYS | A | 32 | 44.513 | −0.438 | 60.792 | 1.00 | 40.55 |
| ATOM | 257 | N | ARG | A | 33 | 47.903 | −4.405 | 60.599 | 1.00 | 45.04 |
| ATOM | 258 | CA | ARG | A | 33 | 48.957 | −5.214 | 61.214 | 1.00 | 47.18 |
| ATOM | 259 | C | ARG | A | 33 | 50.330 | −4.574 | 61.103 | 1.00 | 44.66 |
| ATOM | 260 | O | ARG | A | 33 | 51.142 | −4.652 | 62.029 | 1.00 | 43.21 |
| ATOM | 261 | CB | ARG | A | 33 | 48.993 | −6.622 | 60.597 | 1.00 | 50.33 |
| ATOM | 262 | CG | ARG | A | 33 | 47.642 | −7.333 | 60.664 | 1.00 | 55.89 |
| ATOM | 263 | CD | ARG | A | 33 | 47.679 | −8.868 | 60.565 | 1.00 | 61.15 |
| ATOM | 264 | NE | ARG | A | 33 | 46.343 | −9.403 | 60.854 | 1.00 | 65.45 |
| ATOM | 265 | CZ | ARG | A | 33 | 45.403 | −9.700 | 59.943 | 1.00 | 67.05 |
| ATOM | 266 | NH1 | ARG | A | 33 | 44.218 | −10.151 | 60.357 | 1.00 | 67.41 |
| ATOM | 267 | NH2 | ARG | A | 33 | 45.630 | −9.563 | 58.634 | 1.00 | 64.70 |
| ATOM | 268 | N | VAL | A | 34 | 50.609 | −3.954 | 59.969 | 1.00 | 38.20 |
| ATOM | 269 | CA | VAL | A | 34 | 51.912 | −3.339 | 59.805 | 1.00 | 39.88 |
| ATOM | 270 | C | VAL | A | 34 | 52.086 | −2.196 | 60.820 | 1.00 | 39.12 |
| ATOM | 271 | O | VAL | A | 34 | 53.167 | −2.049 | 61.400 | 1.00 | 35.07 |
| ATOM | 272 | CB | VAL | A | 34 | 52.157 | −2.882 | 58.373 | 1.00 | 40.69 |
| ATOM | 273 | CG1 | VAL | A | 34 | 53.503 | −2.189 | 58.276 | 1.00 | 41.70 |
| ATOM | 274 | CG2 | VAL | A | 34 | 52.103 | −4.098 | 57.427 | 1.00 | 40.83 |
| ATOM | 275 | N | ALA | A | 35 | 51.004 | −1.443 | 61.051 | 1.00 | 36.64 |
| ATOM | 276 | CA | ALA | A | 35 | 51.011 | −0.297 | 61.952 | 1.00 | 40.93 |
| ATOM | 277 | C | ALA | A | 35 | 51.190 | −0.711 | 63.411 | 1.00 | 42.58 |
| ATOM | 278 | O | ALA | A | 35 | 51.694 | 0.073 | 64.208 | 1.00 | 41.51 |
| ATOM | 279 | CB | ALA | A | 35 | 49.711 | 0.511 | 61.814 | 1.00 | 38.22 |
| ATOM | 280 | N | LYS | A | 36 | 50.739 | −1.920 | 63.748 | 1.00 | 42.79 |
| ATOM | 281 | CA | LYS | A | 36 | 50.774 | −2.436 | 65.112 | 1.00 | 42.74 |
| ATOM | 282 | C | LYS | A | 36 | 52.059 | −3.216 | 65.426 | 1.00 | 40.53 |
| ATOM | 283 | O | LYS | A | 36 | 52.229 | −3.678 | 66.545 | 1.00 | 41.91 |
| ATOM | 284 | CB | LYS | A | 36 | 49.539 | −3.311 | 65.388 | 1.00 | 45.34 |
| ATOM | 285 | CG | LYS | A | 36 | 48.189 | −2.565 | 65.347 | 1.00 | 48.30 |
| ATOM | 286 | CD | LYS | A | 36 | 47.948 | −1.770 | 66.633 | 1.00 | 53.46 |
| ATOM | 287 | CE | LYS | A | 36 | 47.424 | −0.354 | 66.388 | 1.00 | 53.76 |
| ATOM | 288 | NZ | LYS | A | 36 | 46.777 | 0.176 | 67.615 | 1.00 | 52.68 |
| ATOM | 289 | N | LEU | A | 37 | 52.970 | −3.338 | 64.465 | 1.00 | 40.84 |
| ATOM | 290 | CA | LEU | A | 37 | 54.249 | −3.991 | 64.716 | 1.00 | 41.82 |
| ATOM | 291 | C | LEU | A | 37 | 55.108 | −3.186 | 65.702 | 1.00 | 48.37 |
| ATOM | 292 | O | LEU | A | 37 | 55.166 | −1.949 | 65.624 | 1.00 | 47.86 |
| ATOM | 293 | CB | LEU | A | 37 | 55.032 | −4.185 | 63.436 | 1.00 | 40.53 |
| ATOM | 294 | CG | LEU | A | 37 | 54.415 | −5.077 | 62.365 | 1.00 | 43.98 |
| ATOM | 295 | CD1 | LEU | A | 37 | 55.328 | −5.082 | 61.158 | 1.00 | 42.81 |
| ATOM | 296 | CD2 | LEU | A | 37 | 54.166 | −6.504 | 62.877 | 1.00 | 48.44 |
| ATOM | 297 | N | PRO | A | 38 | 55.750 | −3.880 | 66.642 | 1.00 | 49.83 |
| ATOM | 298 | CA | PRO | A | 38 | 56.643 | −3.226 | 67.608 | 1.00 | 46.21 |
| ATOM | 299 | C | PRO | A | 38 | 57.711 | −2.334 | 66.992 | 1.00 | 43.54 |
| ATOM | 300 | O | PRO | A | 38 | 57.990 | −1.293 | 67.563 | 1.00 | 39.97 |
| ATOM | 301 | CB | PRO | A | 38 | 57.286 | −4.410 | 68.349 | 1.00 | 48.79 |
| ATOM | 302 | CG | PRO | A | 38 | 56.261 | −5.510 | 68.263 | 1.00 | 50.65 |
| ATOM | 303 | CD | PRO | A | 38 | 55.605 | −5.327 | 66.917 | 1.00 | 50.16 |
| ATOM | 304 | N | LYS | A | 39 | 58.301 | −2.700 | 65.862 | 1.00 | 43.10 |
| ATOM | 305 | CA | LYS | A | 39 | 59.311 | −1.831 | 65.257 | 1.00 | 44.66 |
| ATOM | 306 | C | LYS | A | 39 | 58.752 | −0.485 | 64.732 | 1.00 | 44.89 |
| ATOM | 307 | O | LYS | A | 39 | 59.520 | 0.417 | 64.393 | 1.00 | 43.74 |
| ATOM | 308 | CB | LYS | A | 39 | 60.070 | −2.552 | 64.141 | 1.00 | 46.32 |
| ATOM | 309 | CG | LYS | A | 39 | 59.264 | −2.891 | 62.896 | 1.00 | 48.76 |
| ATOM | 310 | CD | LYS | A | 39 | 60.203 | −3.220 | 61.744 | 1.00 | 51.08 |
| ATOM | 311 | CE | LYS | A | 39 | 59.470 | −3.894 | 60.595 | 1.00 | 53.07 |
| ATOM | 312 | NZ | LYS | A | 39 | 60.386 | −4.156 | 59.448 | 1.00 | 54.38 |
| ATOM | 313 | N | ASN | A | 40 | 57.429 | −0.368 | 64.662 | 1.00 | 42.04 |
| ATOM | 314 | CA | ASN | A | 40 | 56.774 | 0.833 | 64.148 | 1.00 | 43.82 |
| ATOM | 315 | C | ASN | A | 40 | 56.155 | 1.692 | 65.230 | 1.00 | 44.28 |
| ATOM | 316 | O | ASN | A | 40 | 55.560 | 2.719 | 64.923 | 1.00 | 39.51 |
| ATOM | 317 | CB | ASN | A | 40 | 55.692 | 0.449 | 63.125 | 1.00 | 39.92 |
| ATOM | 318 | CG | ASN | A | 40 | 56.278 | −0.081 | 61.847 | 1.00 | 34.23 |
| ATOM | 319 | OD1 | ASN | A | 40 | 57.368 | 0.324 | 61.435 | 1.00 | 38.32 |
| ATOM | 320 | ND2 | ASN | A | 40 | 55.560 | −0.989 | 61.201 | 1.00 | 35.81 |
| ATOM | 321 | N | LYS | A | 41 | 56.294 | 1.284 | 66.490 | 1.00 | 45.17 |
| ATOM | 322 | CA | LYS | A | 41 | 55.645 | 2.002 | 67.589 | 1.00 | 47.23 |
| ATOM | 323 | C | LYS | A | 41 | 56.064 | 3.467 | 67.635 | 1.00 | 42.51 |
| ATOM | 324 | O | LYS | A | 41 | 55.239 | 4.324 | 67.897 | 1.00 | 40.36 |
| ATOM | 325 | CB | LYS | A | 41 | 55.919 | 1.337 | 68.949 | 1.00 | 52.38 |
| ATOM | 326 | CG | LYS | A | 41 | 54.780 | 1.518 | 69.972 | 1.00 | 57.24 |
| ATOM | 327 | CD | LYS | A | 41 | 55.276 | 2.101 | 71.301 | 1.00 | 63.21 |
| ATOM | 328 | CE | LYS | A | 41 | 54.238 | 1.968 | 72.427 | 1.00 | 64.88 |
| ATOM | 329 | NZ | LYS | A | 41 | 54.694 | 2.635 | 73.696 | 1.00 | 66.35 |
| ATOM | 330 | N | ASN | A | 42 | 57.335 | 3.746 | 67.366 | 1.00 | 41.10 |
| ATOM | 331 | CA | ASN | A | 42 | 57.827 | 5.129 | 67.386 | 1.00 | 44.23 |
| ATOM | 332 | C | ASN | A | 42 | 57.461 | 5.950 | 66.128 | 1.00 | 40.34 |
| ATOM | 333 | O | ASN | A | 42 | 57.751 | 7.142 | 66.061 | 1.00 | 37.49 |
| ATOM | 334 | CB | ASN | A | 42 | 59.348 | 5.198 | 67.698 | 1.00 | 45.89 |
| ATOM | 335 | CG | ASN | A | 42 | 60.232 | 4.486 | 66.650 | 1.00 | 54.46 |
| ATOM | 336 | OD1 | ASN | A | 42 | 59.751 | 3.951 | 65.641 | 1.00 | 60.22 |
| ATOM | 337 | ND2 | ASN | A | 42 | 61.545 | 4.476 | 66.906 | 1.00 | 57.61 |
| ATOM | 338 | N | ARG | A | 43 | 56.821 | 5.308 | 65.149 | 1.00 | 36.12 |
| ATOM | 339 | CA | ARG | A | 43 | 56.379 | 5.971 | 63.923 | 1.00 | 32.01 |
| ATOM | 340 | C | ARG | A | 43 | 54.916 | 6.387 | 63.959 | 1.00 | 30.04 |
| ATOM | 341 | O | ARG | A | 43 | 54.437 | 7.037 | 63.032 | 1.00 | 29.73 |
| ATOM | 342 | CB | ARG | A | 43 | 56.679 | 5.111 | 62.708 | 1.00 | 30.74 |
| ATOM | 343 | CG | ARG | A | 43 | 58.164 | 4.956 | 62.499 | 1.00 | 28.56 |
| ATOM | 344 | CD | ARG | A | 43 | 58.527 | 4.042 | 61.392 | 1.00 | 32.76 |
| ATOM | 345 | NE | ARG | A | 43 | 59.966 | 4.062 | 61.163 | 1.00 | 33.60 |
| ATOM | 346 | CZ | ARG | A | 43 | 60.558 | 3.850 | 60.006 | 1.00 | 35.25 |
| ATOM | 347 | NH1 | ARG | A | 43 | 59.851 | 3.602 | 58.915 | 1.00 | 38.60 |
| ATOM | 348 | NH2 | ARG | A | 43 | 61.880 | 3.888 | 59.934 | 1.00 | 38.43 |
| ATOM | 349 | N | ASN | A | 44 | 54.236 | 6.060 | 65.053 | 1.00 | 30.11 |
| ATOM | 350 | CA | ASN | A | 44 | 52.833 | 6.357 | 65.219 | 1.00 | 29.93 |
| ATOM | 351 | C | ASN | A | 44 | 52.574 | 7.372 | 66.299 | 1.00 | 32.80 |
| ATOM | 352 | O | ASN | A | 44 | 53.110 | 7.257 | 67.387 | 1.00 | 30.67 |
| ATOM | 353 | CB | ASN | A | 44 | 52.074 | 5.077 | 65.583 | 1.00 | 32.74 |
| ATOM | 354 | CG | ASN | A | 44 | 51.984 | 4.121 | 64.421 | 1.00 | 38.06 |
| ATOM | 355 | OD1 | ASN | A | 44 | 51.661 | 4.522 | 63.290 | 1.00 | 35.12 |
| ATOM | 356 | ND2 | ASN | A | 44 | 52.307 | 2.860 | 64.674 | 1.00 | 34.92 |
| ATOM | 357 | N | ARG | A | 45 | 51.693 | 8.324 | 66.003 | 1.00 | 32.08 |
| ATOM | 358 | CA | ARG | A | 45 | 51.336 | 9.386 | 66.931 | 1.00 | 34.13 |
| ATOM | 359 | C | ARG | A | 45 | 50.227 | 8.864 | 67.836 | 1.00 | 37.52 |
| ATOM | 360 | O | ARG | A | 45 | 49.263 | 8.252 | 67.367 | 1.00 | 32.83 |
| ATOM | 361 | CB | ARG | A | 45 | 50.906 | 10.639 | 66.148 | 1.00 | 30.80 |
| ATOM | 362 | CG | ARG | A | 45 | 50.324 | 11.778 | 66.956 | 1.00 | 33.11 |
| ATOM | 363 | CD | ARG | A | 45 | 49.976 | 13.022 | 66.097 | 1.00 | 31.47 |
| ATOM | 364 | NE | ARG | A | 45 | 51.175 | 13.653 | 65.566 | 1.00 | 31.60 |
| ATOM | 365 | CZ | ARG | A | 45 | 52.045 | 14.366 | 66.295 | 1.00 | 30.87 |
| ATOM | 366 | NH1 | ARG | A | 45 | 51.844 | 14.584 | 67.583 | 1.00 | 32.36 |
| ATOM | 367 | NH2 | ARG | A | 45 | 53.126 | 14.869 | 65.725 | 1.00 | 31.31 |
| ATOM | 368 | N | TYR | A | 46 | 50.377 | 9.110 | 69.136 | 1.00 | 42.46 |
| ATOM | 369 | CA | TYR | A | 46 | 49.474 | 8.579 | 70.144 | 1.00 | 47.44 |
| ATOM | 370 | C | TYR | A | 46 | 48.146 | 9.272 | 69.956 | 1.00 | 48.50 |
| ATOM | 371 | O | TYR | A | 46 | 48.109 | 10.419 | 69.504 | 1.00 | 47.96 |
| ATOM | 372 | CB | TYR | A | 46 | 50.027 | 8.827 | 71.560 | 1.00 | 50.82 |
| ATOM | 373 | CG | TYR | A | 46 | 49.268 | 8.129 | 72.680 | 1.00 | 54.90 |
| ATOM | 374 | CD1 | TYR | A | 46 | 49.341 | 6.744 | 72.851 | 1.00 | 59.72 |
| ATOM | 375 | CD2 | TYR | A | 46 | 48.488 | 8.857 | 73.583 | 1.00 | 59.64 |
| ATOM | 376 | CE1 | TYR | A | 46 | 48.652 | 6.101 | 73.899 | 1.00 | 61.54 |
| ATOM | 377 | CE2 | TYR | A | 46 | 47.795 | 8.225 | 74.632 | 1.00 | 59.70 |
| ATOM | 378 | CZ | TYR | A | 46 | 47.883 | 6.852 | 74.785 | 1.00 | 62.30 |
| ATOM | 379 | OH | TYR | A | 46 | 47.202 | 6.232 | 75.817 | 1.00 | 63.70 |
| ATOM | 380 | N | ARG | A | 47 | 47.062 | 8.560 | 70.263 | 1.00 | 50.57 |
| ATOM | 381 | CA | ARG | A | 47 | 45.694 | 9.060 | 70.089 | 1.00 | 52.62 |
| ATOM | 382 | C | ARG | A | 47 | 45.345 | 9.364 | 68.630 | 1.00 | 49.86 |
| ATOM | 383 | O | ARG | A | 47 | 44.298 | 9.964 | 68.353 | 1.00 | 52.74 |
| ATOM | 384 | CB | ARG | A | 47 | 45.430 | 10.286 | 70.977 | 1.00 | 57.04 |
| ATOM | 385 | CG | ARG | A | 47 | 45.252 | 9.948 | 72.451 | 1.00 | 63.95 |
| ATOM | 386 | CD | ARG | A | 47 | 45.456 | 11.129 | 73.399 | 1.00 | 67.89 |
| ATOM | 387 | NE | ARG | A | 47 | 45.300 | 10.715 | 74.793 | 1.00 | 72.16 |
| ATOM | 388 | CZ | ARG | A | 47 | 44.134 | 10.532 | 75.414 | 1.00 | 76.00 |
| ATOM | 389 | NH1 | ARG | A | 47 | 44.121 | 10.144 | 76.685 | 1.00 | 77.94 |
| ATOM | 390 | NH2 | ARG | A | 47 | 42.980 | 10.745 | 74.788 | 1.00 | 76.37 |
| ATOM | 391 | N | ASP | A | 48 | 46.200 | 8.931 | 67.703 | 1.00 | 46.81 |
| ATOM | 392 | CA | ASP | A | 48 | 45.918 | 9.062 | 66.275 | 1.00 | 46.76 |
| ATOM | 393 | C | ASP | A | 48 | 45.454 | 7.728 | 65.664 | 1.00 | 43.75 |
| ATOM | 394 | O | ASP | A | 48 | 45.634 | 6.647 | 66.228 | 1.00 | 43.52 |
| ATOM | 395 | CB | ASP | A | 48 | 47.139 | 9.590 | 65.505 | 1.00 | 46.15 |
| ATOM | 396 | CG | ASP | A | 48 | 46.753 | 10.511 | 64.337 | 1.00 | 46.12 |
| ATOM | 397 | OD1 | ASP | A | 48 | 45.610 | 10.448 | 63.865 | 1.00 | 47.80 |
| ATOM | 398 | OD2 | ASP | A | 48 | 47.520 | 11.345 | 63.824 | 1.00 | 45.97 |
| ATOM | 399 | N | VAL | A | 49 | 44.837 | 7.855 | 64.503 | 1.00 | 39.86 |
| ATOM | 400 | CA | VAL | A | 49 | 44.386 | 6.746 | 63.695 | 1.00 | 40.09 |
| ATOM | 401 | C | VAL | A | 49 | 45.550 | 5.875 | 63.214 | 1.00 | 35.44 |
| ATOM | 402 | O | VAL | A | 49 | 46.690 | 6.335 | 63.105 | 1.00 | 34.85 |
| ATOM | 403 | CB | VAL | A | 49 | 43.565 | 7.342 | 62.512 | 1.00 | 45.24 |
| ATOM | 404 | CG1 | VAL | A | 49 | 43.677 | 6.530 | 61.240 | 1.00 | 46.89 |
| ATOM | 405 | CG2 | VAL | A | 49 | 42.111 | 7.550 | 62.942 | 1.00 | 44.64 |
| ATOM | 406 | N | SER | A | 50 | 45.270 | 4.599 | 62.963 | 1.00 | 30.48 |
| ATOM | 407 | CA | SER | A | 50 | 46.239 | 3.721 | 62.311 | 1.00 | 32.31 |
| ATOM | 408 | C | SER | A | 50 | 45.821 | 3.522 | 60.858 | 1.00 | 30.62 |
| ATOM | 409 | O | SER | A | 50 | 44.628 | 3.445 | 60.566 | 1.00 | 27.62 |
| ATOM | 410 | CB | SER | A | 50 | 46.301 | 2.353 | 63.001 | 1.00 | 36.08 |
| ATOM | 411 | OG | SER | A | 50 | 46.626 | 2.460 | 64.373 | 1.00 | 40.07 |
| ATOM | 412 | N | PRO | A | 51 | 46.788 | 3.414 | 59.950 | 1.00 | 28.51 |
| ATOM | 413 | CA | PRO | A | 51 | 46.485 | 3.159 | 58.547 | 1.00 | 28.46 |
| ATOM | 414 | C | PRO | A | 51 | 46.182 | 1.679 | 58.272 | 1.00 | 33.24 |
| ATOM | 415 | O | PRO | A | 51 | 46.963 | 0.814 | 58.738 | 1.00 | 31.33 |
| ATOM | 416 | CB | PRO | A | 51 | 47.784 | 3.521 | 57.852 | 1.00 | 24.78 |
| ATOM | 417 | CG | PRO | A | 51 | 48.813 | 3.248 | 58.844 | 1.00 | 26.09 |
| ATOM | 418 | CD | PRO | A | 51 | 48.238 | 3.489 | 60.180 | 1.00 | 26.48 |
| ATOM | 419 | N | PHE | A | 52 | 45.120 | 1.401 | 57.501 | 1.00 | 30.61 |
| ATOM | 420 | CA | PHE | A | 52 | 44.864 | 0.029 | 57.026 | 1.00 | 28.44 |
| ATOM | 421 | C | PHE | A | 52 | 46.004 | −0.422 | 56.175 | 1.00 | 29.63 |
| ATOM | 422 | O | PHE | A | 52 | 46.594 | 0.370 | 55.429 | 1.00 | 32.01 |
| ATOM | 423 | CB | PHE | A | 52 | 43.626 | −0.068 | 56.169 | 1.00 | 24.75 |
| ATOM | 424 | CG | PHE | A | 52 | 42.387 | 0.405 | 56.831 | 1.00 | 22.32 |
| ATOM | 425 | CD1 | PHE | A | 52 | 42.085 | 0.040 | 58.124 | 1.00 | 23.62 |
| ATOM | 426 | CD2 | PHE | A | 52 | 41.491 | 1.199 | 56.125 | 1.00 | 25.33 |
| ATOM | 427 | CE1 | PHE | A | 52 | 40.904 | 0.463 | 58.713 | 1.00 | 23.34 |
| ATOM | 428 | CE2 | PHE | A | 52 | 40.328 | 1.612 | 56.683 | 1.00 | 21.27 |
| ATOM | 429 | CZ | PHE | A | 52 | 40.024 | 1.254 | 57.986 | 1.00 | 28.10 |
| ATOM | 430 | N | ASP | A | 53 | 46.322 | −1.704 | 56.279 | 1.00 | 28.69 |
| ATOM | 431 | CA | ASP | A | 53 | 47.356 | −2.294 | 55.451 | 1.00 | 29.49 |
| ATOM | 432 | C | ASP | A | 53 | 47.130 | −2.099 | 53.960 | 1.00 | 27.77 |
| ATOM | 433 | O | ASP | A | 53 | 48.064 | −1.788 | 53.237 | 1.00 | 32.33 |
| ATOM | 434 | CB | ASP | A | 53 | 47.477 | −3.792 | 55.743 | 1.00 | 35.42 |
| ATOM | 435 | CG | ASP | A | 53 | 48.004 | −4.065 | 57.126 | 1.00 | 38.63 |
| ATOM | 436 | OD1 | ASP | A | 53 | 48.904 | −3.317 | 57.572 | 1.00 | 39.43 |
| ATOM | 437 | OD2 | ASP | A | 53 | 47.570 | −4.992 | 57.835 | 1.00 | 37.94 |
| ATOM | 438 | N | HIS | A | 54 | 45.905 | −2.295 | 53.496 | 1.00 | 30.15 |
| ATOM | 439 | CA | HIS | A | 54 | 45.671 | −2.394 | 52.057 | 1.00 | 32.40 |
| ATOM | 440 | C | HIS | A | 54 | 45.927 | −1.071 | 51.339 | 1.00 | 34.34 |
| ATOM | 441 | O | HIS | A | 54 | 46.338 | −1.063 | 50.174 | 1.00 | 32.57 |
| ATOM | 442 | CB | HIS | A | 54 | 44.253 | −2.920 | 51.757 | 1.00 | 30.82 |
| ATOM | 443 | CG | HIS | A | 54 | 43.144 | −1.926 | 51.968 | 1.00 | 31.84 |
| ATOM | 444 | ND1 | HIS | A | 54 | 42.695 | −1.081 | 50.973 | 1.00 | 31.38 |
| ATOM | 445 | CD2 | HIS | A | 54 | 42.342 | −1.700 | 53.032 | 1.00 | 29.25 |
| ATOM | 446 | CE1 | HIS | A | 54 | 41.682 | −0.367 | 51.423 | 1.00 | 25.81 |
| ATOM | 447 | NE2 | HIS | A | 54 | 41.450 | −0.718 | 52.673 | 1.00 | 28.42 |
| ATOM | 448 | N | SER | A | 55 | 45.691 | 0.034 | 52.052 | 1.00 | 33.04 |
| ATOM | 449 | CA | SER | A | 55 | 45.764 | 1.360 | 51.463 | 1.00 | 31.70 |
| ATOM | 450 | C | SER | A | 55 | 46.975 | 2.163 | 51.953 | 1.00 | 32.19 |
| ATOM | 451 | O | SER | A | 55 | 47.131 | 3.315 | 51.575 | 1.00 | 31.89 |
| ATOM | 452 | CB | SER | A | 55 | 44.472 | 2.125 | 51.740 | 1.00 | 27.88 |
| ATOM | 453 | OG | SER | A | 55 | 44.171 | 2.174 | 53.135 | 1.00 | 29.64 |
| ATOM | 454 | N | ARG | A | 56 | 47.846 | 1.564 | 52.758 | 1.00 | 29.31 |
| ATOM | 455 | CA | ARG | A | 56 | 48.968 | 2.309 | 53.310 | 1.00 | 28.34 |
| ATOM | 456 | C | ARG | A | 56 | 49.965 | 2.696 | 52.244 | 1.00 | 30.31 |
| ATOM | 457 | O | ARG | A | 56 | 50.159 | 1.980 | 51.272 | 1.00 | 32.53 |
| ATOM | 458 | CB | ARG | A | 56 | 49.653 | 1.555 | 54.452 | 1.00 | 29.43 |
| ATOM | 459 | CG | ARG | A | 56 | 50.655 | 0.459 | 54.047 | 1.00 | 29.42 |
| ATOM | 460 | CD | ARG | A | 56 | 51.106 | −0.405 | 55.255 | 1.00 | 31.42 |
| ATOM | 461 | NE | ARG | A | 56 | 52.173 | −1.315 | 54.865 | 1.00 | 32.84 |
| ATOM | 462 | CZ | ARG | A | 56 | 53.458 | −1.039 | 54.853 | 1.00 | 35.87 |
| ATOM | 463 | NH1 | ARG | A | 56 | 53.933 | 0.127 | 55.264 | 1.00 | 39.34 |
| ATOM | 464 | NH2 | ARG | A | 56 | 54.296 | −1.972 | 54.431 | 1.00 | 39.78 |
| ATOM | 465 | N | ILE | A | 57 | 50.582 | 3.860 | 52.430 | 1.00 | 33.20 |
| ATOM | 466 | CA | ILE | A | 57 | 51.638 | 4.334 | 51.552 | 1.00 | 32.52 |
| ATOM | 467 | C | ILE | A | 57 | 52.938 | 3.718 | 52.035 | 1.00 | 33.53 |
| ATOM | 468 | O | ILE | A | 57 | 53.244 | 3.740 | 53.230 | 1.00 | 33.31 |
| ATOM | 469 | CB | ILE | A | 57 | 51.767 | 5.902 | 51.604 | 1.00 | 32.19 |
| ATOM | 470 | CG1 | ILE | A | 57 | 50.527 | 6.591 | 51.022 | 1.00 | 29.83 |
| ATOM | 471 | CG2 | ILE | A | 57 | 53.014 | 6.365 | 50.887 | 1.00 | 27.84 |
| ATOM | 472 | CD1 | ILE | A | 57 | 50.344 | 6.439 | 49.507 | 1.00 | 28.71 |
| ATOM | 473 | N | LYS | A | 58 | 53.718 | 3.200 | 51.103 | 1.00 | 33.28 |
| ATOM | 474 | CA | LYS | A | 58 | 54.993 | 2.598 | 51.439 | 1.00 | 35.42 |
| ATOM | 475 | C | LYS | A | 58 | 56.109 | 3.545 | 51.072 | 1.00 | 33.07 |
| ATOM | 476 | O | LYS | A | 58 | 56.159 | 4.072 | 49.958 | 1.00 | 33.66 |
| ATOM | 477 | CB | LYS | A | 58 | 55.170 | 1.264 | 50.708 | 1.00 | 39.50 |
| ATOM | 478 | CG | LYS | A | 58 | 54.212 | 0.177 | 51.179 | 1.00 | 41.43 |
| ATOM | 479 | CD | LYS | A | 58 | 54.570 | −1.156 | 50.547 | 1.00 | 47.70 |
| ATOM | 480 | CE | LYS | A | 58 | 53.434 | −2.167 | 50.648 | 1.00 | 49.64 |
| ATOM | 481 | NZ | LYS | A | 58 | 53.870 | −3.471 | 50.068 | 1.00 | 53.45 |
| ATOM | 482 | N | LEU | A | 59 | 56.983 | 3.794 | 52.039 | 1.00 | 29.97 |
| ATOM | 483 | CA | LEU | A | 59 | 58.233 | 4.473 | 51.773 | 1.00 | 34.56 |
| ATOM | 484 | C | LEU | A | 59 | 59.072 | 3.533 | 50.916 | 1.00 | 36.28 |
| ATOM | 485 | O | LEU | A | 59 | 58.980 | 2.325 | 51.075 | 1.00 | 37.81 |
| ATOM | 486 | CB | LEU | A | 59 | 58.942 | 4.833 | 53.091 | 1.00 | 32.21 |
| ATOM | 487 | CG | LEU | A | 59 | 58.153 | 5.722 | 54.071 | 1.00 | 29.92 |
| ATOM | 488 | CD1 | LEU | A | 59 | 58.894 | 5.903 | 55.399 | 1.00 | 32.91 |
| ATOM | 489 | CD2 | LEU | A | 59 | 57.875 | 7.066 | 53.428 | 1.00 | 27.92 |
| ATOM | 490 | N | HIS | A | 60 | 59.853 | 4.090 | 49.991 | 1.00 | 41.69 |
| ATOM | 491 | CA | HIS | A | 60 | 60.690 | 3.310 | 49.084 | 1.00 | 45.51 |
| ATOM | 492 | C | HIS | A | 60 | 62.100 | 3.252 | 49.671 | 1.00 | 50.69 |
| ATOM | 493 | O | HIS | A | 60 | 63.052 | 3.731 | 49.062 | 1.00 | 55.71 |
| ATOM | 494 | CB | HIS | A | 60 | 60.738 | 3.935 | 47.670 | 1.00 | 46.25 |
| ATOM | 495 | CG | HIS | A | 60 | 59.400 | 4.042 | 46.996 | 1.00 | 47.90 |
| ATOM | 496 | ND1 | HIS | A | 60 | 59.099 | 5.043 | 46.097 | 1.00 | 48.42 |
| ATOM | 497 | CD2 | HIS | A | 60 | 58.286 | 3.277 | 47.089 | 1.00 | 51.06 |
| ATOM | 498 | CE1 | HIS | A | 60 | 57.859 | 4.892 | 45.667 | 1.00 | 45.41 |
| ATOM | 499 | NE2 | HIS | A | 60 | 57.341 | 3.832 | 46.257 | 1.00 | 49.77 |
| ATOM | 500 | N | GLN | A | 61 | 62.217 | 2.721 | 50.880 | 1.00 | 55.19 |
| ATOM | 501 | CA | GLN | A | 61 | 63.515 | 2.435 | 51.486 | 1.00 | 61.19 |
| ATOM | 502 | C | GLN | A | 61 | 63.514 | 0.957 | 51.874 | 1.00 | 64.61 |
| ATOM | 503 | O | GLN | A | 61 | 62.447 | 0.359 | 52.077 | 1.00 | 64.15 |
| ATOM | 504 | CB | GLN | A | 61 | 63.813 | 3.359 | 52.689 | 1.00 | 62.20 |
| ATOM | 505 | CG | GLN | A | 61 | 62.705 | 3.478 | 53.743 | 1.00 | 63.36 |
| ATOM | 506 | CD | GLN | A | 61 | 63.115 | 4.319 | 54.981 | 1.00 | 66.07 |
| ATOM | 507 | OE1 | GLN | A | 61 | 63.334 | 3.767 | 56.069 | 1.00 | 68.99 |
| ATOM | 508 | NE2 | GLN | A | 61 | 63.193 | 5.642 | 54.817 | 1.00 | 57.39 |
| ATOM | 509 | N | GLU | A | 62 | 64.698 | 0.358 | 51.931 | 1.00 | 68.33 |
| ATOM | 510 | CA | GLU | A | 62 | 64.806 | −1.077 | 52.212 | 1.00 | 71.80 |
| ATOM | 511 | C | GLU | A | 62 | 64.616 | −1.382 | 53.702 | 1.00 | 71.23 |
| ATOM | 512 | O | GLU | A | 62 | 64.096 | −2.438 | 54.066 | 1.00 | 71.32 |
| ATOM | 513 | CB | GLU | A | 62 | 66.152 | −1.627 | 51.712 | 1.00 | 74.26 |
| ATOM | 514 | CG | GLU | A | 62 | 66.105 | −2.165 | 50.282 | 1.00 | 78.26 |
| ATOM | 515 | CD | GLU | A | 62 | 67.440 | −2.074 | 49.551 | 1.00 | 81.64 |
| ATOM | 516 | OE1 | GLU | A | 62 | 68.503 | −1.974 | 50.214 | 1.00 | 81.39 |
| ATOM | 517 | OE2 | GLU | A | 62 | 67.424 | −2.107 | 48.298 | 1.00 | 84.40 |
| ATOM | 518 | N | ASP | A | 63 | 65.010 | −0.435 | 54.551 | 1.00 | 71.07 |
| ATOM | 519 | CA | ASP | A | 63 | 65.050 | −0.633 | 56.002 | 1.00 | 70.34 |
| ATOM | 520 | C | ASP | A | 63 | 63.651 | −0.881 | 56.598 | 1.00 | 65.10 |
| ATOM | 521 | O | ASP | A | 63 | 63.319 | −2.008 | 56.975 | 1.00 | 65.91 |
| ATOM | 522 | CB | ASP | A | 63 | 65.750 | 0.572 | 56.670 | 1.00 | 73.08 |
| ATOM | 523 | CG | ASP | A | 63 | 66.320 | 0.243 | 58.042 | 1.00 | 77.45 |
| ATOM | 524 | OD1 | ASP | A | 63 | 67.352 | −0.468 | 58.109 | 1.00 | 79.35 |
| ATOM | 525 | OD2 | ASP | A | 63 | 65.813 | 0.668 | 59.107 | 1.00 | 80.87 |
| ATOM | 526 | N | ASN | A | 64 | 62.844 | 0.174 | 56.675 | 1.00 | 58.43 |
| ATOM | 527 | CA | ASN | A | 64 | 61.477 | 0.099 | 57.193 | 1.00 | 52.04 |
| ATOM | 528 | C | ASN | A | 64 | 60.627 | 1.023 | 56.332 | 1.00 | 48.60 |
| ATOM | 529 | O | ASN | A | 64 | 60.852 | 2.233 | 56.303 | 1.00 | 47.24 |
| ATOM | 530 | CB | ASN | A | 64 | 61.413 | 0.522 | 58.668 | 1.00 | 48.57 |
| ATOM | 531 | CG | ASN | A | 64 | 60.047 | 0.296 | 59.277 | 1.00 | 49.39 |
| ATOM | 532 | OD1 | ASN | A | 64 | 59.097 | −0.023 | 58.567 | 1.00 | 52.11 |
| ATOM | 533 | ND2 | ASN | A | 64 | 59.934 | 0.459 | 60.597 | 1.00 | 43.17 |
| ATOM | 534 | N | ASP | A | 65 | 59.671 | 0.447 | 55.614 | 1.00 | 44.68 |
| ATOM | 535 | CA | ASP | A | 65 | 58.893 | 1.200 | 54.640 | 1.00 | 41.56 |
| ATOM | 536 | C | ASP | A | 65 | 57.637 | 1.859 | 55.230 | 1.00 | 37.75 |
| ATOM | 537 | O | ASP | A | 65 | 56.816 | 2.386 | 54.488 | 1.00 | 37.27 |
| ATOM | 538 | CB | ASP | A | 65 | 58.546 | 0.312 | 53.424 | 1.00 | 42.30 |
| ATOM | 539 | CG | ASP | A | 65 | 57.516 | −0.766 | 53.730 | 1.00 | 41.84 |
| ATOM | 540 | OD1 | ASP | A | 65 | 56.979 | −0.819 | 54.847 | 1.00 | 42.12 |
| ATOM | 541 | OD2 | ASP | A | 65 | 57.175 | −1.614 | 52.887 | 1.00 | 47.21 |
| ATOM | 542 | N | TYR | A | 66 | 57.495 | 1.836 | 56.553 | 1.00 | 32.69 |
| ATOM | 543 | CA | TYR | A | 66 | 56.263 | 2.258 | 57.185 | 1.00 | 34.98 |
| ATOM | 544 | C | TYR | A | 66 | 56.171 | 3.765 | 57.523 | 1.00 | 30.91 |
| ATOM | 545 | O | TYR | A | 66 | 57.059 | 4.359 | 58.106 | 1.00 | 30.40 |
| ATOM | 546 | CB | TYR | A | 66 | 55.983 | 1.446 | 58.443 | 1.00 | 29.07 |
| ATOM | 547 | CG | TYR | A | 66 | 54.739 | 1.934 | 59.146 | 1.00 | 31.86 |
| ATOM | 548 | CD1 | TYR | A | 66 | 53.481 | 1.556 | 58.712 | 1.00 | 29.85 |
| ATOM | 549 | CD2 | TYR | A | 66 | 54.821 | 2.799 | 60.228 | 1.00 | 31.65 |
| ATOM | 550 | CE1 | TYR | A | 66 | 52.339 | 2.006 | 59.347 | 1.00 | 27.49 |
| ATOM | 551 | CE2 | TYR | A | 66 | 53.682 | 3.257 | 60.874 | 1.00 | 30.06 |
| ATOM | 552 | CZ | TYR | A | 66 | 52.442 | 2.867 | 60.426 | 1.00 | 27.92 |
| ATOM | 553 | OH | TYR | A | 66 | 51.298 | 3.317 | 61.057 | 1.00 | 23.79 |
| ATOM | 554 | N | ILE | A | 67 | 55.041 | 4.342 | 57.148 | 1.00 | 31.87 |
| ATOM | 555 | CA | ILE | A | 67 | 54.637 | 5.669 | 57.594 | 1.00 | 28.25 |
| ATOM | 556 | C | ILE | A | 67 | 53.140 | 5.603 | 57.818 | 1.00 | 26.62 |
| ATOM | 557 | O | ILE | A | 67 | 52.426 | 4.932 | 57.063 | 1.00 | 27.32 |
| ATOM | 558 | CB | ILE | A | 67 | 55.010 | 6.737 | 56.519 | 1.00 | 26.62 |
| ATOM | 559 | CG1 | ILE | A | 67 | 54.582 | 8.146 | 56.964 | 1.00 | 25.26 |
| ATOM | 560 | CG2 | ILE | A | 67 | 54.383 | 6.378 | 55.166 | 1.00 | 27.78 |
| ATOM | 561 | CD1 | ILE | A | 67 | 55.183 | 9.282 | 56.135 | 1.00 | 23.21 |
| ATOM | 562 | N | ASN | A | 68 | 52.663 | 6.274 | 58.856 | 1.00 | 24.43 |
| ATOM | 563 | CA | ASN | A | 68 | 51.248 | 6.399 | 59.090 | 1.00 | 26.84 |
| ATOM | 564 | C | ASN | A | 68 | 50.614 | 7.339 | 58.044 | 1.00 | 29.79 |
| ATOM | 565 | O | ASN | A | 68 | 50.396 | 8.529 | 58.301 | 1.00 | 27.07 |
| ATOM | 566 | CB | ASN | A | 68 | 50.966 | 6.891 | 60.504 | 1.00 | 23.80 |
| ATOM | 567 | CG | ASN | A | 68 | 49.514 | 6.815 | 60.844 | 1.00 | 27.62 |
| ATOM | 568 | OD1 | ASN | A | 68 | 48.672 | 6.723 | 59.950 | 1.00 | 33.26 |
| ATOM | 569 | ND2 | ASN | A | 68 | 49.190 | 6.842 | 62.127 | 1.00 | 29.87 |
| ATOM | 570 | N | ALA | A | 69 | 50.335 | 6.776 | 56.869 | 1.00 | 26.62 |
| ATOM | 571 | CA | ALA | A | 69 | 49.661 | 7.473 | 55.785 | 1.00 | 26.37 |
| ATOM | 572 | C | ALA | A | 69 | 48.863 | 6.476 | 54.931 | 1.00 | 28.38 |
| ATOM | 573 | O | ALA | A | 69 | 49.277 | 5.316 | 54.764 | 1.00 | 25.53 |
| ATOM | 574 | CB | ALA | A | 69 | 50.660 | 8.224 | 54.934 | 1.00 | 25.04 |
| ATOM | 575 | N | SER | A | 70 | 47.728 | 6.943 | 54.403 | 1.00 | 26.26 |
| ATOM | 576 | CA | SER | A | 70 | 46.806 | 6.118 | 53.629 | 1.00 | 26.54 |
| ATOM | 577 | C | SER | A | 70 | 46.455 | 6.824 | 52.323 | 1.00 | 28.75 |
| ATOM | 578 | O | SER | A | 70 | 46.271 | 8.039 | 52.310 | 1.00 | 25.27 |
| ATOM | 579 | CB | SER | A | 70 | 45.511 | 5.891 | 54.401 | 1.00 | 23.26 |
| ATOM | 580 | OG | SER | A | 70 | 45.728 | 5.431 | 55.728 | 1.00 | 26.58 |
| ATOM | 581 | N | LEU | A | 71 | 46.347 | 6.052 | 51.246 | 1.00 | 26.40 |
| ATOM | 582 | CA | LEU | A | 71 | 45.835 | 6.527 | 49.969 | 1.00 | 26.16 |
| ATOM | 583 | C | LEU | A | 71 | 44.329 | 6.317 | 49.851 | 1.00 | 27.58 |
| ATOM | 584 | O | LEU | A | 71 | 43.828 | 5.190 | 49.930 | 1.00 | 31.42 |
| ATOM | 585 | CB | LEU | A | 71 | 46.564 | 5.863 | 48.817 | 1.00 | 27.02 |
| ATOM | 586 | CG | LEU | A | 71 | 46.149 | 6.190 | 47.374 | 1.00 | 27.68 |
| ATOM | 587 | CD1 | LEU | A | 71 | 46.751 | 5.131 | 46.382 | 1.00 | 29.17 |
| ATOM | 588 | CD2 | LEU | A | 71 | 46.580 | 7.597 | 46.956 | 1.00 | 30.23 |
| ATOM | 589 | N | ILE | A | 72 | 43.614 | 7.429 | 49.704 | 1.00 | 27.27 |
| ATOM | 590 | CA | ILE | A | 72 | 42.190 | 7.452 | 49.457 | 1.00 | 29.78 |
| ATOM | 591 | C | ILE | A | 72 | 42.054 | 7.655 | 47.948 | 1.00 | 33.13 |
| ATOM | 592 | O | ILE | A | 72 | 42.507 | 8.664 | 47.413 | 1.00 | 29.74 |
| ATOM | 593 | CB | ILE | A | 72 | 41.530 | 8.616 | 50.182 | 1.00 | 29.24 |
| ATOM | 594 | CG1 | ILE | A | 72 | 41.497 | 8.436 | 51.698 | 1.00 | 33.27 |
| ATOM | 595 | CG2 | ILE | A | 72 | 40.098 | 8.791 | 49.713 | 1.00 | 31.42 |
| ATOM | 596 | CD1 | ILE | A | 72 | 42.801 | 8.343 | 52.352 | 1.00 | 39.18 |
| ATOM | 597 | N | LYS | A | 73 | 41.459 | 6.690 | 47.262 | 1.00 | 31.14 |
| ATOM | 598 | CA | LYS | A | 73 | 41.370 | 6.744 | 45.810 | 1.00 | 35.01 |
| ATOM | 599 | C | LYS | A | 73 | 39.910 | 6.820 | 45.454 | 1.00 | 31.26 |
| ATOM | 600 | O | LYS | A | 73 | 39.156 | 5.874 | 45.708 | 1.00 | 32.26 |
| ATOM | 601 | CB | LYS | A | 73 | 42.032 | 5.526 | 45.175 | 1.00 | 38.92 |
| ATOM | 602 | CG | LYS | A | 73 | 42.707 | 5.838 | 43.842 | 1.00 | 49.66 |
| ATOM | 603 | CD | LYS | A | 73 | 43.222 | 4.576 | 43.129 | 1.00 | 56.02 |
| ATOM | 604 | CE | LYS | A | 73 | 44.527 | 4.066 | 43.749 | 1.00 | 59.01 |
| ATOM | 605 | NZ | LYS | A | 73 | 44.757 | 2.611 | 43.510 | 1.00 | 62.37 |
| ATOM | 606 | N | MET | A | 74 | 39.481 | 7.966 | 44.934 | 1.00 | 26.18 |
| ATOM | 607 | CA | MET | A | 74 | 38.069 | 8.129 | 44.575 | 1.00 | 27.64 |
| ATOM | 608 | C | MET | A | 74 | 37.944 | 7.987 | 43.069 | 1.00 | 29.42 |
| ATOM | 609 | O | MET | A | 74 | 38.197 | 8.931 | 42.309 | 1.00 | 30.17 |
| ATOM | 610 | CB | MET | A | 74 | 37.512 | 9.441 | 45.096 | 1.00 | 27.81 |
| ATOM | 611 | CG | MET | A | 74 | 37.712 | 9.614 | 46.593 | 1.00 | 30.72 |
| ATOM | 612 | SD | MET | A | 74 | 36.826 | 8.410 | 47.617 | 1.00 | 31.58 |
| ATOM | 613 | CE | MET | A | 74 | 35.155 | 8.853 | 47.354 | 1.00 | 27.53 |
| ATOM | 614 | N | GLU | A | 75 | 37.611 | 6.764 | 42.651 | 1.00 | 32.89 |
| ATOM | 615 | CA | GLU | A | 75 | 37.606 | 6.352 | 41.238 | 1.00 | 35.37 |
| ATOM | 616 | C | GLU | A | 75 | 36.704 | 7.213 | 40.343 | 1.00 | 27.34 |
| ATOM | 617 | O | GLU | A | 75 | 37.151 | 7.727 | 39.330 | 1.00 | 31.16 |
| ATOM | 618 | CB | GLU | A | 75 | 37.191 | 4.867 | 41.129 | 1.00 | 40.62 |
| ATOM | 619 | CG | GLU | A | 75 | 37.470 | 4.231 | 39.774 | 1.00 | 48.74 |
| ATOM | 620 | CD | GLU | A | 75 | 37.322 | 2.715 | 39.787 | 1.00 | 54.93 |
| ATOM | 621 | OE1 | GLU | A | 75 | 36.174 | 2.224 | 39.768 | 1.00 | 60.04 |
| ATOM | 622 | OE2 | GLU | A | 75 | 38.357 | 2.008 | 39.815 | 1.00 | 63.82 |
| ATOM | 623 | N | GLU | A | 76 | 35.440 | 7.362 | 40.711 | 1.00 | 28.64 |
| ATOM | 624 | CA | GLU | A | 76 | 34.497 | 8.117 | 39.893 | 1.00 | 29.79 |
| ATOM | 625 | C | GLU | A | 76 | 34.881 | 9.603 | 39.808 | 1.00 | 33.33 |
| ATOM | 626 | O | GLU | A | 76 | 34.785 | 10.206 | 38.742 | 1.00 | 31.81 |
| ATOM | 627 | CB | GLU | A | 76 | 33.079 | 7.998 | 40.443 | 1.00 | 31.29 |
| ATOM | 628 | CG | GLU | A | 76 | 32.031 | 8.574 | 39.505 | 1.00 | 32.55 |
| ATOM | 629 | CD | GLU | A | 76 | 30.689 | 8.782 | 40.159 | 1.00 | 33.85 |
| ATOM | 630 | OE1 | GLU | A | 76 | 30.479 | 8.342 | 41.316 | 1.00 | 32.93 |
| ATOM | 631 | OE2 | GLU | A | 76 | 29.838 | 9.410 | 39.502 | 1.00 | 37.55 |
| ATOM | 632 | N | ALA | A | 77 | 35.311 | 10.187 | 40.931 | 1.00 | 31.97 |
| ATOM | 633 | CA | ALA | A | 77 | 35.668 | 11.599 | 40.964 | 1.00 | 28.17 |
| ATOM | 634 | C | ALA | A | 77 | 37.032 | 11.856 | 40.349 | 1.00 | 26.78 |
| ATOM | 635 | O | ALA | A | 77 | 37.390 | 12.990 | 40.085 | 1.00 | 31.00 |
| ATOM | 636 | CB | ALA | A | 77 | 35.627 | 12.113 | 42.400 | 1.00 | 29.56 |
| ATOM | 637 | N | GLN | A | 78 | 37.803 | 10.793 | 40.137 | 1.00 | 27.94 |
| ATOM | 638 | CA | GLN | A | 78 | 39.180 | 10.886 | 39.667 | 1.00 | 29.55 |
| ATOM | 639 | C | GLN | A | 78 | 40.055 | 11.791 | 40.550 | 1.00 | 28.24 |
| ATOM | 640 | O | GLN | A | 78 | 40.840 | 12.595 | 40.066 | 1.00 | 29.87 |
| ATOM | 641 | CB | GLN | A | 78 | 39.232 | 11.263 | 38.167 | 1.00 | 36.39 |
| ATOM | 642 | CG | GLN | A | 78 | 38.797 | 10.096 | 37.229 | 1.00 | 40.91 |
| ATOM | 643 | CD | GLN | A | 78 | 39.758 | 8.891 | 37.277 | 1.00 | 44.12 |
| ATOM | 644 | OE1 | GLN | A | 78 | 40.866 | 8.955 | 36.741 | 1.00 | 45.28 |
| ATOM | 645 | NE2 | GLN | A | 78 | 39.333 | 7.805 | 37.926 | 1.00 | 44.61 |
| ATOM | 646 | N | ARG | A | 79 | 39.940 | 11.604 | 41.859 | 1.00 | 26.27 |
| ATOM | 647 | CA | ARG | A | 79 | 40.784 | 12.298 | 42.834 | 1.00 | 27.36 |
| ATOM | 648 | C | ARG | A | 79 | 41.427 | 11.294 | 43.735 | 1.00 | 27.37 |
| ATOM | 649 | O | ARG | A | 79 | 40.787 | 10.349 | 44.172 | 1.00 | 25.21 |
| ATOM | 650 | CB | ARG | A | 79 | 39.953 | 13.206 | 43.734 | 1.00 | 25.02 |
| ATOM | 651 | CG | ARG | A | 79 | 39.619 | 14.494 | 43.106 | 1.00 | 27.59 |
| ATOM | 652 | CD | ARG | A | 79 | 40.706 | 15.532 | 43.201 | 1.00 | 24.51 |
| ATOM | 653 | NE | ARG | A | 79 | 40.107 | 16.836 | 43.414 | 1.00 | 29.17 |
| ATOM | 654 | CZ | ARG | A | 79 | 40.020 | 17.805 | 42.517 | 1.00 | 27.84 |
| ATOM | 655 | NH1 | ARG | A | 79 | 39.449 | 18.944 | 42.876 | 1.00 | 30.27 |
| ATOM | 656 | NH2 | ARG | A | 79 | 40.478 | 17.662 | 41.284 | 1.00 | 27.06 |
| ATOM | 657 | N | SER | A | 80 | 42.686 | 11.547 | 44.056 | 1.00 | 24.62 |
| ATOM | 658 | CA | SER | A | 80 | 43.380 | 10.826 | 45.086 | 1.00 | 26.77 |
| ATOM | 659 | C | SER | A | 80 | 43.861 | 11.806 | 46.151 | 1.00 | 26.33 |
| ATOM | 660 | O | SER | A | 80 | 44.269 | 12.931 | 45.837 | 1.00 | 24.64 |
| ATOM | 661 | CB | SER | A | 80 | 44.560 | 10.082 | 44.471 | 1.00 | 28.33 |
| ATOM | 662 | OG | SER | A | 80 | 44.095 | 9.006 | 43.697 | 1.00 | 33.53 |
| ATOM | 663 | N | TYR | A | 81 | 43.821 | 11.361 | 47.405 | 1.00 | 24.21 |
| ATOM | 664 | CA | TYR | A | 81 | 44.408 | 12.085 | 48.531 | 1.00 | 23.65 |
| ATOM | 665 | C | TYR | A | 81 | 45.216 | 11.094 | 49.387 | 1.00 | 24.13 |
| ATOM | 666 | O | TYR | A | 81 | 44.814 | 9.938 | 49.555 | 1.00 | 24.40 |
| ATOM | 667 | CB | TYR | A | 81 | 43.311 | 12.713 | 49.401 | 1.00 | 22.88 |
| ATOM | 668 | CG | TYR | A | 81 | 42.102 | 13.270 | 48.670 | 1.00 | 23.10 |
| ATOM | 669 | CD1 | TYR | A | 81 | 41.044 | 12.452 | 48.286 | 1.00 | 23.35 |
| ATOM | 670 | CD2 | TYR | A | 81 | 42.000 | 14.630 | 48.390 | 1.00 | 21.42 |
| ATOM | 671 | CE1 | TYR | A | 81 | 39.916 | 12.980 | 47.623 | 1.00 | 18.91 |
| ATOM | 672 | CE2 | TYR | A | 81 | 40.887 | 15.156 | 47.741 | 1.00 | 22.29 |
| ATOM | 673 | CZ | TYR | A | 81 | 39.854 | 14.320 | 47.353 | 1.00 | 21.87 |
| ATOM | 674 | OH | TYR | A | 81 | 38.757 | 14.845 | 46.716 | 1.00 | 20.42 |
| ATOM | 675 | N | ILE | A | 82 | 46.355 | 11.532 | 49.916 | 1.00 | 20.24 |
| ATOM | 676 | CA | ILE | A | 82 | 47.060 | 10.802 | 50.963 | 1.00 | 20.61 |
| ATOM | 677 | C | ILE | A | 82 | 46.759 | 11.527 | 52.265 | 1.00 | 24.67 |
| ATOM | 678 | O | ILE | A | 82 | 47.032 | 12.728 | 52.355 | 1.00 | 24.93 |
| ATOM | 679 | CB | ILE | A | 82 | 48.581 | 10.810 | 50.725 | 1.00 | 22.20 |
| ATOM | 680 | CG1 | ILE | A | 82 | 48.965 | 9.944 | 49.529 | 1.00 | 23.16 |
| ATOM | 681 | CG2 | ILE | A | 82 | 49.309 | 10.327 | 51.984 | 1.00 | 22.16 |
| ATOM | 682 | CD1 | ILE | A | 82 | 50.328 | 10.228 | 49.001 | 1.00 | 23.55 |
| ATOM | 683 | N | LEU | A | 83 | 46.177 | 10.820 | 53.245 | 1.00 | 22.39 |
| ATOM | 684 | CA | LEU | A | 83 | 45.939 | 11.359 | 54.574 | 1.00 | 22.02 |
| ATOM | 685 | C | LEU | A | 83 | 46.951 | 10.755 | 55.513 | 1.00 | 23.73 |
| ATOM | 686 | O | LEU | A | 83 | 47.179 | 9.560 | 55.520 | 1.00 | 22.83 |
| ATOM | 687 | CB | LEU | A | 83 | 44.519 | 11.096 | 55.082 | 1.00 | 23.84 |
| ATOM | 688 | CG | LEU | A | 83 | 43.488 | 12.171 | 54.725 | 1.00 | 26.43 |
| ATOM | 689 | CD1 | LEU | A | 83 | 43.340 | 12.273 | 53.201 | 1.00 | 29.89 |
| ATOM | 690 | CD2 | LEU | A | 83 | 42.141 | 11.863 | 55.353 | 1.00 | 30.50 |
| ATOM | 691 | N | THR | A | 84 | 47.575 | 11.604 | 56.307 | 1.00 | 21.81 |
| ATOM | 692 | CA | THR | A | 84 | 48.629 | 11.163 | 57.189 | 1.00 | 21.57 |
| ATOM | 693 | C | THR | A | 84 | 48.499 | 11.897 | 58.510 | 1.00 | 22.44 |
| ATOM | 694 | O | THR | A | 84 | 47.605 | 12.757 | 58.680 | 1.00 | 20.53 |
| ATOM | 695 | CB | THR | A | 84 | 49.983 | 11.320 | 56.482 | 1.00 | 21.64 |
| ATOM | 696 | OG1 | THR | A | 84 | 51.020 | 10.742 | 57.262 | 1.00 | 24.20 |
| ATOM | 697 | CG2 | THR | A | 84 | 50.368 | 12.811 | 56.253 | 1.00 | 23.01 |
| ATOM | 698 | N | GLN | A | 85 | 49.285 | 11.453 | 59.488 | 1.00 | 22.81 |
| ATOM | 699 | CA | GLN | A | 85 | 49.354 | 12.121 | 60.780 | 1.00 | 23.95 |
| ATOM | 700 | C | GLN | A | 85 | 50.307 | 13.321 | 60.659 | 1.00 | 23.08 |
| ATOM | 701 | O | GLN | A | 85 | 51.226 | 13.346 | 59.798 | 1.00 | 23.89 |
| ATOM | 702 | CB | GLN | A | 85 | 49.860 | 11.149 | 61.874 | 1.00 | 21.50 |
| ATOM | 703 | CG | GLN | A | 85 | 51.301 | 10.780 | 61.733 | 1.00 | 22.55 |
| ATOM | 704 | CD | GLN | A | 85 | 51.762 | 9.690 | 62.700 | 1.00 | 25.69 |
| ATOM | 705 | OE1 | GLN | A | 85 | 50.951 | 8.958 | 63.282 | 1.00 | 26.39 |
| ATOM | 706 | NE2 | GLN | A | 85 | 53.072 | 9.580 | 62.858 | 1.00 | 23.52 |
| ATOM | 707 | N | GLY | A | 86 | 50.133 | 14.278 | 61.551 | 1.00 | 23.87 |
| ATOM | 708 | CA | GLY | A | 86 | 51.092 | 15.359 | 61.690 | 1.00 | 27.12 |
| ATOM | 709 | C | GLY | A | 86 | 52.425 | 14.728 | 62.032 | 1.00 | 24.81 |
| ATOM | 710 | O | GLY | A | 86 | 52.469 | 13.820 | 62.884 | 1.00 | 25.33 |
| ATOM | 711 | N | PRO | A | 87 | 53.482 | 15.108 | 61.317 | 1.00 | 23.19 |
| ATOM | 712 | CA | PRO | A | 87 | 54.812 | 14.559 | 61.561 | 1.00 | 25.56 |
| ATOM | 713 | C | PRO | A | 87 | 55.268 | 14.636 | 63.017 | 1.00 | 25.86 |
| ATOM | 714 | O | PRO | A | 87 | 54.982 | 15.603 | 63.721 | 1.00 | 23.39 |
| ATOM | 715 | CB | PRO | A | 87 | 55.703 | 15.417 | 60.673 | 1.00 | 27.59 |
| ATOM | 716 | CG | PRO | A | 87 | 54.831 | 15.854 | 59.576 | 1.00 | 25.39 |
| ATOM | 717 | CD | PRO | A | 87 | 53.468 | 15.982 | 60.131 | 1.00 | 25.14 |
| ATOM | 718 | N | LEU | A | 88 | 55.952 | 13.585 | 63.453 | 1.00 | 26.57 |
| ATOM | 719 | CA | LEU | A | 88 | 56.540 | 13.514 | 64.775 | 1.00 | 25.79 |
| ATOM | 720 | C | LEU | A | 88 | 57.967 | 14.044 | 64.697 | 1.00 | 27.23 |
| ATOM | 721 | O | LEU | A | 88 | 58.534 | 14.117 | 63.608 | 1.00 | 27.06 |
| ATOM | 722 | CB | LEU | A | 88 | 56.552 | 12.065 | 65.251 | 1.00 | 29.02 |
| ATOM | 723 | CG | LEU | A | 88 | 55.215 | 11.489 | 65.702 | 1.00 | 27.24 |
| ATOM | 724 | CD1 | LEU | A | 88 | 54.878 | 11.952 | 67.109 | 1.00 | 23.82 |
| ATOM | 725 | CD2 | LEU | A | 88 | 55.257 | 9.959 | 65.661 | 1.00 | 29.51 |
| ATOM | 726 | N | PRO | A | 89 | 58.563 | 14.397 | 65.837 | 1.00 | 26.76 |
| ATOM | 727 | CA | PRO | A | 89 | 59.963 | 14.848 | 65.859 | 1.00 | 28.66 |
| ATOM | 728 | C | PRO | A | 89 | 60.894 | 13.964 | 65.022 | 1.00 | 28.76 |
| ATOM | 729 | O | PRO | A | 89 | 61.788 | 14.484 | 64.331 | 1.00 | 30.30 |
| ATOM | 730 | CB | PRO | A | 89 | 60.324 | 14.796 | 67.346 | 1.00 | 27.52 |
| ATOM | 731 | CG | PRO | A | 89 | 59.040 | 15.032 | 68.049 | 1.00 | 26.96 |
| ATOM | 732 | CD | PRO | A | 89 | 57.969 | 14.400 | 67.185 | 1.00 | 28.31 |
| ATOM | 733 | N | ASN | A | 90 | 60.635 | 12.656 | 65.018 | 1.00 | 30.36 |
| ATOM | 734 | CA | ASN | A | 90 | 61.493 | 11.693 | 64.324 | 1.00 | 29.50 |
| ATOM | 735 | C | ASN | A | 90 | 60.937 | 11.167 | 63.000 | 1.00 | 28.62 |
| ATOM | 736 | O | ASN | A | 90 | 61.581 | 10.330 | 62.381 | 1.00 | 29.73 |
| ATOM | 737 | CB | ASN | A | 90 | 61.829 | 10.514 | 65.265 | 1.00 | 32.07 |
| ATOM | 738 | CG | ASN | A | 90 | 62.631 | 10.954 | 66.491 | 1.00 | 36.69 |
| ATOM | 739 | OD1 | ASN | A | 90 | 63.659 | 11.634 | 66.364 | 1.00 | 37.94 |
| ATOM | 740 | ND2 | ASN | A | 90 | 62.140 | 10.609 | 67.683 | 1.00 | 32.87 |
| ATOM | 741 | N | THR | A | 91 | 59.779 | 11.648 | 62.534 | 1.00 | 26.35 |
| ATOM | 742 | CA | THR | A | 91 | 59.263 | 11.209 | 61.223 | 1.00 | 25.63 |
| ATOM | 743 | C | THR | A | 91 | 59.124 | 12.313 | 60.193 | 1.00 | 26.37 |
| ATOM | 744 | O | THR | A | 91 | 58.444 | 12.134 | 59.194 | 1.00 | 25.56 |
| ATOM | 745 | CB | THR | A | 91 | 57.909 | 10.456 | 61.348 | 1.00 | 25.35 |
| ATOM | 746 | OG1 | THR | A | 91 | 56.882 | 11.342 | 61.805 | 1.00 | 24.06 |
| ATOM | 747 | CG2 | THR | A | 91 | 57.979 | 9.383 | 62.408 | 1.00 | 23.43 |
| ATOM | 748 | N | CYS | A | 92 | 59.770 | 13.451 | 60.420 | 1.00 | 25.07 |
| ATOM | 749 | CA | CYS | A | 92 | 59.765 | 14.538 | 59.439 | 1.00 | 25.34 |
| ATOM | 750 | C | CYS | A | 92 | 60.488 | 14.139 | 58.152 | 1.00 | 24.31 |
| ATOM | 751 | O | CYS | A | 92 | 60.055 | 14.490 | 57.043 | 1.00 | 23.94 |
| ATOM | 752 | CB | CYS | A | 92 | 60.410 | 15.802 | 60.039 | 1.00 | 27.99 |
| ATOM | 753 | SG | CYS | A | 92 | 59.459 | 16.529 | 61.381 | 1.00 | 24.33 |
| ATOM | 754 | N | GLY | A | 93 | 61.599 | 13.429 | 58.291 | 1.00 | 24.10 |
| ATOM | 755 | CA | GLY | A | 93 | 62.336 | 12.934 | 57.132 | 1.00 | 24.88 |
| ATOM | 756 | C | GLY | A | 93 | 61.498 | 11.989 | 56.288 | 1.00 | 28.13 |
| ATOM | 757 | O | GLY | A | 93 | 61.445 | 12.113 | 55.064 | 1.00 | 27.40 |
| ATOM | 758 | N | HIS | A | 94 | 60.812 | 11.075 | 56.978 | 1.00 | 28.43 |
| ATOM | 759 | CA | HIS | A | 94 | 59.881 | 10.133 | 56.372 | 1.00 | 28.20 |
| ATOM | 760 | C | HIS | A | 94 | 58.780 | 10.869 | 55.654 | 1.00 | 24.88 |
| ATOM | 761 | O | HIS | A | 94 | 58.410 | 10.517 | 54.542 | 1.00 | 22.84 |
| ATOM | 762 | CB | HIS | A | 94 | 59.216 | 9.248 | 57.443 | 1.00 | 28.80 |
| ATOM | 763 | CG | HIS | A | 94 | 60.172 | 8.458 | 58.275 | 1.00 | 29.78 |
| ATOM | 764 | ND1 | HIS | A | 94 | 59.756 | 7.676 | 59.328 | 1.00 | 33.11 |
| ATOM | 765 | CD2 | HIS | A | 94 | 61.518 | 8.321 | 58.213 | 1.00 | 34.96 |
| ATOM | 766 | CE1 | HIS | A | 94 | 60.806 | 7.093 | 59.880 | 1.00 | 33.93 |
| ATOM | 767 | NE2 | HIS | A | 94 | 61.887 | 7.470 | 59.225 | 1.00 | 29.90 |
| ATOM | 768 | N | PHE | A | 95 | 58.222 | 11.879 | 56.320 | 1.00 | 23.02 |
| ATOM | 769 | CA | PHE | A | 95 | 57.123 | 12.623 | 55.739 | 1.00 | 19.63 |
| ATOM | 770 | C | PHE | A | 95 | 57.539 | 13.220 | 54.399 | 1.00 | 21.27 |
| ATOM | 771 | O | PHE | A | 95 | 56.855 | 13.033 | 53.387 | 1.00 | 22.51 |
| ATOM | 772 | CB | PHE | A | 95 | 56.588 | 13.662 | 56.732 | 1.00 | 20.48 |
| ATOM | 773 | CG | PHE | A | 95 | 55.586 | 14.618 | 56.142 | 1.00 | 18.33 |
| ATOM | 774 | CD1 | PHE | A | 95 | 54.233 | 14.401 | 56.299 | 1.00 | 20.98 |
| ATOM | 775 | CD2 | PHE | A | 95 | 56.011 | 15.736 | 55.429 | 1.00 | 20.21 |
| ATOM | 776 | CE1 | PHE | A | 95 | 53.305 | 15.278 | 55.771 | 1.00 | 25.19 |
| ATOM | 777 | CE2 | PHE | A | 95 | 55.091 | 16.636 | 54.899 | 1.00 | 20.58 |
| ATOM | 778 | CZ | PHE | A | 95 | 53.732 | 16.408 | 55.073 | 1.00 | 25.85 |
| ATOM | 779 | N | TRP | A | 96 | 58.668 | 13.916 | 54.370 | 1.00 | 23.70 |
| ATOM | 780 | CA | TRP | A | 96 | 59.111 | 14.547 | 53.134 | 1.00 | 21.44 |
| ATOM | 781 | C | TRP | A | 96 | 59.583 | 13.514 | 52.113 | 1.00 | 26.85 |
| ATOM | 782 | O | TRP | A | 96 | 59.481 | 13.734 | 50.904 | 1.00 | 25.37 |
| ATOM | 783 | CB | TRP | A | 96 | 60.180 | 15.601 | 53.428 | 1.00 | 24.68 |
| ATOM | 784 | CG | TRP | A | 96 | 59.561 | 16.795 | 54.090 | 1.00 | 21.64 |
| ATOM | 785 | CD1 | TRP | A | 96 | 59.737 | 17.221 | 55.376 | 1.00 | 21.49 |
| ATOM | 786 | CD2 | TRP | A | 96 | 58.602 | 17.666 | 53.508 | 1.00 | 23.92 |
| ATOM | 787 | NE1 | TRP | A | 96 | 58.955 | 18.327 | 55.616 | 1.00 | 26.29 |
| ATOM | 788 | CE2 | TRP | A | 96 | 58.257 | 18.625 | 54.479 | 1.00 | 21.97 |
| ATOM | 789 | CE3 | TRP | A | 96 | 58.012 | 17.754 | 52.237 | 1.00 | 25.15 |
| ATOM | 790 | CZ2 | TRP | A | 96 | 57.353 | 19.630 | 54.230 | 1.00 | 23.22 |
| ATOM | 791 | CZ3 | TRP | A | 96 | 57.112 | 18.749 | 51.996 | 1.00 | 20.27 |
| ATOM | 792 | CH2 | TRP | A | 96 | 56.778 | 19.667 | 52.985 | 1.00 | 22.62 |
| ATOM | 793 | N | GLU | A | 97 | 60.038 | 12.365 | 52.588 | 1.00 | 30.30 |
| ATOM | 794 | CA | GLU | A | 97 | 60.350 | 11.246 | 51.698 | 1.00 | 31.44 |
| ATOM | 795 | C | GLU | A | 97 | 59.100 | 10.819 | 50.950 | 1.00 | 28.25 |
| ATOM | 796 | O | GLU | A | 97 | 59.127 | 10.632 | 49.745 | 1.00 | 26.11 |
| ATOM | 797 | CB | GLU | A | 97 | 60.900 | 10.031 | 52.459 | 1.00 | 32.06 |
| ATOM | 798 | CG | GLU | A | 97 | 61.199 | 8.868 | 51.521 | 1.00 | 31.62 |
| ATOM | 799 | CD | GLU | A | 97 | 61.797 | 7.647 | 52.196 | 1.00 | 33.54 |
| ATOM | 800 | OE1 | GLU | A | 97 | 62.047 | 7.685 | 53.414 | 1.00 | 33.60 |
| ATOM | 801 | OE2 | GLU | A | 97 | 62.002 | 6.630 | 51.490 | 1.00 | 33.83 |
| ATOM | 802 | N | MET | A | 98 | 58.009 | 10.654 | 51.680 | 1.00 | 28.99 |
| ATOM | 803 | CA | MET | A | 98 | 56.746 | 10.295 | 51.066 | 1.00 | 25.04 |
| ATOM | 804 | C | MET | A | 98 | 56.325 | 11.357 | 50.057 | 1.00 | 28.35 |
| ATOM | 805 | O | MET | A | 98 | 55.939 | 11.011 | 48.947 | 1.00 | 29.80 |
| ATOM | 806 | CB | MET | A | 98 | 55.678 | 10.092 | 52.123 | 1.00 | 22.99 |
| ATOM | 807 | CG | MET | A | 98 | 54.289 | 9.880 | 51.562 | 1.00 | 27.35 |
| ATOM | 808 | SD | MET | A | 98 | 53.081 | 9.519 | 52.796 | 1.00 | 26.88 |
| ATOM | 809 | CE | MET | A | 98 | 52.976 | 11.131 | 53.675 | 1.00 | 28.20 |
| ATOM | 810 | N | VAL | A | 99 | 56.394 | 12.641 | 50.422 | 1.00 | 27.18 |
| ATOM | 811 | CA | VAL | A | 99 | 56.030 | 13.718 | 49.486 | 1.00 | 25.66 |
| ATOM | 812 | C | VAL | A | 99 | 56.845 | 13.637 | 48.186 | 1.00 | 30.66 |
| ATOM | 813 | O | VAL | A | 99 | 56.286 | 13.772 | 47.091 | 1.00 | 28.50 |
| ATOM | 814 | CB | VAL | A | 99 | 56.212 | 15.108 | 50.100 | 1.00 | 25.29 |
| ATOM | 815 | CG1 | VAL | A | 99 | 56.054 | 16.177 | 49.051 | 1.00 | 25.73 |
| ATOM | 816 | CG2 | VAL | A | 99 | 55.207 | 15.348 | 51.238 | 1.00 | 24.18 |
| ATOM | 817 | N | TRP | A | 100 | 58.151 | 13.396 | 48.319 | 1.00 | 29.79 |
| ATOM | 818 | CA | TRP | A | 100 | 59.046 | 13.220 | 47.179 | 1.00 | 30.99 |
| ATOM | 819 | C | TRP | A | 100 | 58.619 | 12.052 | 46.269 | 1.00 | 30.01 |
| ATOM | 820 | O | TRP | A | 100 | 58.342 | 12.241 | 45.095 | 1.00 | 32.13 |
| ATOM | 821 | CB | TRP | A | 100 | 60.498 | 12.973 | 47.655 | 1.00 | 34.76 |
| ATOM | 822 | CG | TRP | A | 100 | 61.439 | 12.866 | 46.498 | 1.00 | 37.10 |
| ATOM | 823 | CD1 | TRP | A | 100 | 61.774 | 11.730 | 45.802 | 1.00 | 40.72 |
| ATOM | 824 | CD2 | TRP | A | 100 | 62.097 | 13.941 | 45.846 | 1.00 | 41.47 |
| ATOM | 825 | NE1 | TRP | A | 100 | 62.620 | 12.044 | 44.766 | 1.00 | 36.22 |
| ATOM | 826 | CE2 | TRP | A | 100 | 62.832 | 13.395 | 44.765 | 1.00 | 44.00 |
| ATOM | 827 | CE3 | TRP | A | 100 | 62.144 | 15.323 | 46.057 | 1.00 | 41.72 |
| ATOM | 828 | CZ2 | TRP | A | 100 | 63.608 | 14.184 | 43.912 | 1.00 | 45.14 |
| ATOM | 829 | CZ3 | TRP | A | 100 | 62.919 | 16.101 | 45.218 | 1.00 | 45.12 |
| ATOM | 830 | CH2 | TRP | A | 100 | 63.639 | 15.530 | 44.154 | 1.00 | 46.67 |
| ATOM | 831 | N | GLU | A | 101 | 58.559 | 10.858 | 46.841 | 1.00 | 29.76 |
| ATOM | 832 | CA | GLU | A | 101 | 58.296 | 9.623 | 46.109 | 1.00 | 31.91 |
| ATOM | 833 | C | GLU | A | 101 | 56.897 | 9.542 | 45.500 | 1.00 | 33.10 |
| ATOM | 834 | O | GLU | A | 101 | 56.717 | 8.953 | 44.436 | 1.00 | 31.24 |
| ATOM | 835 | CB | GLU | A | 101 | 58.513 | 8.428 | 47.042 | 1.00 | 33.49 |
| ATOM | 836 | CG | GLU | A | 101 | 59.986 | 8.235 | 47.416 | 1.00 | 35.52 |
| ATOM | 837 | CD | GLU | A | 101 | 60.198 | 7.324 | 48.613 | 1.00 | 36.36 |
| ATOM | 838 | OE1 | GLU | A | 101 | 59.208 | 6.722 | 49.114 | 1.00 | 34.30 |
| ATOM | 839 | OE2 | GLU | A | 101 | 61.372 | 7.210 | 49.045 | 1.00 | 33.40 |
| ATOM | 840 | N | GLN | A | 102 | 55.910 | 10.126 | 46.175 | 1.00 | 28.75 |
| ATOM | 841 | CA | GLN | A | 102 | 54.538 | 10.120 | 45.677 | 1.00 | 29.37 |
| ATOM | 842 | C | GLN | A | 102 | 54.295 | 11.220 | 44.663 | 1.00 | 28.29 |
| ATOM | 843 | O | GLN | A | 102 | 53.213 | 11.300 | 44.086 | 1.00 | 31.13 |
| ATOM | 844 | CB | GLN | A | 102 | 53.544 | 10.212 | 46.842 | 1.00 | 30.91 |
| ATOM | 845 | CG | GLN | A | 102 | 53.617 | 9.000 | 47.798 | 1.00 | 31.45 |
| ATOM | 846 | CD | GLN | A | 102 | 53.496 | 7.656 | 47.061 | 1.00 | 32.80 |
| ATOM | 847 | OE1 | GLN | A | 102 | 52.626 | 7.504 | 46.214 | 1.00 | 31.63 |
| ATOM | 848 | NE2 | GLN | A | 102 | 54.365 | 6.702 | 47.384 | 1.00 | 32.99 |
| ATOM | 849 | N | LYS | A | 103 | 55.299 | 12.076 | 44.466 | 1.00 | 30.72 |
| ATOM | 850 | CA | LYS | A | 103 | 55.263 | 13.155 | 43.475 | 1.00 | 32.47 |
| ATOM | 851 | C | LYS | A | 103 | 54.163 | 14.179 | 43.750 | 1.00 | 27.03 |
| ATOM | 852 | O | LYS | A | 103 | 53.636 | 14.823 | 42.850 | 1.00 | 25.82 |
| ATOM | 853 | CB | LYS | A | 103 | 55.192 | 12.578 | 42.045 | 1.00 | 36.98 |
| ATOM | 854 | CG | LYS | A | 103 | 56.481 | 11.826 | 41.653 | 1.00 | 41.87 |
| ATOM | 855 | CD | LYS | A | 103 | 56.364 | 11.121 | 40.298 | 1.00 | 49.14 |
| ATOM | 856 | CE | LYS | A | 103 | 57.734 | 10.605 | 39.815 | 1.00 | 51.95 |
| ATOM | 857 | NZ | LYS | A | 103 | 58.541 | 10.021 | 40.935 | 1.00 | 54.94 |
| ATOM | 858 | N | SER | A | 104 | 53.831 | 14.326 | 45.020 | 1.00 | 25.84 |
| ATOM | 859 | CA | SER | A | 104 | 52.865 | 15.318 | 45.453 | 1.00 | 25.52 |
| ATOM | 860 | C | SER | A | 104 | 53.319 | 16.754 | 45.131 | 1.00 | 25.82 |
| ATOM | 861 | O | SER | A | 104 | 54.497 | 17.102 | 45.208 | 1.00 | 26.75 |
| ATOM | 862 | CB | SER | A | 104 | 52.632 | 15.179 | 46.958 | 1.00 | 26.43 |
| ATOM | 863 | OG | SER | A | 104 | 52.328 | 13.840 | 47.299 | 1.00 | 30.28 |
| ATOM | 864 | N | ARG | A | 105 | 52.353 | 17.577 | 44.781 | 1.00 | 27.47 |
| ATOM | 865 | CA | ARG | A | 105 | 52.569 | 18.977 | 44.524 | 1.00 | 29.78 |
| ATOM | 866 | C | ARG | A | 105 | 52.226 | 19.799 | 45.771 | 1.00 | 27.49 |
| ATOM | 867 | O | ARG | A | 105 | 52.850 | 20.808 | 46.046 | 1.00 | 26.55 |
| ATOM | 868 | CB | ARG | A | 105 | 51.668 | 19.378 | 43.358 | 1.00 | 32.18 |
| ATOM | 869 | CG | ARG | A | 105 | 52.147 | 20.517 | 42.532 | 1.00 | 42.19 |
| ATOM | 870 | CD | ARG | A | 105 | 51.696 | 21.835 | 43.034 | 1.00 | 48.41 |
| ATOM | 871 | NE | ARG | A | 105 | 52.155 | 22.954 | 42.219 | 1.00 | 55.25 |
| ATOM | 872 | CZ | ARG | A | 105 | 53.418 | 23.361 | 42.124 | 1.00 | 59.03 |
| ATOM | 873 | NH1 | ARG | A | 105 | 54.400 | 22.750 | 42.773 | 1.00 | 62.40 |
| ATOM | 874 | NH2 | ARG | A | 105 | 53.695 | 24.420 | 41.378 | 1.00 | 62.61 |
| ATOM | 875 | N | GLY | A | 106 | 51.217 | 19.364 | 46.509 | 1.00 | 28.11 |
| ATOM | 876 | CA | GLY | A | 106 | 50.689 | 20.120 | 47.634 | 1.00 | 25.59 |
| ATOM | 877 | C | GLY | A | 106 | 50.640 | 19.324 | 48.925 | 1.00 | 22.98 |
| ATOM | 878 | O | GLY | A | 106 | 50.481 | 18.092 | 48.917 | 1.00 | 22.43 |
| ATOM | 879 | N | VAL | A | 107 | 50.776 | 20.044 | 50.035 | 1.00 | 21.47 |
| ATOM | 880 | CA | VAL | A | 107 | 50.560 | 19.526 | 51.380 | 1.00 | 17.55 |
| ATOM | 881 | C | VAL | A | 107 | 49.532 | 20.450 | 51.987 | 1.00 | 21.98 |
| ATOM | 882 | O | VAL | A | 107 | 49.667 | 21.681 | 51.883 | 1.00 | 21.61 |
| ATOM | 883 | CB | VAL | A | 107 | 51.864 | 19.550 | 52.205 | 1.00 | 19.48 |
| ATOM | 884 | CG1 | VAL | A | 107 | 51.606 | 19.250 | 53.665 | 1.00 | 20.01 |
| ATOM | 885 | CG2 | VAL | A | 107 | 52.867 | 18.578 | 51.632 | 1.00 | 19.65 |
| ATOM | 886 | N | VAL | A | 108 | 48.476 | 19.862 | 52.548 | 1.00 | 20.07 |
| ATOM | 887 | CA | VAL | A | 108 | 47.370 | 20.582 | 53.155 | 1.00 | 21.81 |
| ATOM | 888 | C | VAL | A | 108 | 47.395 | 20.313 | 54.658 | 1.00 | 23.26 |
| ATOM | 889 | O | VAL | A | 108 | 47.252 | 19.163 | 55.102 | 1.00 | 21.40 |
| ATOM | 890 | CB | VAL | A | 108 | 46.023 | 20.162 | 52.562 | 1.00 | 22.26 |
| ATOM | 891 | CG1 | VAL | A | 108 | 44.880 | 20.866 | 53.269 | 1.00 | 26.46 |
| ATOM | 892 | CG2 | VAL | A | 108 | 45.984 | 20.479 | 51.065 | 1.00 | 22.10 |
| ATOM | 893 | N | MET | A | 109 | 47.623 | 21.380 | 55.431 | 1.00 | 22.08 |
| ATOM | 894 | CA | MET | A | 109 | 47.732 | 21.306 | 56.881 | 1.00 | 22.68 |
| ATOM | 895 | C | MET | A | 109 | 46.526 | 21.981 | 57.504 | 1.00 | 21.07 |
| ATOM | 896 | O | MET | A | 109 | 46.304 | 23.171 | 57.312 | 1.00 | 23.17 |
| ATOM | 897 | CB | MET | A | 109 | 49.024 | 21.972 | 57.340 | 1.00 | 23.07 |
| ATOM | 898 | CG | MET | A | 109 | 49.233 | 22.019 | 58.858 | 1.00 | 22.88 |
| ATOM | 899 | SD | MET | A | 109 | 50.928 | 22.520 | 59.237 | 1.00 | 24.38 |
| ATOM | 900 | CE | MET | A | 109 | 50.925 | 22.587 | 61.036 | 1.00 | 27.77 |
| ATOM | 901 | N | LEU | A | 110 | 45.740 | 21.224 | 58.259 | 1.00 | 21.69 |
| ATOM | 902 | CA | LEU | A | 110 | 44.481 | 21.755 | 58.778 | 1.00 | 20.78 |
| ATOM | 903 | C | LEU | A | 110 | 44.515 | 22.017 | 60.269 | 1.00 | 21.23 |
| ATOM | 904 | O | LEU | A | 110 | 43.485 | 22.050 | 60.916 | 1.00 | 29.08 |
| ATOM | 905 | CB | LEU | A | 110 | 43.347 | 20.786 | 58.448 | 1.00 | 24.47 |
| ATOM | 906 | CG | LEU | A | 110 | 43.108 | 20.528 | 56.966 | 1.00 | 24.58 |
| ATOM | 907 | CD1 | LEU | A | 110 | 42.139 | 19.388 | 56.762 | 1.00 | 24.62 |
| ATOM | 908 | CD2 | LEU | A | 110 | 42.602 | 21.776 | 56.325 | 1.00 | 29.12 |
| ATOM | 909 | N | ASN | A | 111 | 45.707 | 22.205 | 60.809 | 1.00 | 24.23 |
| ATOM | 910 | CA | ASN | A | 111 | 45.887 | 22.492 | 62.221 | 1.00 | 25.10 |
| ATOM | 911 | C | ASN | A | 111 | 47.004 | 23.514 | 62.423 | 1.00 | 23.03 |
| ATOM | 912 | O | ASN | A | 111 | 47.825 | 23.738 | 61.553 | 1.00 | 23.77 |
| ATOM | 913 | CB | ASN | A | 111 | 46.232 | 21.200 | 62.977 | 1.00 | 24.84 |
| ATOM | 914 | CG | ASN | A | 111 | 47.540 | 20.620 | 62.544 | 1.00 | 24.46 |
| ATOM | 915 | OD1 | ASN | A | 111 | 47.638 | 20.087 | 61.457 | 1.00 | 24.90 |
| ATOM | 916 | ND2 | ASN | A | 111 | 48.577 | 20.764 | 63.371 | 1.00 | 23.98 |
| ATOM | 917 | N | ARG | A | 112 | 47.025 | 24.109 | 63.601 | 1.00 | 24.79 |
| ATOM | 918 | CA | ARG | A | 112 | 48.175 | 24.832 | 64.074 | 1.00 | 22.03 |
| ATOM | 919 | C | ARG | A | 112 | 49.122 | 23.888 | 64.775 | 1.00 | 23.38 |
| ATOM | 920 | O | ARG | A | 112 | 48.713 | 22.857 | 65.340 | 1.00 | 21.21 |
| ATOM | 921 | CB | ARG | A | 112 | 47.736 | 25.940 | 65.023 | 1.00 | 26.48 |
| ATOM | 922 | CG | ARG | A | 112 | 47.170 | 27.143 | 64.305 | 1.00 | 27.70 |
| ATOM | 923 | CD | ARG | A | 112 | 46.483 | 28.152 | 65.225 | 1.00 | 32.52 |
| ATOM | 924 | NE | ARG | A | 112 | 45.281 | 27.582 | 65.821 | 1.00 | 35.60 |
| ATOM | 925 | CZ | ARG | A | 112 | 45.009 | 27.531 | 67.128 | 1.00 | 43.32 |
| ATOM | 926 | NH1 | ARG | A | 112 | 45.823 | 28.057 | 68.050 | 1.00 | 46.30 |
| ATOM | 927 | NH2 | ARG | A | 112 | 43.873 | 26.965 | 67.523 | 1.00 | 47.45 |
| ATOM | 928 | N | VAL | A | 113 | 50.396 | 24.256 | 64.745 | 1.00 | 25.41 |
| ATOM | 929 | CA | VAL | A | 113 | 51.439 | 23.532 | 65.433 | 1.00 | 26.91 |
| ATOM | 930 | C | VAL | A | 113 | 51.116 | 23.437 | 66.921 | 1.00 | 27.74 |
| ATOM | 931 | O | VAL | A | 113 | 51.286 | 22.379 | 67.532 | 1.00 | 25.36 |
| ATOM | 932 | CB | VAL | A | 113 | 52.804 | 24.185 | 65.182 | 1.00 | 29.50 |
| ATOM | 933 | CG1 | VAL | A | 113 | 53.875 | 23.615 | 66.110 | 1.00 | 26.54 |
| ATOM | 934 | CG2 | VAL | A | 113 | 53.201 | 23.978 | 63.717 | 1.00 | 30.55 |
| ATOM | 935 | N | MET | A | 114 | 50.585 | 24.520 | 67.475 | 1.00 | 26.52 |
| ATOM | 936 | CA | MET | A | 114 | 50.077 | 24.510 | 68.842 | 1.00 | 27.11 |
| ATOM | 937 | C | MET | A | 114 | 48.601 | 24.775 | 68.849 | 1.00 | 23.26 |
| ATOM | 938 | O | MET | A | 114 | 48.158 | 25.780 | 68.294 | 1.00 | 24.63 |
| ATOM | 939 | CB | MET | A | 114 | 50.757 | 25.584 | 69.708 | 1.00 | 26.78 |
| ATOM | 940 | CG | MET | A | 114 | 50.258 | 25.541 | 71.159 | 1.00 | 28.56 |
| ATOM | 941 | SD | MET | A | 114 | 51.448 | 26.265 | 72.371 | 1.00 | 31.45 |
| ATOM | 942 | CE | MET | A | 114 | 52.537 | 24.917 | 72.614 | 1.00 | 33.42 |
| ATOM | 943 | N | GLU | A | 115 | 47.841 | 23.879 | 69.485 | 1.00 | 22.46 |
| ATOM | 944 | CA | GLU | A | 115 | 46.419 | 24.094 | 69.734 | 1.00 | 24.62 |
| ATOM | 945 | C | GLU | A | 115 | 46.117 | 23.653 | 71.138 | 1.00 | 23.07 |
| ATOM | 946 | O | GLU | A | 115 | 46.683 | 22.674 | 71.597 | 1.00 | 23.93 |
| ATOM | 947 | CB | GLU | A | 115 | 45.570 | 23.238 | 68.757 | 1.00 | 27.38 |
| ATOM | 948 | CG | GLU | A | 115 | 45.668 | 23.711 | 67.323 | 1.00 | 30.60 |
| ATOM | 949 | CD | GLU | A | 115 | 44.909 | 22.854 | 66.330 | 1.00 | 30.65 |
| ATOM | 950 | OE1 | GLU | A | 115 | 44.586 | 21.681 | 66.612 | 1.00 | 31.07 |
| ATOM | 951 | OE2 | GLU | A | 115 | 44.672 | 23.377 | 65.242 | 1.00 | 28.57 |
| ATOM | 952 | N | LYS | A | 116 | 45.218 | 24.347 | 71.824 | 1.00 | 27.40 |
| ATOM | 953 | CA | LYS | A | 116 | 44.861 | 23.978 | 73.204 | 1.00 | 29.17 |
| ATOM | 954 | C | LYS | A | 116 | 46.099 | 23.920 | 74.107 | 1.00 | 26.13 |
| ATOM | 955 | O | LYS | A | 116 | 46.189 | 23.078 | 75.015 | 1.00 | 30.38 |
| ATOM | 956 | CB | LYS | A | 116 | 44.120 | 22.629 | 73.250 | 1.00 | 29.07 |
| ATOM | 957 | CG | LYS | A | 116 | 42.741 | 22.624 | 72.571 | 1.00 | 37.49 |
| ATOM | 958 | CD | LYS | A | 116 | 42.321 | 21.181 | 72.213 | 1.00 | 43.81 |
| ATOM | 959 | CE | LYS | A | 116 | 40.821 | 20.938 | 72.347 | 1.00 | 48.07 |
| ATOM | 960 | NZ | LYS | A | 116 | 40.464 | 19.530 | 71.998 | 1.00 | 49.95 |
| ATOM | 961 | N | GLY | A | 117 | 47.067 | 24.788 | 73.835 | 1.00 | 26.78 |
| ATOM | 962 | CA | GLY | A | 117 | 48.298 | 24.847 | 74.625 | 1.00 | 28.90 |
| ATOM | 963 | C | GLY | A | 117 | 49.222 | 23.641 | 74.523 | 1.00 | 27.34 |
| ATOM | 964 | O | GLY | A | 117 | 50.115 | 23.452 | 75.353 | 1.00 | 27.25 |
| ATOM | 965 | N | SER | A | 118 | 49.035 | 22.834 | 73.489 | 1.00 | 23.86 |
| ATOM | 966 | CA | SER | A | 118 | 49.773 | 21.587 | 73.355 | 1.00 | 26.52 |
| ATOM | 967 | C | SER | A | 118 | 50.291 | 21.438 | 71.917 | 1.00 | 24.21 |
| ATOM | 968 | O | SER | A | 118 | 49.675 | 21.899 | 70.973 | 1.00 | 24.79 |
| ATOM | 969 | CB | SER | A | 118 | 48.856 | 20.424 | 73.750 | 1.00 | 27.56 |
| ATOM | 970 | OG | SER | A | 118 | 49.518 | 19.184 | 73.637 | 1.00 | 40.87 |
| ATOM | 971 | N | LEU | A | 119 | 51.444 | 20.817 | 71.760 | 1.00 | 26.60 |
| ATOM | 972 | CA | LEU | A | 119 | 52.003 | 20.587 | 70.438 | 1.00 | 26.38 |
| ATOM | 973 | C | LEU | A | 119 | 51.266 | 19.490 | 69.683 | 1.00 | 27.04 |
| ATOM | 974 | O | LEU | A | 119 | 51.092 | 18.388 | 70.184 | 1.00 | 27.29 |
| ATOM | 975 | CB | LEU | A | 119 | 53.488 | 20.261 | 70.544 | 1.00 | 25.60 |
| ATOM | 976 | CG | LEU | A | 119 | 54.357 | 21.413 | 71.084 | 1.00 | 31.75 |
| ATOM | 977 | CD1 | LEU | A | 119 | 55.806 | 20.985 | 71.131 | 1.00 | 34.60 |
| ATOM | 978 | CD2 | LEU | A | 119 | 54.215 | 22.704 | 70.274 | 1.00 | 32.35 |
| ATOM | 979 | N | LYS | A | 120 | 50.856 | 19.806 | 68.456 | 1.00 | 24.78 |
| ATOM | 980 | CA | LYS | A | 120 | 50.049 | 18.922 | 67.638 | 1.00 | 28.04 |
| ATOM | 981 | C | LYS | A | 120 | 50.870 | 18.254 | 66.542 | 1.00 | 27.21 |
| ATOM | 982 | O | LYS | A | 120 | 50.482 | 17.214 | 66.037 | 1.00 | 30.85 |
| ATOM | 983 | CB | LYS | A | 120 | 48.868 | 19.698 | 67.043 | 1.00 | 27.25 |
| ATOM | 984 | CG | LYS | A | 120 | 47.934 | 20.246 | 68.108 | 1.00 | 30.57 |
| ATOM | 985 | CD | LYS | A | 120 | 47.238 | 19.108 | 68.911 | 1.00 | 33.63 |
| ATOM | 986 | CE | LYS | A | 120 | 46.840 | 19.569 | 70.300 | 1.00 | 33.92 |
| ATOM | 987 | NZ | LYS | A | 120 | 46.227 | 18.461 | 71.090 | 1.00 | 32.12 |
| ATOM | 988 | N | CYS | A | 121 | 51.982 | 18.873 | 66.170 | 1.00 | 28.54 |
| ATOM | 989 | CA | CYS | A | 121 | 52.980 | 18.258 | 65.309 | 1.00 | 29.45 |
| ATOM | 990 | C | CYS | A | 121 | 54.322 | 18.956 | 65.421 | 1.00 | 24.55 |
| ATOM | 991 | O | CYS | A | 121 | 54.432 | 20.038 | 66.003 | 1.00 | 26.49 |
| ATOM | 992 | CB | CYS | A | 121 | 52.536 | 18.229 | 63.829 | 1.00 | 33.36 |
| ATOM | 993 | SG | CYS | A | 121 | 52.300 | 19.820 | 63.026 | 1.00 | 31.09 |
| ATOM | 994 | N | ALA | A | 122 | 55.341 | 18.312 | 64.866 | 1.00 | 24.17 |
| ATOM | 995 | CA | ALA | A | 122 | 56.663 | 18.898 | 64.777 | 1.00 | 25.15 |
| ATOM | 996 | C | ALA | A | 122 | 56.625 | 20.091 | 63.815 | 1.00 | 24.39 |
| ATOM | 997 | O | ALA | A | 122 | 55.740 | 20.189 | 62.952 | 1.00 | 21.50 |
| ATOM | 998 | CB | ALA | A | 122 | 57.656 | 17.869 | 64.326 | 1.00 | 24.68 |
| ATOM | 999 | N | GLN | A | 123 | 57.534 | 21.041 | 64.024 | 1.00 | 30.52 |
| ATOM | 1000 | CA | GLN | A | 123 | 57.748 | 22.139 | 63.077 | 1.00 | 26.76 |
| ATOM | 1001 | C | GLN | A | 123 | 58.493 | 21.514 | 61.920 | 1.00 | 27.16 |
| ATOM | 1002 | O | GLN | A | 123 | 59.722 | 21.496 | 61.900 | 1.00 | 26.04 |
| ATOM | 1003 | CB | GLN | A | 123 | 58.555 | 23.275 | 63.738 | 1.00 | 30.31 |
| ATOM | 1004 | CG | GLN | A | 123 | 58.749 | 24.529 | 62.865 | 1.00 | 26.95 |
| ATOM | 1005 | CD | GLN | A | 123 | 57.434 | 25.088 | 62.373 | 1.00 | 25.90 |
| ATOM | 1006 | OE1 | GLN | A | 123 | 56.534 | 25.382 | 63.178 | 1.00 | 28.50 |
| ATOM | 1007 | NE2 | GLN | A | 123 | 57.294 | 25.203 | 61.052 | 1.00 | 19.25 |
| ATOM | 1008 | N | TYR | A | 124 | 57.745 | 20.969 | 60.955 | 1.00 | 26.91 |
| ATOM | 1009 | CA | TYR | A | 124 | 58.350 | 20.112 | 59.933 | 1.00 | 23.80 |
| ATOM | 1010 | C | TYR | A | 124 | 58.804 | 20.845 | 58.663 | 1.00 | 22.10 |
| ATOM | 1011 | O | TYR | A | 124 | 59.398 | 20.234 | 57.781 | 1.00 | 21.86 |
| ATOM | 1012 | CB | TYR | A | 124 | 57.425 | 18.921 | 59.611 | 1.00 | 22.76 |
| ATOM | 1013 | CG | TYR | A | 124 | 56.099 | 19.261 | 58.983 | 1.00 | 17.20 |
| ATOM | 1014 | CD1 | TYR | A | 124 | 55.971 | 19.316 | 57.598 | 1.00 | 20.74 |
| ATOM | 1015 | CD2 | TYR | A | 124 | 54.974 | 19.499 | 59.753 | 1.00 | 18.39 |
| ATOM | 1016 | CE1 | TYR | A | 124 | 54.752 | 19.606 | 56.989 | 1.00 | 17.90 |
| ATOM | 1017 | CE2 | TYR | A | 124 | 53.752 | 19.822 | 59.154 | 1.00 | 19.29 |
| ATOM | 1018 | CZ | TYR | A | 124 | 53.649 | 19.841 | 57.756 | 1.00 | 17.31 |
| ATOM | 1019 | OH | TYR | A | 124 | 52.464 | 20.152 | 57.113 | 1.00 | 16.79 |
| ATOM | 1020 | N | TRP | A | 125 | 58.540 | 22.147 | 58.593 | 1.00 | 25.93 |
| ATOM | 1021 | CA | TRP | A | 125 | 58.997 | 22.995 | 57.496 | 1.00 | 23.88 |
| ATOM | 1022 | C | TRP | A | 125 | 59.736 | 24.240 | 58.052 | 1.00 | 27.60 |
| ATOM | 1023 | O | TRP | A | 125 | 59.483 | 24.677 | 59.195 | 1.00 | 26.13 |
| ATOM | 1024 | CB | TRP | A | 125 | 57.808 | 23.402 | 56.617 | 1.00 | 22.08 |
| ATOM | 1025 | CG | TRP | A | 125 | 56.944 | 24.458 | 57.214 | 1.00 | 25.66 |
| ATOM | 1026 | CD1 | TRP | A | 125 | 57.013 | 25.801 | 56.973 | 1.00 | 26.72 |
| ATOM | 1027 | CD2 | TRP | A | 125 | 55.880 | 24.280 | 58.152 | 1.00 | 26.67 |
| ATOM | 1028 | NE1 | TRP | A | 125 | 56.085 | 26.468 | 57.727 | 1.00 | 22.95 |
| ATOM | 1029 | CE2 | TRP | A | 125 | 55.366 | 25.562 | 58.453 | 1.00 | 24.22 |
| ATOM | 1030 | CE3 | TRP | A | 125 | 55.315 | 23.167 | 58.785 | 1.00 | 23.11 |
| ATOM | 1031 | CZ2 | TRP | A | 125 | 54.314 | 25.760 | 59.347 | 1.00 | 25.50 |
| ATOM | 1032 | CZ3 | TRP | A | 125 | 54.280 | 23.367 | 59.677 | 1.00 | 24.70 |
| ATOM | 1033 | CH2 | TRP | A | 125 | 53.783 | 24.646 | 59.945 | 1.00 | 26.16 |
| ATOM | 1034 | N | PRO | A | 126 | 60.658 | 24.804 | 57.273 | 1.00 | 27.12 |
| ATOM | 1035 | CA | PRO | A | 126 | 61.423 | 25.971 | 57.737 | 1.00 | 28.25 |
| ATOM | 1036 | C | PRO | A | 126 | 60.554 | 27.216 | 57.804 | 1.00 | 24.48 |
| ATOM | 1037 | O | PRO | A | 126 | 59.742 | 27.466 | 56.922 | 1.00 | 28.44 |
| ATOM | 1038 | CB | PRO | A | 126 | 62.544 | 26.097 | 56.704 | 1.00 | 30.18 |
| ATOM | 1039 | CG | PRO | A | 126 | 62.052 | 25.416 | 55.499 | 1.00 | 31.77 |
| ATOM | 1040 | CD | PRO | A | 126 | 61.074 | 24.373 | 55.933 | 1.00 | 28.76 |
| ATOM | 1041 | N | GLN | A | 127 | 60.709 | 27.968 | 58.878 | 1.00 | 27.22 |
| ATOM | 1042 | CA | GLN | A | 127 | 59.988 | 29.207 | 59.082 | 1.00 | 30.34 |
| ATOM | 1043 | C | GLN | A | 127 | 60.672 | 30.438 | 58.437 | 1.00 | 31.05 |
| ATOM | 1044 | O | GLN | A | 127 | 60.057 | 31.509 | 58.331 | 1.00 | 28.64 |
| ATOM | 1045 | CB | GLN | A | 127 | 59.815 | 29.441 | 60.584 | 1.00 | 34.08 |
| ATOM | 1046 | CG | GLN | A | 127 | 58.761 | 28.522 | 61.214 | 1.00 | 40.26 |
| ATOM | 1047 | CD | GLN | A | 127 | 58.758 | 28.603 | 62.722 | 1.00 | 41.02 |
| ATOM | 1048 | OE1 | GLN | A | 127 | 59.767 | 28.319 | 63.360 | 1.00 | 43.41 |
| ATOM | 1049 | NE2 | GLN | A | 127 | 57.628 | 29.003 | 63.294 | 1.00 | 45.74 |
| ATOM | 1050 | N | LYS | A | 128 | 61.925 | 30.275 | 58.015 | 1.00 | 30.38 |
| ATOM | 1051 | CA | LYS | A | 128 | 62.733 | 31.371 | 57.449 | 1.00 | 33.35 |
| ATOM | 1052 | C | LYS | A | 128 | 63.452 | 30.923 | 56.191 | 1.00 | 26.98 |
| ATOM | 1053 | O | LYS | A | 128 | 64.059 | 29.872 | 56.175 | 1.00 | 26.69 |
| ATOM | 1054 | CB | LYS | A | 128 | 63.761 | 31.857 | 58.481 | 1.00 | 32.67 |
| ATOM | 1055 | CG | LYS | A | 128 | 63.168 | 32.830 | 59.483 | 1.00 | 40.12 |
| ATOM | 1056 | CD | LYS | A | 128 | 64.055 | 33.017 | 60.694 | 1.00 | 46.31 |
| ATOM | 1057 | CE | LYS | A | 128 | 63.381 | 33.939 | 61.717 | 1.00 | 49.48 |
| ATOM | 1058 | NZ | LYS | A | 128 | 64.329 | 34.401 | 62.776 | 1.00 | 49.69 |
| ATOM | 1059 | N | GLU | A | 129 | 63.375 | 31.725 | 55.137 | 1.00 | 26.51 |
| ATOM | 1060 | CA | GLU | A | 129 | 64.084 | 31.448 | 53.881 | 1.00 | 27.16 |
| ATOM | 1061 | C | GLU | A | 129 | 65.546 | 31.034 | 54.099 | 1.00 | 27.84 |
| ATOM | 1062 | O | GLU | A | 129 | 66.048 | 30.082 | 53.484 | 1.00 | 27.85 |
| ATOM | 1063 | CB | GLU | A | 129 | 64.028 | 32.696 | 52.973 | 1.00 | 28.24 |
| ATOM | 1064 | CG | GLU | A | 129 | 62.675 | 32.999 | 52.330 | 1.00 | 30.95 |
| ATOM | 1065 | CD | GLU | A | 129 | 61.739 | 33.832 | 53.186 | 1.00 | 29.81 |
| ATOM | 1066 | OE1 | GLU | A | 129 | 61.945 | 33.907 | 54.416 | 1.00 | 31.49 |
| ATOM | 1067 | OE2 | GLU | A | 129 | 60.801 | 34.434 | 52.619 | 1.00 | 28.03 |
| ATOM | 1068 | N | GLU | A | 130 | 66.228 | 31.733 | 55.010 | 1.00 | 33.63 |
| ATOM | 1069 | CA | GLU | A | 130 | 67.688 | 31.606 | 55.172 | 1.00 | 33.03 |
| ATOM | 1070 | C | GLU | A | 130 | 68.097 | 30.442 | 56.079 | 1.00 | 34.75 |
| ATOM | 1071 | O | GLU | A | 130 | 69.285 | 30.087 | 56.150 | 1.00 | 34.36 |
| ATOM | 1072 | CB | GLU | A | 130 | 68.316 | 32.938 | 55.648 | 1.00 | 33.23 |
| ATOM | 1073 | CG | GLU | A | 130 | 67.894 | 33.433 | 57.028 | 1.00 | 32.78 |
| ATOM | 1074 | CD | GLU | A | 130 | 66.624 | 34.256 | 57.027 | 1.00 | 35.80 |
| ATOM | 1075 | OE1 | GLU | A | 130 | 66.364 | 34.966 | 58.027 | 1.00 | 39.02 |
| ATOM | 1076 | OE2 | GLU | A | 130 | 65.857 | 34.187 | 56.045 | 1.00 | 41.84 |
| ATOM | 1077 | N | LYS | A | 131 | 67.123 | 29.826 | 56.741 | 1.00 | 36.67 |
| ATOM | 1078 | CA | LYS | A | 131 | 67.401 | 28.665 | 57.593 | 1.00 | 38.30 |
| ATOM | 1079 | C | LYS | A | 131 | 66.648 | 27.442 | 57.095 | 1.00 | 36.19 |
| ATOM | 1080 | O | LYS | A | 131 | 65.550 | 27.105 | 57.558 | 1.00 | 34.88 |
| ATOM | 1081 | CB | LYS | A | 131 | 67.103 | 28.975 | 59.062 | 1.00 | 41.34 |
| ATOM | 1082 | CG | LYS | A | 131 | 68.134 | 29.955 | 59.674 | 1.00 | 45.05 |
| ATOM | 1083 | CD | LYS | A | 131 | 68.300 | 29.778 | 61.178 | 1.00 | 48.43 |
| ATOM | 1084 | CE | LYS | A | 131 | 69.408 | 30.682 | 61.734 | 1.00 | 50.09 |
| ATOM | 1085 | NZ | LYS | A | 131 | 68.964 | 31.471 | 62.922 | 1.00 | 50.14 |
| ATOM | 1086 | N | GLU | A | 132 | 67.270 | 26.798 | 56.119 | 1.00 | 34.42 |
| ATOM | 1087 | CA | GLU | A | 132 | 66.750 | 25.587 | 55.514 | 1.00 | 35.74 |
| ATOM | 1088 | C | GLU | A | 132 | 66.892 | 24.395 | 56.466 | 1.00 | 37.34 |
| ATOM | 1089 | O | GLU | A | 132 | 67.619 | 24.460 | 57.460 | 1.00 | 33.65 |
| ATOM | 1090 | CB | GLU | A | 132 | 67.446 | 25.316 | 54.176 | 1.00 | 34.20 |
| ATOM | 1091 | CG | GLU | A | 132 | 68.853 | 24.745 | 54.267 | 1.00 | 38.61 |
| ATOM | 1092 | CD | GLU | A | 132 | 69.952 | 25.792 | 54.436 | 1.00 | 39.48 |
| ATOM | 1093 | OE1 | GLU | A | 132 | 69.668 | 26.973 | 54.719 | 1.00 | 36.98 |
| ATOM | 1094 | OE2 | GLU | A | 132 | 71.122 | 25.412 | 54.291 | 1.00 | 45.87 |
| ATOM | 1095 | N | MET | A | 133 | 66.190 | 23.312 | 56.138 | 1.00 | 35.30 |
| ATOM | 1096 | CA | MET | A | 133 | 66.177 | 22.110 | 56.957 | 1.00 | 34.88 |
| ATOM | 1097 | C | MET | A | 133 | 66.792 | 20.981 | 56.178 | 1.00 | 33.80 |
| ATOM | 1098 | O | MET | A | 133 | 66.522 | 20.830 | 54.989 | 1.00 | 33.74 |
| ATOM | 1099 | CB | MET | A | 133 | 64.744 | 21.748 | 57.319 | 1.00 | 33.26 |
| ATOM | 1100 | CG | MET | A | 133 | 64.176 | 22.544 | 58.438 | 1.00 | 33.35 |
| ATOM | 1101 | SD | MET | A | 133 | 62.443 | 22.059 | 58.728 | 1.00 | 34.77 |
| ATOM | 1102 | CE | MET | A | 133 | 62.621 | 21.053 | 60.084 | 1.00 | 36.12 |
| ATOM | 1103 | N | ILE | A | 134 | 67.632 | 20.198 | 56.845 | 1.00 | 35.34 |
| ATOM | 1104 | CA | ILE | A | 134 | 68.234 | 19.020 | 56.243 | 1.00 | 37.22 |
| ATOM | 1105 | C | ILE | A | 134 | 67.722 | 17.787 | 56.974 | 1.00 | 34.26 |
| ATOM | 1106 | O | ILE | A | 134 | 67.808 | 17.718 | 58.186 | 1.00 | 36.80 |
| ATOM | 1107 | CB | ILE | A | 134 | 69.779 | 19.065 | 56.348 | 1.00 | 40.26 |
| ATOM | 1108 | CG1 | ILE | A | 134 | 70.341 | 20.444 | 55.945 | 1.00 | 43.15 |
| ATOM | 1109 | CG2 | ILE | A | 134 | 70.397 | 17.908 | 55.544 | 1.00 | 39.51 |
| ATOM | 1110 | CD1 | ILE | A | 134 | 70.767 | 20.560 | 54.500 | 1.00 | 45.41 |
| ATOM | 1111 | N | PHE | A | 135 | 67.186 | 16.823 | 56.235 | 1.00 | 33.93 |
| ATOM | 1112 | CA | PHE | A | 135 | 66.764 | 15.537 | 56.802 | 1.00 | 36.30 |
| ATOM | 1113 | C | PHE | A | 135 | 67.762 | 14.494 | 56.339 | 1.00 | 39.30 |
| ATOM | 1114 | O | PHE | A | 135 | 67.670 | 13.984 | 55.222 | 1.00 | 38.06 |
| ATOM | 1115 | CB | PHE | A | 135 | 65.329 | 15.193 | 56.372 | 1.00 | 33.62 |
| ATOM | 1116 | CG | PHE | A | 135 | 64.340 | 16.272 | 56.728 | 1.00 | 33.99 |
| ATOM | 1117 | CD1 | PHE | A | 135 | 63.905 | 16.420 | 58.022 | 1.00 | 32.90 |
| ATOM | 1118 | CD2 | PHE | A | 135 | 63.897 | 17.171 | 55.776 | 1.00 | 32.67 |
| ATOM | 1119 | CE1 | PHE | A | 135 | 63.017 | 17.430 | 58.355 | 1.00 | 34.12 |
| ATOM | 1120 | CE2 | PHE | A | 135 | 63.007 | 18.176 | 56.113 | 1.00 | 30.63 |
| ATOM | 1121 | CZ | PHE | A | 135 | 62.586 | 18.310 | 57.397 | 1.00 | 30.45 |
| ATOM | 1122 | N | GLU | A | 136 | 68.726 | 14.208 | 57.214 | 1.00 | 43.37 |
| ATOM | 1123 | CA | GLU | A | 136 | 69.899 | 13.395 | 56.877 | 1.00 | 47.63 |
| ATOM | 1124 | C | GLU | A | 136 | 69.494 | 11.943 | 56.705 | 1.00 | 44.75 |
| ATOM | 1125 | O | GLU | A | 136 | 70.035 | 11.249 | 55.851 | 1.00 | 45.05 |
| ATOM | 1126 | CB | GLU | A | 136 | 70.990 | 13.499 | 57.971 | 1.00 | 52.42 |
| ATOM | 1127 | CG | GLU | A | 136 | 71.085 | 14.858 | 58.671 | 1.00 | 58.67 |
| ATOM | 1128 | CD | GLU | A | 136 | 72.496 | 15.207 | 59.119 | 1.00 | 64.68 |
| ATOM | 1129 | OE1 | GLU | A | 136 | 72.979 | 14.607 | 60.109 | 1.00 | 68.18 |
| ATOM | 1130 | OE2 | GLU | A | 136 | 73.119 | 16.089 | 58.479 | 1.00 | 69.15 |
| ATOM | 1131 | N | ASP | A | 137 | 68.530 | 11.504 | 57.514 | 1.00 | 42.45 |
| ATOM | 1132 | CA | ASP | A | 137 | 68.008 | 10.137 | 57.436 | 1.00 | 42.01 |
| ATOM | 1133 | C | ASP | A | 137 | 67.372 | 9.759 | 56.080 | 1.00 | 43.10 |
| ATOM | 1134 | O | ASP | A | 137 | 67.449 | 8.597 | 55.679 | 1.00 | 46.70 |
| ATOM | 1135 | CB | ASP | A | 137 | 67.052 | 9.841 | 58.613 | 1.00 | 40.00 |
| ATOM | 1136 | CG | ASP | A | 137 | 65.732 | 10.645 | 58.559 | 1.00 | 43.12 |
| ATOM | 1137 | OD1 | ASP | A | 137 | 65.703 | 11.803 | 58.068 | 1.00 | 39.61 |
| ATOM | 1138 | OD2 | ASP | A | 137 | 64.662 | 10.187 | 59.017 | 1.00 | 38.14 |
| ATOM | 1139 | N | THR | A | 138 | 66.755 | 10.711 | 55.374 | 1.00 | 42.85 |
| ATOM | 1140 | CA | THR | A | 138 | 66.156 | 10.412 | 54.053 | 1.00 | 42.62 |
| ATOM | 1141 | C | THR | A | 138 | 66.784 | 11.165 | 52.892 | 1.00 | 41.22 |
| ATOM | 1142 | O | THR | A | 138 | 66.286 | 11.099 | 51.766 | 1.00 | 41.62 |
| ATOM | 1143 | CB | THR | A | 138 | 64.617 | 10.641 | 54.034 | 1.00 | 41.82 |
| ATOM | 1144 | OG1 | THR | A | 138 | 64.303 | 11.968 | 54.472 | 1.00 | 37.95 |
| ATOM | 1145 | CG2 | THR | A | 138 | 63.905 | 9.699 | 55.017 | 1.00 | 41.84 |
| ATOM | 1146 | N | ASN | A | 139 | 67.873 | 11.872 | 53.168 | 1.00 | 45.49 |
| ATOM | 1147 | CA | ASN | A | 139 | 68.651 | 12.566 | 52.133 | 1.00 | 47.20 |
| ATOM | 1148 | C | ASN | A | 139 | 67.884 | 13.663 | 51.411 | 1.00 | 44.12 |
| ATOM | 1149 | O | ASN | A | 139 | 67.917 | 13.745 | 50.178 | 1.00 | 41.55 |
| ATOM | 1150 | CB | ASN | A | 139 | 69.213 | 11.561 | 51.111 | 1.00 | 49.65 |
| ATOM | 1151 | CG | ASN | A | 139 | 70.600 | 11.952 | 50.608 | 1.00 | 55.72 |
| ATOM | 1152 | OD1 | ASN | A | 139 | 70.827 | 12.077 | 49.394 | 1.00 | 56.14 |
| ATOM | 1153 | ND2 | ASN | A | 139 | 71.539 | 12.141 | 51.542 | 1.00 | 53.88 |
| ATOM | 1154 | N | LEU | A | 140 | 67.190 | 14.501 | 52.180 | 1.00 | 40.44 |
| ATOM | 1155 | CA | LEU | A | 140 | 66.466 | 15.627 | 51.612 | 1.00 | 37.75 |
| ATOM | 1156 | C | LEU | A | 140 | 66.819 | 16.927 | 52.288 | 1.00 | 37.73 |
| ATOM | 1157 | O | LEU | A | 140 | 67.133 | 16.955 | 53.469 | 1.00 | 37.57 |
| ATOM | 1158 | CB | LEU | A | 140 | 64.964 | 15.408 | 51.703 | 1.00 | 35.68 |
| ATOM | 1159 | CG | LEU | A | 140 | 64.456 | 14.197 | 50.920 | 1.00 | 37.88 |
| ATOM | 1160 | CD1 | LEU | A | 140 | 63.131 | 13.764 | 51.497 | 1.00 | 38.08 |
| ATOM | 1161 | CD2 | LEU | A | 140 | 64.330 | 14.516 | 49.425 | 1.00 | 40.15 |
| ATOM | 1162 | N | LYS | A | 141 | 66.778 | 17.996 | 51.499 | 1.00 | 37.25 |
| ATOM | 1163 | CA | LYS | A | 141 | 66.857 | 19.355 | 51.980 | 1.00 | 36.58 |
| ATOM | 1164 | C | LYS | A | 141 | 65.536 | 20.017 | 51.659 | 1.00 | 33.66 |
| ATOM | 1165 | O | LYS | A | 141 | 64.922 | 19.709 | 50.645 | 1.00 | 30.42 |
| ATOM | 1166 | CB | LYS | A | 141 | 67.982 | 20.095 | 51.256 | 1.00 | 41.02 |
| ATOM | 1167 | CG | LYS | A | 141 | 68.130 | 21.559 | 51.664 | 1.00 | 45.52 |
| ATOM | 1168 | CD | LYS | A | 141 | 69.555 | 22.076 | 51.460 | 1.00 | 46.16 |
| ATOM | 1169 | CE | LYS | A | 141 | 69.785 | 22.629 | 50.076 | 1.00 | 47.51 |
| ATOM | 1170 | NZ | LYS | A | 141 | 71.203 | 23.109 | 49.957 | 1.00 | 50.68 |
| ATOM | 1171 | N | LEU | A | 142 | 65.121 | 20.944 | 52.514 | 1.00 | 30.65 |
| ATOM | 1172 | CA | LEU | A | 142 | 63.871 | 21.664 | 52.356 | 1.00 | 30.54 |
| ATOM | 1173 | C | LEU | A | 142 | 64.083 | 23.155 | 52.644 | 1.00 | 29.07 |
| ATOM | 1174 | O | LEU | A | 142 | 64.603 | 23.522 | 53.697 | 1.00 | 27.92 |
| ATOM | 1175 | CB | LEU | A | 142 | 62.844 | 21.095 | 53.342 | 1.00 | 31.26 |
| ATOM | 1176 | CG | LEU | A | 142 | 61.456 | 21.702 | 53.417 | 1.00 | 29.15 |
| ATOM | 1177 | CD1 | LEU | A | 142 | 60.715 | 21.477 | 52.097 | 1.00 | 31.49 |
| ATOM | 1178 | CD2 | LEU | A | 142 | 60.676 | 21.086 | 54.576 | 1.00 | 26.99 |
| ATOM | 1179 | N | THR | A | 143 | 63.599 | 24.003 | 51.742 | 1.00 | 30.42 |
| ATOM | 1180 | CA | THR | A | 143 | 63.812 | 25.449 | 51.808 | 1.00 | 26.87 |
| ATOM | 1181 | C | THR | A | 143 | 62.512 | 26.195 | 51.640 | 1.00 | 25.51 |
| ATOM | 1182 | O | THR | A | 143 | 61.757 | 25.946 | 50.709 | 1.00 | 27.09 |
| ATOM | 1183 | CB | THR | A | 143 | 64.787 | 25.857 | 50.664 | 1.00 | 29.15 |
| ATOM | 1184 | OG1 | THR | A | 143 | 65.962 | 25.047 | 50.743 | 1.00 | 29.13 |
| ATOM | 1185 | CG2 | THR | A | 143 | 65.295 | 27.292 | 50.837 | 1.00 | 31.43 |
| ATOM | 1186 | N | LEU | A | 144 | 62.233 | 27.108 | 52.553 | 1.00 | 24.97 |
| ATOM | 1187 | CA | LEU | A | 144 | 61.146 | 28.032 | 52.385 | 1.00 | 25.26 |
| ATOM | 1188 | C | LEU | A | 144 | 61.511 | 29.055 | 51.286 | 1.00 | 29.95 |
| ATOM | 1189 | O | LEU | A | 144 | 62.464 | 29.826 | 51.432 | 1.00 | 31.10 |
| ATOM | 1190 | CB | LEU | A | 144 | 60.885 | 28.736 | 53.705 | 1.00 | 27.54 |
| ATOM | 1191 | CG | LEU | A | 144 | 59.827 | 29.820 | 53.656 | 1.00 | 27.82 |
| ATOM | 1192 | CD1 | LEU | A | 144 | 58.487 | 29.210 | 53.283 | 1.00 | 31.82 |
| ATOM | 1193 | CD2 | LEU | A | 144 | 59.759 | 30.513 | 54.977 | 1.00 | 27.49 |
| ATOM | 1194 | N | ILE | A | 145 | 60.739 | 29.053 | 50.203 | 1.00 | 32.40 |
| ATOM | 1195 | CA | ILE | A | 145 | 60.906 | 29.972 | 49.068 | 1.00 | 30.54 |
| ATOM | 1196 | C | ILE | A | 145 | 60.122 | 31.250 | 49.246 | 1.00 | 30.05 |
| ATOM | 1197 | O | ILE | A | 145 | 60.586 | 32.331 | 48.913 | 1.00 | 31.49 |
| ATOM | 1198 | CB | ILE | A | 145 | 60.443 | 29.274 | 47.791 | 1.00 | 32.14 |
| ATOM | 1199 | CG1 | ILE | A | 145 | 61.303 | 28.044 | 47.537 | 1.00 | 34.16 |
| ATOM | 1200 | CG2 | ILE | A | 145 | 60.468 | 30.206 | 46.577 | 1.00 | 31.43 |
| ATOM | 1201 | CD1 | ILE | A | 145 | 62.792 | 28.298 | 47.576 | 1.00 | 37.81 |
| ATOM | 1202 | N | SER | A | 146 | 58.916 | 31.123 | 49.756 | 1.00 | 30.51 |
| ATOM | 1203 | CA | SER | A | 146 | 58.053 | 32.263 | 49.941 | 1.00 | 31.90 |
| ATOM | 1204 | C | SER | A | 146 | 56.887 | 31.862 | 50.814 | 1.00 | 34.24 |
| ATOM | 1205 | O | SER | A | 146 | 56.577 | 30.676 | 50.963 | 1.00 | 34.61 |
| ATOM | 1206 | CB | SER | A | 146 | 57.510 | 32.748 | 48.601 | 1.00 | 34.65 |
| ATOM | 1207 | OG | SER | A | 146 | 56.477 | 31.885 | 48.152 | 1.00 | 36.04 |
| ATOM | 1208 | N | GLU | A | 147 | 56.216 | 32.868 | 51.344 | 1.00 | 34.68 |
| ATOM | 1209 | CA | GLU | A | 147 | 55.179 | 32.676 | 52.336 | 1.00 | 37.65 |
| ATOM | 1210 | C | GLU | A | 147 | 54.228 | 33.848 | 52.217 | 1.00 | 40.62 |
| ATOM | 1211 | O | GLU | A | 147 | 54.655 | 34.997 | 52.214 | 1.00 | 43.39 |
| ATOM | 1212 | CB | GLU | A | 147 | 55.808 | 32.617 | 53.732 | 1.00 | 39.51 |
| ATOM | 1213 | CG | GLU | A | 147 | 54.842 | 32.766 | 54.894 | 1.00 | 41.67 |
| ATOM | 1214 | CD | GLU | A | 147 | 55.473 | 32.406 | 56.226 | 1.00 | 42.88 |
| ATOM | 1215 | OE1 | GLU | A | 147 | 55.544 | 31.209 | 56.547 | 1.00 | 46.46 |
| ATOM | 1216 | OE2 | GLU | A | 147 | 55.895 | 33.313 | 56.964 | 1.00 | 44.93 |
| ATOM | 1217 | N | ASP | A | 148 | 52.936 | 33.556 | 52.150 | 1.00 | 40.95 |
| ATOM | 1218 | CA | ASP | A | 148 | 51.919 | 34.570 | 52.011 | 1.00 | 37.92 |
| ATOM | 1219 | C | ASP | A | 148 | 50.864 | 34.328 | 53.083 | 1.00 | 39.88 |
| ATOM | 1220 | O | ASP | A | 148 | 50.037 | 33.408 | 52.969 | 1.00 | 32.95 |
| ATOM | 1221 | CB | ASP | A | 148 | 51.335 | 34.509 | 50.605 | 1.00 | 43.73 |
| ATOM | 1222 | CG | ASP | A | 148 | 50.246 | 35.545 | 50.362 | 1.00 | 47.38 |
| ATOM | 1223 | OD1 | ASP | A | 148 | 50.191 | 36.577 | 51.073 | 1.00 | 49.14 |
| ATOM | 1224 | OD2 | ASP | A | 148 | 49.396 | 35.388 | 49.463 | 1.00 | 50.53 |
| ATOM | 1225 | N | ILE | A | 149 | 50.910 | 35.162 | 54.128 | 1.00 | 36.98 |
| ATOM | 1226 | CA | ILE | A | 149 | 50.009 | 35.055 | 55.265 | 1.00 | 37.31 |
| ATOM | 1227 | C | ILE | A | 149 | 48.723 | 35.843 | 55.006 | 1.00 | 39.45 |
| ATOM | 1228 | O | ILE | A | 149 | 48.749 | 37.056 | 54.802 | 1.00 | 41.38 |
| ATOM | 1229 | CB | ILE | A | 149 | 50.698 | 35.565 | 56.541 | 1.00 | 38.10 |
| ATOM | 1230 | CG1 | ILE | A | 149 | 52.047 | 34.857 | 56.742 | 1.00 | 40.59 |
| ATOM | 1231 | CG2 | ILE | A | 149 | 49.789 | 35.382 | 57.748 | 1.00 | 38.89 |
| ATOM | 1232 | CD1 | ILE | A | 149 | 52.857 | 35.334 | 57.940 | 1.00 | 41.10 |
| ATOM | 1233 | N | LYS | A | 150 | 47.601 | 35.135 | 55.016 | 1.00 | 36.60 |
| ATOM | 1234 | CA | LYS | A | 150 | 46.286 | 35.743 | 54.929 | 1.00 | 36.95 |
| ATOM | 1235 | C | LYS | A | 150 | 45.510 | 35.490 | 56.214 | 1.00 | 36.61 |
| ATOM | 1236 | O | LYS | A | 150 | 45.993 | 34.827 | 57.139 | 1.00 | 36.14 |
| ATOM | 1237 | CB | LYS | A | 150 | 45.531 | 35.231 | 53.692 | 1.00 | 38.49 |
| ATOM | 1238 | CG | LYS | A | 150 | 46.350 | 35.379 | 52.394 | 1.00 | 43.90 |
| ATOM | 1239 | CD | LYS | A | 150 | 45.515 | 35.799 | 51.170 | 1.00 | 51.08 |
| ATOM | 1240 | CE | LYS | A | 150 | 45.565 | 37.321 | 50.910 | 1.00 | 55.46 |
| ATOM | 1241 | NZ | LYS | A | 150 | 45.129 | 37.689 | 49.512 | 1.00 | 57.60 |
| ATOM | 1242 | N | SER | A | 151 | 44.300 | 36.028 | 56.272 | 1.00 | 37.57 |
| ATOM | 1243 | CA | SER | A | 151 | 43.549 | 36.067 | 57.519 | 1.00 | 39.40 |
| ATOM | 1244 | C | SER | A | 151 | 43.190 | 34.674 | 58.020 | 1.00 | 39.24 |
| ATOM | 1245 | O | SER | A | 151 | 43.321 | 34.401 | 59.211 | 1.00 | 36.49 |
| ATOM | 1246 | CB | SER | A | 151 | 42.283 | 36.926 | 57.368 | 1.00 | 41.83 |
| ATOM | 1247 | OG | SER | A | 151 | 42.038 | 37.267 | 56.013 | 1.00 | 50.33 |
| ATOM | 1248 | N | TYR | A | 152 | 42.767 | 33.788 | 57.114 | 1.00 | 38.17 |
| ATOM | 1249 | CA | TYR | A | 152 | 42.261 | 32.478 | 57.513 | 1.00 | 41.75 |
| ATOM | 1250 | C | TYR | A | 152 | 43.190 | 31.317 | 57.146 | 1.00 | 39.57 |
| ATOM | 1251 | O | TYR | A | 152 | 42.959 | 30.185 | 57.564 | 1.00 | 36.58 |
| ATOM | 1252 | CB | TYR | A | 152 | 40.848 | 32.251 | 56.945 | 1.00 | 46.83 |
| ATOM | 1253 | CG | TYR | A | 152 | 39.816 | 33.184 | 57.546 | 1.00 | 56.16 |
| ATOM | 1254 | CD1 | TYR | A | 152 | 39.525 | 34.415 | 56.944 | 1.00 | 61.10 |
| ATOM | 1255 | CD2 | TYR | A | 152 | 39.146 | 32.856 | 58.732 | 1.00 | 60.48 |
| ATOM | 1256 | CE1 | TYR | A | 152 | 38.588 | 35.291 | 57.497 | 1.00 | 64.02 |
| ATOM | 1257 | CE2 | TYR | A | 152 | 38.208 | 33.729 | 59.296 | 1.00 | 65.39 |
| ATOM | 1258 | CZ | TYR | A | 152 | 37.933 | 34.943 | 58.669 | 1.00 | 66.00 |
| ATOM | 1259 | OH | TYR | A | 152 | 37.007 | 35.811 | 59.206 | 1.00 | 71.91 |
| ATOM | 1260 | N | TYR | A | 153 | 44.230 | 31.592 | 56.369 | 1.00 | 37.51 |
| ATOM | 1261 | CA | TYR | A | 153 | 45.165 | 30.559 | 55.973 | 1.00 | 35.07 |
| ATOM | 1262 | C | TYR | A | 153 | 46.472 | 31.154 | 55.481 | 1.00 | 31.45 |
| ATOM | 1263 | O | TYR | A | 153 | 46.514 | 32.318 | 55.122 | 1.00 | 29.67 |
| ATOM | 1264 | CB | TYR | A | 153 | 44.538 | 29.674 | 54.880 | 1.00 | 36.12 |
| ATOM | 1265 | CG | TYR | A | 153 | 44.435 | 30.333 | 53.529 | 1.00 | 38.08 |
| ATOM | 1266 | CD1 | TYR | A | 153 | 43.311 | 31.085 | 53.180 | 1.00 | 44.22 |
| ATOM | 1267 | CD2 | TYR | A | 153 | 45.449 | 30.194 | 52.591 | 1.00 | 41.96 |
| ATOM | 1268 | CE1 | TYR | A | 153 | 43.213 | 31.695 | 51.933 | 1.00 | 46.04 |
| ATOM | 1269 | CE2 | TYR | A | 153 | 45.362 | 30.801 | 51.346 | 1.00 | 46.25 |
| ATOM | 1270 | CZ | TYR | A | 153 | 44.252 | 31.556 | 51.027 | 1.00 | 48.63 |
| ATOM | 1271 | OH | TYR | A | 153 | 44.196 | 32.146 | 49.781 | 1.00 | 55.82 |
| ATOM | 1272 | N | THR | A | 154 | 47.523 | 30.336 | 55.443 | 1.00 | 24.30 |
| ATOM | 1273 | CA | THR | A | 154 | 48.794 | 30.726 | 54.845 | 1.00 | 27.50 |
| ATOM | 1274 | C | THR | A | 154 | 49.209 | 29.758 | 53.718 | 1.00 | 26.17 |
| ATOM | 1275 | O | THR | A | 154 | 48.947 | 28.566 | 53.796 | 1.00 | 28.65 |
| ATOM | 1276 | CB | THR | A | 154 | 49.857 | 30.786 | 55.956 | 1.00 | 26.73 |
| ATOM | 1277 | OG1 | THR | A | 154 | 49.487 | 31.798 | 56.899 | 1.00 | 27.84 |
| ATOM | 1278 | CG2 | THR | A | 154 | 51.191 | 31.230 | 55.441 | 1.00 | 27.67 |
| ATOM | 1279 | N | VAL | A | 155 | 49.816 | 30.286 | 52.658 | 1.00 | 26.64 |
| ATOM | 1280 | CA | VAL | A | 155 | 50.359 | 29.475 | 51.569 | 1.00 | 25.67 |
| ATOM | 1281 | C | VAL | A | 155 | 51.839 | 29.717 | 51.506 | 1.00 | 27.10 |
| ATOM | 1282 | O | VAL | A | 155 | 52.289 | 30.875 | 51.433 | 1.00 | 30.22 |
| ATOM | 1283 | CB | VAL | A | 155 | 49.787 | 29.857 | 50.174 | 1.00 | 27.77 |
| ATOM | 1284 | CG1 | VAL | A | 155 | 50.170 | 28.802 | 49.128 | 1.00 | 32.39 |
| ATOM | 1285 | CG2 | VAL | A | 155 | 48.332 | 30.020 | 50.238 | 1.00 | 35.84 |
| ATOM | 1286 | N | ARG | A | 156 | 52.597 | 28.633 | 51.494 | 1.00 | 25.08 |
| ATOM | 1287 | CA | ARG | A | 156 | 54.028 | 28.689 | 51.385 | 1.00 | 26.10 |
| ATOM | 1288 | C | ARG | A | 156 | 54.488 | 27.919 | 50.151 | 1.00 | 29.29 |
| ATOM | 1289 | O | ARG | A | 156 | 53.908 | 26.917 | 49.778 | 1.00 | 28.61 |
| ATOM | 1290 | CB | ARG | A | 156 | 54.675 | 28.110 | 52.642 | 1.00 | 27.56 |
| ATOM | 1291 | CG | ARG | A | 156 | 54.185 | 28.760 | 53.943 | 1.00 | 30.11 |
| ATOM | 1292 | CD | ARG | A | 156 | 54.898 | 28.293 | 55.213 | 1.00 | 31.28 |
| ATOM | 1293 | NE | ARG | A | 156 | 54.310 | 28.872 | 56.431 | 1.00 | 32.23 |
| ATOM | 1294 | CZ | ARG | A | 156 | 53.240 | 28.402 | 57.079 | 1.00 | 30.33 |
| ATOM | 1295 | NH1 | ARG | A | 156 | 52.609 | 27.313 | 56.676 | 1.00 | 33.27 |
| ATOM | 1296 | NH2 | ARG | A | 156 | 52.799 | 29.024 | 58.165 | 1.00 | 32.37 |
| ATOM | 1297 | N | GLN | A | 157 | 55.526 | 28.415 | 49.500 | 1.00 | 27.76 |
| ATOM | 1298 | CA | GLN | A | 157 | 56.202 | 27.651 | 48.491 | 1.00 | 26.23 |
| ATOM | 1299 | C | GLN | A | 157 | 57.474 | 27.135 | 49.121 | 1.00 | 25.68 |
| ATOM | 1300 | O | GLN | A | 157 | 58.165 | 27.842 | 49.831 | 1.00 | 27.03 |
| ATOM | 1301 | CB | GLN | A | 157 | 56.438 | 28.528 | 47.266 | 1.00 | 28.63 |
| ATOM | 1302 | CG | GLN | A | 157 | 57.220 | 27.861 | 46.190 | 1.00 | 35.97 |
| ATOM | 1303 | CD | GLN | A | 157 | 57.376 | 28.715 | 44.949 | 1.00 | 38.81 |
| ATOM | 1304 | OE1 | GLN | A | 157 | 56.665 | 29.716 | 44.776 | 1.00 | 39.09 |
| ATOM | 1305 | NE2 | GLN | A | 157 | 58.311 | 28.322 | 44.080 | 1.00 | 37.90 |
| ATOM | 1306 | N | LEU | A | 158 | 57.761 | 25.867 | 48.892 | 1.00 | 26.47 |
| ATOM | 1307 | CA | LEU | A | 158 | 58.905 | 25.202 | 49.481 | 1.00 | 26.97 |
| ATOM | 1308 | C | LEU | A | 158 | 59.626 | 24.509 | 48.366 | 1.00 | 26.82 |
| ATOM | 1309 | O | LEU | A | 158 | 59.021 | 24.177 | 47.349 | 1.00 | 30.23 |
| ATOM | 1310 | CB | LEU | A | 158 | 58.474 | 24.169 | 50.528 | 1.00 | 25.90 |
| ATOM | 1311 | CG | LEU | A | 158 | 57.530 | 24.616 | 51.648 | 1.00 | 28.92 |
| ATOM | 1312 | CD1 | LEU | A | 158 | 56.875 | 23.410 | 52.328 | 1.00 | 31.53 |
| ATOM | 1313 | CD2 | LEU | A | 158 | 58.277 | 25.462 | 52.668 | 1.00 | 32.05 |
| ATOM | 1314 | N | GLU | A | 159 | 60.923 | 24.310 | 48.534 | 1.00 | 28.10 |
| ATOM | 1315 | CA | GLU | A | 159 | 61.701 | 23.530 | 47.585 | 1.00 | 30.18 |
| ATOM | 1316 | C | GLU | A | 159 | 62.301 | 22.366 | 48.320 | 1.00 | 30.18 |
| ATOM | 1317 | O | GLU | A | 159 | 62.841 | 22.529 | 49.400 | 1.00 | 30.29 |
| ATOM | 1318 | CB | GLU | A | 159 | 62.803 | 24.358 | 46.938 | 1.00 | 31.30 |
| ATOM | 1319 | CG | GLU | A | 159 | 63.487 | 23.633 | 45.789 | 1.00 | 40.31 |
| ATOM | 1320 | CD | GLU | A | 159 | 64.505 | 24.503 | 45.054 | 1.00 | 44.60 |
| ATOM | 1321 | OE1 | GLU | A | 159 | 65.664 | 24.081 | 44.931 | 1.00 | 48.32 |
| ATOM | 1322 | OE2 | GLU | A | 159 | 64.144 | 25.601 | 44.603 | 1.00 | 49.88 |
| ATOM | 1323 | N | LEU | A | 160 | 62.195 | 21.197 | 47.710 | 1.00 | 31.42 |
| ATOM | 1324 | CA | LEU | A | 160 | 62.685 | 19.953 | 48.249 | 1.00 | 35.20 |
| ATOM | 1325 | C | LEU | A | 160 | 63.812 | 19.485 | 47.335 | 1.00 | 39.41 |
| ATOM | 1326 | O | LEU | A | 160 | 63.610 | 19.381 | 46.134 | 1.00 | 39.24 |
| ATOM | 1327 | CB | LEU | A | 160 | 61.511 | 18.967 | 48.221 | 1.00 | 38.43 |
| ATOM | 1328 | CG | LEU | A | 160 | 61.364 | 17.751 | 49.128 | 1.00 | 38.81 |
| ATOM | 1329 | CD1 | LEU | A | 160 | 59.970 | 17.156 | 48.856 | 1.00 | 34.93 |
| ATOM | 1330 | CD2 | LEU | A | 160 | 61.552 | 18.064 | 50.614 | 1.00 | 34.53 |
| ATOM | 1331 | N | GLU | A | 161 | 64.997 | 19.221 | 47.886 | 1.00 | 44.11 |
| ATOM | 1332 | CA | GLU | A | 161 | 66.130 | 18.742 | 47.097 | 1.00 | 48.70 |
| ATOM | 1333 | C | GLU | A | 161 | 66.536 | 17.335 | 47.516 | 1.00 | 49.27 |
| ATOM | 1334 | O | GLU | A | 161 | 66.820 | 17.090 | 48.683 | 1.00 | 47.95 |
| ATOM | 1335 | CB | GLU | A | 161 | 67.339 | 19.683 | 47.242 | 1.00 | 51.56 |
| ATOM | 1336 | CG | GLU | A | 161 | 68.406 | 19.487 | 46.163 | 1.00 | 54.78 |
| ATOM | 1337 | CD | GLU | A | 161 | 69.724 | 20.166 | 46.481 | 1.00 | 56.70 |
| ATOM | 1338 | OE1 | GLU | A | 161 | 69.708 | 21.310 | 46.956 | 1.00 | 57.99 |
| ATOM | 1339 | OE2 | GLU | A | 161 | 70.785 | 19.552 | 46.244 | 1.00 | 64.66 |
| ATOM | 1340 | N | ASN | A | 162 | 66.548 | 16.405 | 46.568 | 1.00 | 54.08 |
| ATOM | 1341 | CA | ASN | A | 162 | 67.179 | 15.107 | 46.789 | 1.00 | 56.50 |
| ATOM | 1342 | C | ASN | A | 162 | 68.695 | 15.304 | 46.821 | 1.00 | 58.99 |
| ATOM | 1343 | O | ASN | A | 162 | 69.323 | 15.397 | 45.779 | 1.00 | 61.68 |
| ATOM | 1344 | CB | ASN | A | 162 | 66.775 | 14.119 | 45.693 | 1.00 | 57.69 |
| ATOM | 1345 | CG | ASN | A | 162 | 67.313 | 12.711 | 45.939 | 1.00 | 59.88 |
| ATOM | 1346 | OD1 | ASN | A | 162 | 68.520 | 12.511 | 46.145 | 1.00 | 58.62 |
| ATOM | 1347 | ND2 | ASN | A | 162 | 66.413 | 11.728 | 45.918 | 1.00 | 56.89 |
| ATOM | 1348 | N | LEU | A | 163 | 69.267 | 15.380 | 48.020 | 1.00 | 59.13 |
| ATOM | 1349 | CA | LEU | A | 163 | 70.678 | 15.724 | 48.202 | 1.00 | 63.54 |
| ATOM | 1350 | C | LEU | A | 163 | 71.705 | 14.880 | 47.410 | 1.00 | 67.69 |
| ATOM | 1351 | O | LEU | A | 163 | 72.784 | 15.382 | 47.075 | 1.00 | 66.84 |
| ATOM | 1352 | CB | LEU | A | 163 | 71.040 | 15.688 | 49.696 | 1.00 | 62.10 |
| ATOM | 1353 | CG | LEU | A | 163 | 70.544 | 16.860 | 50.554 | 1.00 | 62.17 |
| ATOM | 1354 | CD1 | LEU | A | 163 | 71.139 | 16.782 | 51.945 | 1.00 | 60.89 |
| ATOM | 1355 | CD2 | LEU | A | 163 | 70.853 | 18.219 | 49.914 | 1.00 | 63.68 |
| ATOM | 1356 | N | THR | A | 164 | 71.381 | 13.619 | 47.125 | 1.00 | 71.50 |
| ATOM | 1357 | CA | THR | A | 164 | 72.294 | 12.743 | 46.388 | 1.00 | 75.23 |
| ATOM | 1358 | C | THR | A | 164 | 72.340 | 13.146 | 44.912 | 1.00 | 76.98 |
| ATOM | 1359 | O | THR | A | 164 | 73.420 | 13.347 | 44.353 | 1.00 | 78.26 |
| ATOM | 1360 | CB | THR | A | 164 | 71.869 | 11.255 | 46.529 | 1.00 | 76.31 |
| ATOM | 1361 | OG1 | THR | A | 164 | 72.214 | 10.768 | 47.831 | 1.00 | 77.12 |
| ATOM | 1362 | CG2 | THR | A | 164 | 72.671 | 10.347 | 45.589 | 1.00 | 77.98 |
| ATOM | 1363 | N | THR | A | 165 | 71.155 | 13.280 | 44.309 | 1.00 | 77.26 |
| ATOM | 1364 | CA | THR | A | 165 | 71.001 | 13.529 | 42.871 | 1.00 | 75.84 |
| ATOM | 1365 | C | THR | A | 165 | 70.880 | 15.016 | 42.483 | 1.00 | 73.80 |
| ATOM | 1366 | O | THR | A | 165 | 70.856 | 15.342 | 41.299 | 1.00 | 75.32 |
| ATOM | 1367 | CB | THR | A | 165 | 69.759 | 12.769 | 42.347 | 1.00 | 76.34 |
| ATOM | 1368 | OG1 | THR | A | 165 | 68.565 | 13.344 | 42.894 | 1.00 | 78.11 |
| ATOM | 1369 | CG2 | THR | A | 165 | 69.733 | 11.322 | 42.853 | 1.00 | 76.64 |
| ATOM | 1370 | N | GLN | A | 166 | 70.801 | 15.900 | 43.476 | 1.00 | 69.63 |
| ATOM | 1371 | CA | GLN | A | 166 | 70.591 | 17.348 | 43.288 | 1.00 | 66.56 |
| ATOM | 1372 | C | GLN | A | 166 | 69.306 | 17.803 | 42.546 | 1.00 | 61.69 |
| ATOM | 1373 | O | GLN | A | 166 | 69.146 | 19.003 | 42.296 | 1.00 | 58.47 |
| ATOM | 1374 | CB | GLN | A | 166 | 71.836 | 18.000 | 42.656 | 1.00 | 69.16 |
| ATOM | 1375 | CG | GLN | A | 166 | 73.145 | 17.762 | 43.440 | 1.00 | 71.16 |
| ATOM | 1376 | CD | GLN | A | 166 | 74.080 | 18.970 | 43.451 | 1.00 | 72.58 |
| ATOM | 1377 | OE1 | GLN | A | 166 | 74.818 | 19.185 | 44.415 | 1.00 | 74.04 |
| ATOM | 1378 | NE2 | GLN | A | 166 | 74.052 | 19.751 | 42.386 | 1.00 | 74.28 |
| ATOM | 1379 | N | GLU | A | 167 | 68.390 | 16.881 | 42.226 | 1.00 | 57.39 |
| ATOM | 1380 | CA | GLU | A | 167 | 67.062 | 17.257 | 41.692 | 1.00 | 57.78 |
| ATOM | 1381 | C | GLU | A | 167 | 66.280 | 18.111 | 42.705 | 1.00 | 53.55 |
| ATOM | 1382 | O | GLU | A | 167 | 66.476 | 17.992 | 43.914 | 1.00 | 51.23 |
| ATOM | 1383 | CB | GLU | A | 167 | 66.178 | 16.035 | 41.387 | 1.00 | 59.44 |
| ATOM | 1384 | CG | GLU | A | 167 | 66.720 | 14.998 | 40.420 | 1.00 | 64.57 |
| ATOM | 1385 | CD | GLU | A | 167 | 66.137 | 13.621 | 40.700 | 1.00 | 68.48 |
| ATOM | 1386 | OE1 | GLU | A | 167 | 64.902 | 13.441 | 40.554 | 1.00 | 71.00 |
| ATOM | 1387 | OE2 | GLU | A | 167 | 66.909 | 12.723 | 41.093 | 1.00 | 70.28 |
| ATOM | 1388 | N | THR | A | 168 | 65.383 | 18.948 | 42.200 | 1.00 | 50.37 |
| ATOM | 1389 | CA | THR | A | 168 | 64.550 | 19.809 | 43.034 | 1.00 | 47.15 |
| ATOM | 1390 | C | THR | A | 168 | 63.097 | 19.658 | 42.641 | 1.00 | 45.69 |
| ATOM | 1391 | O | THR | A | 168 | 62.799 | 19.382 | 41.487 | 1.00 | 46.95 |
| ATOM | 1392 | CB | THR | A | 168 | 64.922 | 21.271 | 42.843 | 1.00 | 46.52 |
| ATOM | 1393 | OG1 | THR | A | 168 | 64.870 | 21.595 | 41.446 | 1.00 | 45.51 |
| ATOM | 1394 | CG2 | THR | A | 168 | 66.361 | 21.534 | 43.269 | 1.00 | 46.99 |
| ATOM | 1395 | N | ARG | A | 169 | 62.199 | 19.873 | 43.594 | 1.00 | 41.85 |
| ATOM | 1396 | CA | ARG | A | 169 | 60.769 | 19.945 | 43.309 | 1.00 | 39.92 |
| ATOM | 1397 | C | ARG | A | 169 | 60.167 | 21.092 | 44.097 | 1.00 | 36.00 |
| ATOM | 1398 | O | ARG | A | 169 | 60.542 | 21.301 | 45.234 | 1.00 | 36.05 |
| ATOM | 1399 | CB | ARG | A | 169 | 60.091 | 18.631 | 43.678 | 1.00 | 41.63 |
| ATOM | 1400 | CG | ARG | A | 169 | 60.529 | 17.453 | 42.803 | 1.00 | 47.97 |
| ATOM | 1401 | CD | ARG | A | 169 | 60.037 | 16.084 | 43.258 | 1.00 | 53.84 |
| ATOM | 1402 | NE | ARG | A | 169 | 59.410 | 15.355 | 42.159 | 1.00 | 61.38 |
| ATOM | 1403 | CZ | ARG | A | 169 | 58.147 | 15.510 | 41.770 | 1.00 | 65.71 |
| ATOM | 1404 | NH1 | ARG | A | 169 | 57.685 | 14.806 | 40.743 | 1.00 | 69.78 |
| ATOM | 1405 | NH2 | ARG | A | 169 | 57.334 | 16.350 | 42.402 | 1.00 | 66.33 |
| ATOM | 1406 | N | GLU | A | 170 | 59.266 | 21.842 | 43.473 | 1.00 | 31.05 |
| ATOM | 1407 | CA | GLU | A | 170 | 58.476 | 22.866 | 44.130 | 1.00 | 34.08 |
| ATOM | 1408 | C | GLU | A | 170 | 57.255 | 22.215 | 44.822 | 1.00 | 32.33 |
| ATOM | 1409 | O | GLU | A | 170 | 56.479 | 21.527 | 44.184 | 1.00 | 31.31 |
| ATOM | 1410 | CB | GLU | A | 170 | 58.004 | 23.911 | 43.091 | 1.00 | 38.29 |
| ATOM | 1411 | CG | GLU | A | 170 | 57.032 | 24.969 | 43.634 | 1.00 | 47.97 |
| ATOM | 1412 | CD | GLU | A | 170 | 56.421 | 25.896 | 42.573 | 1.00 | 52.73 |
| ATOM | 1413 | OE1 | GLU | A | 170 | 56.787 | 25.827 | 41.372 | 1.00 | 60.61 |
| ATOM | 1414 | OE2 | GLU | A | 170 | 55.562 | 26.720 | 42.954 | 1.00 | 55.45 |
| ATOM | 1415 | N | ILE | A | 171 | 57.093 | 22.444 | 46.122 | 1.00 | 31.01 |
| ATOM | 1416 | CA | ILE | A | 171 | 55.911 | 22.019 | 46.866 | 1.00 | 25.68 |
| ATOM | 1417 | C | ILE | A | 171 | 55.143 | 23.212 | 47.391 | 1.00 | 24.75 |
| ATOM | 1418 | O | ILE | A | 171 | 55.738 | 24.165 | 47.917 | 1.00 | 27.08 |
| ATOM | 1419 | CB | ILE | A | 171 | 56.324 | 21.139 | 48.055 | 1.00 | 27.41 |
| ATOM | 1420 | CG1 | ILE | A | 171 | 57.228 | 19.983 | 47.605 | 1.00 | 28.28 |
| ATOM | 1421 | CG2 | ILE | A | 171 | 55.093 | 20.620 | 48.752 | 1.00 | 31.23 |
| ATOM | 1422 | CD1 | ILE | A | 171 | 56.592 | 19.051 | 46.602 | 1.00 | 30.57 |
| ATOM | 1423 | N | LEU | A | 172 | 53.821 | 23.172 | 47.286 | 1.00 | 22.95 |
| ATOM | 1424 | CA | LEU | A | 172 | 52.987 | 24.192 | 47.915 | 1.00 | 24.79 |
| ATOM | 1425 | C | LEU | A | 172 | 52.448 | 23.652 | 49.233 | 1.00 | 27.69 |
| ATOM | 1426 | O | LEU | A | 172 | 51.962 | 22.526 | 49.287 | 1.00 | 27.52 |
| ATOM | 1427 | CB | LEU | A | 172 | 51.840 | 24.622 | 46.980 | 1.00 | 25.78 |
| ATOM | 1428 | CG | LEU | A | 172 | 52.298 | 25.213 | 45.637 | 1.00 | 28.93 |
| ATOM | 1429 | CD1 | LEU | A | 172 | 51.119 | 25.492 | 44.738 | 1.00 | 33.95 |
| ATOM | 1430 | CD2 | LEU | A | 172 | 53.075 | 26.505 | 45.846 | 1.00 | 33.75 |
| ATOM | 1431 | N | HIS | A | 173 | 52.550 | 24.467 | 50.279 | 1.00 | 25.46 |
| ATOM | 1432 | CA | HIS | A | 173 | 52.099 | 24.163 | 51.619 | 1.00 | 25.40 |
| ATOM | 1433 | C | HIS | A | 173 | 50.884 | 25.038 | 51.895 | 1.00 | 27.85 |
| ATOM | 1434 | O | HIS | A | 173 | 50.992 | 26.255 | 51.960 | 1.00 | 25.57 |
| ATOM | 1435 | CB | HIS | A | 173 | 53.219 | 24.451 | 52.635 | 1.00 | 27.52 |
| ATOM | 1436 | CG | HIS | A | 173 | 52.962 | 23.882 | 54.003 | 1.00 | 28.74 |
| ATOM | 1437 | ND1 | HIS | A | 173 | 52.361 | 24.604 | 55.009 | 1.00 | 32.01 |
| ATOM | 1438 | CD2 | HIS | A | 173 | 53.234 | 22.666 | 54.531 | 1.00 | 28.14 |
| ATOM | 1439 | CE1 | HIS | A | 173 | 52.263 | 23.856 | 56.094 | 1.00 | 31.02 |
| ATOM | 1440 | NE2 | HIS | A | 173 | 52.776 | 22.670 | 55.828 | 1.00 | 25.05 |
| ATOM | 1441 | N | PHE | A | 174 | 49.722 | 24.416 | 52.052 | 1.00 | 23.77 |
| ATOM | 1442 | CA | PHE | A | 174 | 48.488 | 25.129 | 52.292 | 1.00 | 23.14 |
| ATOM | 1443 | C | PHE | A | 174 | 48.088 | 24.933 | 53.718 | 1.00 | 25.18 |
| ATOM | 1444 | O | PHE | A | 174 | 47.684 | 23.847 | 54.099 | 1.00 | 25.93 |
| ATOM | 1445 | CB | PHE | A | 174 | 47.398 | 24.634 | 51.357 | 1.00 | 23.57 |
| ATOM | 1446 | CG | PHE | A | 174 | 47.553 | 25.144 | 49.978 | 1.00 | 26.89 |
| ATOM | 1447 | CD1 | PHE | A | 174 | 47.054 | 26.388 | 49.633 | 1.00 | 31.06 |
| ATOM | 1448 | CD2 | PHE | A | 174 | 48.221 | 24.407 | 49.030 | 1.00 | 29.99 |
| ATOM | 1449 | CE1 | PHE | A | 174 | 47.233 | 26.882 | 48.353 | 1.00 | 33.42 |
| ATOM | 1450 | CE2 | PHE | A | 174 | 48.396 | 24.907 | 47.767 | 1.00 | 30.05 |
| ATOM | 1451 | CZ | PHE | A | 174 | 47.916 | 26.138 | 47.438 | 1.00 | 28.42 |
| ATOM | 1452 | N | HIS | A | 175 | 48.179 | 25.998 | 54.509 | 1.00 | 26.31 |
| ATOM | 1453 | CA | HIS | A | 175 | 48.073 | 25.891 | 55.949 | 1.00 | 26.03 |
| ATOM | 1454 | C | HIS | A | 175 | 46.837 | 26.628 | 56.434 | 1.00 | 26.92 |
| ATOM | 1455 | O | HIS | A | 175 | 46.803 | 27.846 | 56.488 | 1.00 | 30.80 |
| ATOM | 1456 | CB | HIS | A | 175 | 49.373 | 26.377 | 56.597 | 1.00 | 26.25 |
| ATOM | 1457 | CG | HIS | A | 175 | 49.434 | 26.186 | 58.079 | 1.00 | 23.87 |
| ATOM | 1458 | ND1 | HIS | A | 175 | 50.325 | 26.874 | 58.877 | 1.00 | 21.48 |
| ATOM | 1459 | CD2 | HIS | A | 175 | 48.735 | 25.374 | 58.910 | 1.00 | 26.98 |
| ATOM | 1460 | CE1 | HIS | A | 175 | 50.164 | 26.503 | 60.135 | 1.00 | 25.22 |
| ATOM | 1461 | NE2 | HIS | A | 175 | 49.203 | 25.596 | 60.186 | 1.00 | 24.19 |
| ATOM | 1462 | N | TYR | A | 176 | 45.787 | 25.869 | 56.738 | 1.00 | 27.05 |
| ATOM | 1463 | CA | TYR | A | 176 | 44.594 | 26.441 | 57.306 | 1.00 | 28.27 |
| ATOM | 1464 | C | TYR | A | 176 | 44.851 | 26.679 | 58.777 | 1.00 | 29.68 |
| ATOM | 1465 | O | TYR | A | 176 | 45.037 | 25.740 | 59.532 | 1.00 | 28.65 |
| ATOM | 1466 | CB | TYR | A | 176 | 43.400 | 25.532 | 57.116 | 1.00 | 29.77 |
| ATOM | 1467 | CG | TYR | A | 176 | 42.088 | 26.207 | 57.393 | 1.00 | 28.59 |
| ATOM | 1468 | CD1 | TYR | A | 176 | 41.374 | 25.942 | 58.551 | 1.00 | 32.85 |
| ATOM | 1469 | CD2 | TYR | A | 176 | 41.552 | 27.096 | 56.484 | 1.00 | 28.77 |
| ATOM | 1470 | CE1 | TYR | A | 176 | 40.143 | 26.570 | 58.799 | 1.00 | 35.34 |
| ATOM | 1471 | CE2 | TYR | A | 176 | 40.334 | 27.717 | 56.714 | 1.00 | 33.98 |
| ATOM | 1472 | CZ | TYR | A | 176 | 39.634 | 27.454 | 57.869 | 1.00 | 32.54 |
| ATOM | 1473 | OH | TYR | A | 176 | 38.437 | 28.080 | 58.072 | 1.00 | 34.52 |
| ATOM | 1474 | N | THR | A | 177 | 44.841 | 27.949 | 59.173 | 1.00 | 32.09 |
| ATOM | 1475 | CA | THR | A | 177 | 45.247 | 28.369 | 60.514 | 1.00 | 33.80 |
| ATOM | 1476 | C | THR | A | 177 | 44.111 | 28.787 | 61.469 | 1.00 | 34.23 |
| ATOM | 1477 | O | THR | A | 177 | 44.388 | 29.192 | 62.602 | 1.00 | 39.48 |
| ATOM | 1478 | CB | THR | A | 177 | 46.229 | 29.542 | 60.376 | 1.00 | 33.44 |
| ATOM | 1479 | OG1 | THR | A | 177 | 45.597 | 30.629 | 59.676 | 1.00 | 29.45 |
| ATOM | 1480 | CG2 | THR | A | 177 | 47.426 | 29.157 | 59.495 | 1.00 | 28.83 |
| ATOM | 1481 | N | THR | A | 178 | 42.853 | 28.682 | 61.055 | 1.00 | 35.48 |
| ATOM | 1482 | CA | THR | A | 178 | 41.752 | 29.239 | 61.860 | 1.00 | 36.85 |
| ATOM | 1483 | C | THR | A | 178 | 40.661 | 28.270 | 62.290 | 1.00 | 36.36 |
| ATOM | 1484 | O | THR | A | 178 | 39.655 | 28.701 | 62.837 | 1.00 | 41.00 |
| ATOM | 1485 | CB | THR | A | 178 | 41.113 | 30.430 | 61.130 | 1.00 | 38.52 |
| ATOM | 1486 | OG1 | THR | A | 178 | 40.857 | 30.078 | 59.768 | 1.00 | 39.50 |
| ATOM | 1487 | CG2 | THR | A | 178 | 42.102 | 31.614 | 61.060 | 1.00 | 39.30 |
| ATOM | 1488 | N | TRP | A | 179 | 40.849 | 26.974 | 62.074 | 1.00 | 36.04 |
| ATOM | 1489 | CA | TRP | A | 179 | 39.917 | 25.989 | 62.619 | 1.00 | 36.03 |
| ATOM | 1490 | C | TRP | A | 179 | 39.908 | 26.136 | 64.143 | 1.00 | 35.88 |
| ATOM | 1491 | O | TRP | A | 179 | 40.960 | 26.030 | 64.783 | 1.00 | 38.24 |
| ATOM | 1492 | CB | TRP | A | 179 | 40.320 | 24.552 | 62.238 | 1.00 | 32.03 |
| ATOM | 1493 | CG | TRP | A | 179 | 39.235 | 23.560 | 62.491 | 1.00 | 32.00 |
| ATOM | 1494 | CD1 | TRP | A | 179 | 38.794 | 23.135 | 63.697 | 1.00 | 33.75 |
| ATOM | 1495 | CD2 | TRP | A | 179 | 38.443 | 22.866 | 61.509 | 1.00 | 29.81 |
| ATOM | 1496 | NE1 | TRP | A | 179 | 37.775 | 22.227 | 63.545 | 1.00 | 36.67 |
| ATOM | 1497 | CE2 | TRP | A | 179 | 37.539 | 22.039 | 62.210 | 1.00 | 34.91 |
| ATOM | 1498 | CE3 | TRP | A | 179 | 38.413 | 22.853 | 60.111 | 1.00 | 32.08 |
| ATOM | 1499 | CZ2 | TRP | A | 179 | 36.614 | 21.212 | 61.566 | 1.00 | 35.91 |
| ATOM | 1500 | CZ3 | TRP | A | 179 | 37.493 | 22.023 | 59.460 | 1.00 | 30.25 |
| ATOM | 1501 | CH2 | TRP | A | 179 | 36.603 | 21.222 | 60.190 | 1.00 | 36.85 |
| ATOM | 1502 | N | PRO | A | 180 | 38.740 | 26.345 | 64.738 | 1.00 | 37.20 |
| ATOM | 1503 | CA | PRO | A | 180 | 38.683 | 26.624 | 66.185 | 1.00 | 38.24 |
| ATOM | 1504 | C | PRO | A | 180 | 39.171 | 25.439 | 67.020 | 1.00 | 36.65 |
| ATOM | 1505 | O | PRO | A | 180 | 39.023 | 24.290 | 66.613 | 1.00 | 36.67 |
| ATOM | 1506 | CB | PRO | A | 180 | 37.194 | 26.907 | 66.441 | 1.00 | 37.11 |
| ATOM | 1507 | CG | PRO | A | 180 | 36.475 | 26.197 | 65.334 | 1.00 | 38.41 |
| ATOM | 1508 | CD | PRO | A | 180 | 37.398 | 26.288 | 64.129 | 1.00 | 37.33 |
| ATOM | 1509 | N | ASP | A | 181 | 39.749 | 25.730 | 68.178 | 1.00 | 39.69 |
| ATOM | 1510 | CA | ASP | A | 181 | 40.157 | 24.698 | 69.123 | 1.00 | 42.88 |
| ATOM | 1511 | C | ASP | A | 181 | 39.020 | 23.744 | 69.460 | 1.00 | 41.80 |
| ATOM | 1512 | O | ASP | A | 181 | 39.243 | 22.543 | 69.561 | 1.00 | 45.13 |
| ATOM | 1513 | CB | ASP | A | 181 | 40.700 | 25.326 | 70.414 | 1.00 | 46.18 |
| ATOM | 1514 | CG | ASP | A | 181 | 42.201 | 25.576 | 70.368 | 1.00 | 49.21 |
| ATOM | 1515 | OD1 | ASP | A | 181 | 42.857 | 25.191 | 69.371 | 1.00 | 51.80 |
| ATOM | 1516 | OD2 | ASP | A | 181 | 42.814 | 26.151 | 71.296 | 1.00 | 52.55 |
| ATOM | 1517 | N | PHE | A | 182 | 37.810 | 24.273 | 69.629 | 1.00 | 42.38 |
| ATOM | 1518 | CA | PHE | A | 182 | 36.635 | 23.454 | 69.943 | 1.00 | 44.84 |
| ATOM | 1519 | C | PHE | A | 182 | 35.550 | 23.631 | 68.881 | 1.00 | 45.23 |
| ATOM | 1520 | O | PHE | A | 182 | 35.287 | 24.733 | 68.415 | 1.00 | 45.26 |
| ATOM | 1521 | CB | PHE | A | 182 | 36.082 | 23.791 | 71.341 | 1.00 | 45.04 |
| ATOM | 1522 | CG | PHE | A | 182 | 36.969 | 23.323 | 72.471 | 1.00 | 49.01 |
| ATOM | 1523 | CD1 | PHE | A | 182 | 36.824 | 22.046 | 73.005 | 1.00 | 49.36 |
| ATOM | 1524 | CD2 | PHE | A | 182 | 37.959 | 24.152 | 72.989 | 1.00 | 50.26 |
| ATOM | 1525 | CE1 | PHE | A | 182 | 37.648 | 21.607 | 74.040 | 1.00 | 50.70 |
| ATOM | 1526 | CE2 | PHE | A | 182 | 38.789 | 23.712 | 74.023 | 1.00 | 51.25 |
| ATOM | 1527 | CZ | PHE | A | 182 | 38.632 | 22.441 | 74.545 | 1.00 | 49.16 |
| ATOM | 1528 | N | GLY | A | 183 | 34.926 | 22.527 | 68.500 | 1.00 | 45.75 |
| ATOM | 1529 | CA | GLY | A | 183 | 33.852 | 22.562 | 67.533 | 1.00 | 47.78 |
| ATOM | 1530 | C | GLY | A | 183 | 34.334 | 22.753 | 66.110 | 1.00 | 47.38 |
| ATOM | 1531 | O | GLY | A | 183 | 35.456 | 22.381 | 65.752 | 1.00 | 45.73 |
| ATOM | 1532 | N | VAL | A | 184 | 33.464 | 23.345 | 65.301 | 1.00 | 45.77 |
| ATOM | 1533 | CA | VAL | A | 184 | 33.718 | 23.551 | 63.887 | 1.00 | 44.43 |
| ATOM | 1534 | C | VAL | A | 184 | 33.565 | 25.035 | 63.616 | 1.00 | 45.59 |
| ATOM | 1535 | O | VAL | A | 184 | 33.027 | 25.751 | 64.461 | 1.00 | 44.78 |
| ATOM | 1536 | CB | VAL | A | 184 | 32.735 | 22.704 | 63.045 | 1.00 | 44.45 |
| ATOM | 1537 | CG1 | VAL | A | 184 | 32.956 | 21.226 | 63.322 | 1.00 | 40.88 |
| ATOM | 1538 | CG2 | VAL | A | 184 | 31.269 | 23.055 | 63.346 | 1.00 | 45.29 |
| ATOM | 1539 | N | PRO | A | 185 | 34.042 | 25.523 | 62.471 | 1.00 | 44.94 |
| ATOM | 1540 | CA | PRO | A | 185 | 33.797 | 26.928 | 62.106 | 1.00 | 44.70 |
| ATOM | 1541 | C | PRO | A | 185 | 32.292 | 27.188 | 62.004 | 1.00 | 46.19 |
| ATOM | 1542 | O | PRO | A | 185 | 31.550 | 26.280 | 61.623 | 1.00 | 42.32 |
| ATOM | 1543 | CB | PRO | A | 185 | 34.484 | 27.069 | 60.749 | 1.00 | 45.46 |
| ATOM | 1544 | CG | PRO | A | 185 | 35.485 | 25.917 | 60.694 | 1.00 | 44.08 |
| ATOM | 1545 | CD | PRO | A | 185 | 34.820 | 24.806 | 61.445 | 1.00 | 43.98 |
| ATOM | 1546 | N | GLU | A | 186 | 31.836 | 28.379 | 62.378 | 1.00 | 50.16 |
| ATOM | 1547 | CA | GLU | A | 186 | 30.396 | 28.656 | 62.360 | 1.00 | 54.17 |
| ATOM | 1548 | C | GLU | A | 186 | 29.870 | 28.699 | 60.925 | 1.00 | 50.98 |
| ATOM | 1549 | O | GLU | A | 186 | 28.792 | 28.171 | 60.632 | 1.00 | 49.93 |
| ATOM | 1550 | CB | GLU | A | 186 | 30.077 | 29.966 | 63.074 | 1.00 | 59.44 |
| ATOM | 1551 | CG | GLU | A | 186 | 28.600 | 30.124 | 63.403 | 1.00 | 65.31 |
| ATOM | 1552 | CD | GLU | A | 186 | 28.330 | 31.335 | 64.283 | 1.00 | 70.87 |
| ATOM | 1553 | OE1 | GLU | A | 186 | 28.812 | 31.351 | 65.441 | 1.00 | 75.04 |
| ATOM | 1554 | OE2 | GLU | A | 186 | 27.641 | 32.271 | 63.817 | 1.00 | 72.57 |
| ATOM | 1555 | N | SER | A | 187 | 30.655 | 29.325 | 60.048 | 1.00 | 47.88 |
| ATOM | 1556 | CA | SER | A | 187 | 30.385 | 29.367 | 58.613 | 1.00 | 45.71 |
| ATOM | 1557 | C | SER | A | 187 | 31.494 | 28.638 | 57.814 | 1.00 | 45.42 |
| ATOM | 1558 | O | SER | A | 187 | 32.686 | 28.883 | 58.043 | 1.00 | 40.50 |
| ATOM | 1559 | CB | SER | A | 187 | 30.329 | 30.821 | 58.163 | 1.00 | 44.41 |
| ATOM | 1560 | OG | SER | A | 187 | 30.507 | 30.944 | 56.768 | 1.00 | 45.78 |
| ATOM | 1561 | N | PRO | A | 188 | 31.103 | 27.796 | 56.848 | 1.00 | 43.60 |
| ATOM | 1562 | CA | PRO | A | 188 | 32.063 | 27.084 | 55.997 | 1.00 | 43.07 |
| ATOM | 1563 | C | PRO | A | 188 | 32.758 | 27.951 | 54.960 | 1.00 | 41.00 |
| ATOM | 1564 | O | PRO | A | 188 | 33.626 | 27.446 | 54.244 | 1.00 | 38.20 |
| ATOM | 1565 | CB | PRO | A | 188 | 31.183 | 26.057 | 55.287 | 1.00 | 43.32 |
| ATOM | 1566 | CG | PRO | A | 188 | 29.891 | 26.726 | 55.172 | 1.00 | 44.98 |
| ATOM | 1567 | CD | PRO | A | 188 | 29.719 | 27.445 | 56.475 | 1.00 | 44.84 |
| ATOM | 1568 | N | ALA | A | 189 | 32.394 | 29.227 | 54.883 | 1.00 | 39.03 |
| ATOM | 1569 | CA | ALA | A | 189 | 32.904 | 30.115 | 53.848 | 1.00 | 37.90 |
| ATOM | 1570 | C | ALA | A | 189 | 34.442 | 30.141 | 53.726 | 1.00 | 35.97 |
| ATOM | 1571 | O | ALA | A | 189 | 34.974 | 30.046 | 52.620 | 1.00 | 34.80 |
| ATOM | 1572 | CB | ALA | A | 189 | 32.349 | 31.531 | 54.055 | 1.00 | 38.82 |
| ATOM | 1573 | N | SER | A | 190 | 35.168 | 30.284 | 54.830 | 1.00 | 34.20 |
| ATOM | 1574 | CA | SER | A | 190 | 36.636 | 30.415 | 54.738 | 1.00 | 31.49 |
| ATOM | 1575 | C | SER | A | 190 | 37.293 | 29.079 | 54.379 | 1.00 | 27.72 |
| ATOM | 1576 | O | SER | A | 190 | 38.306 | 29.027 | 53.688 | 1.00 | 30.72 |
| ATOM | 1577 | CB | SER | A | 190 | 37.233 | 30.936 | 56.050 | 1.00 | 32.25 |
| ATOM | 1578 | OG | SER | A | 190 | 37.062 | 29.998 | 57.103 | 1.00 | 33.72 |
| ATOM | 1579 | N | PHE | A | 191 | 36.712 | 28.005 | 54.881 | 1.00 | 28.68 |
| ATOM | 1580 | CA | PHE | A | 191 | 37.176 | 26.665 | 54.599 | 1.00 | 29.10 |
| ATOM | 1581 | C | PHE | A | 191 | 36.991 | 26.346 | 53.121 | 1.00 | 28.15 |
| ATOM | 1582 | O | PHE | A | 191 | 37.870 | 25.771 | 52.494 | 1.00 | 26.66 |
| ATOM | 1583 | CB | PHE | A | 191 | 36.388 | 25.668 | 55.447 | 1.00 | 31.55 |
| ATOM | 1584 | CG | PHE | A | 191 | 36.931 | 24.272 | 55.395 | 1.00 | 34.47 |
| ATOM | 1585 | CD1 | PHE | A | 191 | 38.130 | 23.966 | 56.004 | 1.00 | 37.54 |
| ATOM | 1586 | CD2 | PHE | A | 191 | 36.245 | 23.270 | 54.729 | 1.00 | 35.95 |
| ATOM | 1587 | CE1 | PHE | A | 191 | 38.628 | 22.668 | 55.965 | 1.00 | 40.99 |
| ATOM | 1588 | CE2 | PHE | A | 191 | 36.742 | 21.990 | 54.684 | 1.00 | 34.54 |
| ATOM | 1589 | CZ | PHE | A | 191 | 37.927 | 21.692 | 55.303 | 1.00 | 34.82 |
| ATOM | 1590 | N | LEU | A | 192 | 35.841 | 26.721 | 52.565 | 1.00 | 31.93 |
| ATOM | 1591 | CA | LEU | A | 192 | 35.586 | 26.533 | 51.132 | 1.00 | 31.68 |
| ATOM | 1592 | C | LEU | A | 192 | 36.542 | 27.355 | 50.297 | 1.00 | 31.83 |
| ATOM | 1593 | O | LEU | A | 192 | 37.108 | 26.873 | 49.311 | 1.00 | 27.47 |
| ATOM | 1594 | CB | LEU | A | 192 | 34.163 | 26.916 | 50.780 | 1.00 | 31.47 |
| ATOM | 1595 | CG | LEU | A | 192 | 33.109 | 25.921 | 51.215 | 1.00 | 34.18 |
| ATOM | 1596 | CD1 | LEU | A | 192 | 31.755 | 26.613 | 51.110 | 1.00 | 35.84 |
| ATOM | 1597 | CD2 | LEU | A | 192 | 33.155 | 24.638 | 50.370 | 1.00 | 32.29 |
| ATOM | 1598 | N | ASN | A | 193 | 36.731 | 28.603 | 50.702 | 1.00 | 33.12 |
| ATOM | 1599 | CA | ASN | A | 193 | 37.713 | 29.459 | 50.062 | 1.00 | 35.21 |
| ATOM | 1600 | C | ASN | A | 193 | 39.100 | 28.850 | 50.050 | 1.00 | 33.12 |
| ATOM | 1601 | O | ASN | A | 193 | 39.830 | 28.976 | 49.074 | 1.00 | 30.96 |
| ATOM | 1602 | CB | ASN | A | 193 | 37.775 | 30.824 | 50.749 | 1.00 | 40.63 |
| ATOM | 1603 | CG | ASN | A | 193 | 37.632 | 31.935 | 49.777 | 1.00 | 50.07 |
| ATOM | 1604 | OD1 | ASN | A | 193 | 38.629 | 32.546 | 49.377 | 1.00 | 60.40 |
| ATOM | 1605 | ND2 | ASN | A | 193 | 36.396 | 32.179 | 49.329 | 1.00 | 49.93 |
| ATOM | 1606 | N | PHE | A | 194 | 39.469 | 28.220 | 51.162 | 1.00 | 30.27 |
| ATOM | 1607 | CA | PHE | A | 194 | 40.768 | 27.578 | 51.291 | 1.00 | 28.81 |
| ATOM | 1608 | C | PHE | A | 194 | 40.816 | 26.381 | 50.331 | 1.00 | 24.31 |
| ATOM | 1609 | O | PHE | A | 194 | 41.797 | 26.195 | 49.636 | 1.00 | 24.03 |
| ATOM | 1610 | CB | PHE | A | 194 | 41.005 | 27.182 | 52.766 | 1.00 | 29.31 |
| ATOM | 1611 | CG | PHE | A | 194 | 42.205 | 26.305 | 53.001 | 1.00 | 26.73 |
| ATOM | 1612 | CD1 | PHE | A | 194 | 43.466 | 26.852 | 53.082 | 1.00 | 25.58 |
| ATOM | 1613 | CD2 | PHE | A | 194 | 42.051 | 24.930 | 53.201 | 1.00 | 25.09 |
| ATOM | 1614 | CE1 | PHE | A | 194 | 44.566 | 26.057 | 53.320 | 1.00 | 24.03 |
| ATOM | 1615 | CE2 | PHE | A | 194 | 43.137 | 24.126 | 53.436 | 1.00 | 25.00 |
| ATOM | 1616 | CZ | PHE | A | 194 | 44.401 | 24.680 | 53.515 | 1.00 | 24.82 |
| ATOM | 1617 | N | LEU | A | 195 | 39.755 | 25.588 | 50.285 | 1.00 | 26.00 |
| ATOM | 1618 | CA | LEU | A | 195 | 39.680 | 24.467 | 49.344 | 1.00 | 26.38 |
| ATOM | 1619 | C | LEU | A | 195 | 39.827 | 24.956 | 47.902 | 1.00 | 25.60 |
| ATOM | 1620 | O | LEU | A | 195 | 40.530 | 24.350 | 47.113 | 1.00 | 26.37 |
| ATOM | 1621 | CB | LEU | A | 195 | 38.375 | 23.694 | 49.513 | 1.00 | 27.12 |
| ATOM | 1622 | CG | LEU | A | 195 | 38.052 | 22.667 | 48.408 | 1.00 | 28.68 |
| ATOM | 1623 | CD1 | LEU | A | 195 | 39.144 | 21.589 | 48.319 | 1.00 | 24.87 |
| ATOM | 1624 | CD2 | LEU | A | 195 | 36.690 | 22.068 | 48.674 | 1.00 | 29.35 |
| ATOM | 1625 | N | PHE | A | 196 | 39.204 | 26.077 | 47.570 | 1.00 | 28.88 |
| ATOM | 1626 | CA | PHE | A | 196 | 39.290 | 26.609 | 46.209 | 1.00 | 28.69 |
| ATOM | 1627 | C | PHE | A | 196 | 40.691 | 27.057 | 45.894 | 1.00 | 26.91 |
| ATOM | 1628 | O | PHE | A | 196 | 41.165 | 26.825 | 44.786 | 1.00 | 26.27 |
| ATOM | 1629 | CB | PHE | A | 196 | 38.280 | 27.732 | 45.944 | 1.00 | 27.89 |
| ATOM | 1630 | CG | PHE | A | 196 | 36.848 | 27.333 | 46.152 | 1.00 | 33.40 |
| ATOM | 1631 | CD1 | PHE | A | 196 | 36.413 | 26.023 | 45.947 | 1.00 | 37.87 |
| ATOM | 1632 | CD2 | PHE | A | 196 | 35.923 | 28.273 | 46.566 | 1.00 | 37.20 |
| ATOM | 1633 | CE1 | PHE | A | 196 | 35.089 | 25.675 | 46.158 | 1.00 | 35.01 |
| ATOM | 1634 | CE2 | PHE | A | 196 | 34.606 | 27.920 | 46.775 | 1.00 | 37.70 |
| ATOM | 1635 | CZ | PHE | A | 196 | 34.192 | 26.617 | 46.565 | 1.00 | 36.87 |
| ATOM | 1636 | N | LYS | A | 197 | 41.382 | 27.654 | 46.861 | 1.00 | 29.22 |
| ATOM | 1637 | CA | LYS | A | 197 | 42.794 | 28.014 | 46.666 | 1.00 | 28.21 |
| ATOM | 1638 | C | LYS | A | 197 | 43.696 | 26.804 | 46.447 | 1.00 | 28.32 |
| ATOM | 1639 | O | LYS | A | 197 | 44.608 | 26.849 | 45.628 | 1.00 | 27.69 |
| ATOM | 1640 | CB | LYS | A | 197 | 43.325 | 28.844 | 47.846 | 1.00 | 34.00 |
| ATOM | 1641 | CG | LYS | A | 197 | 42.631 | 30.211 | 47.978 | 1.00 | 39.87 |
| ATOM | 1642 | CD | LYS | A | 197 | 43.145 | 31.239 | 46.948 | 1.00 | 46.22 |
| ATOM | 1643 | CE | LYS | A | 197 | 42.217 | 32.474 | 46.852 | 1.00 | 51.05 |
| ATOM | 1644 | NZ | LYS | A | 197 | 42.007 | 32.935 | 45.439 | 1.00 | 52.01 |
| ATOM | 1645 | N | VAL | A | 198 | 43.478 | 25.723 | 47.193 | 1.00 | 22.40 |
| ATOM | 1646 | CA | VAL | A | 198 | 44.247 | 24.500 | 46.941 | 1.00 | 23.35 |
| ATOM | 1647 | C | VAL | A | 198 | 43.965 | 23.996 | 45.514 | 1.00 | 17.98 |
| ATOM | 1648 | O | VAL | A | 198 | 44.864 | 23.626 | 44.791 | 1.00 | 22.30 |
| ATOM | 1649 | CB | VAL | A | 198 | 43.920 | 23.386 | 47.985 | 1.00 | 24.72 |
| ATOM | 1650 | CG1 | VAL | A | 198 | 44.705 | 22.142 | 47.699 | 1.00 | 23.99 |
| ATOM | 1651 | CG2 | VAL | A | 198 | 44.216 | 23.864 | 49.419 | 1.00 | 26.35 |
| ATOM | 1652 | N | ARG | A | 199 | 42.702 | 24.005 | 45.113 | 1.00 | 20.52 |
| ATOM | 1653 | CA | ARG | A | 199 | 42.321 | 23.564 | 43.770 | 1.00 | 24.64 |
| ATOM | 1654 | C | ARG | A | 199 | 43.010 | 24.403 | 42.702 | 1.00 | 26.05 |
| ATOM | 1655 | O | ARG | A | 199 | 43.632 | 23.864 | 41.779 | 1.00 | 25.25 |
| ATOM | 1656 | CB | ARG | A | 199 | 40.820 | 23.671 | 43.573 | 1.00 | 21.92 |
| ATOM | 1657 | CG | ARG | A | 199 | 40.002 | 22.546 | 44.139 | 1.00 | 22.65 |
| ATOM | 1658 | CD | ARG | A | 199 | 38.543 | 22.750 | 43.832 | 1.00 | 24.05 |
| ATOM | 1659 | NE | ARG | A | 199 | 37.716 | 21.612 | 44.177 | 1.00 | 22.40 |
| ATOM | 1660 | CZ | ARG | A | 199 | 36.489 | 21.428 | 43.719 | 1.00 | 25.49 |
| ATOM | 1661 | NH1 | ARG | A | 199 | 35.934 | 22.318 | 42.896 | 1.00 | 28.08 |
| ATOM | 1662 | NH2 | ARG | A | 199 | 35.809 | 20.343 | 44.068 | 1.00 | 24.68 |
| ATOM | 1663 | N | GLU | A | 200 | 42.934 | 25.722 | 42.876 | 1.00 | 29.34 |
| ATOM | 1664 | CA | GLU | A | 200 | 43.434 | 26.682 | 41.892 | 1.00 | 33.45 |
| ATOM | 1665 | C | GLU | A | 200 | 44.917 | 26.519 | 41.649 | 1.00 | 32.84 |
| ATOM | 1666 | O | GLU | A | 200 | 45.398 | 26.834 | 40.574 | 1.00 | 36.40 |
| ATOM | 1667 | CB | GLU | A | 200 | 43.109 | 28.113 | 42.330 | 1.00 | 34.88 |
| ATOM | 1668 | CG | GLU | A | 200 | 41.639 | 28.441 | 42.163 | 1.00 | 39.29 |
| ATOM | 1669 | CD | GLU | A | 200 | 41.224 | 29.742 | 42.834 | 1.00 | 45.70 |
| ATOM | 1670 | OE1 | GLU | A | 200 | 42.125 | 30.496 | 43.266 | 1.00 | 46.97 |
| ATOM | 1671 | OE2 | GLU | A | 200 | 39.994 | 29.997 | 42.918 | 1.00 | 45.60 |
| ATOM | 1672 | N | SER | A | 201 | 45.616 | 25.972 | 42.642 | 1.00 | 32.36 |
| ATOM | 1673 | CA | SER | A | 201 | 47.037 | 25.742 | 42.573 | 1.00 | 26.44 |
| ATOM | 1674 | C | SER | A | 201 | 47.454 | 24.601 | 41.686 | 1.00 | 30.06 |
| ATOM | 1675 | O | SER | A | 201 | 48.628 | 24.493 | 41.358 | 1.00 | 32.56 |
| ATOM | 1676 | CB | SER | A | 201 | 47.575 | 25.487 | 43.985 | 1.00 | 31.12 |
| ATOM | 1677 | OG | SER | A | 201 | 47.425 | 24.135 | 44.415 | 1.00 | 28.72 |
| ATOM | 1678 | N | GLY | A | 202 | 46.533 | 23.709 | 41.331 | 1.00 | 30.71 |
| ATOM | 1679 | CA | GLY | A | 202 | 46.897 | 22.485 | 40.627 | 1.00 | 28.34 |
| ATOM | 1680 | C | GLY | A | 202 | 47.249 | 21.292 | 41.510 | 1.00 | 30.55 |
| ATOM | 1681 | O | GLY | A | 202 | 47.507 | 20.199 | 41.000 | 1.00 | 28.65 |
| ATOM | 1682 | N | SER | A | 203 | 47.244 | 21.482 | 42.828 | 1.00 | 30.37 |
| ATOM | 1683 | CA | SER | A | 203 | 47.741 | 20.460 | 43.763 | 1.00 | 29.05 |
| ATOM | 1684 | C | SER | A | 203 | 46.834 | 19.237 | 43.882 | 1.00 | 28.76 |
| ATOM | 1685 | O | SER | A | 203 | 47.301 | 18.176 | 44.310 | 1.00 | 31.80 |
| ATOM | 1686 | CB | SER | A | 203 | 47.947 | 21.049 | 45.163 | 1.00 | 29.41 |
| ATOM | 1687 | OG | SER | A | 203 | 49.010 | 21.988 | 45.207 | 1.00 | 29.86 |
| ATOM | 1688 | N | LEU | A | 204 | 45.548 | 19.389 | 43.557 | 1.00 | 28.79 |
| ATOM | 1689 | CA | LEU | A | 204 | 44.591 | 18.272 | 43.554 | 1.00 | 29.64 |
| ATOM | 1690 | C | LEU | A | 204 | 44.403 | 17.627 | 42.171 | 1.00 | 31.08 |
| ATOM | 1691 | O | LEU | A | 204 | 43.657 | 16.661 | 42.036 | 1.00 | 32.01 |
| ATOM | 1692 | CB | LEU | A | 204 | 43.233 | 18.740 | 44.066 | 1.00 | 29.85 |
| ATOM | 1693 | CG | LEU | A | 204 | 43.216 | 19.303 | 45.483 | 1.00 | 33.56 |
| ATOM | 1694 | CD1 | LEU | A | 204 | 41.887 | 19.909 | 45.803 | 1.00 | 31.20 |
| ATOM | 1695 | CD2 | LEU | A | 204 | 43.549 | 18.204 | 46.483 | 1.00 | 38.36 |
| ATOM | 1696 | N | SER | A | 205 | 45.076 | 18.168 | 41.158 | 1.00 | 29.37 |
| ATOM | 1697 | CA | SER | A | 205 | 44.866 | 17.798 | 39.772 | 1.00 | 28.36 |
| ATOM | 1698 | C | SER | A | 205 | 45.673 | 16.562 | 39.344 | 1.00 | 31.37 |
| ATOM | 1699 | O | SER | A | 205 | 46.700 | 16.258 | 39.949 | 1.00 | 31.75 |
| ATOM | 1700 | CB | SER | A | 205 | 45.189 | 18.986 | 38.871 | 1.00 | 30.29 |
| ATOM | 1701 | OG | SER | A | 205 | 44.411 | 20.111 | 39.263 | 1.00 | 34.52 |
| ATOM | 1702 | N | PRO | A | 206 | 45.191 | 15.856 | 38.307 | 1.00 | 34.16 |
| ATOM | 1703 | CA | PRO | A | 206 | 45.768 | 14.574 | 37.870 | 1.00 | 34.58 |
| ATOM | 1704 | C | PRO | A | 206 | 47.210 | 14.593 | 37.379 | 1.00 | 31.19 |
| ATOM | 1705 | O | PRO | A | 206 | 47.836 | 13.557 | 37.458 | 1.00 | 35.03 |
| ATOM | 1706 | CB | PRO | A | 206 | 44.843 | 14.146 | 36.701 | 1.00 | 36.26 |
| ATOM | 1707 | CG | PRO | A | 206 | 43.597 | 14.874 | 36.907 | 1.00 | 36.19 |
| ATOM | 1708 | CD | PRO | A | 206 | 44.003 | 16.205 | 37.496 | 1.00 | 34.78 |
| ATOM | 1709 | N | GLU | A | 207 | 47.716 | 15.717 | 36.896 | 1.00 | 33.37 |
| ATOM | 1710 | CA | GLU | A | 207 | 49.132 | 15.830 | 36.501 | 1.00 | 37.01 |
| ATOM | 1711 | C | GLU | A | 207 | 50.115 | 15.606 | 37.666 | 1.00 | 36.38 |
| ATOM | 1712 | O | GLU | A | 207 | 51.309 | 15.423 | 37.437 | 1.00 | 36.45 |
| ATOM | 1713 | CB | GLU | A | 207 | 49.449 | 17.205 | 35.886 | 1.00 | 40.05 |
| ATOM | 1714 | CG | GLU | A | 207 | 48.399 | 17.773 | 34.933 | 1.00 | 46.50 |
| ATOM | 1715 | CD | GLU | A | 207 | 47.438 | 18.725 | 35.629 | 1.00 | 52.97 |
| ATOM | 1716 | OE1 | GLU | A | 207 | 47.805 | 19.912 | 35.885 | 1.00 | 59.90 |
| ATOM | 1717 | OE2 | GLU | A | 207 | 46.320 | 18.275 | 35.939 | 1.00 | 50.42 |
| ATOM | 1718 | N | HIS | A | 208 | 49.643 | 15.656 | 38.911 | 1.00 | 33.01 |
| ATOM | 1719 | CA | HIS | A | 208 | 50.538 | 15.446 | 40.049 | 1.00 | 27.65 |
| ATOM | 1720 | C | HIS | A | 208 | 50.116 | 14.231 | 40.821 | 1.00 | 25.96 |
| ATOM | 1721 | O | HIS | A | 208 | 48.999 | 13.747 | 40.669 | 1.00 | 27.36 |
| ATOM | 1722 | CB | HIS | A | 208 | 50.536 | 16.695 | 40.941 | 1.00 | 26.63 |
| ATOM | 1723 | CG | HIS | A | 208 | 50.900 | 17.943 | 40.198 | 1.00 | 26.81 |
| ATOM | 1724 | ND1 | HIS | A | 208 | 49.957 | 18.821 | 39.713 | 1.00 | 28.46 |
| ATOM | 1725 | CD2 | HIS | A | 208 | 52.107 | 18.441 | 39.823 | 1.00 | 28.59 |
| ATOM | 1726 | CE1 | HIS | A | 208 | 50.564 | 19.805 | 39.069 | 1.00 | 29.60 |
| ATOM | 1727 | NE2 | HIS | A | 208 | 51.869 | 19.605 | 39.132 | 1.00 | 29.80 |
| ATOM | 1728 | N | GLY | A | 209 | 51.015 | 13.727 | 41.647 | 1.00 | 25.77 |
| ATOM | 1729 | CA | GLY | A | 209 | 50.648 | 12.757 | 42.652 | 1.00 | 25.91 |
| ATOM | 1730 | C | GLY | A | 209 | 49.670 | 13.353 | 43.646 | 1.00 | 25.50 |
| ATOM | 1731 | O | GLY | A | 209 | 49.354 | 14.552 | 43.614 | 1.00 | 26.79 |
| ATOM | 1732 | N | PRO | A | 210 | 49.134 | 12.501 | 44.502 | 1.00 | 25.06 |
| ATOM | 1733 | CA | PRO | A | 210 | 48.108 | 12.930 | 45.456 | 1.00 | 26.32 |
| ATOM | 1734 | C | PRO | A | 210 | 48.597 | 13.973 | 46.457 | 1.00 | 21.20 |
| ATOM | 1735 | O | PRO | A | 210 | 49.714 | 13.902 | 46.951 | 1.00 | 20.44 |
| ATOM | 1736 | CB | PRO | A | 210 | 47.710 | 11.632 | 46.187 | 1.00 | 24.37 |
| ATOM | 1737 | CG | PRO | A | 210 | 48.700 | 10.614 | 45.789 | 1.00 | 28.61 |
| ATOM | 1738 | CD | PRO | A | 210 | 49.418 | 11.061 | 44.574 | 1.00 | 27.20 |
| ATOM | 1739 | N | VAL | A | 211 | 47.740 | 14.942 | 46.719 | 1.00 | 23.27 |
| ATOM | 1740 | CA | VAL | A | 211 | 47.928 | 15.872 | 47.816 | 1.00 | 22.75 |
| ATOM | 1741 | C | VAL | A | 211 | 48.129 | 15.103 | 49.131 | 1.00 | 24.67 |
| ATOM | 1742 | O | VAL | A | 211 | 47.508 | 14.043 | 49.337 | 1.00 | 23.47 |
| ATOM | 1743 | CB | VAL | A | 211 | 46.725 | 16.840 | 47.866 | 1.00 | 23.08 |
| ATOM | 1744 | CG1 | VAL | A | 211 | 45.451 | 16.164 | 48.401 | 1.00 | 25.62 |
| ATOM | 1745 | CG2 | VAL | A | 211 | 47.064 | 18.080 | 48.646 | 1.00 | 29.23 |
| ATOM | 1746 | N | VAL | A | 212 | 49.011 | 15.612 | 49.994 | 1.00 | 20.82 |
| ATOM | 1747 | CA | VAL | A | 212 | 49.220 | 15.070 | 51.334 | 1.00 | 20.51 |
| ATOM | 1748 | C | VAL | A | 212 | 48.486 | 15.952 | 52.328 | 1.00 | 25.29 |
| ATOM | 1749 | O | VAL | A | 212 | 48.712 | 17.142 | 52.368 | 1.00 | 26.61 |
| ATOM | 1750 | CB | VAL | A | 212 | 50.699 | 14.957 | 51.691 | 1.00 | 21.70 |
| ATOM | 1751 | CG1 | VAL | A | 212 | 50.893 | 14.460 | 53.118 | 1.00 | 23.78 |
| ATOM | 1752 | CG2 | VAL | A | 212 | 51.388 | 14.002 | 50.748 | 1.00 | 21.34 |
| ATOM | 1753 | N | VAL | A | 213 | 47.585 | 15.356 | 53.107 | 1.00 | 22.18 |
| ATOM | 1754 | CA | VAL | A | 213 | 46.700 | 16.087 | 53.990 | 1.00 | 20.71 |
| ATOM | 1755 | C | VAL | A | 213 | 46.904 | 15.583 | 55.393 | 1.00 | 21.82 |
| ATOM | 1756 | O | VAL | A | 213 | 46.973 | 14.369 | 55.600 | 1.00 | 21.30 |
| ATOM | 1757 | CB | VAL | A | 213 | 45.214 | 15.855 | 53.618 | 1.00 | 18.46 |
| ATOM | 1758 | CG1 | VAL | A | 213 | 44.314 | 16.704 | 54.468 | 1.00 | 17.92 |
| ATOM | 1759 | CG2 | VAL | A | 213 | 44.961 | 16.130 | 52.136 | 1.00 | 18.57 |
| ATOM | 1760 | N | HIS | A | 214 | 47.009 | 16.499 | 56.360 | 1.00 | 21.27 |
| ATOM | 1761 | CA | HIS | A | 214 | 47.013 | 16.107 | 57.780 | 1.00 | 18.49 |
| ATOM | 1762 | C | HIS | A | 214 | 46.329 | 17.127 | 58.658 | 1.00 | 22.46 |
| ATOM | 1763 | O | HIS | A | 214 | 46.217 | 18.292 | 58.266 | 1.00 | 19.52 |
| ATOM | 1764 | CB | HIS | A | 214 | 48.424 | 15.861 | 58.255 | 1.00 | 22.66 |
| ATOM | 1765 | CG | HIS | A | 214 | 49.242 | 17.103 | 58.440 | 1.00 | 23.62 |
| ATOM | 1766 | ND1 | HIS | A | 214 | 49.465 | 17.670 | 59.677 | 1.00 | 21.41 |
| ATOM | 1767 | CD2 | HIS | A | 214 | 49.868 | 17.899 | 57.542 | 1.00 | 24.31 |
| ATOM | 1768 | CE1 | HIS | A | 214 | 50.247 | 18.724 | 59.536 | 1.00 | 21.76 |
| ATOM | 1769 | NE2 | HIS | A | 214 | 50.494 | 18.893 | 58.251 | 1.00 | 21.07 |
| ATOM | 1770 | N | ACYS | A | 215 | 45.982 | 16.650 | 59.851 | 0.50 | 21.29 |
| ATOM | 1771 | CA | ACYS | A | 215 | 45.553 | 17.584 | 60.885 | 0.50 | 21.43 |
| ATOM | 1772 | C | ACYS | A | 215 | 46.211 | 17.303 | 62.215 | 0.50 | 24.05 |
| ATOM | 1773 | O | ACYS | A | 215 | 47.355 | 16.913 | 62.307 | 0.50 | 22.32 |
| ATOM | 1774 | CB | ACYS | A | 215 | 44.037 | 17.520 | 61.166 | 0.50 | 22.73 |
| ATOM | 1775 | SG | ACYS | A | 215 | 43.916 | 18.029 | 62.855 | 0.50 | 30.96 |
| ATOM | 1776 | N | BCYS | A | 215 | 45.982 | 16.650 | 59.851 | 0.50 | 21.29 |
| ATOM | 1777 | CA | BCYS | A | 215 | 45.553 | 17.584 | 60.885 | 0.50 | 21.43 |
| ATOM | 1778 | C | BCYS | A | 215 | 46.211 | 17.303 | 62.215 | 0.50 | 24.05 |
| ATOM | 1779 | O | BCYS | A | 215 | 47.355 | 16.913 | 62.307 | 0.50 | 22.32 |
| ATOM | 1780 | CB | BCYS | A | 215 | 44.037 | 17.520 | 61.166 | 0.50 | 22.73 |
| ATOM | 1781 | SG | BCYS | A | 215 | 43.916 | 18.029 | 62.855 | 0.50 | 30.96 |
| ATOM | 1782 | N | ASER | A | 216 | 45.432 | 17.553 | 63.271 | 0.50 | 28.96 |
| ATOM | 1783 | CA | ASER | A | 216 | 45.887 | 17.111 | 64.593 | 0.50 | 33.95 |
| ATOM | 1784 | C | ASER | A | 216 | 45.405 | 15.709 | 64.892 | 0.50 | 38.49 |
| ATOM | 1785 | O | ASER | A | 216 | 44.329 | 15.318 | 64.490 | 0.50 | 38.68 |
| ATOM | 1786 | CB | ASER | A | 216 | 45.451 | 18.035 | 65.735 | 0.50 | 34.57 |
| ATOM | 1787 | OG | ASER | A | 216 | 45.116 | 19.351 | 65.362 | 0.50 | 36.62 |
| ATOM | 1788 | N | BSER | A | 216 | 45.432 | 17.553 | 63.271 | 0.50 | 28.96 |
| ATOM | 1789 | CA | BSER | A | 216 | 45.887 | 17.111 | 64.593 | 0.50 | 33.95 |
| ATOM | 1790 | C | BSER | A | 216 | 45.405 | 15.709 | 64.892 | 0.50 | 38.49 |
| ATOM | 1791 | O | BSER | A | 216 | 44.329 | 15.318 | 64.490 | 0.50 | 38.68 |
| ATOM | 1792 | CB | BSER | A | 216 | 45.451 | 18.035 | 65.735 | 0.50 | 34.57 |
| ATOM | 1793 | OG | BSER | A | 216 | 45.116 | 19.351 | 65.362 | 0.50 | 36.62 |
| ATOM | 1794 | N | ALA | A | 217 | 45.830 | 14.927 | 65.877 | 1.00 | 39.80 |
| ATOM | 1795 | CA | ALA | A | 217 | 45.628 | 13.542 | 66.296 | 1.00 | 39.86 |
| ATOM | 1796 | C | ALA | A | 217 | 44.166 | 13.315 | 66.672 | 1.00 | 39.03 |
| ATOM | 1797 | O | ALA | A | 217 | 43.627 | 14.011 | 67.529 | 1.00 | 36.63 |
| ATOM | 1798 | CB | ALA | A | 217 | 46.534 | 13.209 | 67.476 | 1.00 | 39.80 |
| ATOM | 1799 | N | GLY | A | 218 | 43.518 | 12.380 | 65.978 | 1.00 | 44.45 |
| ATOM | 1800 | CA | GLY | A | 218 | 42.248 | 11.824 | 66.419 | 1.00 | 44.76 |
| ATOM | 1801 | C | GLY | A | 218 | 41.011 | 12.697 | 66.321 | 1.00 | 48.15 |
| ATOM | 1802 | O | GLY | A | 218 | 39.953 | 12.245 | 66.742 | 1.00 | 50.53 |
| ATOM | 1803 | N | ILE | A | 219 | 41.106 | 13.918 | 65.777 | 1.00 | 47.19 |
| ATOM | 1804 | CA | ILE | A | 219 | 39.928 | 14.807 | 65.728 | 1.00 | 47.39 |
| ATOM | 1805 | C | ILE | A | 219 | 39.495 | 15.264 | 64.317 | 1.00 | 43.76 |
| ATOM | 1806 | O | ILE | A | 219 | 38.817 | 16.268 | 64.172 | 1.00 | 43.45 |
| ATOM | 1807 | CB | ILE | A | 219 | 40.050 | 16.012 | 66.745 | 1.00 | 50.42 |
| ATOM | 1808 | CG1 | ILE | A | 219 | 40.920 | 17.169 | 66.222 | 1.00 | 51.81 |
| ATOM | 1809 | CG2 | ILE | A | 219 | 40.529 | 15.526 | 68.118 | 1.00 | 49.25 |
| ATOM | 1810 | CD1 | ILE | A | 219 | 42.192 | 16.787 | 65.649 | 1.00 | 52.65 |
| ATOM | 1811 | N | GLY | A | 220 | 39.883 | 14.508 | 63.291 | 1.00 | 43.30 |
| ATOM | 1812 | CA | GLY | A | 220 | 39.321 | 14.664 | 61.954 | 1.00 | 39.26 |
| ATOM | 1813 | C | GLY | A | 220 | 39.890 | 15.811 | 61.140 | 1.00 | 37.46 |
| ATOM | 1814 | O | GLY | A | 220 | 41.113 | 15.952 | 61.045 | 1.00 | 40.16 |
| ATOM | 1815 | N | ARG | A | 221 | 38.991 | 16.631 | 60.582 | 1.00 | 30.80 |
| ATOM | 1816 | CA | ARG | A | 221 | 39.298 | 17.738 | 59.668 | 1.00 | 29.00 |
| ATOM | 1817 | C | ARG | A | 221 | 39.765 | 17.340 | 58.251 | 1.00 | 24.28 |
| ATOM | 1818 | O | ARG | A | 221 | 39.248 | 17.862 | 57.274 | 1.00 | 24.99 |
| ATOM | 1819 | CB | ARG | A | 221 | 40.301 | 18.743 | 60.277 | 1.00 | 31.09 |
| ATOM | 1820 | CG | ARG | A | 221 | 39.907 | 19.330 | 61.619 | 1.00 | 28.54 |
| ATOM | 1821 | CD | ARG | A | 221 | 40.937 | 20.316 | 62.157 | 1.00 | 26.55 |
| ATOM | 1822 | NE | ARG | A | 221 | 40.869 | 20.481 | 63.600 | 1.00 | 25.85 |
| ATOM | 1823 | CZ | ARG | A | 221 | 41.695 | 21.278 | 64.294 | 1.00 | 28.63 |
| ATOM | 1824 | NH1 | ARG | A | 221 | 42.662 | 21.955 | 63.686 | 1.00 | 23.62 |
| ATOM | 1825 | NH2 | ARG | A | 221 | 41.558 | 21.385 | 65.604 | 1.00 | 27.34 |
| ATOM | 1826 | N | SER | A | 222 | 40.767 | 16.476 | 58.156 | 1.00 | 24.21 |
| ATOM | 1827 | CA | SER | A | 222 | 41.286 | 15.989 | 56.887 | 1.00 | 26.35 |
| ATOM | 1828 | C | SER | A | 222 | 40.179 | 15.345 | 56.042 | 1.00 | 27.07 |
| ATOM | 1829 | O | SER | A | 222 | 40.097 | 15.574 | 54.838 | 1.00 | 26.61 |
| ATOM | 1830 | CB | SER | A | 222 | 42.397 | 14.972 | 57.125 | 1.00 | 24.83 |
| ATOM | 1831 | OG | SER | A | 222 | 43.374 | 15.456 | 58.013 | 1.00 | 27.62 |
| ATOM | 1832 | N | GLY | A | 223 | 39.323 | 14.560 | 56.693 | 1.00 | 25.96 |
| ATOM | 1833 | CA | GLY | A | 223 | 38.229 | 13.895 | 56.029 | 1.00 | 26.22 |
| ATOM | 1834 | C | GLY | A | 223 | 37.211 | 14.872 | 55.489 | 1.00 | 27.52 |
| ATOM | 1835 | O | GLY | A | 223 | 36.677 | 14.671 | 54.407 | 1.00 | 29.73 |
| ATOM | 1836 | N | THR | A | 224 | 36.952 | 15.934 | 56.239 | 1.00 | 27.07 |
| ATOM | 1837 | CA | THR | A | 224 | 36.051 | 16.987 | 55.800 | 1.00 | 26.92 |
| ATOM | 1838 | C | THR | A | 224 | 36.540 | 17.673 | 54.532 | 1.00 | 23.15 |
| ATOM | 1839 | O | THR | A | 224 | 35.758 | 17.901 | 53.621 | 1.00 | 23.86 |
| ATOM | 1840 | CB | THR | A | 224 | 35.887 | 18.013 | 56.927 | 1.00 | 31.61 |
| ATOM | 1841 | OG1 | THR | A | 224 | 35.214 | 17.394 | 58.031 | 1.00 | 31.13 |
| ATOM | 1842 | CG2 | THR | A | 224 | 34.975 | 19.146 | 56.522 | 1.00 | 34.93 |
| ATOM | 1843 | N | PHE | A | 225 | 37.828 | 18.009 | 54.486 | 1.00 | 23.12 |
| ATOM | 1844 | CA | PHE | A | 225 | 38.448 | 18.612 | 53.315 | 1.00 | 22.32 |
| ATOM | 1845 | C | PHE | A | 225 | 38.251 | 17.747 | 52.060 | 1.00 | 24.02 |
| ATOM | 1846 | O | PHE | A | 225 | 37.782 | 18.238 | 51.027 | 1.00 | 23.24 |
| ATOM | 1847 | CB | PHE | A | 225 | 39.948 | 18.816 | 53.557 | 1.00 | 22.48 |
| ATOM | 1848 | CG | PHE | A | 225 | 40.664 | 19.484 | 52.414 | 1.00 | 22.57 |
| ATOM | 1849 | CD1 | PHE | A | 225 | 41.323 | 18.732 | 51.449 | 1.00 | 22.46 |
| ATOM | 1850 | CD2 | PHE | A | 225 | 40.673 | 20.865 | 52.300 | 1.00 | 25.95 |
| ATOM | 1851 | CE1 | PHE | A | 225 | 41.993 | 19.347 | 50.404 | 1.00 | 24.23 |
| ATOM | 1852 | CE2 | PHE | A | 225 | 41.349 | 21.490 | 51.248 | 1.00 | 23.96 |
| ATOM | 1853 | CZ | PHE | A | 225 | 42.003 | 20.738 | 50.308 | 1.00 | 23.02 |
| ATOM | 1854 | N | CYS | A | 226 | 38.599 | 16.468 | 52.161 | 1.00 | 22.58 |
| ATOM | 1855 | CA | CYS | A | 226 | 38.488 | 15.528 | 51.028 | 1.00 | 24.24 |
| ATOM | 1856 | C | CYS | A | 226 | 37.036 | 15.217 | 50.638 | 1.00 | 25.48 |
| ATOM | 1857 | O | CYS | A | 226 | 36.689 | 15.170 | 49.452 | 1.00 | 24.77 |
| ATOM | 1858 | CB | CYS | A | 226 | 39.243 | 14.232 | 51.338 | 1.00 | 24.74 |
| ATOM | 1859 | SG | CYS | A | 226 | 40.990 | 14.548 | 51.722 | 1.00 | 26.51 |
| ATOM | 1860 | N | LEU | A | 227 | 36.191 | 15.025 | 51.635 | 1.00 | 25.13 |
| ATOM | 1861 | CA | LEU | A | 227 | 34.780 | 14.744 | 51.409 | 1.00 | 26.81 |
| ATOM | 1862 | C | LEU | A | 227 | 34.098 | 15.862 | 50.643 | 1.00 | 26.57 |
| ATOM | 1863 | O | LEU | A | 227 | 33.355 | 15.623 | 49.693 | 1.00 | 28.06 |
| ATOM | 1864 | CB | LEU | A | 227 | 34.055 | 14.572 | 52.741 | 1.00 | 28.52 |
| ATOM | 1865 | CG | LEU | A | 227 | 32.563 | 14.216 | 52.682 | 1.00 | 27.60 |
| ATOM | 1866 | CD1 | LEU | A | 227 | 32.319 | 12.963 | 51.831 | 1.00 | 26.20 |
| ATOM | 1867 | CD2 | LEU | A | 227 | 32.017 | 14.037 | 54.086 | 1.00 | 30.02 |
| ATOM | 1868 | N | ALA | A | 228 | 34.340 | 17.082 | 51.086 | 1.00 | 24.59 |
| ATOM | 1869 | CA | ALA | A | 228 | 33.752 | 18.262 | 50.465 | 1.00 | 25.53 |
| ATOM | 1870 | C | ALA | A | 228 | 34.244 | 18.410 | 49.041 | 1.00 | 24.66 |
| ATOM | 1871 | O | ALA | A | 228 | 33.460 | 18.678 | 48.136 | 1.00 | 26.93 |
| ATOM | 1872 | CB | ALA | A | 228 | 34.104 | 19.502 | 51.267 | 1.00 | 24.92 |
| ATOM | 1873 | N | ASP | A | 229 | 35.548 | 18.200 | 48.848 | 1.00 | 21.07 |
| ATOM | 1874 | CA | ASP | A | 229 | 36.158 | 18.294 | 47.535 | 1.00 | 21.55 |
| ATOM | 1875 | C | ASP | A | 229 | 35.554 | 17.307 | 46.529 | 1.00 | 24.44 |
| ATOM | 1876 | O | ASP | A | 229 | 35.207 | 17.677 | 45.400 | 1.00 | 23.83 |
| ATOM | 1877 | CB | ASP | A | 229 | 37.650 | 18.058 | 47.655 | 1.00 | 21.03 |
| ATOM | 1878 | CG | ASP | A | 229 | 38.359 | 18.170 | 46.342 | 1.00 | 24.70 |
| ATOM | 1879 | OD1 | ASP | A | 229 | 38.129 | 19.175 | 45.649 | 1.00 | 22.45 |
| ATOM | 1880 | OD2 | ASP | A | 229 | 39.145 | 17.295 | 45.910 | 1.00 | 24.46 |
| ATOM | 1881 | N | THR | A | 230 | 35.473 | 16.051 | 46.939 | 1.00 | 22.53 |
| ATOM | 1882 | CA | THR | A | 230 | 34.986 | 14.979 | 46.095 | 1.00 | 26.13 |
| ATOM | 1883 | C | THR | A | 230 | 33.515 | 15.178 | 45.771 | 1.00 | 26.21 |
| ATOM | 1884 | O | THR | A | 230 | 33.144 | 15.076 | 44.615 | 1.00 | 27.32 |
| ATOM | 1885 | CB | THR | A | 230 | 35.234 | 13.602 | 46.762 | 1.00 | 25.54 |
| ATOM | 1886 | OG1 | THR | A | 230 | 36.600 | 13.243 | 46.572 | 1.00 | 23.46 |
| ATOM | 1887 | CG2 | THR | A | 230 | 34.455 | 12.461 | 46.073 | 1.00 | 28.27 |
| ATOM | 1888 | N | CYS | A | 231 | 32.699 | 15.472 | 46.782 | 1.00 | 26.34 |
| ATOM | 1889 | CA | CYS | A | 231 | 31.268 | 15.706 | 46.593 | 1.00 | 28.13 |
| ATOM | 1890 | C | CYS | A | 231 | 31.019 | 16.827 | 45.591 | 1.00 | 27.64 |
| ATOM | 1891 | O | CYS | A | 231 | 30.196 | 16.693 | 44.706 | 1.00 | 31.51 |
| ATOM | 1892 | CB | CYS | A | 231 | 30.573 | 16.046 | 47.926 | 1.00 | 30.75 |
| ATOM | 1893 | SG | CYS | A | 231 | 30.324 | 14.640 | 49.046 | 1.00 | 31.45 |
| ATOM | 1894 | N | LEU | A | 232 | 31.745 | 17.926 | 45.713 | 1.00 | 27.97 |
| ATOM | 1895 | CA | LEU | A | 232 | 31.550 | 19.047 | 44.794 | 1.00 | 28.23 |
| ATOM | 1896 | C | LEU | A | 232 | 31.942 | 18.679 | 43.369 | 1.00 | 29.48 |
| ATOM | 1897 | O | LEU | A | 232 | 31.265 | 19.052 | 42.401 | 1.00 | 26.16 |
| ATOM | 1898 | CB | LEU | A | 232 | 32.331 | 20.254 | 45.260 | 1.00 | 27.13 |
| ATOM | 1899 | CG | LEU | A | 232 | 31.816 | 20.889 | 46.547 | 1.00 | 28.98 |
| ATOM | 1900 | CD1 | LEU | A | 232 | 32.805 | 21.934 | 47.040 | 1.00 | 28.37 |
| ATOM | 1901 | CD2 | LEU | A | 232 | 30.443 | 21.492 | 46.345 | 1.00 | 32.86 |
| ATOM | 1902 | N | LEU | A | 233 | 33.019 | 17.914 | 43.254 | 1.00 | 27.18 |
| ATOM | 1903 | CA | LEU | A | 233 | 33.473 | 17.442 | 41.957 | 1.00 | 28.48 |
| ATOM | 1904 | C | LEU | A | 233 | 32.429 | 16.527 | 41.298 | 1.00 | 29.51 |
| ATOM | 1905 | O | LEU | A | 233 | 32.143 | 16.664 | 40.110 | 1.00 | 27.14 |
| ATOM | 1906 | CB | LEU | A | 233 | 34.809 | 16.741 | 42.110 | 1.00 | 30.57 |
| ATOM | 1907 | CG | LEU | A | 233 | 35.788 | 16.847 | 40.964 | 1.00 | 35.34 |
| ATOM | 1908 | CD1 | LEU | A | 233 | 36.159 | 18.310 | 40.635 | 1.00 | 39.45 |
| ATOM | 1909 | CD2 | LEU | A | 233 | 37.003 | 16.021 | 41.327 | 1.00 | 39.50 |
| ATOM | 1910 | N | LEU | A | 234 | 31.830 | 15.630 | 42.077 | 1.00 | 26.58 |
| ATOM | 1911 | CA | LEU | A | 234 | 30.833 | 14.711 | 41.555 | 1.00 | 29.13 |
| ATOM | 1912 | C | LEU | A | 234 | 29.574 | 15.443 | 41.127 | 1.00 | 29.39 |
| ATOM | 1913 | O | LEU | A | 234 | 28.915 | 15.025 | 40.190 | 1.00 | 29.61 |
| ATOM | 1914 | CB | LEU | A | 234 | 30.463 | 13.643 | 42.589 | 1.00 | 28.81 |
| ATOM | 1915 | CG | LEU | A | 234 | 31.527 | 12.619 | 42.998 | 1.00 | 29.20 |
| ATOM | 1916 | CD1 | LEU | A | 234 | 31.822 | 11.669 | 41.849 | 1.00 | 32.34 |
| ATOM | 1917 | CD2 | LEU | A | 234 | 31.057 | 11.839 | 44.210 | 1.00 | 26.85 |
| ATOM | 1918 | N | MET | A | 235 | 29.227 | 16.522 | 41.826 | 1.00 | 33.19 |
| ATOM | 1919 | CA | MET | A | 235 | 28.057 | 17.314 | 41.464 | 1.00 | 33.17 |
| ATOM | 1920 | C | MET | A | 235 | 28.224 | 17.968 | 40.088 | 1.00 | 34.16 |
| ATOM | 1921 | O | MET | A | 235 | 27.267 | 18.060 | 39.335 | 1.00 | 37.58 |
| ATOM | 1922 | CB | MET | A | 235 | 27.752 | 18.354 | 42.533 | 1.00 | 33.08 |
| ATOM | 1923 | CG | MET | A | 235 | 27.218 | 17.733 | 43.794 | 1.00 | 37.93 |
| ATOM | 1924 | SD | MET | A | 235 | 26.992 | 18.903 | 45.116 | 1.00 | 38.83 |
| ATOM | 1925 | CE | MET | A | 235 | 26.505 | 17.871 | 46.353 | 1.00 | 40.67 |
| ATOM | 1926 | N | ASP | A | 236 | 29.443 | 18.378 | 39.760 | 1.00 | 34.42 |
| ATOM | 1927 | CA | ASP | A | 236 | 29.801 | 18.860 | 38.424 | 1.00 | 35.78 |
| ATOM | 1928 | C | ASP | A | 236 | 29.906 | 17.793 | 37.323 | 1.00 | 37.28 |
| ATOM | 1929 | O | ASP | A | 236 | 29.743 | 18.119 | 36.154 | 1.00 | 36.30 |
| ATOM | 1930 | CB | ASP | A | 236 | 31.166 | 19.551 | 38.467 | 1.00 | 40.86 |
| ATOM | 1931 | CG | ASP | A | 236 | 31.060 | 21.045 | 38.568 | 1.00 | 45.84 |
| ATOM | 1932 | OD1 | ASP | A | 236 | 30.172 | 21.666 | 37.909 | 1.00 | 49.22 |
| ATOM | 1933 | OD2 | ASP | A | 236 | 31.855 | 21.681 | 39.276 | 1.00 | 49.02 |
| ATOM | 1934 | N | LYS | A | 237 | 30.232 | 16.553 | 37.686 | 1.00 | 36.32 |
| ATOM | 1935 | CA | LYS | A | 237 | 30.475 | 15.475 | 36.723 | 1.00 | 35.45 |
| ATOM | 1936 | C | LYS | A | 237 | 29.181 | 14.789 | 36.283 | 1.00 | 33.73 |
| ATOM | 1937 | O | LYS | A | 237 | 28.992 | 14.476 | 35.105 | 1.00 | 28.67 |
| ATOM | 1938 | CB | LYS | A | 237 | 31.404 | 14.422 | 37.347 | 1.00 | 36.84 |
| ATOM | 1939 | CG | LYS | A | 237 | 32.262 | 13.614 | 36.365 | 1.00 | 41.35 |
| ATOM | 1940 | CD | LYS | A | 237 | 33.411 | 12.887 | 37.086 | 1.00 | 41.67 |
| ATOM | 1941 | CE | LYS | A | 237 | 34.476 | 12.316 | 36.122 | 1.00 | 47.97 |
| ATOM | 1942 | NZ | LYS | A | 237 | 35.847 | 13.013 | 36.172 | 1.00 | 45.85 |
| ATOM | 1943 | N | ARG | A | 238 | 28.299 | 14.534 | 37.237 | 1.00 | 32.18 |
| ATOM | 1944 | CA | ARG | A | 238 | 27.157 | 13.655 | 36.993 | 1.00 | 34.57 |
| ATOM | 1945 | C | ARG | A | 238 | 26.034 | 14.365 | 36.262 | 1.00 | 35.08 |
| ATOM | 1946 | O | ARG | A | 238 | 25.798 | 15.562 | 36.470 | 1.00 | 32.96 |
| ATOM | 1947 | CB | ARG | A | 238 | 26.584 | 13.143 | 38.303 | 1.00 | 35.62 |
| ATOM | 1948 | CG | ARG | A | 238 | 27.366 | 12.041 | 38.912 | 1.00 | 33.98 |
| ATOM | 1949 | CD | ARG | A | 238 | 26.764 | 11.583 | 40.190 | 1.00 | 32.14 |
| ATOM | 1950 | NE | ARG | A | 238 | 27.503 | 10.469 | 40.780 | 1.00 | 29.90 |
| ATOM | 1951 | CZ | ARG | A | 238 | 27.108 | 9.846 | 41.875 | 1.00 | 31.16 |
| ATOM | 1952 | NH1 | ARG | A | 238 | 26.009 | 10.228 | 42.496 | 1.00 | 36.13 |
| ATOM | 1953 | NH2 | ARG | A | 238 | 27.833 | 8.857 | 42.383 | 1.00 | 37.35 |
| ATOM | 1954 | N | LYS | A | 239 | 25.327 | 13.587 | 35.452 | 1.00 | 34.64 |
| ATOM | 1955 | CA | LYS | A | 239 | 24.099 | 14.019 | 34.795 | 1.00 | 37.99 |
| ATOM | 1956 | C | LYS | A | 239 | 23.025 | 14.391 | 35.810 | 1.00 | 37.55 |
| ATOM | 1957 | O | LYS | A | 239 | 22.217 | 15.277 | 35.558 | 1.00 | 35.27 |
| ATOM | 1958 | CB | LYS | A | 239 | 23.591 | 12.917 | 33.857 | 1.00 | 39.88 |
| ATOM | 1959 | CG | LYS | A | 239 | 24.489 | 12.698 | 32.626 | 1.00 | 45.02 |
| ATOM | 1960 | CD | LYS | A | 239 | 24.059 | 11.472 | 31.773 | 1.00 | 52.00 |
| ATOM | 1961 | CE | LYS | A | 239 | 23.645 | 11.832 | 30.330 | 1.00 | 54.36 |
| ATOM | 1962 | NZ | LYS | A | 239 | 24.672 | 11.399 | 29.329 | 1.00 | 57.23 |
| ATOM | 1963 | N | ASP | A | 240 | 23.042 | 13.721 | 36.962 | 1.00 | 39.09 |
| ATOM | 1964 | CA | ASP | A | 240 | 22.107 | 13.970 | 38.057 | 1.00 | 39.45 |
| ATOM | 1965 | C | ASP | A | 240 | 22.853 | 14.406 | 39.351 | 1.00 | 36.92 |
| ATOM | 1966 | O | ASP | A | 240 | 23.147 | 13.578 | 40.211 | 1.00 | 38.63 |
| ATOM | 1967 | CB | ASP | A | 240 | 21.308 | 12.688 | 38.291 | 1.00 | 39.46 |
| ATOM | 1968 | CG | ASP | A | 240 | 20.200 | 12.847 | 39.313 | 1.00 | 39.85 |
| ATOM | 1969 | OD1 | ASP | A | 240 | 19.876 | 13.993 | 39.740 | 1.00 | 38.51 |
| ATOM | 1970 | OD2 | ASP | A | 240 | 19.594 | 11.843 | 39.742 | 1.00 | 42.08 |
| ATOM | 1971 | N | PRO | A | 241 | 23.176 | 15.690 | 39.479 | 1.00 | 36.36 |
| ATOM | 1972 | CA | PRO | A | 241 | 23.862 | 16.215 | 40.674 | 1.00 | 37.38 |
| ATOM | 1973 | C | PRO | A | 241 | 23.215 | 15.841 | 42.022 | 1.00 | 39.56 |
| ATOM | 1974 | O | PRO | A | 241 | 23.926 | 15.723 | 43.021 | 1.00 | 39.31 |
| ATOM | 1975 | CB | PRO | A | 241 | 23.785 | 17.740 | 40.492 | 1.00 | 37.29 |
| ATOM | 1976 | CG | PRO | A | 241 | 23.527 | 17.971 | 39.061 | 1.00 | 37.23 |
| ATOM | 1977 | CD | PRO | A | 241 | 22.932 | 16.735 | 38.475 | 1.00 | 37.85 |
| ATOM | 1978 | N | SER | A | 242 | 21.893 | 15.666 | 42.043 | 1.00 | 38.69 |
| ATOM | 1979 | CA | SER | A | 242 | 21.138 | 15.431 | 43.282 | 1.00 | 37.48 |
| ATOM | 1980 | C | SER | A | 242 | 21.262 | 14.001 | 43.772 | 1.00 | 35.92 |
| ATOM | 1981 | O | SER | A | 242 | 20.826 | 13.694 | 44.868 | 1.00 | 36.57 |
| ATOM | 1982 | CB | SER | A | 242 | 19.652 | 15.764 | 43.066 | 1.00 | 41.14 |
| ATOM | 1983 | OG | SER | A | 242 | 18.980 | 14.695 | 42.410 | 1.00 | 43.22 |
| ATOM | 1984 | N | SER | A | 243 | 21.821 | 13.121 | 42.943 | 1.00 | 34.07 |
| ATOM | 1985 | CA | SER | A | 243 | 22.116 | 11.752 | 43.336 | 1.00 | 37.77 |
| ATOM | 1986 | C | SER | A | 243 | 23.381 | 11.645 | 44.197 | 1.00 | 36.66 |
| ATOM | 1987 | O | SER | A | 243 | 23.640 | 10.589 | 44.763 | 1.00 | 40.77 |
| ATOM | 1988 | CB | SER | A | 243 | 22.270 | 10.852 | 42.096 | 1.00 | 38.01 |
| ATOM | 1989 | OG | SER | A | 243 | 23.450 | 11.164 | 41.363 | 1.00 | 40.41 |
| ATOM | 1990 | N | VAL | A | 244 | 24.172 | 12.717 | 44.266 | 1.00 | 37.13 |
| ATOM | 1991 | CA | VAL | A | 244 | 25.385 | 12.747 | 45.092 | 1.00 | 36.10 |
| ATOM | 1992 | C | VAL | A | 244 | 24.947 | 12.748 | 46.556 | 1.00 | 35.80 |
| ATOM | 1993 | O | VAL | A | 244 | 24.359 | 13.718 | 47.049 | 1.00 | 32.02 |
| ATOM | 1994 | CB | VAL | A | 244 | 26.303 | 13.972 | 44.773 | 1.00 | 36.81 |
| ATOM | 1995 | CG1 | VAL | A | 244 | 27.495 | 14.047 | 45.735 | 1.00 | 39.14 |
| ATOM | 1996 | CG2 | VAL | A | 244 | 26.804 | 13.911 | 43.319 | 1.00 | 35.55 |
| ATOM | 1997 | N | ASP | A | 245 | 25.225 | 11.630 | 47.220 | 1.00 | 36.84 |
| ATOM | 1998 | CA | ASP | A | 245 | 24.843 | 11.383 | 48.608 | 1.00 | 37.92 |
| ATOM | 1999 | C | ASP | A | 245 | 26.101 | 11.444 | 49.471 | 1.00 | 34.42 |
| ATOM | 2000 | O | ASP | A | 245 | 26.954 | 10.564 | 49.395 | 1.00 | 36.20 |
| ATOM | 2001 | CB | ASP | A | 245 | 24.190 | 10.000 | 48.692 | 1.00 | 40.11 |
| ATOM | 2002 | CG | ASP | A | 245 | 23.606 | 9.684 | 50.071 | 1.00 | 43.89 |
| ATOM | 2003 | OD1 | ASP | A | 245 | 23.995 | 10.292 | 51.104 | 1.00 | 46.19 |
| ATOM | 2004 | OD2 | ASP | A | 245 | 22.732 | 8.815 | 50.194 | 1.00 | 48.65 |
| ATOM | 2005 | N | ILE | A | 246 | 26.226 | 12.496 | 50.268 | 1.00 | 34.58 |
| ATOM | 2006 | CA | ILE | A | 246 | 27.421 | 12.712 | 51.100 | 1.00 | 34.24 |
| ATOM | 2007 | C | ILE | A | 246 | 27.746 | 11.547 | 52.049 | 1.00 | 34.35 |
| ATOM | 2008 | O | ILE | A | 246 | 28.913 | 11.284 | 52.334 | 1.00 | 34.47 |
| ATOM | 2009 | CB | ILE | A | 246 | 27.297 | 14.049 | 51.866 | 1.00 | 36.01 |
| ATOM | 2010 | CG1 | ILE | A | 246 | 28.607 | 14.400 | 52.578 | 1.00 | 38.62 |
| ATOM | 2011 | CG2 | ILE | A | 246 | 26.156 | 14.011 | 52.868 | 1.00 | 40.14 |
| ATOM | 2012 | CD1 | ILE | A | 246 | 28.676 | 15.858 | 53.015 | 1.00 | 41.03 |
| ATOM | 2013 | N | LYS | A | 247 | 26.717 | 10.836 | 52.502 | 1.00 | 33.97 |
| ATOM | 2014 | CA | LYS | A | 247 | 26.883 | 9.675 | 53.372 | 1.00 | 35.91 |
| ATOM | 2015 | C | LYS | A | 247 | 27.561 | 8.541 | 52.618 | 1.00 | 34.60 |
| ATOM | 2016 | O | LYS | A | 247 | 28.478 | 7.899 | 53.130 | 1.00 | 33.85 |
| ATOM | 2017 | CB | LYS | A | 247 | 25.533 | 9.210 | 53.949 | 1.00 | 37.68 |
| ATOM | 2018 | CG | LYS | A | 247 | 24.698 | 10.317 | 54.636 | 1.00 | 41.97 |
| ATOM | 2019 | CD | LYS | A | 247 | 23.300 | 9.831 | 55.115 | 1.00 | 45.11 |
| ATOM | 2020 | CE | LYS | A | 247 | 22.342 | 9.488 | 53.965 | 1.00 | 45.33 |
| ATOM | 2021 | NZ | LYS | A | 247 | 21.941 | 10.653 | 53.150 | 1.00 | 44.37 |
| ATOM | 2022 | N | LYS | A | 248 | 27.119 | 8.316 | 51.386 | 1.00 | 37.62 |
| ATOM | 2023 | CA | LYS | A | 248 | 27.696 | 7.291 | 50.525 | 1.00 | 37.40 |
| ATOM | 2024 | C | LYS | A | 248 | 29.133 | 7.619 | 50.101 | 1.00 | 34.78 |
| ATOM | 2025 | O | LYS | A | 248 | 29.984 | 6.725 | 50.010 | 1.00 | 32.91 |
| ATOM | 2026 | CB | LYS | A | 248 | 26.803 | 7.072 | 49.300 | 1.00 | 41.83 |
| ATOM | 2027 | CG | LYS | A | 248 | 25.439 | 6.441 | 49.645 | 1.00 | 47.35 |
| ATOM | 2028 | CD | LYS | A | 248 | 24.879 | 5.571 | 48.509 | 1.00 | 51.20 |
| ATOM | 2029 | CE | LYS | A | 248 | 23.390 | 5.239 | 48.712 | 1.00 | 52.76 |
| ATOM | 2030 | NZ | LYS | A | 248 | 22.490 | 6.336 | 48.233 | 1.00 | 51.45 |
| ATOM | 2031 | N | VAL | A | 249 | 29.413 | 8.900 | 49.859 | 1.00 | 34.70 |
| ATOM | 2032 | CA | VAL | A | 249 | 30.768 | 9.315 | 49.505 | 1.00 | 30.71 |
| ATOM | 2033 | C | VAL | A | 249 | 31.673 | 9.130 | 50.714 | 1.00 | 28.52 |
| ATOM | 2034 | O | VAL | A | 249 | 32.768 | 8.614 | 50.582 | 1.00 | 24.38 |
| ATOM | 2035 | CB | VAL | A | 249 | 30.832 | 10.768 | 48.997 | 1.00 | 28.72 |
| ATOM | 2036 | CG1 | VAL | A | 249 | 32.284 | 11.191 | 48.743 | 1.00 | 32.77 |
| ATOM | 2037 | CG2 | VAL | A | 249 | 30.040 | 10.918 | 47.729 | 1.00 | 30.80 |
| ATOM | 2038 | N | LEU | A | 250 | 31.203 | 9.546 | 51.889 | 1.00 | 28.31 |
| ATOM | 2039 | CA | LEU | A | 250 | 31.950 | 9.355 | 53.128 | 1.00 | 30.22 |
| ATOM | 2040 | C | LEU | A | 250 | 32.248 | 7.869 | 53.379 | 1.00 | 29.53 |
| ATOM | 2041 | O | LEU | A | 250 | 33.384 | 7.502 | 53.691 | 1.00 | 28.46 |
| ATOM | 2042 | CB | LEU | A | 250 | 31.195 | 9.940 | 54.324 | 1.00 | 30.09 |
| ATOM | 2043 | CG | LEU | A | 250 | 31.816 | 9.700 | 55.715 | 1.00 | 31.42 |
| ATOM | 2044 | CD1 | LEU | A | 250 | 33.252 | 10.149 | 55.775 | 1.00 | 31.57 |
| ATOM | 2045 | CD2 | LEU | A | 250 | 31.025 | 10.411 | 56.795 | 1.00 | 30.92 |
| ATOM | 2046 | N | LEU | A | 251 | 31.243 | 7.018 | 53.206 | 1.00 | 30.43 |
| ATOM | 2047 | CA | LEU | A | 251 | 31.421 | 5.583 | 53.441 | 1.00 | 31.75 |
| ATOM | 2048 | C | LEU | A | 251 | 32.414 | 5.003 | 52.479 | 1.00 | 28.77 |
| ATOM | 2049 | O | LEU | A | 251 | 33.220 | 4.167 | 52.861 | 1.00 | 27.12 |
| ATOM | 2050 | CB | LEU | A | 251 | 30.100 | 4.825 | 53.353 | 1.00 | 35.92 |
| ATOM | 2051 | CG | LEU | A | 251 | 29.236 | 4.875 | 54.618 | 1.00 | 39.34 |
| ATOM | 2052 | CD1 | LEU | A | 251 | 27.823 | 4.403 | 54.280 | 1.00 | 43.05 |
| ATOM | 2053 | CD2 | LEU | A | 251 | 29.846 | 4.037 | 55.743 | 1.00 | 38.51 |
| ATOM | 2054 | N | GLU | A | 252 | 32.411 | 5.477 | 51.237 | 1.00 | 29.11 |
| ATOM | 2055 | CA | GLU | A | 252 | 33.403 | 5.015 | 50.258 | 1.00 | 27.20 |
| ATOM | 2056 | C | GLU | A | 252 | 34.828 | 5.444 | 50.641 | 1.00 | 30.00 |
| ATOM | 2057 | O | GLU | A | 252 | 35.787 | 4.689 | 50.516 | 1.00 | 31.16 |
| ATOM | 2058 | CB | GLU | A | 252 | 33.035 | 5.548 | 48.882 | 1.00 | 32.72 |
| ATOM | 2059 | CG | GLU | A | 252 | 34.048 | 5.232 | 47.792 | 1.00 | 35.50 |
| ATOM | 2060 | CD | GLU | A | 252 | 34.041 | 3.768 | 47.382 | 1.00 | 40.85 |
| ATOM | 2061 | OE1 | GLU | A | 252 | 33.055 | 3.059 | 47.707 | 1.00 | 37.49 |
| ATOM | 2062 | OE2 | GLU | A | 252 | 35.020 | 3.343 | 46.719 | 1.00 | 39.81 |
| ATOM | 2063 | N | MET | A | 253 | 34.961 | 6.682 | 51.099 | 1.00 | 30.83 |
| ATOM | 2064 | CA | MET | A | 253 | 36.247 | 7.219 | 51.533 | 1.00 | 30.96 |
| ATOM | 2065 | C | MET | A | 253 | 36.742 | 6.424 | 52.736 | 1.00 | 28.97 |
| ATOM | 2066 | O | MET | A | 253 | 37.912 | 6.091 | 52.851 | 1.00 | 28.87 |
| ATOM | 2067 | CB | MET | A | 253 | 36.065 | 8.691 | 51.930 | 1.00 | 29.25 |
| ATOM | 2068 | CG | MET | A | 253 | 37.065 | 9.640 | 51.346 | 1.00 | 35.75 |
| ATOM | 2069 | SD | MET | A | 253 | 36.473 | 11.363 | 51.342 | 1.00 | 34.89 |
| ATOM | 2070 | CE | MET | A | 253 | 36.648 | 11.727 | 49.596 | 1.00 | 33.07 |
| ATOM | 2071 | N | ARG | A | 254 | 35.807 | 6.136 | 53.629 | 1.00 | 30.28 |
| ATOM | 2072 | CA | ARG | A | 254 | 36.061 | 5.427 | 54.875 | 1.00 | 32.59 |
| ATOM | 2073 | C | ARG | A | 254 | 36.654 | 4.013 | 54.659 | 1.00 | 32.55 |
| ATOM | 2074 | O | ARG | A | 254 | 37.296 | 3.460 | 55.542 | 1.00 | 30.27 |
| ATOM | 2075 | CB | ARG | A | 254 | 34.745 | 5.422 | 55.652 | 1.00 | 38.30 |
| ATOM | 2076 | CG | ARG | A | 254 | 34.776 | 4.884 | 57.034 | 1.00 | 45.57 |
| ATOM | 2077 | CD | ARG | A | 254 | 35.742 | 5.588 | 57.979 | 1.00 | 50.87 |
| ATOM | 2078 | NE | ARG | A | 254 | 35.742 | 4.925 | 59.277 | 1.00 | 49.69 |
| ATOM | 2079 | CZ | ARG | A | 254 | 36.213 | 3.707 | 59.497 | 1.00 | 48.16 |
| ATOM | 2080 | NH1 | ARG | A | 254 | 36.156 | 3.199 | 60.719 | 1.00 | 52.85 |
| ATOM | 2081 | NH2 | ARG | A | 254 | 36.750 | 2.993 | 58.516 | 1.00 | 47.70 |
| ATOM | 2082 | N | LYS | A | 255 | 36.511 | 3.458 | 53.458 | 1.00 | 29.98 |
| ATOM | 2083 | CA | LYS | A | 255 | 37.164 | 2.192 | 53.128 | 1.00 | 29.31 |
| ATOM | 2084 | C | LYS | A | 255 | 38.700 | 2.267 | 53.131 | 1.00 | 31.32 |
| ATOM | 2085 | O | LYS | A | 255 | 39.363 | 1.226 | 53.227 | 1.00 | 31.34 |
| ATOM | 2086 | CB | LYS | A | 255 | 36.712 | 1.716 | 51.734 | 1.00 | 30.37 |
| ATOM | 2087 | CG | LYS | A | 255 | 35.222 | 1.384 | 51.608 | 1.00 | 26.24 |
| ATOM | 2088 | CD | LYS | A | 255 | 34.791 | 1.347 | 50.151 | 1.00 | 26.82 |
| ATOM | 2089 | CE | LYS | A | 255 | 33.323 | 0.845 | 49.937 | 1.00 | 27.52 |
| ATOM | 2090 | NZ | LYS | A | 255 | 33.067 | 0.534 | 48.456 | 1.00 | 24.46 |
| ATOM | 2091 | N | PHE | A | 256 | 39.253 | 3.481 | 52.964 | 1.00 | 27.58 |
| ATOM | 2092 | CA | PHE | A | 256 | 40.690 | 3.693 | 52.796 | 1.00 | 25.60 |
| ATOM | 2093 | C | PHE | A | 256 | 41.392 | 4.191 | 54.054 | 1.00 | 23.14 |
| ATOM | 2094 | O | PHE | A | 256 | 42.600 | 4.017 | 54.221 | 1.00 | 25.79 |
| ATOM | 2095 | CB | PHE | A | 256 | 40.938 | 4.656 | 51.634 | 1.00 | 28.49 |
| ATOM | 2096 | CG | PHE | A | 256 | 40.416 | 4.151 | 50.301 | 1.00 | 25.93 |
| ATOM | 2097 | CD1 | PHE | A | 256 | 41.181 | 3.311 | 49.516 | 1.00 | 28.34 |
| ATOM | 2098 | CD2 | PHE | A | 256 | 39.152 | 4.503 | 49.857 | 1.00 | 25.47 |
| ATOM | 2099 | CE1 | PHE | A | 256 | 40.702 | 2.846 | 48.288 | 1.00 | 28.01 |
| ATOM | 2100 | CE2 | PHE | A | 256 | 38.677 | 4.044 | 48.649 | 1.00 | 23.45 |
| ATOM | 2101 | CZ | PHE | A | 256 | 39.452 | 3.211 | 47.865 | 1.00 | 25.89 |
| ATOM | 2102 | N | ARG | A | 257 | 40.649 | 4.847 | 54.928 | 1.00 | 24.88 |
| ATOM | 2103 | CA | ARG | A | 257 | 41.203 | 5.234 | 56.199 | 1.00 | 26.69 |
| ATOM | 2104 | C | ARG | A | 257 | 40.084 | 5.522 | 57.173 | 1.00 | 27.56 |
| ATOM | 2105 | O | ARG | A | 257 | 39.029 | 6.033 | 56.797 | 1.00 | 29.01 |
| ATOM | 2106 | CB | ARG | A | 257 | 42.120 | 6.461 | 56.045 | 1.00 | 26.90 |
| ATOM | 2107 | CG | ARG | A | 257 | 42.932 | 6.827 | 57.294 | 1.00 | 28.05 |
| ATOM | 2108 | CD | ARG | A | 257 | 43.883 | 8.013 | 57.024 | 1.00 | 26.37 |
| ATOM | 2109 | NE | ARG | A | 257 | 44.621 | 8.479 | 58.194 | 1.00 | 19.25 |
| ATOM | 2110 | CZ | ARG | A | 257 | 45.812 | 8.058 | 58.607 | 1.00 | 23.43 |
| ATOM | 2111 | NH1 | ARG | A | 257 | 46.448 | 7.059 | 58.019 | 1.00 | 22.28 |
| ATOM | 2112 | NH2 | ARG | A | 257 | 46.361 | 8.639 | 59.663 | 1.00 | 28.19 |
| ATOM | 2113 | N | MET | A | 258 | 40.352 | 5.204 | 58.433 | 1.00 | 29.28 |
| ATOM | 2114 | CA | MET | A | 258 | 39.426 | 5.426 | 59.535 | 1.00 | 33.32 |
| ATOM | 2115 | C | MET | A | 258 | 39.523 | 6.871 | 60.069 | 1.00 | 33.40 |
| ATOM | 2116 | O | MET | A | 258 | 40.485 | 7.575 | 59.789 | 1.00 | 24.99 |
| ATOM | 2117 | CB | MET | A | 258 | 39.711 | 4.399 | 60.656 | 1.00 | 36.62 |
| ATOM | 2118 | CG | MET | A | 258 | 41.087 | 4.534 | 61.324 | 1.00 | 43.85 |
| ATOM | 2119 | SD | MET | A | 258 | 41.460 | 3.404 | 62.774 | 1.00 | 51.60 |
| ATOM | 2120 | CE | MET | A | 258 | 42.050 | 1.920 | 61.953 | 1.00 | 45.75 |
| ATOM | 2121 | N | GLY | A | 259 | 38.513 | 7.300 | 60.814 | 1.00 | 32.82 |
| ATOM | 2122 | CA | GLY | A | 259 | 38.548 | 8.586 | 61.494 | 1.00 | 36.92 |
| ATOM | 2123 | C | GLY | A | 259 | 38.456 | 9.798 | 60.576 | 1.00 | 38.56 |
| ATOM | 2124 | O | GLY | A | 259 | 39.091 | 10.842 | 60.820 | 1.00 | 35.70 |
| ATOM | 2125 | N | LEU | A | 260 | 37.679 | 9.656 | 59.509 | 1.00 | 34.91 |
| ATOM | 2126 | CA | LEU | A | 260 | 37.484 | 10.742 | 58.571 | 1.00 | 35.45 |
| ATOM | 2127 | C | LEU | A | 260 | 36.578 | 11.823 | 59.155 | 1.00 | 36.54 |
| ATOM | 2128 | O | LEU | A | 260 | 36.900 | 13.010 | 59.087 | 1.00 | 37.25 |
| ATOM | 2129 | CB | LEU | A | 260 | 36.923 | 10.201 | 57.266 | 1.00 | 33.19 |
| ATOM | 2130 | CG | LEU | A | 260 | 37.904 | 9.287 | 56.528 | 1.00 | 33.16 |
| ATOM | 2131 | CD1 | LEU | A | 260 | 37.319 | 8.911 | 55.197 | 1.00 | 35.18 |
| ATOM | 2132 | CD2 | LEU | A | 260 | 39.285 | 9.907 | 56.353 | 1.00 | 34.68 |
| ATOM | 2133 | N | ILE | A | 261 | 35.452 | 11.418 | 59.724 | 1.00 | 37.56 |
| ATOM | 2134 | CA | ILE | A | 261 | 34.574 | 12.345 | 60.429 | 1.00 | 40.12 |
| ATOM | 2135 | C | ILE | A | 261 | 34.443 | 11.802 | 61.846 | 1.00 | 42.88 |
| ATOM | 2136 | O | ILE | A | 261 | 33.956 | 10.685 | 62.034 | 1.00 | 42.56 |
| ATOM | 2137 | CB | ILE | A | 261 | 33.195 | 12.447 | 59.730 | 1.00 | 41.51 |
| ATOM | 2138 | CG1 | ILE | A | 261 | 33.358 | 12.831 | 58.255 | 1.00 | 44.63 |
| ATOM | 2139 | CG2 | ILE | A | 261 | 32.290 | 13.474 | 60.423 | 1.00 | 41.66 |
| ATOM | 2140 | CD1 | ILE | A | 261 | 33.854 | 14.256 | 58.003 | 1.00 | 45.20 |
| ATOM | 2141 | N | GLN | A | 262 | 34.904 | 12.582 | 62.822 | 1.00 | 44.63 |
| ATOM | 2142 | CA | GLN | A | 262 | 35.013 | 12.132 | 64.213 | 1.00 | 46.99 |
| ATOM | 2143 | C | GLN | A | 262 | 33.830 | 12.505 | 65.113 | 1.00 | 47.40 |
| ATOM | 2144 | O | GLN | A | 262 | 33.650 | 11.900 | 66.164 | 1.00 | 49.81 |
| ATOM | 2145 | CB | GLN | A | 262 | 36.305 | 12.674 | 64.851 | 1.00 | 46.30 |
| ATOM | 2146 | CG | GLN | A | 262 | 37.565 | 11.989 | 64.392 | 1.00 | 48.97 |
| ATOM | 2147 | CD | GLN | A | 262 | 37.666 | 10.520 | 64.830 | 1.00 | 51.67 |
| ATOM | 2148 | OE1 | GLN | A | 262 | 36.752 | 9.738 | 64.612 | 1.00 | 59.14 |
| ATOM | 2149 | NE2 | GLN | A | 262 | 38.794 | 10.148 | 65.416 | 1.00 | 56.82 |
| ATOM | 2150 | N | THR | A | 263 | 33.049 | 13.512 | 64.736 | 1.00 | 48.72 |
| ATOM | 2151 | CA | THR | A | 263 | 31.879 | 13.894 | 65.526 | 1.00 | 48.51 |
| ATOM | 2152 | C | THR | A | 263 | 30.688 | 14.228 | 64.645 | 1.00 | 49.93 |
| ATOM | 2153 | O | THR | A | 263 | 30.832 | 14.483 | 63.445 | 1.00 | 46.32 |
| ATOM | 2154 | CB | THR | A | 263 | 32.190 | 15.105 | 66.405 | 1.00 | 49.79 |
| ATOM | 2155 | OG1 | THR | A | 263 | 32.603 | 16.202 | 65.587 | 1.00 | 48.64 |
| ATOM | 2156 | CG2 | THR | A | 263 | 33.388 | 14.852 | 67.325 | 1.00 | 50.03 |
| ATOM | 2157 | N | ALA | A | 264 | 29.512 | 14.218 | 65.269 | 1.00 | 48.46 |
| ATOM | 2158 | CA | ALA | A | 264 | 28.264 | 14.619 | 64.635 | 1.00 | 48.90 |
| ATOM | 2159 | C | ALA | A | 264 | 28.331 | 16.034 | 64.080 | 1.00 | 47.97 |
| ATOM | 2160 | O | ALA | A | 264 | 27.724 | 16.307 | 63.051 | 1.00 | 46.14 |
| ATOM | 2161 | CB | ALA | A | 264 | 27.096 | 14.514 | 65.637 | 1.00 | 50.13 |
| ATOM | 2162 | N | ASP | A | 265 | 29.054 | 16.922 | 64.764 | 1.00 | 46.13 |
| ATOM | 2163 | CA | ASP | A | 265 | 29.173 | 18.312 | 64.341 | 1.00 | 49.36 |
| ATOM | 2164 | C | ASP | A | 265 | 30.000 | 18.452 | 63.072 | 1.00 | 46.31 |
| ATOM | 2165 | O | ASP | A | 265 | 29.739 | 19.329 | 62.261 | 1.00 | 47.97 |
| ATOM | 2166 | CB | ASP | A | 265 | 29.832 | 19.163 | 65.427 | 1.00 | 53.22 |
| ATOM | 2167 | CG | ASP | A | 265 | 28.992 | 19.277 | 66.681 | 1.00 | 61.01 |
| ATOM | 2168 | OD1 | ASP | A | 265 | 27.747 | 19.406 | 66.572 | 1.00 | 68.83 |
| ATOM | 2169 | OD2 | ASP | A | 265 | 29.505 | 19.259 | 67.827 | 1.00 | 65.34 |
| ATOM | 2170 | N | GLN | A | 266 | 31.027 | 17.623 | 62.938 | 1.00 | 42.43 |
| ATOM | 2171 | CA | GLN | A | 266 | 31.876 | 17.634 | 61.755 | 1.00 | 41.84 |
| ATOM | 2172 | C | GLN | A | 266 | 31.103 | 17.162 | 60.543 | 1.00 | 39.69 |
| ATOM | 2173 | O | GLN | A | 266 | 31.287 | 17.675 | 59.461 | 1.00 | 39.79 |
| ATOM | 2174 | CB | GLN | A | 266 | 33.084 | 16.722 | 61.942 | 1.00 | 42.22 |
| ATOM | 2175 | CG | GLN | A | 266 | 34.123 | 17.234 | 62.905 | 1.00 | 42.76 |
| ATOM | 2176 | CD | GLN | A | 266 | 35.476 | 16.563 | 62.702 | 1.00 | 45.93 |
| ATOM | 2177 | OE1 | GLN | A | 266 | 35.558 | 15.387 | 62.298 | 1.00 | 42.79 |
| ATOM | 2178 | NE2 | GLN | A | 266 | 36.541 | 17.310 | 62.974 | 1.00 | 43.52 |
| ATOM | 2179 | N | LEU | A | 267 | 30.255 | 16.160 | 60.737 | 1.00 | 40.23 |
| ATOM | 2180 | CA | LEU | A | 267 | 29.353 | 15.704 | 59.695 | 1.00 | 41.23 |
| ATOM | 2181 | C | LEU | A | 267 | 28.433 | 16.830 | 59.275 | 1.00 | 40.80 |
| ATOM | 2182 | O | LEU | A | 267 | 28.297 | 17.117 | 58.097 | 1.00 | 38.81 |
| ATOM | 2183 | CB | LEU | A | 267 | 28.520 | 14.537 | 60.193 | 1.00 | 41.88 |
| ATOM | 2184 | CG | LEU | A | 267 | 27.542 | 13.904 | 59.208 | 1.00 | 42.25 |
| ATOM | 2185 | CD1 | LEU | A | 267 | 28.282 | 13.277 | 58.039 | 1.00 | 41.79 |
| ATOM | 2186 | CD2 | LEU | A | 267 | 26.695 | 12.866 | 59.939 | 1.00 | 43.40 |
| ATOM | 2187 | N | ARG | A | 268 | 27.813 | 17.481 | 60.250 | 1.00 | 43.33 |
| ATOM | 2188 | CA | ARG | A | 268 | 26.912 | 18.585 | 59.957 | 1.00 | 42.44 |
| ATOM | 2189 | C | ARG | A | 268 | 27.662 | 19.638 | 59.152 | 1.00 | 39.47 |
| ATOM | 2190 | O | ARG | A | 268 | 27.170 | 20.110 | 58.136 | 1.00 | 36.25 |
| ATOM | 2191 | CB | ARG | A | 268 | 26.339 | 19.199 | 61.245 | 1.00 | 44.59 |
| ATOM | 2192 | CG | ARG | A | 268 | 25.365 | 20.357 | 60.992 | 1.00 | 47.22 |
| ATOM | 2193 | CD | ARG | A | 268 | 24.719 | 20.937 | 62.253 | 1.00 | 51.04 |
| ATOM | 2194 | NE | ARG | A | 268 | 23.755 | 21.996 | 61.925 | 1.00 | 49.26 |
| ATOM | 2195 | CZ | ARG | A | 268 | 24.052 | 23.284 | 61.786 | 1.00 | 50.43 |
| ATOM | 2196 | NH1 | ARG | A | 268 | 25.293 | 23.726 | 61.914 | 1.00 | 50.57 |
| ATOM | 2197 | NH2 | ARG | A | 268 | 23.091 | 24.147 | 61.487 | 1.00 | 54.11 |
| ATOM | 2198 | N | PHE | A | 269 | 28.858 | 19.987 | 59.621 | 1.00 | 36.52 |
| ATOM | 2199 | CA | PHE | A | 269 | 29.717 | 20.935 | 58.938 | 1.00 | 35.76 |
| ATOM | 2200 | C | PHE | A | 269 | 30.059 | 20.521 | 57.498 | 1.00 | 35.94 |
| ATOM | 2201 | O | PHE | A | 269 | 30.199 | 21.370 | 56.646 | 1.00 | 39.34 |
| ATOM | 2202 | CB | PHE | A | 269 | 31.018 | 21.117 | 59.696 | 1.00 | 32.56 |
| ATOM | 2203 | CG | PHE | A | 269 | 31.893 | 22.188 | 59.119 | 1.00 | 30.04 |
| ATOM | 2204 | CD1 | PHE | A | 269 | 33.037 | 21.865 | 58.414 | 1.00 | 29.12 |
| ATOM | 2205 | CD2 | PHE | A | 269 | 31.546 | 23.523 | 59.261 | 1.00 | 32.32 |
| ATOM | 2206 | CE1 | PHE | A | 269 | 33.849 | 22.857 | 57.884 | 1.00 | 29.89 |
| ATOM | 2207 | CE2 | PHE | A | 269 | 32.337 | 24.526 | 58.731 | 1.00 | 30.18 |
| ATOM | 2208 | CZ | PHE | A | 269 | 33.491 | 24.193 | 58.030 | 1.00 | 34.06 |
| ATOM | 2209 | N | SER | A | 270 | 30.224 | 19.228 | 57.248 | 1.00 | 34.50 |
| ATOM | 2210 | CA | SER | A | 270 | 30.543 | 18.737 | 55.913 | 1.00 | 34.36 |
| ATOM | 2211 | C | SER | A | 270 | 29.373 | 18.941 | 54.953 | 1.00 | 34.34 |
| ATOM | 2212 | O | SER | A | 270 | 29.558 | 19.437 | 53.839 | 1.00 | 30.23 |
| ATOM | 2213 | CB | SER | A | 270 | 30.923 | 17.262 | 55.975 | 1.00 | 32.60 |
| ATOM | 2214 | OG | SER | A | 270 | 32.077 | 17.101 | 56.773 | 1.00 | 37.71 |
| ATOM | 2215 | N | TYR | A | 271 | 28.176 | 18.540 | 55.388 | 1.00 | 36.71 |
| ATOM | 2216 | CA | TYR | A | 271 | 26.929 | 18.830 | 54.678 | 1.00 | 35.83 |
| ATOM | 2217 | C | TYR | A | 271 | 26.874 | 20.295 | 54.345 | 1.00 | 36.60 |
| ATOM | 2218 | O | TYR | A | 271 | 26.634 | 20.682 | 53.213 | 1.00 | 37.68 |
| ATOM | 2219 | CB | TYR | A | 271 | 25.716 | 18.539 | 55.563 | 1.00 | 38.12 |
| ATOM | 2220 | CG | TYR | A | 271 | 25.160 | 17.142 | 55.497 | 1.00 | 34.68 |
| ATOM | 2221 | CD1 | TYR | A | 271 | 25.791 | 16.096 | 56.143 | 1.00 | 37.80 |
| ATOM | 2222 | CD2 | TYR | A | 271 | 23.981 | 16.874 | 54.809 | 1.00 | 40.16 |
| ATOM | 2223 | CE1 | TYR | A | 271 | 25.279 | 14.805 | 56.102 | 1.00 | 39.89 |
| ATOM | 2224 | CE2 | TYR | A | 271 | 23.465 | 15.582 | 54.754 | 1.00 | 42.27 |
| ATOM | 2225 | CZ | TYR | A | 271 | 24.114 | 14.557 | 55.411 | 1.00 | 42.20 |
| ATOM | 2226 | OH | TYR | A | 271 | 23.612 | 13.277 | 55.375 | 1.00 | 49.03 |
| ATOM | 2227 | N | LEU | A | 272 | 27.117 | 21.112 | 55.357 | 1.00 | 38.95 |
| ATOM | 2228 | CA | LEU | A | 272 | 26.998 | 22.553 | 55.225 | 1.00 | 39.72 |
| ATOM | 2229 | C | LEU | A | 272 | 27.988 | 23.097 | 54.212 | 1.00 | 40.36 |
| ATOM | 2230 | O | LEU | A | 272 | 27.638 | 23.981 | 53.432 | 1.00 | 42.33 |
| ATOM | 2231 | CB | LEU | A | 272 | 27.178 | 23.217 | 56.593 | 1.00 | 42.16 |
| ATOM | 2232 | CG | LEU | A | 272 | 27.040 | 24.733 | 56.691 | 1.00 | 46.41 |
| ATOM | 2233 | CD1 | LEU | A | 272 | 25.793 | 25.257 | 55.955 | 1.00 | 48.86 |
| ATOM | 2234 | CD2 | LEU | A | 272 | 27.006 | 25.141 | 58.175 | 1.00 | 49.08 |
| ATOM | 2235 | N | ALA | A | 273 | 29.211 | 22.553 | 54.198 | 1.00 | 36.56 |
| ATOM | 2236 | CA | ALA | A | 273 | 30.250 | 23.024 | 53.281 | 1.00 | 34.47 |
| ATOM | 2237 | C | ALA | A | 273 | 29.931 | 22.604 | 51.851 | 1.00 | 31.70 |
| ATOM | 2238 | O | ALA | A | 273 | 30.176 | 23.341 | 50.913 | 1.00 | 34.20 |
| ATOM | 2239 | CB | ALA | A | 273 | 31.622 | 22.490 | 53.690 | 1.00 | 35.73 |
| ATOM | 2240 | N | VAL | A | 274 | 29.394 | 21.409 | 51.697 | 1.00 | 31.56 |
| ATOM | 2241 | CA | VAL | A | 274 | 29.082 | 20.878 | 50.381 | 1.00 | 34.59 |
| ATOM | 2242 | C | VAL | A | 274 | 27.909 | 21.683 | 49.790 | 1.00 | 38.49 |
| ATOM | 2243 | O | VAL | A | 274 | 27.952 | 22.053 | 48.617 | 1.00 | 39.12 |
| ATOM | 2244 | CB | VAL | A | 274 | 28.817 | 19.341 | 50.454 | 1.00 | 31.94 |
| ATOM | 2245 | CG1 | VAL | A | 274 | 28.213 | 18.791 | 49.172 | 1.00 | 33.30 |
| ATOM | 2246 | CG2 | VAL | A | 274 | 30.111 | 18.592 | 50.765 | 1.00 | 31.69 |
| ATOM | 2247 | N | ILE | A | 275 | 26.908 | 21.999 | 50.610 | 1.00 | 36.26 |
| ATOM | 2248 | CA | ILE | A | 275 | 25.726 | 22.739 | 50.155 | 1.00 | 39.86 |
| ATOM | 2249 | C | ILE | A | 275 | 26.088 | 24.160 | 49.728 | 1.00 | 38.34 |
| ATOM | 2250 | O | ILE | A | 275 | 25.704 | 24.613 | 48.665 | 1.00 | 39.42 |
| ATOM | 2251 | CB | ILE | A | 275 | 24.651 | 22.778 | 51.277 | 1.00 | 41.82 |
| ATOM | 2252 | CG1 | ILE | A | 275 | 24.023 | 21.400 | 51.473 | 1.00 | 43.45 |
| ATOM | 2253 | CG2 | ILE | A | 275 | 23.552 | 23.784 | 50.969 | 1.00 | 44.70 |
| ATOM | 2254 | CD1 | ILE | A | 275 | 23.449 | 21.196 | 52.861 | 1.00 | 45.17 |
| ATOM | 2255 | N | GLU | A | 276 | 26.827 | 24.871 | 50.558 | 1.00 | 37.37 |
| ATOM | 2256 | CA | GLU | A | 276 | 27.189 | 26.238 | 50.227 | 1.00 | 39.70 |
| ATOM | 2257 | C | GLU | A | 276 | 28.234 | 26.289 | 49.120 | 1.00 | 39.46 |
| ATOM | 2258 | O | GLU | A | 276 | 28.251 | 27.222 | 48.305 | 1.00 | 41.95 |
| ATOM | 2259 | CB | GLU | A | 276 | 27.676 | 26.973 | 51.476 | 1.00 | 42.35 |
| ATOM | 2260 | CG | GLU | A | 276 | 26.568 | 27.180 | 52.502 | 1.00 | 48.65 |
| ATOM | 2261 | CD | GLU | A | 276 | 26.923 | 28.196 | 53.583 | 1.00 | 55.14 |
| ATOM | 2262 | OE1 | GLU | A | 276 | 26.335 | 28.108 | 54.695 | 1.00 | 58.67 |
| ATOM | 2263 | OE2 | GLU | A | 276 | 27.787 | 29.080 | 53.330 | 1.00 | 57.40 |
| ATOM | 2264 | N | GLY | A | 277 | 29.115 | 25.294 | 49.083 | 1.00 | 35.14 |
| ATOM | 2265 | CA | GLY | A | 277 | 30.073 | 25.188 | 47.996 | 1.00 | 34.64 |
| ATOM | 2266 | C | GLY | A | 277 | 29.363 | 24.978 | 46.668 | 1.00 | 32.72 |
| ATOM | 2267 | O | GLY | A | 277 | 29.753 | 25.543 | 45.673 | 1.00 | 31.78 |
| ATOM | 2268 | N | ALA | A | 278 | 28.313 | 24.165 | 46.674 | 1.00 | 36.31 |
| ATOM | 2269 | CA | ALA | A | 278 | 27.550 | 23.852 | 45.471 | 1.00 | 39.36 |
| ATOM | 2270 | C | ALA | A | 278 | 26.795 | 25.062 | 44.966 | 1.00 | 43.06 |
| ATOM | 2271 | O | ALA | A | 278 | 26.734 | 25.286 | 43.767 | 1.00 | 45.13 |
| ATOM | 2272 | CB | ALA | A | 278 | 26.583 | 22.727 | 45.736 | 1.00 | 35.74 |
| ATOM | 2273 | N | LYS | A | 279 | 26.228 | 25.840 | 45.884 | 1.00 | 47.70 |
| ATOM | 2274 | CA | LYS | A | 279 | 25.511 | 27.067 | 45.522 | 1.00 | 50.45 |
| ATOM | 2275 | C | LYS | A | 279 | 26.459 | 28.064 | 44.865 | 1.00 | 50.00 |
| ATOM | 2276 | O | LYS | A | 279 | 26.076 | 28.756 | 43.929 | 1.00 | 53.95 |
| ATOM | 2277 | CB | LYS | A | 279 | 24.831 | 27.699 | 46.755 | 1.00 | 49.65 |
| ATOM | 2278 | CG | LYS | A | 279 | 23.598 | 26.941 | 47.224 | 1.00 | 53.64 |
| ATOM | 2279 | CD | LYS | A | 279 | 22.857 | 27.678 | 48.345 | 1.00 | 56.88 |
| ATOM | 2280 | CE | LYS | A | 279 | 21.838 | 26.777 | 49.068 | 1.00 | 58.26 |
| ATOM | 2281 | NZ | LYS | A | 279 | 21.551 | 27.277 | 50.453 | 1.00 | 59.08 |
| ATOM | 2282 | N | PHE | A | 280 | 27.695 | 28.115 | 45.351 | 1.00 | 49.27 |
| ATOM | 2283 | CA | PHE | A | 280 | 28.699 | 29.043 | 44.838 | 1.00 | 49.04 |
| ATOM | 2284 | C | PHE | A | 280 | 29.220 | 28.642 | 43.455 | 1.00 | 49.80 |
| ATOM | 2285 | O | PHE | A | 280 | 29.433 | 29.495 | 42.603 | 1.00 | 49.32 |
| ATOM | 2286 | CB | PHE | A | 280 | 29.869 | 29.147 | 45.826 | 1.00 | 50.39 |
| ATOM | 2287 | CG | PHE | A | 280 | 30.921 | 30.124 | 45.415 | 1.00 | 49.33 |
| ATOM | 2288 | CD1 | PHE | A | 280 | 30.765 | 31.478 | 45.668 | 1.00 | 49.83 |
| ATOM | 2289 | CD2 | PHE | A | 280 | 32.067 | 29.694 | 44.758 | 1.00 | 49.42 |
| ATOM | 2290 | CE1 | PHE | A | 280 | 31.733 | 32.384 | 45.274 | 1.00 | 48.13 |
| ATOM | 2291 | CE2 | PHE | A | 280 | 33.027 | 30.595 | 44.368 | 1.00 | 48.06 |
| ATOM | 2292 | CZ | PHE | A | 280 | 32.863 | 31.944 | 44.631 | 1.00 | 48.21 |
| ATOM | 2293 | N | ILE | A | 281 | 29.430 | 27.347 | 43.240 | 1.00 | 50.12 |
| ATOM | 2294 | CA | ILE | A | 281 | 29.905 | 26.840 | 41.951 | 1.00 | 50.40 |
| ATOM | 2295 | C | ILE | A | 281 | 28.749 | 26.722 | 40.939 | 1.00 | 55.35 |
| ATOM | 2296 | O | ILE | A | 281 | 28.971 | 26.875 | 39.742 | 1.00 | 55.56 |
| ATOM | 2297 | CB | ILE | A | 281 | 30.633 | 25.483 | 42.139 | 1.00 | 49.67 |
| ATOM | 2298 | CG1 | ILE | A | 281 | 31.938 | 25.690 | 42.912 | 1.00 | 48.72 |
| ATOM | 2299 | CG2 | ILE | A | 281 | 30.947 | 24.833 | 40.802 | 1.00 | 50.91 |
| ATOM | 2300 | CD1 | ILE | A | 281 | 32.411 | 24.478 | 43.633 | 1.00 | 47.70 |
| ATOM | 2301 | N | MET | A | 282 | 27.527 | 26.496 | 41.438 | 1.00 | 60.40 |
| ATOM | 2302 | CA | MET | A | 282 | 26.324 | 26.216 | 40.624 | 1.00 | 63.02 |
| ATOM | 2303 | C | MET | A | 282 | 25.115 | 27.081 | 41.102 | 1.00 | 62.62 |
| ATOM | 2304 | O | MET | A | 282 | 25.174 | 28.305 | 41.019 | 1.00 | 61.79 |
| ATOM | 2305 | CB | MET | A | 282 | 25.995 | 24.704 | 40.658 | 1.00 | 65.42 |
| ATOM | 2306 | CG | MET | A | 282 | 27.178 | 23.756 | 40.371 | 1.00 | 67.60 |
| ATOM | 2307 | SD | MET | A | 282 | 26.819 | 21.976 | 40.681 | 1.00 | 70.48 |
| ATOM | 2308 | CE | MET | A | 282 | 26.610 | 21.357 | 38.979 | 1.00 | 68.57 |
| ATOM | 2309 | N | GLY | A | 283 | 24.116 | 26.529 | 41.557 | 1.00 | 60.55 |
| HETATM | 2310 | MG | MG | A | 1282 | 44.422 | −3.071 | 46.899 | 1.00 | 60.24 |
| HETATM | 2311 | O | HOH | Z | 1 | 22.845 | 21.799 | 47.502 | 1.00 | 40.64 |
| HETATM | 2312 | O | HOH | Z | 2 | 15.323 | 16.826 | 46.375 | 1.00 | 49.94 |
| HETATM | 2313 | O | HOH | Z | 3 | 15.473 | 19.469 | 45.770 | 1.00 | 50.98 |
| HETATM | 2314 | O | HOH | Z | 4 | 22.424 | 19.648 | 43.271 | 1.00 | 45.79 |
| HETATM | 2315 | O | HOH | Z | 5 | 15.775 | 14.423 | 51.730 | 1.00 | 55.85 |
| HETATM | 2316 | O | HOH | Z | 6 | 21.510 | 17.929 | 46.342 | 1.00 | 41.90 |
| HETATM | 2317 | O | HOH | Z | 7 | 23.215 | 13.722 | 50.672 | 1.00 | 52.16 |
| HETATM | 2318 | O | HOH | Z | 8 | 21.609 | 15.105 | 67.182 | 1.00 | 55.05 |
| HETATM | 2319 | O | HOH | Z | 9 | 46.068 | −6.909 | 53.961 | 1.00 | 41.54 |
| HETATM | 2320 | O | HOH | Z | 10 | 46.376 | −3.583 | 45.594 | 1.00 | 48.83 |
| HETATM | 2321 | O | HOH | Z | 11 | 37.768 | 1.718 | 44.042 | 1.00 | 47.36 |
| HETATM | 2322 | O | HOH | Z | 12 | 33.307 | 2.262 | 54.833 | 1.00 | 33.08 |
| HETATM | 2323 | O | HOH | Z | 13 | 33.935 | −1.788 | 53.445 | 1.00 | 53.29 |
| HETATM | 2324 | O | HOH | Z | 14 | 34.355 | −4.241 | 57.338 | 1.00 | 37.80 |
| HETATM | 2325 | O | HOH | Z | 15 | 39.461 | −5.904 | 60.525 | 1.00 | 54.82 |
| HETATM | 2326 | O | HOH | Z | 16 | 65.218 | 12.046 | 62.590 | 1.00 | 53.16 |
| HETATM | 2327 | O | HOH | Z | 17 | 43.693 | −3.408 | 55.400 | 1.00 | 30.74 |
| HETATM | 2328 | O | HOH | Z | 18 | 44.621 | −4.890 | 63.128 | 1.00 | 44.34 |
| HETATM | 2329 | O | HOH | Z | 19 | 50.688 | −6.839 | 63.918 | 1.00 | 46.36 |
| HETATM | 2330 | O | HOH | Z | 20 | 53.338 | −0.323 | 66.457 | 1.00 | 42.76 |
| HETATM | 2331 | O | HOH | Z | 21 | 58.119 | −5.709 | 64.896 | 1.00 | 47.61 |
| HETATM | 2332 | O | HOH | Z | 22 | 49.949 | 29.097 | 63.293 | 1.00 | 46.85 |
| HETATM | 2333 | O | HOH | Z | 23 | 59.882 | 1.566 | 67.930 | 1.00 | 52.42 |
| HETATM | 2334 | O | HOH | Z | 24 | 54.549 | 27.023 | 69.066 | 1.00 | 53.35 |
| HETATM | 2335 | O | HOH | Z | 25 | 54.922 | 7.670 | 60.300 | 1.00 | 25.86 |
| HETATM | 2336 | O | HOH | Z | 26 | 55.413 | 8.114 | 69.236 | 1.00 | 55.54 |
| HETATM | 2337 | O | HOH | Z | 27 | 54.620 | 16.584 | 67.844 | 1.00 | 33.49 |
| HETATM | 2338 | O | HOH | Z | 28 | 48.736 | 6.328 | 65.214 | 1.00 | 47.76 |
| HETATM | 2339 | O | HOH | Z | 29 | 48.168 | 5.412 | 67.908 | 1.00 | 51.62 |
| HETATM | 2340 | O | HOH | Z | 30 | 49.368 | 13.438 | 69.668 | 1.00 | 49.93 |
| HETATM | 2341 | O | HOH | Z | 31 | 53.010 | 10.206 | 70.372 | 1.00 | 50.44 |
| HETATM | 2342 | O | HOH | Z | 32 | 45.701 | 11.273 | 61.044 | 1.00 | 32.29 |
| HETATM | 2343 | O | HOH | Z | 33 | 47.764 | 14.051 | 63.278 | 1.00 | 28.70 |
| HETATM | 2344 | O | HOH | Z | 34 | 48.591 | 33.053 | 61.075 | 1.00 | 54.29 |
| HETATM | 2345 | O | HOH | Z | 35 | 59.212 | 38.307 | 51.294 | 1.00 | 36.89 |
| HETATM | 2346 | O | HOH | Z | 36 | 42.763 | 3.390 | 58.521 | 1.00 | 24.82 |
| HETATM | 2347 | O | HOH | Z | 37 | 49.100 | −0.663 | 58.705 | 1.00 | 28.91 |
| HETATM | 2348 | O | HOH | Z | 38 | 47.165 | −7.209 | 56.440 | 1.00 | 51.31 |
| HETATM | 2349 | O | HOH | Z | 39 | 47.633 | 1.624 | 48.231 | 1.00 | 37.22 |
| HETATM | 2350 | O | HOH | Z | 40 | 46.061 | −3.333 | 48.336 | 1.00 | 40.00 |
| HETATM | 2351 | O | HOH | Z | 41 | 43.327 | −0.027 | 48.387 | 1.00 | 50.01 |
| HETATM | 2352 | O | HOH | Z | 42 | 50.571 | −3.228 | 53.736 | 1.00 | 38.96 |
| HETATM | 2353 | O | HOH | Z | 43 | 53.415 | −5.064 | 53.846 | 1.00 | 46.67 |
| HETATM | 2354 | O | HOH | Z | 44 | 50.046 | −0.876 | 50.397 | 1.00 | 49.13 |
| HETATM | 2355 | O | HOH | Z | 45 | 50.046 | 3.190 | 48.165 | 1.00 | 53.01 |
| HETATM | 2356 | O | HOH | Z | 46 | 52.816 | 2.772 | 55.478 | 1.00 | 31.25 |
| HETATM | 2357 | O | HOH | Z | 47 | 52.633 | 2.939 | 48.418 | 1.00 | 39.93 |
| HETATM | 2358 | O | HOH | Z | 48 | 54.727 | 4.247 | 45.978 | 1.00 | 57.14 |
| HETATM | 2359 | O | HOH | Z | 49 | 64.959 | 6.495 | 56.895 | 1.00 | 52.00 |
| HETATM | 2360 | O | HOH | Z | 50 | 57.310 | −1.724 | 58.570 | 1.00 | 41.45 |
| HETATM | 2361 | O | HOH | Z | 51 | 35.848 | 27.712 | 42.926 | 1.00 | 54.37 |
| HETATM | 2362 | O | HOH | Z | 52 | 50.085 | 28.801 | 43.139 | 1.00 | 51.13 |
| HETATM | 2363 | O | HOH | Z | 53 | 60.300 | −2.855 | 54.748 | 1.00 | 41.74 |
| HETATM | 2364 | O | HOH | Z | 54 | 58.529 | −1.199 | 50.605 | 1.00 | 57.32 |
| HETATM | 2365 | O | HOH | Z | 55 | 45.923 | 2.979 | 54.926 | 1.00 | 25.76 |
| HETATM | 2366 | O | HOH | Z | 56 | 44.320 | 2.706 | 48.454 | 1.00 | 38.15 |
| HETATM | 2367 | O | HOH | Z | 57 | 36.785 | 4.672 | 45.000 | 1.00 | 35.08 |
| HETATM | 2368 | O | HOH | Z | 58 | 43.473 | 1.757 | 45.584 | 1.00 | 56.90 |
| HETATM | 2369 | O | HOH | Z | 59 | 41.338 | 8.084 | 41.254 | 1.00 | 53.25 |
| HETATM | 2370 | O | HOH | Z | 60 | 30.433 | 10.290 | 37.115 | 1.00 | 42.75 |
| HETATM | 2371 | O | HOH | Z | 61 | 31.585 | 8.142 | 44.010 | 1.00 | 42.88 |
| HETATM | 2372 | O | HOH | Z | 62 | 34.418 | 5.931 | 43.460 | 1.00 | 45.06 |
| HETATM | 2373 | O | HOH | Z | 63 | 34.334 | 8.999 | 43.667 | 1.00 | 24.43 |
| HETATM | 2374 | O | HOH | Z | 64 | 42.041 | 11.533 | 35.987 | 1.00 | 56.05 |
| HETATM | 2375 | O | HOH | Z | 65 | 41.705 | 15.415 | 39.845 | 1.00 | 40.42 |
| HETATM | 2376 | O | HOH | Z | 66 | 28.818 | 7.428 | 45.833 | 1.00 | 48.27 |
| HETATM | 2377 | O | HOH | Z | 67 | 43.334 | 9.923 | 40.587 | 1.00 | 41.88 |
| HETATM | 2378 | O | HOH | Z | 68 | 45.358 | 15.279 | 44.879 | 1.00 | 20.78 |
| HETATM | 2379 | O | HOH | Z | 69 | 46.213 | 7.669 | 42.907 | 1.00 | 45.16 |
| HETATM | 2380 | O | HOH | Z | 70 | 45.006 | 13.333 | 58.392 | 1.00 | 35.27 |
| HETATM | 2381 | O | HOH | Z | 71 | 53.156 | 12.001 | 58.616 | 1.00 | 21.44 |
| HETATM | 2382 | O | HOH | Z | 72 | 62.274 | 13.778 | 61.807 | 1.00 | 24.83 |
| HETATM | 2383 | O | HOH | Z | 73 | 62.055 | 17.949 | 63.823 | 1.00 | 40.42 |
| HETATM | 2384 | O | HOH | Z | 74 | 59.258 | 11.404 | 67.333 | 1.00 | 33.68 |
| HETATM | 2385 | O | HOH | Z | 75 | 54.744 | 10.535 | 60.395 | 1.00 | 20.48 |
| HETATM | 2386 | O | HOH | Z | 76 | 57.163 | 6.768 | 59.559 | 1.00 | 25.08 |
| HETATM | 2387 | O | HOH | Z | 77 | 63.881 | 8.324 | 47.356 | 1.00 | 49.33 |
| HETATM | 2388 | O | HOH | Z | 78 | 54.853 | 6.749 | 43.141 | 1.00 | 51.35 |
| HETATM | 2389 | O | HOH | Z | 79 | 59.012 | 7.907 | 42.952 | 1.00 | 48.18 |
| HETATM | 2390 | O | HOH | Z | 80 | 56.575 | 6.893 | 49.289 | 1.00 | 32.44 |
| HETATM | 2391 | O | HOH | Z | 81 | 52.021 | 8.983 | 43.755 | 1.00 | 42.49 |
| HETATM | 2392 | O | HOH | Z | 82 | 53.922 | 16.287 | 40.345 | 1.00 | 39.26 |
| HETATM | 2393 | O | HOH | Z | 83 | 59.899 | 11.559 | 42.572 | 1.00 | 49.40 |
| HETATM | 2394 | O | HOH | Z | 84 | 56.645 | 15.814 | 45.124 | 1.00 | 27.98 |
| HETATM | 2395 | O | HOH | Z | 85 | 42.223 | 29.114 | 66.968 | 1.00 | 57.11 |
| HETATM | 2396 | O | HOH | Z | 86 | 51.099 | 26.686 | 63.307 | 1.00 | 33.40 |
| HETATM | 2397 | O | HOH | Z | 87 | 50.936 | 27.431 | 66.552 | 1.00 | 29.14 |
| HETATM | 2398 | O | HOH | Z | 88 | 43.726 | 25.589 | 64.518 | 1.00 | 43.95 |
| HETATM | 2399 | O | HOH | Z | 89 | 43.533 | 20.290 | 68.698 | 1.00 | 42.90 |
| HETATM | 2400 | O | HOH | Z | 90 | 45.850 | 20.417 | 75.681 | 1.00 | 50.53 |
| HETATM | 2401 | O | HOH | Z | 91 | 53.075 | 16.450 | 69.664 | 1.00 | 32.27 |
| HETATM | 2402 | O | HOH | Z | 92 | 45.205 | 18.875 | 73.394 | 1.00 | 48.02 |
| HETATM | 2403 | O | HOH | Z | 93 | 56.319 | 20.734 | 67.582 | 1.00 | 34.40 |
| HETATM | 2404 | O | HOH | Z | 94 | 54.149 | 27.029 | 62.968 | 1.00 | 41.16 |
| HETATM | 2405 | O | HOH | Z | 95 | 59.431 | 20.656 | 66.113 | 1.00 | 30.86 |
| HETATM | 2406 | O | HOH | Z | 96 | 56.743 | 25.775 | 65.812 | 1.00 | 33.38 |
| HETATM | 2407 | O | HOH | Z | 97 | 55.919 | 29.254 | 59.193 | 1.00 | 50.97 |
| HETATM | 2408 | O | HOH | Z | 98 | 61.935 | 26.032 | 62.021 | 1.00 | 59.57 |
| HETATM | 2409 | O | HOH | Z | 99 | 57.636 | 31.734 | 58.490 | 1.00 | 47.61 |
| HETATM | 2410 | O | HOH | Z | 100 | 54.698 | 28.940 | 61.557 | 1.00 | 51.29 |
| HETATM | 2411 | O | HOH | Z | 101 | 63.804 | 37.045 | 61.091 | 1.00 | 55.70 |
| HETATM | 2412 | O | HOH | Z | 102 | 64.563 | 27.770 | 54.244 | 1.00 | 27.13 |
| HETATM | 2413 | O | HOH | Z | 103 | 60.616 | 34.916 | 57.253 | 1.00 | 39.66 |
| HETATM | 2414 | O | HOH | Z | 104 | 60.004 | 37.130 | 53.947 | 1.00 | 48.69 |
| HETATM | 2415 | O | HOH | Z | 105 | 68.398 | 28.837 | 52.842 | 1.00 | 34.34 |
| HETATM | 2416 | O | HOH | Z | 106 | 63.996 | 35.732 | 55.676 | 1.00 | 42.07 |
| HETATM | 2417 | O | HOH | Z | 107 | 63.949 | 27.947 | 59.239 | 1.00 | 50.17 |
| HETATM | 2418 | O | HOH | Z | 108 | 69.301 | 26.294 | 58.726 | 1.00 | 45.15 |
| HETATM | 2419 | O | HOH | Z | 109 | 65.986 | 19.006 | 60.572 | 1.00 | 55.43 |
| HETATM | 2420 | O | HOH | Z | 110 | 67.837 | 21.065 | 59.796 | 1.00 | 48.96 |
| HETATM | 2421 | O | HOH | Z | 111 | 62.227 | 10.934 | 59.636 | 1.00 | 26.45 |
| HETATM | 2422 | O | HOH | Z | 112 | 64.832 | 13.539 | 60.267 | 1.00 | 40.96 |
| HETATM | 2423 | O | HOH | Z | 113 | 65.838 | 22.717 | 49.368 | 1.00 | 34.94 |
| HETATM | 2424 | O | HOH | Z | 114 | 60.813 | 34.849 | 49.705 | 1.00 | 40.82 |
| HETATM | 2425 | O | HOH | Z | 115 | 53.771 | 31.879 | 48.302 | 1.00 | 48.39 |
| HETATM | 2426 | O | HOH | Z | 116 | 56.681 | 36.569 | 53.867 | 1.00 | 47.90 |
| HETATM | 2427 | O | HOH | Z | 117 | 53.642 | 37.108 | 54.653 | 1.00 | 51.95 |
| HETATM | 2428 | O | HOH | Z | 118 | 57.678 | 35.422 | 51.985 | 1.00 | 42.88 |
| HETATM | 2429 | O | HOH | Z | 119 | 48.264 | 38.883 | 52.195 | 1.00 | 53.90 |
| HETATM | 2430 | O | HOH | Z | 120 | 47.164 | 32.550 | 58.275 | 1.00 | 30.05 |
| HETATM | 2431 | O | HOH | Z | 121 | 43.250 | 37.757 | 53.629 | 1.00 | 55.36 |
| HETATM | 2432 | O | HOH | Z | 122 | 41.608 | 33.768 | 54.199 | 1.00 | 49.43 |
| HETATM | 2433 | O | HOH | Z | 123 | 50.840 | 31.733 | 59.258 | 1.00 | 41.85 |
| HETATM | 2434 | O | HOH | Z | 124 | 51.315 | 29.770 | 60.457 | 1.00 | 42.08 |
| HETATM | 2435 | O | HOH | Z | 125 | 60.903 | 24.911 | 43.688 | 1.00 | 46.97 |
| HETATM | 2436 | O | HOH | Z | 126 | 67.041 | 23.302 | 47.013 | 1.00 | 36.30 |
| HETATM | 2437 | O | HOH | Z | 127 | 65.165 | 10.597 | 47.841 | 1.00 | 47.22 |
| HETATM | 2438 | O | HOH | Z | 128 | 65.548 | 19.615 | 38.958 | 1.00 | 50.94 |
| HETATM | 2439 | O | HOH | Z | 129 | 55.720 | 15.392 | 38.656 | 1.00 | 49.13 |
| HETATM | 2440 | O | HOH | Z | 130 | 58.628 | 21.386 | 40.694 | 1.00 | 42.73 |
| HETATM | 2441 | O | HOH | Z | 131 | 43.592 | 25.411 | 61.776 | 1.00 | 33.40 |
| HETATM | 2442 | O | HOH | Z | 132 | 39.165 | 30.389 | 64.577 | 1.00 | 45.28 |
| HETATM | 2443 | O | HOH | Z | 133 | 41.504 | 21.614 | 68.767 | 1.00 | 54.16 |
| HETATM | 2444 | O | HOH | Z | 134 | 40.703 | 28.773 | 68.884 | 1.00 | 46.00 |
| HETATM | 2445 | O | HOH | Z | 135 | 44.797 | 27.868 | 71.294 | 1.00 | 45.84 |
| HETATM | 2446 | O | HOH | Z | 136 | 42.354 | 24.075 | 66.930 | 1.00 | 43.33 |
| HETATM | 2447 | O | HOH | Z | 137 | 37.750 | 27.202 | 69.951 | 1.00 | 46.42 |
| HETATM | 2448 | O | HOH | Z | 138 | 35.848 | 19.568 | 69.764 | 1.00 | 50.41 |
| HETATM | 2449 | O | HOH | Z | 139 | 37.851 | 21.728 | 66.890 | 1.00 | 36.19 |
| HETATM | 2450 | O | HOH | Z | 140 | 30.783 | 23.866 | 66.937 | 1.00 | 50.34 |
| HETATM | 2451 | O | HOH | Z | 141 | 33.726 | 27.401 | 67.001 | 1.00 | 58.33 |
| HETATM | 2452 | O | HOH | Z | 142 | 28.915 | 24.727 | 61.642 | 1.00 | 47.60 |
| HETATM | 2453 | O | HOH | Z | 143 | 33.769 | 31.236 | 57.483 | 1.00 | 46.31 |
| HETATM | 2454 | O | HOH | Z | 144 | 34.891 | 27.864 | 57.438 | 1.00 | 42.43 |
| HETATM | 2455 | O | HOH | Z | 145 | 46.252 | 29.134 | 45.085 | 1.00 | 41.22 |
| HETATM | 2456 | O | HOH | Z | 146 | 33.519 | 21.089 | 41.828 | 1.00 | 28.37 |
| HETATM | 2457 | O | HOH | Z | 147 | 36.513 | 25.357 | 42.533 | 1.00 | 28.71 |
| HETATM | 2458 | O | HOH | Z | 148 | 44.082 | 21.238 | 41.779 | 1.00 | 24.55 |
| HETATM | 2459 | O | HOH | Z | 149 | 44.511 | 30.853 | 43.295 | 1.00 | 52.96 |
| HETATM | 2460 | O | HOH | Z | 150 | 37.321 | 31.395 | 45.097 | 1.00 | 57.97 |
| HETATM | 2461 | O | HOH | Z | 151 | 50.444 | 27.054 | 41.455 | 1.00 | 46.22 |
| HETATM | 2462 | O | HOH | Z | 152 | 50.470 | 23.304 | 40.354 | 1.00 | 46.12 |
| HETATM | 2463 | O | HOH | Z | 153 | 49.473 | 17.014 | 44.938 | 1.00 | 22.23 |
| HETATM | 2464 | O | HOH | Z | 154 | 44.072 | 13.687 | 42.388 | 1.00 | 33.61 |
| HETATM | 2465 | O | HOH | Z | 155 | 46.743 | 15.157 | 42.567 | 1.00 | 28.06 |
| HETATM | 2466 | O | HOH | Z | 156 | 43.696 | 21.518 | 37.147 | 1.00 | 30.08 |
| HETATM | 2467 | O | HOH | Z | 157 | 44.029 | 19.607 | 35.637 | 1.00 | 33.31 |
| HETATM | 2468 | O | HOH | Z | 158 | 49.032 | 22.171 | 37.565 | 1.00 | 50.29 |
| HETATM | 2469 | O | HOH | Z | 159 | 46.521 | 12.392 | 42.140 | 1.00 | 47.31 |
| HETATM | 2470 | O | HOH | Z | 160 | 52.916 | 22.239 | 37.026 | 1.00 | 55.49 |
| HETATM | 2471 | O | HOH | Z | 161 | 45.869 | 13.974 | 60.909 | 1.00 | 34.41 |
| HETATM | 2472 | O | HOH | Z | 162 | 48.083 | 16.068 | 66.708 | 1.00 | 33.89 |
| HETATM | 2473 | O | HOH | Z | 163 | 43.023 | 14.129 | 61.953 | 1.00 | 53.88 |
| HETATM | 2474 | O | HOH | Z | 164 | 38.756 | 19.276 | 65.202 | 1.00 | 36.58 |
| HETATM | 2475 | O | HOH | Z | 165 | 39.907 | 13.260 | 59.270 | 1.00 | 40.54 |
| HETATM | 2476 | O | HOH | Z | 166 | 36.800 | 15.818 | 59.356 | 1.00 | 38.21 |
| HETATM | 2477 | O | HOH | Z | 167 | 30.756 | 21.819 | 41.946 | 1.00 | 52.32 |
| HETATM | 2478 | O | HOH | Z | 168 | 26.321 | 18.194 | 36.951 | 1.00 | 49.33 |
| HETATM | 2479 | O | HOH | Z | 169 | 28.298 | 20.489 | 35.444 | 1.00 | 47.56 |
| HETATM | 2480 | O | HOH | Z | 170 | 34.060 | 21.200 | 39.322 | 1.00 | 37.57 |
| HETATM | 2481 | O | HOH | Z | 171 | 31.024 | 17.705 | 33.835 | 1.00 | 31.46 |
| HETATM | 2482 | O | HOH | Z | 172 | 35.364 | 14.356 | 33.358 | 1.00 | 50.31 |
| HETATM | 2483 | O | HOH | Z | 173 | 37.090 | 14.498 | 37.690 | 1.00 | 47.58 |
| HETATM | 2484 | O | HOH | Z | 174 | 29.883 | 11.426 | 33.922 | 1.00 | 37.75 |
| HETATM | 2485 | O | HOH | Z | 175 | 30.389 | 15.363 | 32.808 | 1.00 | 37.18 |
| HETATM | 2486 | O | HOH | Z | 176 | 25.224 | 10.459 | 26.647 | 1.00 | 58.79 |
| HETATM | 2487 | O | HOH | Z | 177 | 26.104 | 10.611 | 35.365 | 1.00 | 35.22 |
| HETATM | 2488 | O | HOH | Z | 178 | 19.926 | 9.452 | 39.387 | 1.00 | 55.25 |
| HETATM | 2489 | O | HOH | Z | 179 | 19.723 | 16.723 | 39.659 | 1.00 | 52.43 |
| HETATM | 2490 | O | HOH | Z | 180 | 19.071 | 11.323 | 42.461 | 1.00 | 51.41 |
| HETATM | 2491 | O | HOH | Z | 181 | 24.087 | 10.832 | 37.321 | 1.00 | 35.20 |
| HETATM | 2492 | O | HOH | Z | 182 | 23.535 | 16.129 | 45.715 | 1.00 | 43.59 |
| HETATM | 2493 | O | HOH | Z | 183 | 21.411 | 12.400 | 47.935 | 1.00 | 51.82 |
| HETATM | 2494 | O | HOH | Z | 184 | 26.338 | 9.343 | 45.940 | 1.00 | 38.29 |
| HETATM | 2495 | O | HOH | Z | 185 | 28.937 | 4.006 | 49.860 | 1.00 | 46.67 |
| HETATM | 2496 | O | HOH | Z | 186 | 35.592 | 1.182 | 45.532 | 1.00 | 43.21 |
| HETATM | 2497 | O | HOH | Z | 187 | 42.181 | 12.293 | 59.682 | 1.00 | 44.13 |
| HETATM | 2498 | O | HOH | Z | 188 | 40.677 | 11.462 | 62.985 | 1.00 | 35.66 |
| HETATM | 2499 | O | HOH | Z | 189 | 34.830 | 8.329 | 59.655 | 1.00 | 45.13 |
| HETATM | 2500 | O | HOH | Z | 190 | 39.290 | 9.584 | 68.605 | 1.00 | 52.23 |
| HETATM | 2501 | O | HOH | Z | 191 | 29.697 | 13.317 | 68.291 | 1.00 | 52.85 |
| HETATM | 2502 | O | HOH | Z | 192 | 24.761 | 18.714 | 66.413 | 1.00 | 59.04 |
| HETATM | 2503 | O | HOH | Z | 193 | 29.424 | 16.578 | 68.004 | 1.00 | 52.70 |
| HETATM | 2504 | O | HOH | Z | 194 | 28.068 | 22.230 | 62.043 | 1.00 | 49.54 |
| HETATM | 2505 | O | HOH | Z | 195 | 26.825 | 29.550 | 56.806 | 1.00 | 54.77 |
| END | | | | | | | | | | |

TABLE 2
All atoms as in Table 1 (incorporated herein by reference) except for the following
six changes:

1) Delete lines for Atom 1770-1793
2) Replace with:

| | | | | | | | | | | |
|---|---|---|---|---|---|---|---|---|---|---|
| ATOM | 1770 | N | CYS | A | 215 | 45.982 | 16.650 | 59.851 | 1.00 | 21.29 |
| ATOM | 1771 | CA | CYS | A | 215 | 45.553 | 17.584 | 60.885 | 1.00 | 21.43 |
| ATOM | 1772 | C | CYS | A | 215 | 46.211 | 17.303 | 62.215 | 1.00 | 24.05 |
| ATOM | 1773 | O | CYS | A | 215 | 47.355 | 16.913 | 62.307 | 1.00 | 22.32 |
| ATOM | 1774 | CB | CYS | A | 215 | 44.037 | 17.520 | 61.166 | 1.00 | 22.73 |
| ATOM | 1775 | SG | CYS | A | 215 | 43.916 | 18.029 | 62.855 | 1.00 | 30.96 |
| ATOM | 1776 | N | SER | A | 216 | 45.432 | 17.553 | 63.271 | 1.00 | 28.96 |
| ATOM | 1777 | CA | SER | A | 216 | 45.887 | 17.111 | 64.593 | 1.00 | 33.95 |
| ATOM | 1778 | C | SER | A | 216 | 45.405 | 15.709 | 64.892 | 1.00 | 38.49 |
| ATOM | 1779 | O | SER | A | 216 | 44.329 | 15.318 | 64.490 | 1.00 | 38.68 |
| ATOM | 1780 | CB | SER | A | 216 | 45.451 | 18.035 | 65.735 | 1.00 | 34.57 |
| ATOM | 1781 | OG | SER | A | 216 | 45.116 | 19.351 | 65.362 | 1.00 | 36.62 |

3) Renumber following atoms (from ALA 217) accordingly.
4) Insert following line after new ATOM 2297 (old ATOM 2309):

| | | | | |
|---|---|---|---|---|
| TER | 2298 | GLY | A | 283 |

5) Insert following Header to PDB file:

| | | | | | | | | | | |
|---|---|---|---|---|---|---|---|---|---|---|
| LINK | MG | MG | A | 1282 | O | HOH | Z | 106 | 1555 | 4546 |
| LINK | MG | MG | A | 1282 | O | HOH | Z | 10 | 1555 | 1555 |
| LINK | MG | MG | A | 1282 | O | HOH | Z | 40 | 1555 | 1555 |
| LINK | SG | CYS | A | 215 | N | SER | A | 216 | 1555 | 1555 |

6) Insert following Footer instead of END line:

| | | | | |
|---|---|---|---|---|
| CONECT | 1775 | 1776 | | |
| CONECT | 1776 | 1775 | | |
| CONECT | 2299 | 2405 | 2309 | 2339 |
| CONECT | 2309 | 2299 | | |
| CONECT | 2339 | 2299 | | |
| CONECT | 2405 | 2299 | | |

| | | | | | | | | | | | |
|---|---|---|---|---|---|---|---|---|---|---|---|
| MASTER | 498 | 0 | 1 | 9 | 11 | 0 | 1 | 6 | 2493 | 1 | 6 |
| 25 | | | | | | | | | | | |
| END | | | | | | | | | | | |

1. Isolated sulfenyl amide cysteine-containing protein, or a homologue, allelic form, species variant, derivative or mutein thereof.

2-94. (canceled)
